SEQUENCE LISTING (30 sequences)

SEQ ID NO: 1 - DNA, 34 bp, Artificial Sequence
  synthetic construct; primer
gcgcgcgcgctccggaatggaggagccgcagtca

SEQ ID NO: 2 - DNA, 40 bp, Artificial Sequence
  synthetic construct; primer
gcgcgcgcgcggtacctcatggtttcttctttggctgggg

SEQ ID NO: 3 - DNA, 37 bp, Artificial Sequence
  synthetic construct; primer
ccgggcccgcggtccggaacctaccagggcagctacg

SEQ ID NO: 4 - DNA, 41 bp, Artificial Sequence
  synthetic construct; primer
ccgggcccgcggggtacctttcttgcggagattctcttcct

SEQ ID NO: 5 - DNA, 36 bp, Artificial Sequence
  synthetic construct; primer
gcgcgcgcgcggtaccgctcccagaatgccagaggc

SEQ ID NO: 6 - DNA, 36 bp, Artificial Sequence
  synthetic construct; primer
gcgcgcgcgcggatcctttcttgcggagattctctt

SEQ ID NO: 7 - DNA, 45 bp, Artificial Sequence
  synthetic construct; primer
gcgcgcgcgcgggatccggctggatggagaatatttcacccttca

SEQ ID NO: 8 - DNA, 38 bp, Artificial Sequence
  synthetic construct; primer
gcgcgcgcgcgggatcctcacccagcctgggcatcctt

SEQ ID NO: 9 - DNA, 35 bp, Artificial Sequence
  synthetic construct; primer
tcccttcccagaaaaggtaccagggcagctacggt

SEQ ID NO: 10 - DNA, 56 bp, Artificial Sequence
  synthetic construct; primer
tcgagctatggaaacattttcagacctatggaaactacttcctgaacggaattctg

SEQ ID NO: 11 - DNA, 44 bp, Artificial Sequence
  synthetic construct; primer
ttcactgaagacccaggtccatccggagctcccagaatgccaga

SEQ ID NO: 12 - DNA, 35 bp, Artificial Sequence
  synthetic construct; primer
tctcgagctcaagcttcgaattcattggacaatgg

SEQ ID NO: 13 - protein, 30 aa, Artificial Sequence
  synthetic construct; mitochondrial targeting signal
RKGQERFNRWFLTGMTVAGVVLLGSLFSRK

SEQ ID NO: 14 - protein, 27 aa, Artificial Sequence
  synthetic construct; mitochondrial targeting signal
GTPTWQTVTIFVAGVLTASLTIWKKMG

SEQ ID NO: 15 - protein, 28 aa, Artificial Sequence
  synthetic construct; mitochondrial targeting signal
GNGPILNVLVVLGVVLLGQFVVRRFFKS

SEQ ID NO: 16 - DNA, 37 bp, Artificial Sequence; identical to SEQ ID NO: 3
  synthetic construct; primer

SEQ ID NO: 17 - DNA, 41 bp, Artificial Sequence; identical to SEQ ID NO: 4
  synthetic construct; primer

SEQ ID NO: 18 - DNA, 32 bp, Artificial Sequence
  synthetic construct; primer
cattctgggacagccgcgtctgtgacttgcac

SEQ ID NO: 19 - DNA, 39 bp, Artificial Sequence
  synthetic construct; primer
cagttcctgcatgggcggcatgaacgcgaggcccatcct

SEQ ID NO: 20 - DNA, 32 bp, Artificial Sequence
  synthetic construct; primer
tttgtgcctgtcctggggcagaccggcgcaca

SEQ ID NO: 21 - DNA, 29 bp, Artificial Sequence
  synthetic construct; primer
accggcgcacagcggaagcgaatctccgc

SEQ ID NO: 22 - DNA, 36 bp, Artificial Sequence
  synthetic construct; primer
gcattctgggacagccgagtctgtgacttgcacgta

SEQ ID NO: 23 - DNA, 99 bp, Artificial Sequence
  synthetic construct; oligo encoding mitochondrial targeting signal
gatcctcctactttgggacgcccacgtggcagaccgtgaccatctttgtggcgggagtgctcaccgcctcactcaccatctggaagaagatgggctgag

SEQ ID NO: 24 - protein, 393 aa, Artificial Sequence [sequence omitted]

SEQ ID NO: 25 - protein, 201 aa, Artificial Sequence
  synthetic construct; DNA binding domain of p53
SSSVPSQKTYQGSYGFRLGFLHSGTAKSVTCTYSPALNKMFCQLAKTCPVQLWVDSTPPPGTRVRAMAIYKQSQHMTEVVRRCPHHERCSDSDGLAPPQHLIRVEGNLRVEYLDDRNTFRHSVVVPYEPPEVGSDCTTIHYNYMCNSSCMGGMNRRPILTIITLEDSSGNLLGRNSFEVRVCACPGRDRRTEEENLRKKGE

SEQ ID NO: 26 - DNA, 93 bp, Artificial Sequence
  synthetic construct; oligo capable of encoding a mitochondrial targeting signal
gatccggcaatggtcccatcctgaacgtgctggtggttctgggtgtggttctgttgggccagtttgtggtacgaagattcttcaaatcatgag

SEQ ID NO: 27 - DNA, 93 bp, Artificial Sequence
  synthetic construct; oligo capable of encoding a mitochondrial targeting signal
agaaagggccaggagagattcaacagatggttcctgaccggcatgaccgtggccggcgtggtgctgctgggcagcctgttcagcagaaagtga

SEQ ID NO: 28 - DNA, 40 bp, Artificial Sequence
  synthetic construct; primer
gggacggaacagctttgaggtggctgtttgtgcctgtcct

SEQ ID NO: 29 - DNA, 1182 bp, Artificial Sequence
  synthetic construct; nucleic acid sequence of p53
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccccgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 30 - DNA, 570 bp, Artificial Sequence
  synthetic construct; oligo capable of encoding DNA binding domain of p53
acctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaag

(19) United States
(12) Patent Application Publication
(10) Pub. No.: US 2016/0347807 A1
(43) Pub. Date: Dec. 1, 2016

(54) TARGETING P53 AND ITS DNA-BINDING DOMAIN

(71) Applicant: UNIVERSITY OF UTAH RESEARCH DOUNDATION, Salt Lake City, UT (US)

(72) Inventors: Carol Lim, Salt Lake City, UT (US); Karina Matissek, Cambridge, MA (US); Mohanad A. Mossalam, Salt Lake City, UT (US)

(21) Appl. No.: 15/117,047

(22) PCT Filed: Feb. 6, 2015
(86) PCT No.: PCT/US15/14851

§ 371 Date: Aug. 5, 2016

Related U.S. Application Data
(60) Provisional application No. 61/936,790, filed on Feb. 6, 2014.
(60) Provisional application No. 61/944,384, filed on Feb. 25, 2014.

(51) Int. Cl. C07K 14/47; C07K 14/82

(52) U.S. Cl. CPC C07K 14/4746; C07K 14/82; A61K 38/00

(57) ABSTRACT

Disclosed are peptides comprising a full length p53 or a partial p53 and a mitochondrial targeting signal. Also disclosed are nucleic acids that encode peptides comprising a full length p53 or a partial p53 and a mitochondrial targeting signal. Further disclosed are methods of using the peptides and nucleic acids disclosed herein. For example, the peptides and nucleic acids can be used to treat hyperproliferative disorders.

### CROSS-REFERENCE TO RELATED APPLICATION

This application claims benefit of and priority under 35 U.S.C. §371 of PCT/US2015/014851, filed Feb. 6, 2015, which claims the benefit under 35 U.S.C. §119(e) of U.S. Provisional Application No. 61/936,790, filed Feb. 6, 2014, and of U.S. Provisional Application No. 61/944,384, filed Feb. 25, 2014, which are incorporated herein by reference in their entirety.

### STATEMENT REGARDING FEDERALLY SPONSORED RESEARCH

This invention was made with government support under Grant Number CA151847 awarded by the National Institutes of Health. The government has certain rights in the invention.

### REFERENCE TO THE SEQUENCE LISTING

The Sequence Listing submitted Aug. 5, 2016 as a text file named “21101_0297U3_Sequence_Listing.txt,” created on Aug. 4, 2016, and having a size of 14,530 bytes is hereby incorporated by reference pursuant to 37 C.F.R. §1.52(e)(5).

### BACKGROUND

p53 is a transcription factor that stimulates a network of signals through two apoptotic signaling pathways: the extrinsic pathway (nuclear transcriptional activation) through death receptors and the intrinsic pathway through the mitochondria. While much work using p53 has exploited the extrinsic pathway, the intrinsic pathway is more appealing, due to its rapid, direct apoptotic effects at the mitochondria and absence of inactivation by the dominant negative effect (dimerization and inactivation by mutant wt p53 in cancer cells).

The compositions and methods described herein provide a solution to the effective and targeted delivery of p53 to the mitochondria of cells.

### BRIEF SUMMARY

Disclosed are peptides comprising a full length p53 peptide or a partial p53 peptide and a mitochondrial targeting signal (MTS).

Disclosed are peptides comprising a full length p53 peptide and a MTS, wherein the MTS is a Bak or Bax MTS. A Bak MTS can comprise the amino acid sequence comprising GNGPILNVLVVLGVVLLGQFVVRRFFKS (SEQ ID NO:15). A Bax MTS can comprise the amino acid sequence comprising GTPTWQTVTIFVAGVLTASLTIWKKMG (SEQ ID NO:14).

Disclosed are peptides comprising a partial p53 peptide and a MTS. The MTS can comprise a Bcl-XL, Bak, or Bax MTS. In some aspects the MTS can comprise the amino acid sequence of RKGQERFNRWFLTGMTVAGVVLLGSLFSRK (SEQ ID NO:13), GNGPILNVLVVLGVVLLGQFVVRRFFKS (SEQ ID NO:15), or GTPTWQTVTIFVAGVLTASLTIWKKMG (SEQ ID NO:14).

Disclosed are peptides comprising a partial p53 peptide and a MTS, wherein the partial p53 peptide consists of the DNA binding domain of p53. In some aspects, the partial p53 peptide consists of amino acids 102-292 of SEQ ID NO:24. In some aspects, the partial p53 peptide comprises the DNA binding domain of p53.

Disclosed are peptides comprising a partial p53 peptide and a MTS, wherein the DNA binding domain of p53 consists of amino acids 102-292 of SEQ ID NO:24.

Disclosed are peptides comprising a partial p53 peptide and a MTS, wherein the partial p53 peptide further comprises a MDM2 binding domain, a proline-rich domain, a tetramerization domain, or a transactivation domain of p53.

Disclosed are nucleic acid sequences, wherein the nucleic acid sequences are capable of encoding a full length p53 peptide and a MTS, wherein the MTS comprises a Bak or Bax MTS. In some aspects, the nucleic acid sequence is capable of encoding a partial p53 peptide and a MTS. In some aspects, the partial p53 peptide comprises the DNA binding domain of p53. In some aspects, the MTS comprises a Bcl-XL, Bak, or Bax MTS. In some aspects, the nucleic acid sequence capable of encoding the MTS comprises the sequence of SEQ ID NO:13, SEQ ID NO:14, or SEQ ID NO:15.

Disclosed are vectors comprising a nucleic acid sequence, wherein the nucleic acid sequence is capable of encoding a full length p53 peptide and a MTS, wherein the MTS comprises a Bak or Bax MTS.

Also disclosed are vectors comprising a nucleic acid sequence, wherein the nucleic acid sequence is capable of encoding a partial p53 peptide and a MTS. In some aspects, the partial p53 peptide comprises the DNA binding domain of p53. The MTS can comprise a Bcl-XL, Bak, or Bax MTS.

The disclosed vectors can be a viral vectors. In some aspects, the viral vector can be an adenoviral vector.

Disclosed are methods of inducing apoptosis comprising administering a peptide comprising a full length p53 peptide and a MTS, wherein the MTS is a Bak or Bax MTS. Also disclosed are methods of inducing apoptosis comprising administering a peptide comprising a partial p53 peptide and a MTS. The peptide can induce apoptosis through the Bak or Bax pathway. The MTS can be a Bcl-XL, Bak, or Bax MTS.

Disclosed are methods of targeting the disclosed peptides to mitochondria comprising introducing a peptide to a cell, wherein the peptide comprises a full length p53 peptide and a MTS, wherein the MTS comprises a Bak or Bax MTS.

Disclosed are methods of targeting the disclosed peptides to mitochondria comprising introducing a peptide to a cell, wherein the peptide comprises a partial p53 peptide and a MTS. The MTS can comprise a Bcl-XL, Bak, or Bax MTS.

Disclosed are methods of inducing homo-oligomerization of Bak or Bax comprising administering a peptide comprising the DNA binding domain of p53 and a MTS, wherein the MTS is a Bak or Bax MTS. The peptide can be a partial p53 peptide or a full length p53 peptide.

Disclosed are methods of treating a hyperproliferative disorder in a patient comprising administering to the patient a peptide, wherein the peptide comprises a full length p53 peptide and a MTS, wherein the MTS comprises a Bak or Bax MTS.

Disclosed are methods of treating a hyperproliferative disorder in a patient comprising administering to the patient a peptide, wherein the peptide comprises a partial p53 peptide and a MTS. The MTS can comprise a Bcl-XL, Bak, or Bax MTS. The partial p53 peptide can comprise the DNA binding domain of p53.

Disclosed are methods of treating a hyperproliferative disorder in a patient comprising administering to the patient a peptide, wherein the peptide comprises a partial p53 peptide and a MTS, wherein the hyperproliferative disorder is cancer. The cancer can be breast cancer or ovarian cancer.

Disclosed are methods of treating a hyperproliferative disorder in a patient comprising administering to the patient a peptide, wherein the peptide comprises a partial p53 peptide and a MTS, wherein the hyperproliferative disorder is cancer, further comprising co-administering an anti-cancer agent. The anti-cancer agent can be paclitaxel or carboplatin.

Disclosed are methods of treating a hyperproliferative disorder in a patient comprising administering to the patient a nucleic acid sequence, wherein the nucleic acid sequence is capable of encoding a full length p53 peptide and a MTS, wherein the MTS comprises a Bak or Bax MTS.

Also disclosed are methods of treating a hyperproliferative disorder in a patient comprising administering to the patient a nucleic acid sequence, wherein the nucleic acid sequence is capable of encoding a partial p53 peptide and a MTS. The MTS can comprise a Bcl-XL, Bak or Bax MTS.

Disclosed are methods of treating a hyperproliferative disorder in a patient, wherein the nucleic acid is administered to the patient using a viral vector. The viral vector can be an adenoviral vector.

Disclosed are recombinant cells comprising the nucleic acids or the vectors described herein. Also disclosed are recombinant cells comprising a nucleic acid capable of producing any of the peptides described herein.

Disclosed are transgenic, non-human subjects comprising the nucleic acids or the vectors described herein, wherein the nucleic acids are capable of encoding the peptides described herein.

Disclosed are monoclonal antibodies that specifically bind to the peptides described herein.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIGS. 1A and 1B are schematic diagrams of different constructs. A: Schematic representation of wild type p53 (wt p53). The 393 amino acids of p53 are divided into amino terminus, DNA binding domain (DBD), and C-terminal region. The MDM2 binding domain (MBD) and proline-rich domain (PRD) are located in the amino terminus. The tetramerization (TD) domain and the nuclear localization signals (NLSs) are located in the C-terminus. B: Schematic representation of the main experimental constructs and controls including the rational for design. p53-XL shows the structure of full length p53 with the enhanced green fluorescence protein EGFP on the amino terminus and the MTS from Bcl-XL (XL) on the C-terminus. All the other constructs contain various combinations of the different domains of p53, in addition to EGFP and XL. The negative control (E-XL) consists of only EGFP and XL.

FIG. 2 is a bar graph showing the colocalization of EGFP constructs and MitoTracker Red mitochondrial stain in 1471.1 cells. The degree of colocalization is represented by PCC following Costes' approach. All constructs with values higher than 0.6 are considered highly colocalized with mitochondrial stain MitoTracker Red. Statistical analysis was performed by using odds ratio with Pearson's Chi-square. The adjusted odds ratio for PCC value of 0.6 was compared with each sample. *p<0.05, and **p<0.01 comparing odds ratio of lowest value for samples with odds ratio of 1 for PCC of 0.6.

FIG. 3 is a bar graph showing the percent of 7-AAD positive cells. The 7-AAD assay was analyzed in T47D cells 48 h after transfection. Statistical analysis were conducted by one-way ANOVA with Tukey's post test. ***p<0.001. PRD-DBD-XL, DBD-XL, p53ΔC-XL and p53-XL were not statistically significant from each other. MBD-XL, MBD-PRD-XL, PRD-XL, and TD-XL are statistically significantly lower than p53-XL.

FIGS. 4A and 4B are bar graphs of Annexin V positive cells and TUNEL positive cells, respectively. (A) Apoptotic potential was tested in T47D cells 48 h after transfection via annexin V assay. Statistical analysis was performed using one-way ANOVA with Tukey's post test with *p<0.05. (B) Apoptotic potential was tested in T47D cells 48 h after transfection via TUNEL-assay. Statistical analysis was performed using one-way ANOVA with Tukey's post test with *p<0.05, ***p<0.001. p53-XL and DBD-XL were not statistically significant from each other. MBD-XL, PRD-XL, and TD-XL are statistically significantly lower than p53-XL.

FIG. 5 is a bar graph showing relative fluorescence of different constructs. Transformative ability of T47D cells was determined 8 d after transfection of T47D cells via colony forming assay. Statistical analysis was accompanied using one-way ANOVA with Tukey's post test ***p<0.001.

FIGS. 6A, 6B, 6C and 6D show the bar graphs from the 7-AAD assays that were conducted in (A) MCF-7, (B) MDA-MB-231, (C) HeLa and (D) H1373. Statistical analysis was performed using one-way ANOVA with Tukey's post test **p<0.01 and ***p<0.001.

FIGS. 7A and 7B show bar graphs of % MOMP and % Caspase 9, respectively. (A) Mitochondrial depolarization correlates with an increase in MOMP (as measured by TMRE). T47D cells were transfected with mitochondrial constructs and assayed using TMRE 36 h post transfections. (B) The activation of caspase-9 was analyzed 48 h following transfection of T47D cells. Statistical analysis was performed by using one-way ANOVA with Bonferroni's post test **p<0.01 and ***p<0.001.

FIGS. 8A and 8B(A) Representative cropped western blot of protein complexes co-immunoprecipitated using anti-GFP antibody. Lane 1, exogenous p53-XL (75 kDa) which was transfected into T47D cells co-immunoprecipitates with endogenous Bcl-XL (26 kDa). Lane 2, exogenous E-XL (32 kDa) co-immunoprecipitates with exogenous Bcl-XL (26 kDa). Lane 3 exogenous E-CC (35 kDa) fails to co-immunoprecipitate with endogenous Bcl-XL. Unlabeled bands are nonspecific binding. (B) Rescue experiment using Bcl-XL. 7-AAD assay was conducted 48 h post transfection in T47D cells. Statistical analysis was tested via unpaired t-test **p<0.01 and ***p<0.001.

FIG. 9 shows different endogenous expression levels of Bcl-XL.

FIG. 10 shows a rescue experiment including MBD-XL, PRD-XL, DBD-XL, TD-XL, p53-XL, and E-XL.

FIG. 11 shows the Intrinsic mitochondrial pathway (a) Upon apoptotic stimuli, Bak homo-oligomerization allows pore formation and cytochrome c release. (b) Mcl-1 and Bcl-XL sequester Bak and do not allow homo-oligomerization (c) p53-BakMTS binds Bak, releases Bak from both Mcl-1 and Bcl-XL, and allows homo-oligomerization and cytochrome c release which results in apoptosis. On the other hand, Bax is sequestered by Bcl-2 and Bcl-w. p53-BaxMTS binds to Bax, releases Bax from Bcl-2 and Bcl-w and causes apoptosis (pathway for Bax not shown).

FIG. 12 shows a schematic representation of experimental constructs: Wild-type p53 is divided into N-terminus, DNA binding domain (DBD) and C-terminal region. The N-terminus consists of a transactivation domain (TA), nuclear export signal (E), MDM2 binding domain (M) and proline-rich domain (PRD). The C-terminus contains three nuclear localization signals (NLS), nuclear export signal (E) and tetramerization domain (TD). p53-MTS wild-type p53 was fused to the mitochondrial targeting signal (MTS) from Bak or Bax. DBD-MTS DNA-binding domain of p53 was fused to the MTS from Bak or Bax.

FIG. 13 shows the structure of the Bak and Bax mitochondrial targeting signals (MTSs) (a) The Bax protein is mainly found in the cytoplasm of healthy cells. Upon apoptotic stimuli, it translocates to the mitochondrial outer membrane. The MTS of the Bax protein, consisting of the transmembrane domain (TM) and the C-segment (CS), becomes exposed for integration into the mitochondrial outer membrane. (b) Unlike Bax, Bak is always present at the mitochondrial outer membrane via its TM and CS.

FIG. 14 shows mitochondrial localization. PCC values were graphed for each construct. PCC value equal to 0.6 and above is considered to be colocalized. Statistical analysis was performed using odds ratio with Pearson's Chi-square. The adjusted odds ratio for PCC value of 0.6 was compared with each sample (**p<0.01).

FIG. 15 shows the results from a 7-AAD assay in T47D cells, 48 h after transfection. Statistical analysis was performed using one-way ANOVA with Bonferroni's post test; **p<0.01, ***p<0.001. Error bars represent standard deviations from at least three independent experiments (n=3).

FIGS. 16A, 16B, and 16C show Nuclear transcriptional activity, Mitochondrial apoptosis: TMRE assay, Mitochondrial apoptosis: Caspase-9, respectively. (a) Nuclear transcriptional activity: p53 reporter gene assay: all MTS constructs were tested for their ability to activate p53-Luc Cis-Reporter in T47D cells. Wt p53 was used as a positive control and EGFP was considered a negative control. (b) Mitochondrial apoptosis: TMRE assay: all constructs were assayed in T47D cells. Mitochondrial depolarization correlates with an increase in MOMP (measured as loss of TMRE fluorescence). (c) Mitochondrial apoptosis: Caspase-9 activation was analyzed in T47D cells. All statistical analysis for a, b, and c were performed by using one-way ANOVA with Bonferroni's post test; *p<0.05, **p<0.01, ***p<0.001. Error bars represent standard deviations from at least three independent experiments (n=3). For b and c negative controls E-BakMTS and E-BaxMTS were compared to EGFP using one-way ANOVA with Bonferroni's post test; #p<0.05, ##p<0.01, ###p<0.001. p53-BakMTS and p53-BaxMTS were not significantly higher from wt p53.

FIGS. 17A and 17B shows the results of 7-AAD assays in T47D cells: Apoptotic potential of DBD-BakMTS and DBD-BaxMTS were tested. A) BakMTS. B) BaxMTS. Statistical analysis was performed by using one-way ANOVA with Bonferroni's post test; **p<0.01, ***p<0.001 compared to negative controls. Error bars represent standard deviations from at least three independent experiments (n=3).

FIGS. 18A, 18B, 18C, 18D, 18E, 18F show the results 7-AAD assay was conducted in (a) and (b) H1373, (c) and (d) SKOV-3, (e) and (0 HeLa cells. Statistical analysis was performed by using one-way ANOVA with Bonferroni's post test;*p<0.05, **p<0.01, ***p<0.001. Error bars represent standard deviations from at least three independent experiments (n=3).

FIGS. 19A and 19B show a decrease in apoptotic potential caused by (a) K120A, R248A, R273A, R280A, E285A, E287A (m6) mutations and (b) K120E mutation was measured via 7-AAD assay in T47D cells. Statistical analysis was performed by using one-way ANOVA with Bonferroni's post test; **p<0.01, ***p<0.001 compared to E-BakMTS. Error bars represent standard deviations from at least three independent experiments (n=3).

FIG. 20 shows a diagram of the cell death pathway. If wt p53 is defective (as in high grade serous carcinoma), intrinsic and extrinsic apoptosis cannot be triggered, and no synergistic effect is seen with carboplatin and paclitaxel. DBD-MTS alone can robustly trigger intrinsic apoptosis directly at the mitochondria and does not bind to defective/mutated wt p53 in cancer cells. DBD-MTS alone, and synergism with paclitaxel and/or carboplatin will be tested in this proposal.

FIG. 21 shows a schematic diagram of the mitochondrial apoptosis pathway.

FIG. 22 shows a schematic diagram of p53-MTS (p53 with mitochondrial targeting signal attached) and DBD-MTS (DNA binding domain from p53 attached to MTS). Constructs were subcloned with and without EGFP.

FIG. 23 shows the results from a 7-AAD assay (late apoptosis). p53-BakMTS and p53-BaxMTS induce apoptosis similarly to wt p53.

FIG. 24 shows p53 reporter activity (in T47D cells).

FIGS. 25A, 25B and 25C show 7-AAD assays (late stage apoptosis) from (A) HeLa, (B) H1373, and (C) T47D cells. DBD-BakMTS (white stars) induces apoptosis in all 3 cell lines.

FIG. 26 shows that mutations in p53 (“m6”) that abolish interaction with Bak also abolish apoptosis.

FIG. 27 shows that DBD-BakMTS (star) induces late stage apoptosis in SKOV3 cells.

FIG. 28 shows a schematic diagram of the proteasomeal degradation pathway of wt p53. MDM2 monoubiquitinates p53 in the nucleus; cytoplasmic monoubiquitinated p53 can either get polyubiquitinated via MDM2 and E4 factors, E-like molecules, via other E-ligases, or via MDM2/MDMX heterodimers. Polyubiquitinated p53 is targeted to the proteasome and degraded into peptides.

FIG. 29 is a schematic diagram of the transcriptional activity of wt p53. wt p53 translocates to the nucleus, forms a tetramer, binds to DNA and activates gene transactivation. Extrinsic pathway is facilitated via the death receptors Fas, Dr5 and PERP which trigger caspase-8 induction resulting in apoptosis. The Intrinsic pathway is initiated via Puma, Noxa and Bax resulting in MOMP, cytochrome c release, caspase-9, and eventually triggers apoptosis.

FIG. 30 is a schematic diagram showing mitochondrial p53 directly activates the intrinsic apoptotic pathway through a sequential mechanism. First mitochondrial p53 interacts with anti-(Bcl-XL) and then binds to pro-(Bak; Bax) Bcl-2 proteins. Bak or Bax form homo-oligomers causing MOMP and cytochrome c release, activation of caspase 9 and eventually apoptosis.

FIG. 31 shows a schematic of the frequency of TP53 mutations with the most frequent mutations outlined in the DBD. Line length indicates the number of mutations.

FIGS. 32A and 32B are schematics of the mitochondrial apoptotic pathway of p53. When p53 is targeted to the mitochondria, it interacts with anti-apoptotic Bcl-XL, enables Bax and Bak oligomerization, and activates the intrinsic apoptotic pathway. The apoptosome (cytochrome c, APAF-1 and caspase-9) is triggered, leading to apoptosis via activation of caspases-3, -6, and -7. The left side of the diagram indicates the mitochondrial signals (MTSs) used and the different subsections of the mitochondria targeted, including the outer membrane (“XL-MTS” from Bcl-XL; “TOM-MTS” from TOM20), the inner membrane (“CCO-MTS” from cytochrome c oxidase), and the matrix (“OTC-MTS” from ornithine transcarbamylase).

FIGS. 32A, 32, B and 32C show plasmid and virus infected cells.: (A) rescue experiment: p53 null H1373 cells transfected either with or without p53R248Wmut and p53-XL, DBD-XL, E-XL, wt p53 and EGFP. 48 h post transfection 7-AAD assay was conducted. Statistical analysis was performed by using two-way ANOVA with Bonferroni's post test; **p<0.01 (B) MDA-MB-468 were infected with Ad-p53-XL, Ad-DBD-XL and Ad-ZsGreen. 72 h post infection, 7-AAD assay was conducted. Statistical analysis was performed by using one-way ANOVA with Bonferroni's post test; *p<0.05 and **p<0.01. Error bars represent standard deviations from three different independent experiments (n=3). (C) Representative cropped western blot of MDA-MB-468 cell lysates 24 h post infection with Ad-ZsGreen, untreated, Ad-p53-XL and Ad-DBD-XL.

FIGS. 33A, 33B, and 33C show the results of a tumor model: (A) generated tumor model: SQ injection of MDA-MB-468 human breast cancer cells into inguinal area of female nu/nu athymic mouse. 5λ108 pfu was injected intratumaorally on days 0-4 and 7-11 (18). Black arrow indicates tumor location. (B) Tumors harvested from mice after treatment. (C) Tumor size reduction. Statistical analysis was performed by using one-way ANOVA with Bonferroni's post test. Error bars represent standard deviations from at least three independent experiments (n=5).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or to particular reagents unless otherwise specified, and, as such, may vary. It is also to be understood that the terminology used herein is for the purpose of describing particular embodiments only and is not intended to be limiting.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, reference to “a nucleic acid sequence” includes a plurality of such nucleic acid sequences, reference to “the peptide” is a reference to one or more peptides and equivalents thereof known to those skilled in the art, and so forth.

“Peptide” as used herein refers to any polypeptide, oligopeptide, gene product, expression product, or protein. A peptide is comprised of consecutive amino acids. The term “peptide” encompasses recombinant, naturally occurring and synthetic molecules.

In addition, as used herein, the term “peptide” refers to amino acids joined to each other by peptide bonds or modified peptide bonds, e.g., peptide isosteres, etc. and may contain modified amino acids other than the 20 gene-encoded amino acids. The peptides can be modified by either natural processes, such as post-translational processing, or by chemical modification techniques which are well known in the art. Modifications can occur anywhere in the polypeptide, including the peptide backbone, the amino acid side-chains and the amino or carboxyl termini. The same type of modification can be present in the same or varying degrees at several sites in a given peptide. Also, a given peptide can have many types of modifications. Modifications include, without limitation, acetylation, acylation, ADP-ribosylation, amidation, covalent cross-linking or cyclization, covalent attachment of flavin, covalent attachment of a heme moiety, covalent attachment of a nucleotide or nucleotide derivative, covalent attachment of a lipid or lipid derivative, covalent attachment of a phosphytidylinositol, disulfide bond formation, demethylation, formation of cysteine or pyroglutamate, formylation, gamma-carboxylation, glycosylation, GPI anchor formation, hydroxylation, iodination, methylation, myristolyation, oxidation, pergylation, proteolytic processing, phosphorylation, prenylation, racemization, selenoylation, sulfation, and transfer-RNA mediated addition of amino acids to protein such as arginylation. (See Proteins—Structure and Molecular Properties 2nd Ed., T. E. Creighton, W.H. Freeman and Company, New York (1993); Posttranslational Covalent Modification of Proteins, B. C. Johnson, Ed., Academic Press, New York, pp. 1-12 (1983)).

As used herein, the term “amino acid sequence” refers to a list of abbreviations, letters, characters or words representing amino acid residues.

The amino acid abbreviations used herein are conventional one letter codes for the amino acids and are expressed as follows: A, alanine; B, asparagine or aspartic acid; C, cysteine; D aspartic acid; E, glutamate, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; Y, tyrosine; Z, glutamine or glutamic acid.

The phrase “nucleic acid” as used herein refers to a naturally occurring or synthetic oligonucleotide or polynucleotide, whether DNA or RNA or DNA-RNA hybrid, single-stranded or double-stranded, sense or antisense, which is capable of hybridization to a complementary nucleic acid by Watson-Crick base-pairing. Nucleic acids of the invention can also include nucleotide analogs (e.g., BrdU), and non-phosphodiester internucleoside linkages (e.g., peptide nucleic acid (PNA) or thiodiester linkages). In particular, nucleic acids can include, without limitation, DNA, RNA, cDNA, gDNA, ssDNA, dsDNA or any combination thereof

By “sample” is meant an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

By an “effective amount” of a compound as provided herein is meant a sufficient amount of the compound to provide the desired effect. The exact amount required will vary from subject to subject, depending on the species, age, and general condition of the subject, the severity of disease (or underlying genetic defect) that is being treated, the particular compound used, its mode of administration, and the like. Thus, it is not possible to specify an exact “effective amount.” However, an appropriate “effective amount” may be determined by one of ordinary skill in the art using only routine experimentation.

By “transgenic animal” is meant an animal comprising a transgene as described above. Transgenic animals are made by techniques that are well known in the art.

By “treat” is meant to administer a compound or molecule of the invention to a subject, such as a human or other mammal (for example, an animal model), that has an increased susceptibility for developing a hyperproliferative disorder, or that has a hyperproliferative disorder, in order to prevent or delay a worsening of the effects of the disease or condition, or to partially or fully reverse the effects of the disease. For example, the hyperproliferative disorder can be cancer.

By “prevent” is meant to minimize the chance that a subject who has an increased susceptibility for developing a disease will develop the disease.

By “specifically binds” is meant that an antibody recognizes and physically interacts with its cognate antigen (for example, a p53 peptide) and does not significantly recognize and interact with other antigens; such an antibody may be a polyclonal antibody or a monoclonal antibody, which are generated by techniques that are well known in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

The term “wild type p53 (wt p53)” refers to the p53 sequence of SEQ ID NO:24.

(SEQ ID NO: 24)
MEEPQSDPSV EPPLSQETFS DLWKLLPENN VLSPLPSQAM
DDLMLSPDDI EQWFTEDPGP DEAPRMPEAA PPVAPAPAAP
TPAAPAPAPS WPLSSSVPSQ KTYQGSYGFR LGFLHSGTAK
SVTCTYSPAL NKMFCQLAKT CPVQLWVDST PPPGTRVRAM
AIYKQSQHMT EVVRRCPHHE RCSDSDGLAP PQHLIRVEGN
LRVEYLDDRN TFRHSVVVPY EPPEVGSDCT TIHYNYMCNS
SCMGGMNRRP ILTIITLEDS SGNLLGRNSF EVRVCACPGR
DRRTEEENLR KKGEPHHELP PGSTKRALPN NTSSSPQPKK
KPLDGEYFTL QIRGRERFEM FRELNEALEL KDAQAGKEPG
GSRAHSSHLK SKKGQSTSRH KKLMFKTEGP DSD

Wild type p53 can be divided into three regions: an acidic N-terminal region (amino acids 1-101 of SEQ ID NO:24), a DNA binding domain (DBD, amino acids 102-292 of SEQ ID NO:24), and a basic C-terminal region (amino acids 293-393 of SEQ ID NO:24). The acidic N-terminal region contains a transactivation acidic domain (amino acids 1-42 of SEQ ID NO:24), a MDM2 binding domain (MBD, amino acids 17-28 of SEQ ID NO:24), and a proline-rich domain (PRD, amino acids 63-97 of SEQ ID NO:24). The basic C terminal region contains three nuclear localization signals (NLS, acids 305-322 most active NLS of SEQ ID NO:24), a tetramerization domain (TD, amino acids 323-356 of SEQ ID NO:24), and a negative regulatory region (amino acids 363-393 of SEQ ID NO:24).

The term “full-length p53 peptide” refers to the full length wild type p53 peptide. A full-length p53 peptide comprises all of the functional domains of wild type p53. For example, a full-length p53 peptide comprises the DNA binding domain, MDM2 binding domain, proline-rich domain, tetramerization domain, and transactivation domain of wt p53. A full-length p53 peptide can comprise the acidic N-terminal region (amino acids 1-101) of SEQ ID NO:24, a DNA binding domain (DBD, amino acids 102-292) of SEQ ID NO:24, and a basic C-terminal region (amino acids 293-393) of SEQ ID NO:24. The acidic N-terminal region contains a transactivation acidic domain (amino acids 1-42 of SEQ ID NO:24), a MDM2 binding domain (MBD, amino acids 17-28 of SEQ ID NO:24), and a proline-rich domain (PRD, amino acids 63-97 of SEQ ID NO:24). The basic C terminal region contains three nuclear localization signals (NLS, acids 305-322 most active NLS of SEQ ID NO:24), a tetramerization domain (TD, amino acids 323-356 of SEQ ID NO:24), and a negative regulatory region (amino acids 363-393 of SEQ ID NO:24).

The term “partial p53 peptide” refers to a p53 sequence peptide that has less than the full length wild type p53 peptide sequence. In some instances, a partial p53 peptide can lack one or more of the wild type p53 domains. Thus, a partial p53 peptide can comprise one or more domains of p53 without comprising all of the domains of wild type p53. For example, a partial p53 peptide can be a peptide comprising only the DNA binding domain of p53 (amino acids 102-292 of SEQ ID NO:24) or a combination of the DNA binding domain of p53 with one or more of the other p53 peptide domains, but not all of the p53 peptide domains. A partial p53 peptide comprising the DNA binding domain of p53 with one or more of the other p53 peptide domains, but not all of the p53 peptide domains, can comprise a DNA binding domain and at least one other p53 peptide domain wherein the at least one other p53 peptide domain comprises the transactivation domain (amino acids 1-42 of SEQ ID NO:24), MDM2 binding domain (MBD, amino acids 17-28 of SEQ ID NO:24), proline-rich domain (PRD, amino acids 63-97 of SEQ ID NO:24), tetramerization domain (TD, amino acids 323-356 of SEQ ID NO:24) of wt p53. In some aspects the DNA binding domain and the other p53 peptide domain comprise one, two, three, four, five, six, seven, eight, or nine additional amino acids on the C-terminal end of the domain, N-terminal end of the domain, or a combination.

The phrase “mitochondrial targeting sequence (MTS)” refers to a sequence that directs a molecule to the mitochondria. The sequence can be a peptide or nucleic acid sequence.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range—from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### B. Peptides

Disclosed are peptides comprising a full length p53 peptide and a mitochondrial targeting signal (MTS), wherein the MTS is a Bak or Bax MTS. A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the Bak or Bax MTS is SEQ ID NO:15 or 14, respectively.

Disclosed are peptides comprising a partial p53 peptide and a MTS. In some aspects, the MTS comprises a Bcl-XL, Bak, or Bax MTS. For example, a Bcl-XL MTS can comprise the amino acid sequence RKGQERFNRWFLTGMTVAGVVLLGSLFSRK (SEQ ID NO:13). A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the MTS is a Bcl-XL, Bak, or Bax MTS. For example, the Bcl-Xl, Bak, or Bax MTS can be SEQ ID NO:13, 15, or 14, respectively.

Disclosed are peptides comprising a partial p53 peptide and a MTS, wherein the MTS is not a TOM, OTC, or CCO MTS.

Disclosed are peptides comprising a partial p53 peptide and a MTS, wherein the partial p53 peptide consists of the DNA binding domain of p53. In some aspects, the partial p53 peptide consists of amino acids 102-292 of SEQ ID NO:24. In some aspects, the partial p53 peptide consists of amino acids 102-292 of SEQ ID NO:24. In some aspects, the partial p53 peptide consists of the DNA binding domain (amino acids 102-292 of SEQ ID NO:24) plus an additional one, two, three, four, five, six, seven, eight, or nine amino acids on the N-terminal end, C-terminal end, or a combination.

Disclosed are peptides comprising a partial p53 peptide and a MTS, wherein the partial p53 peptide comprises the DNA binding domain of p53. In some aspects, the DNA binding domain of p53 consists of amino acids 102-292 of SEQ ID NO:24.

Disclosed are peptides comprising a partial p53 peptide and a MTS, wherein the partial p53 peptide comprises the DNA binding domain of p53, wherein the partial p53 peptide further comprises a MDM2 binding domain, a proline-rich domain, a tetramerization domain, or a transactivation domain of p53.

Protein variants and derivatives are well understood to those of skill in the art and can involve amino acid sequence modifications. For example, amino acid sequence modifications typically fall into one or more of three classes: substitutional, insertional or deletional variants. Polypeptide variants generally encompassed by the present invention will typically exhibit at least about 70%, 75%, 80%, 85%, 90%, 91%, 92%, 93%, 94%, 95%, 96%, 97%, 98%, or 99% or more identity (determined as described below), along its length, to a polypeptide sequences set forth herein.

1. Compositions Comprising Peptides

Disclosed are compositions comprising one or more of the peptides described herein. For example, disclosed are compositions comprising a peptide, wherein the peptide comprises a full length p53 peptide and a MTS, wherein the MTS is a Bak or Bax MTS. The Bak MTS can comprise SEQ ID NO:15. The Bax MTS can comprise SEQ ID NO:14.

Disclosed are compositions comprising a peptide, wherein the peptide comprises a partial p53 peptide and a MTS. In some aspects, the MTS comprises a Bcl-XL, Bak, or Bax MTS. For example, a Bcl-XL MTS can comprise the amino acid sequence RKGQERFNRWFLTGMTVAGVVLLGSLFSRK (SEQ ID NO:13). A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the MTS is a Bcl-XL, Bak, or Bax MTS. For example, the Bcl-Xl, Bak, or Bax MTS can be SEQ ID NO:13, 15, or 14, respectively.

Disclosed are compositions comprising a peptide, wherein the peptide comprises a partial p53 peptide and a MTS, wherein the MTS is not a TOM, OTC or CCO MTS.

Disclosed are compositions comprising a peptide, wherein the peptide comprises a partial p53 peptide and a MTS, wherein the partial p53 peptide consists of the DNA binding domain of p53. In some aspects, the partial p53 peptide consists of amino acids 102-292 of SEQ ID NO:24.

Disclosed are compositions comprising a peptide, wherein the peptide comprises a partial p53 peptide and a MTS, wherein the partial p53 peptide comprises the DNA binding domain of p53. In some aspects, the DNA binding domain of p53 consists of amino acids 102-292 of SEQ ID NO:24.

Disclosed are compositions comprising a peptide, wherein the peptide comprises a partial p53 peptide and a MTS, wherein the partial p53 peptide comprises the DNA binding domain of p53, wherein the partial p53 peptide further comprises a MDM2 binding domain, a proline-rich domain, a tetramerization domain, or a transactivation domain of p53. The partial p53 peptide can also comprise the N-terminal region (amino acids 1-101 of SEQ ID NO:24), C-terminal region (amino acids 293-393 of SEQ ID NO:24), nuclear localization signals (for example, amino acids 305-322 of SEQ ID NO:24), or a negative regulatory region (amino acids 363-393 of SEQ ID NO:24).

2. Peptide Variants

Also disclosed herein are peptide variants of the peptides disclosed herein. Peptide variants and derivatives are well understood by those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Deletions are characterized by the removal of one or more amino acid residues from the peptide sequence. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place.

Conservative and non-conservative substitutions can be made. For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that one way to define the variants and derivatives of the disclosed peptides herein is through defining the variants and derivatives in terms of homology/identity to specific known sequences. For example, the sequence of wild type p53 is known. Specifically disclosed are variants of these and other peptides herein disclosed which have at least, 70% or 75% or 80% or 85% or 90% or 95% homology to the full length or a fragment of wild type sequence. Wherein a sequence is said to have at least about 70% sequence identity, it is understood to also have at least about 75%, 80%, 85%, 90%, 92%, 92%, 93%, 94%, 95%, 96%, 97%, 98%, 99%, or 100% sequence identity.

### C. Nucleic Acids

Also disclosed herein are nucleic acid sequences capable of encoding the peptides disclosed herein. For example, disclosed are nucleic acid sequences, wherein the nucleic acid sequences are capable of encoding a full length p53 peptide and a MTS, wherein the MTS comprises a Bak or Bax MTS. In some aspects, the MTS is a Bak, or Bax MTS. A nucleic acid sequence capable of encoding a Bak MTS can comprise 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). A nucleic acid sequence capable of encoding a Bax MTS can comprise 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23).

Disclosed are nucleic acid sequences, wherein the nucleic acid sequences are capable of encoding a partial p53 peptide and a MTS. Disclosed are nucleic acid sequences, wherein the nucleic acid sequences are capable of encoding a partial p53 peptide and a MTS, wherein the MTS is not a TOM, OTC, or CCO MTS.

Disclosed are nucleic acid sequences, wherein the nucleic acid sequences are capable of encoding a partial p53 peptide and a MTS, wherein the partial p53 peptide comprises the DNA binding domain of p53. In some aspects, the DNA binding domain of p53 consists of amino acids 102-292 of SEQ ID NO:24. For example, the DNA binding domain of p53 can be SSSVPSQ KTYQGSYGFR LGFLHSGTAK SVTCTYSPAL NKMFCQLAKT CPVQLWVDST PPPGTRVRAM AIYKQSQHMT EVVRRCPHHE RCSDSDGLAP PQHLIRVEGN LRVEYLDDRN TFRHSVVVPY EPPEVGSDCT TIHYNYMCNS SCMGGMNRRP ILTIITLEDS SGNLLGRNSF EVRVCACPGR DRRTEEENLR KKGE (SEQ ID NO:25).

Disclosed are nucleic acid sequences, wherein the nucleic acid sequences are capable of encoding a partial p53 peptide and a MTS, wherein the MTS comprises a Bcl-XL, Bak, or Bax MTS. In some aspects, the MTS is a Bcl-XL, Bak, or Bax MTS. Nucleic acid sequences capable of encoding a MTS can comprise the sequences of 5′AGAAAGGGCCAGGAGAGATTCAACAGATGGTTCCTGACCGGCATGACCGTGGC CGGCGTGGTGCTGCTGGGCAGCCTGTTCAGCAGAAAGTGA-3′ (SEQ ID NO:27), 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23), or 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). In some aspects, the Bcl-XL, Bak, or Bax MTS is encoded by SEQ ID NO:27, 26, or 23, respectively.

Also disclosed herein are nucleic acid sequences, wherein the nucleic acid sequences are capable of encoding a peptide comprising a full length p53 peptide and a MTS, wherein the MTS comprises a Bak or Bax MTS. In some aspects, the MTS is a Bak, or Bax MTS. A nucleic acid sequence capable of encoding a Bak MTS can comprise 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). A nucleic acid sequence capable of encoding a Bax MTS can comprise 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23).

Disclosed are nucleic acid sequences, wherein the nucleic acid sequences are capable of encoding a peptide comprising a partial p53 peptide and a MTS. Disclosed are nucleic acid sequences, wherein the nucleic acid sequences are capable of encoding a peptide comprising a partial p53 peptide and a MTS, wherein the MTS is not a TOM, OTC, or CCO MTS.

Disclosed are nucleic acid sequences, wherein the nucleic acid sequences are capable of encoding a peptide comprising a partial p53 peptide and a MTS, wherein the partial p53 peptide comprises the DNA binding domain of p53. In some aspects, the DNA binding domain of p53 consists of amino acids 102-292 of SEQ ID NO:24. For example, the DNA binding domain of p53 can be SSSVPSQ KTYQGSYGFR LGFLHSGTAK SVTCTYSPAL NKMFCQLAKT CPVQLWVDST PPPGTRVRAM AIYKQSQHMT EVVRRCPHHE RCSDSDGLAP PQHLIRVEGN LRVEYLDDRN TFRHSVVVPY EPPEVGSDCT TIHYNYMCNS SCMGGMNRRP ILTIITLEDS SGNLLGRNSF EVRVCACPGR DRRTEEENLR KKGE (SEQ ID NO:25).

Disclosed are nucleic acid sequences, wherein the nucleic acid sequences are capable of encoding a peptide comprising a partial p53 peptide and a MTS, wherein the MTS comprises a Bcl-XL, Bak, or Bax MTS. In some aspects, the MTS is a Bcl-XL, Bak, or Bax MTS. Nucleic acid sequences capable of encoding a MTS can comprise the sequences of S′AGAAAGGGCCAGGAGAGATTCAACAGATGGTTCCTGACCGGCATGACCGTGGC CGGCGTGGTGCTGCTGGGCAGCCTGTTCAGCAGAAAGTGA-3′ (SEQ ID NO:27), 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23), or 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). In some aspects, the Bcl-XL, Bak, or Bax MTS is encoded by SEQ ID NO:27, 26, or 23, respectively.

Also disclosed herein are nucleic acid sequences comprising the nucleic acid sequence of p53 and the nucleic acid sequence of a Bak or Bax MTS. For example, disclosed are nucleic acid sequences comprising the nucleic acid sequence of atggaggagccgcagtcagatcctagcgtcgagccccctctgagtcaggaaacattttcagacctatggaaactacttcctgaaaaca acgttctgtcccccttgccgtcccaagcaatggatgatttgatgctgtccccggacgatattgaacaatggttcactgaagacccaggtc cagatgaagctcccagaatgccagaggctgctccccccgtggcccctgcaccagcagctcctacaccggcggcccctgcaccagc cccctcctggcccctgtcatcttctgtcccttcccagaaaacctaccagggcagctacggtttccgtctgggcttcttgcattctgggaca gccaagtctgtgacttgcacgtactcccctgccctcaacaagatgttttgccaactggccaagacctgccctgtgcagctgtgggttgat tccacacccccgcccggcacccgcgtccgcgccatggccatctacaagcagtcacagcacatgacggaggttgtgaggcgctgcc cccaccatgagcgctgctcagatagcgatggtctggcccctcctcagcatcttatccgagtggaaggaaatttgcgtgtggagtatttgg atgacagaaacacttttcgacatagtgtggtggtgccctatgagccgcctgaggttggctctgactgtaccaccatccactacaactaca tgtgtaacagttcctgcatgggcggcatgaaccggaggcccatcctcaccatcatcacactggaagactccagtggtaatctactggg acggaacagctttgaggtgcgtgtttgtgcctgtcctgggagagaccggcgcacagaggaagagaatctccgcaagaaaggggag cctcaccacgagctgcccccagggagcactaagcgagcactgcccaacaacaccagctcctctccccagccaaagaagaaaccac tggatggagaatatttcacccttcagatccgtgggcgtgagcgcttcgagatgttccgagagctgaatgaggccttggaactcaaggat gcccaggctgggaaggagccaggggggagcagggctcactccagccacctgaagtccaaaaagggtcagtctacctcccgccata aaaaactcatgttcaagacagaagggcctgactcagactga (SEQ ID NO:29) and the nucleic acid sequence of SEQ ID NO:23 or SEQ ID NO:26.

Disclosed are nucleic acid sequences comprising a partial nucleic acid sequence of p53 and the nucleic acid sequence of a MTS. In some instances the MTS is not a TOM, OTC, or CCO MTS. The partial p53 nucleic acid sequences can comprise the nucleic acid sequence that encodes for the DNA binding domain of p53. For example, the partial nucleic acid sequence of p53 can comprise acctaccagggcagctacggfficcgtctgggcttcttgcattctgggacagccaagtctgtgacttgcacgtactcccctgccctcaac aagatgttttgccaactggccaagacctgccctgtgcagctgtgggttgattccacacccccgcccggcacccgcgtccgcgccatgg ccatctacaagcagtcacagcacatgacggaggttgtgaggcgctgcccccaccatgagcgctgctcagatagcgatggtctggccc ctcctcagcatcttatccgagtggaaggaaatttgcgtgtggagtatttggatgacagaaacacttttcgacatagtgtggtggtgccctat gagccgcctgaggttggctctgactgtaccaccatccactacaactacatgtgtaacagttcctgcatgggcggcatgaaccggaggc ccatcctcaccatcatcacactggaagactccagtggtaatctactgggacggaacagctttgaggtgcgtgtttgtgcctgtcctggga gagaccggcgcacagaggaagagaatctccgcaag (SEQ ID NO:30)

In some instances, the MTS comprises a Bcl-XL, Bak, or Bax MTS. In some aspects, the MTS is a Bcl-XL, Bak, or Bax MTS. Nucleic acid sequences capable of encoding a MTS can comprise the sequences of 5′AGAAAGGGCCAGGAGAGATTCAACAGATGGTTCCTGACCGGCATGACCGTGGC CGGCGTGGTGCTGCTGGGCAGCCTGTTCAGCAGAAAGTGA-3′ (SEQ ID NO:27), 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23), or 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). In some aspects, the Bcl-XL, Bak, or Bax MTS is encoded by SEQ ID NO:27, 26, or 23, respectively.

It would be routine for one with ordinary skill in the art to make a nucleic acid that encodes the peptides disclosed herein since codons for each of the amino acids that make up the peptides are known.

Also, disclosed are compositions including primers and probes, which are capable of interacting with the polynucleotide sequences disclosed herein. For example, disclosed are primers/probes capable of amplifying a nucleic acid capable of encoding one or more of the disclosed peptides. The disclosed primers can used to support DNA amplification reactions. Typically the primers will be capable of being extended in a sequence specific manner. Extension of a primer in a sequence specific manner includes any methods wherein the sequence or composition of the nucleic acid molecule to which the primer is hybridized or otherwise associated directs or influences the composition or sequence of the product produced by the extension of the primer. Extension of the primer in a sequence specific manner therefore includes, but is not limited to, PCR, DNA sequencing, DNA extension, DNA polymerization, RNA transcription, or reverse transcription. Techniques and conditions that amplify the primer in a sequence specific manner are preferred. In certain embodiments the primers are used for the DNA amplification reactions, such as PCR or direct sequencing. It is understood that in certain embodiments the primers can also be extended using non-enzymatic techniques, where for example, the nucleotides or oligonucleotides used to extend the primer are modified such that they will chemically react to extend the primer in a sequence specific manner. Typically the disclosed primers hybridize with the polynucleotide sequences disclosed herein or region of the polynucleotide sequences disclosed herein or they hybridize with the complement of the polynucleotide sequences disclosed herein or complement of a region of the polynucleotide sequences disclosed herein.

The size of the primers or probes for interaction with the polynucleotide sequences disclosed herein in certain embodiments can be any size that supports the desired enzymatic manipulation of the primer, such as DNA amplification or the simple hybridization of the probe or primer. A typical primer or probe would be at least 6, 7, 8, 9, 10, 20, 30, 40, 50, 60, 70, 80, 90, 100, 125, 150, 175, 200, 225, 250, 275, 300, 325, 350, 375, 400, 425, 450, 475, 500, 550, 600, 650, 700, 750, 800, 850, 900, 950, 1000, 1250, 1500, 1750, 2000, 2250, 2500, 2750, 3000, 3500, or 4000 nucleotides long or any length in between.

Also disclosed are functional nucleic acids that can interact with the disclosed polynucleotides. Functional nucleic acids are nucleic acid molecules that have a specific function, such as binding a target molecule or catalyzing a specific reaction. Functional nucleic acid molecules can be divided into the following categories, which are not meant to be limiting. For example, functional nucleic acids include antisense molecules, aptamers, ribozymes, triplex forming molecules, and external guide sequences. The functional nucleic acid molecules can act as affectors, inhibitors, modulators, and stimulators of a specific activity possessed by a target molecule, or the functional nucleic acid molecules can possess a de novo activity independent of any other molecules.

Functional nucleic acid molecules can interact with any macromolecule, such as DNA, RNA, polypeptides, or carbohydrate chains. Thus, functional nucleic acids can interact with the mRNA of polynucleotide sequences disclosed herein or the genomic DNA of the polynucleotide sequences disclosed herein or they can interact with the polypeptide encoded by the polynucleotide sequences disclosed herein. Often functional nucleic acids are designed to interact with other nucleic acids based on sequence homology between the target molecule and the functional nucleic acid molecule. In other situations, the specific recognition between the functional nucleic acid molecule and the target molecule is not based on sequence homology between the functional nucleic acid molecule and the target molecule, but rather is based on the formation of tertiary structure that allows specific recognition to take place.

Optionally, isolated peptides or isolated nucleotides can also be purified, e.g., are at least about 90% pure, more preferably at least about 95% pure and most preferably at least about 99% pure.

By “isolated peptide” or “purified peptide” is meant a peptide (or a fragment thereof) that is substantially free from the materials with which the peptide is normally associated in nature. The peptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “isolated nucleic acid” or “purified nucleic acid” is meant DNA that is free of the genes that, in the naturally-occurring genome of the organism from which the DNA of the invention is derived, flank the gene. The term therefore includes, for example, a recombinant DNA which is incorporated into a vector, such as an autonomously replicating plasmid or virus; or incorporated into the genomic DNA of a prokaryote or eukaryote (e.g., a transgene); or which exists as a separate molecule (for example, a cDNA or a genomic or cDNA fragment produced by PCR, restriction endonuclease digestion, or chemical or in vitro synthesis). It also includes a recombinant DNA which is part of a hybrid gene encoding additional polypeptide sequence. The term “isolated nucleic acid” also refers to RNA, e.g., an mRNA molecule that is encoded by an isolated DNA molecule, or that is chemically synthesized, or that is separated or substantially free from at least some cellular components, for example, other types of RNA molecules or polypeptide molecules.

It is understood that one way to define any known variants and derivatives or those that might arise, of the disclosed genes and proteins herein is through defining the variants and derivatives in terms of homology to specific known sequences. Specifically disclosed are variants of the genes and proteins herein disclosed which have at least, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. MoL Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

1. Compositions Comprising Nucleic Acids

Also disclosed are compositions comprising the nucleic acid sequences described herein. For example, disclosed are compositions comprising nucleic acid sequences, wherein the nucleic acid sequences are capable of encoding a full length p53 peptide and a MTS, wherein the MTS comprises a Bak or Bax MTS. In some aspects, the MTS is a Bak, or Bax MTS. A nucleic acid sequence capable of encoding a Bak MTS can comprise 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). A nucleic acid sequence capable of encoding a Bax MTS can comprise 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23). Thus, in some aspects, the Bak and Bax MTS is encoded by SEQ ID NO:26 and 23, respectively.

Disclosed are compositions comprising nucleic acid sequences, wherein the nucleic acid sequences are capable of encoding a partial p53 peptide and a MTS. Disclosed are compositions comprising nucleic acid sequences, wherein the nucleic acid sequences are capable of encoding a partial p53 peptide and a MTS, wherein the MTS is not a TOM, OTC, or CCO MTS.

Disclosed are compositions comprising nucleic acid sequences, wherein the nucleic acid sequences are capable of encoding a partial p53 peptide and a MTS, wherein the partial p53 peptide comprises the DNA binding domain of p53. In some aspects, the DNA binding domain of p53 consists of amino acids 102-292 of SEQ ID NO:24. For example, the DNA binding domain of p53 can be SSSVPSQ KTYQGSYGFR LGFLHSGTAK SVTCTYSPAL NKMFCQLAKT CPVQLWVDST PPPGTRVRAM AIYKQSQHMT EVVRRCPHHE RCSDSDGLAP PQHLIRVEGN LRVEYLDDRN TFRHSVVVPY EPPEVGSDCT TIHYNYMCNS SCMGGMNRRP ILTIITLEDS SGNLLGRNSF EVRVCACPGR DRRTEEENLR KKGE (SEQ ID NO:25).

Disclosed are compositions comprising a nucleic acid sequence, wherein the nucleic acid sequence is capable of encoding a partial p53 peptide and a MTS, wherein the MTS comprises a Bcl-XL, Bak, or Bax MTS. In some aspects, the MTS is a Bcl-XL, Bak, or Bax MTS. Compositions can include those comprising a nucleic acid sequence capable of encoding a MTS can comprise the sequences of 5′AGAAAGGGCCAGGAGAGATTCAACAGATGGTTCCTGACCGGCATGACCGTGGC CGGCGTGGTGCTGCTGGGCAGCCTGTTCAGCAGAAAGTGA-3′ (SEQ ID NO:27), 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23), or 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). Thus, in some aspects, the Bcl-XL, Bak, or Bax MTS is encoded by SEQ ID NO:27, 23, or 26, respectively.

It would be routine for one with ordinary skill in the art to make a nucleic acid that encodes the peptides disclosed herein since codons for each of the amino acids that make up the peptides are known.

### D. Vectors

Also disclosed are vectors comprising the nucleic acid sequences disclosed herein. For example, disclosed are vectors comprising a nucleic acid sequence, wherein the nucleic acid sequence is capable of encoding a full length p53 peptide and a MTS, wherein the MTS comprises a Bak or Bax MTS. In some aspects, the MTS is a Bak, or Bax MTS.

Also disclosed are vectors comprising a nucleic acid sequence, wherein the nucleic acid sequence is capable of encoding a partial p53 peptide and a MTS.

Disclosed are vectors comprising a nucleic acid sequence, wherein the nucleic acid sequence is capable of encoding a partial p53 peptide and a MTS, wherein the partial p53 peptide comprises the DNA binding domain of p53.

Disclosed are vectors comprising a nucleic acid sequence, wherein the nucleic acid sequence is capable of encoding a partial p53 peptide and a MTS, wherein the MTS comprises a Bcl-XL, Bak, or Bax MTS. In some aspects, the MTS is a Bcl-XL, Bak, or Bax MTS.

The disclosed vectors thus can provide DNA molecules that are capable of integration into a mammalian chromosome without substantial toxicity.

Vectors can be viral vectors. For example, the viral vector can be, but is not limited to, an adenoviral vector, lentiviral vector or adeno-associated virus vector.

Also disclosed are non-viral vectors comprising any of the disclosed nucleic acid sequences.

For example, disclosed are the following vectors: p53-BakMTS, E-BakMTS, p53-BaxMTS, E-BaxMTS, DBD-BakMTS, DBD-BaxMTS, p53K120E-BakMTS, DBDK120E-BakMTS, p53m6-BakMTS, DBDm6-BakMTS,

1. Compositions Comprising Vectors

Also disclosed are composition comprising the vectors described herein. For example, disclosed are compositions comprising vectors, wherein the vectors comprise any of the disclosed peptide or nucleic acid sequences herein. For example, disclosed are compositions comprising a vector, wherein the vector comprises a nucleic acid sequence, wherein the nucleic acid sequence is capable of encoding a full length p53 peptide and a MTS, wherein the MTS comprises a Bak or Bax MTS. In some aspects, the MTS is a Bak, or Bax MTS.

Also disclosed are compositions comprising a vector, wherein the vector comprises a nucleic acid sequence, wherein the nucleic acid sequence is capable of encoding a partial p53 peptide and a MTS.

2. Viral and Non-Viral Vectors

There are a number of compositions and methods which can be used to deliver nucleic acids to cells, either in vitro or in vivo. These methods and compositions can largely be broken down into two classes: viral based delivery systems and non-viral based delivery systems. For example, the nucleic acids can be delivered through a number of direct delivery systems such as, electroporation, lipofection, calcium phosphate precipitation, plasmids, viral vectors, viral nucleic acids, phage nucleic acids, phages, cosmids, or via transfer of genetic material in cells or carriers such as cationic liposomes. Appropriate means for transfection, including viral vectors, chemical transfectants, or physico-mechanical methods such as electroporation and direct diffusion of DNA, are described by, for example, Wolff, J. A., et al., Science, 247, 1465-1468, (1990); and Wolff, J. A. Nature, 352, 815-818, (1991). Such methods are well known in the art and readily adaptable for use with the compositions and methods described herein. In certain cases, the methods will be modified to specifically function with large DNA molecules. Further, these methods can be used to target certain diseases and cell populations by using the targeting characteristics of the carrier.

Expression vectors can be any nucleotide construction used to deliver genes or gene fragments into cells (e.g., a plasmid), or as part of a general strategy to deliver genes or gene fragments, e.g., as part of recombinant retrovirus or adenovirus (Ram et al. Cancer Res. 53:83-88, (1993)). For example, disclosed herein are expression vectors comprising a nucleic acid sequence capable of encoding one or more of the disclosed peptides operably linked to a control element.

The “control elements” present in an expression vector are those non-translated regions of the vector—enhancers, promoters, 5′ and 3′ untranslated regions—which interact with host cellular proteins to carry out transcription and translation. Such elements may vary in their strength and specificity. Depending on the vector system and host utilized, any number of suitable transcription and translation elements, including constitutive and inducible promoters, may be used. For example, when cloning in bacterial systems, inducible promoters such as the hybrid lacZ promoter of the pBLUESCRIPT phagemid (Stratagene, La Jolla, Calif.) or pSPORT1 plasmid (Gibco BRL, Gaithersburg, Md.) and the like may be used. In mammalian cell systems, promoters from mammalian genes or from mammalian viruses are generally preferred. If it is necessary to generate a cell line that contains multiple copies of the sequence encoding a polypeptide, vectors based on SV40 or EBV may be advantageously used with an appropriate selectable marker.

Preferred promoters controlling transcription from vectors in mammalian host cells may be obtained from various sources, for example, the genomes of viruses such as polyoma, Simian Virus 40 (SV40), adenovirus, retroviruses, hepatitis-B virus and most preferably cytomegalovirus, or from heterologous mammalian promoters (e.g., beta actin promoter). The early and late promoters of the SV40 virus are conveniently obtained as an SV40 restriction fragment, which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Additionally, promoters from the host cell or related species can also be used.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Preferred examples are the SV40 enhancer on the late side of the replication origin (bp 100-270), the cytomegalovirus early promoter enhancer, the polyoma enhancer on the late side of the replication origin, and adenovirus enhancers.

The promoter or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

Optionally, the promoter or enhancer region can act as a constitutive promoter or enhancer to maximize expression of the polynucleotides of the invention. In certain constructs the promoter or enhancer region be active in all eukaryotic cell types, even if it is only expressed in a particular type of cell at a particular time. A preferred promoter of this type is the CMV promoter (650 bases). Other preferred promoters are SV40 promoters, cytomegalovirus (full length promoter), and retroviral vector LTR.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. It is preferred that the transcription unit also contains a polyadenylation region. One benefit of this region is that it increases the likelihood that the transcribed unit will be processed and transported like mRNA. The identification and use of polyadenylation signals in expression constructs is well established. It is preferred that homologous polyadenylation signals be used in the transgene constructs. In certain transcription units, the polyadenylation region is derived from the SV40 early polyadenylation signal and consists of about 400 bases.

The expression vectors can include a nucleic acid sequence encoding a marker product. This marker product is used to determine if the gene has been delivered to the cell and once delivered is being expressed. Preferred marker genes are the E. coli lacZ gene, which encodes β-galactosidase, and the gene encoding the green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

As used herein, plasmid or viral vectors are agents that transport the disclosed nucleic acids, such as a nucleic acid sequence capable of encoding one or more of the disclosed peptides into the cell without degradation and include a promoter yielding expression of the gene in the cells into which it is delivered. In some embodiments the nucleic acid sequences disclosed herein are derived from either a virus or a retrovirus. Viral vectors are, for example, Adenovirus, Adeno-associated virus, Herpes virus, Vaccinia virus, Polio virus, AIDS virus, neuronal trophic virus, Sindbis and other RNA viruses, including these viruses with the HIV backbone. Also preferred are any viral families which share the properties of these viruses which make them suitable for use as vectors. Retroviruses include Murine Maloney Leukemia virus, MMLV, and retroviruses that express the desirable properties of MMLV as a vector. Retroviral vectors are able to carry a larger genetic payload, i.e., a transgene or marker gene, than other viral vectors, and for this reason are a commonly used vector. However, they are not as useful in non-proliferating cells. Adenovirus vectors are relatively stable and easy to work with, have high titers, and can be delivered in aerosol formulation, and can transfect non-dividing cells. Pox viral vectors are large and have several sites for inserting genes, they are thermostable and can be stored at room temperature. A preferred embodiment is a viral vector which has been engineered so as to suppress the immune response of the host organism, elicited by the viral antigens. Preferred vectors of this type will carry coding regions for Interleukin 8 or 10.

Viral vectors can have higher transaction abilities (i.e., ability to introduce genes) than chemical or physical methods of introducing genes into cells. Typically, viral vectors contain, nonstructural early genes, structural late genes, an RNA polymerase III transcript, inverted terminal repeats necessary for replication and encapsidation, and promoters to control the transcription and replication of the viral genome. When engineered as vectors, viruses typically have one or more of the early genes removed and a gene or gene/promoter cassette is inserted into the viral genome in place of the removed viral DNA. Constructs of this type can carry up to about 8 kb of foreign genetic material. The necessary functions of the removed early genes are typically supplied by cell lines which have been engineered to express the gene products of the early genes in trans.

Retroviral vectors, in general, are described by Verma, I. M., Retroviral vectors for gene transfer. In Microbiology, Amer. Soc. for Microbiology, pp. 229-232, Washington, (1985), which is hereby incorporated by reference in its entirety. Examples of methods for using retroviral vectors for gene therapy are described in U.S. Pat. Nos. 4,868,116 and 4,980,286; PCT applications WO 90/02806 and WO 89/07136; and Mulligan, (Science 260:926-932 (1993)); the teachings of which are incorporated herein by reference in their entirety for their teaching of methods for using retroviral vectors for gene therapy.

A retrovirus is essentially a package which has packed into it nucleic acid cargo. The nucleic acid cargo carries with it a packaging signal, which ensures that the replicated daughter molecules will be efficiently packaged within the package coat. In addition to the package signal, there are a number of molecules which are needed in cis, for the replication, and packaging of the replicated virus. Typically a retroviral genome contains the gag, pol, and env genes which are involved in the making of the protein coat. It is the gag, pol, and env genes which are typically replaced by the foreign DNA that it is to be transferred to the target cell. Retrovirus vectors typically contain a packaging signal for incorporation into the package coat, a sequence which signals the start of the gag transcription unit, elements necessary for reverse transcription, including a primer binding site to bind the tRNA primer of reverse transcription, terminal repeat sequences that guide the switch of RNA strands during DNA synthesis, a purine rich sequence 5′ to the 3′ LTR that serves as the priming site for the synthesis of the second strand of DNA synthesis, and specific sequences near the ends of the LTRs that enable the insertion of the DNA state of the retrovirus to insert into the host genome. This amount of nucleic acid is sufficient for the delivery of a one to many genes depending on the size of each transcript. It is preferable to include either positive or negative selectable markers along with other genes in the insert.

Since the replication machinery and packaging proteins in most retroviral vectors have been removed (gag, pol, and env), the vectors are typically generated by placing them into a packaging cell line. A packaging cell line is a cell line which has been transfected or transformed with a retrovirus that contains the replication and packaging machinery but lacks any packaging signal. When the vector carrying the DNA of choice is transfected into these cell lines, the vector containing the gene of interest is replicated and packaged into new retroviral particles, by the machinery provided in cis by the helper cell. The genomes for the machinery are not packaged because they lack the necessary signals.

The construction of replication-defective adenoviruses has been described (Berkner et al., J. Virology 61:1213-1220 (1987); Massie et al., Mol. Cell. Biol. 6:2872-2883 (1986); Haj-Ahmad et al., J. Virology 57:267-274 (1986); Davidson et al., J. Virology 61:1226-1239 (1987); Zhang “Generation and identification of recombinant adenovirus by liposome-mediated transfection and PCR analysis” BioTechniques 15:868-872 (1993)). The benefit of the use of these viruses as vectors is that they are limited in the extent to which they can spread to other cell types, since they can replicate within an initial infected cell but are unable to form new infectious viral particles. Recombinant adenoviruses have been shown to achieve high efficiency gene transfer after direct, in vivo delivery to airway epithelium, hepatocytes, vascular endothelium, CNS parenchyma and a number of other tissue sites (Morsy, J. Clin. Invest. 92:1580-1586 (1993); Kirshenbaum, J. Clin. Invest. 92:381-387 (1993); Roessler, J. Clin. Invest. 92:1085-1092 (1993); Moullier, Nature Genetics 4:154-159 (1993); La Salle, Science 259:988-990 (1993); Gomez-Foix, J. Biol. Chem. 267:25129-25134 (1992); Rich, Human Gene Therapy 4:461-476 (1993); Zabner, Nature Genetics 6:75-83 (1994); Guzman, Circulation Research 73:1201-1207 (1993); Bout, Human Gene Therapy 5:3-10 (1994); Zabner, Cell 75:207-216 (1993); Caillaud, Eur. J. Neuroscience 5:1287-1291 (1993); and Ragot, J. Gen. Virology 74:501-507 (1993)) the teachings of which are incorporated herein by reference in their entirety for their teaching of methods for using retroviral vectors for gene therapy. Recombinant adenoviruses achieve gene transduction by binding to specific cell surface receptors, after which the virus is internalized by receptor-mediated endocytosis, in the same manner as wild type or replication-defective adenovirus (Chardonnet and Dales, Virology 40:462-477 (1970); Brown and Burlingham, J. Virology 12:386-396 (1973); Svensson and Persson, J. Virology 55:442-449 (1985); Seth, et al., J. Virol. 51:650-655 (1984); Seth, et al., Mol. Cell. Biol., 4:1528-1533 (1984); Varga et al., J. Virology 65:6061-6070 (1991); Wickham et al., Cell 73:309-319 (1993)).

A viral vector can be one based on an adenovirus which has had the E1 gene removed and these virons are generated in a cell line such as the human 293 cell line. Optionally, both the E1 and E3 genes are removed from the adenovirus genome.

Another type of viral vector that can be used to introduce the polynucleotides of the invention into a cell is based on an adeno-associated virus (AAV). This defective parvovirus is a preferred vector because it can infect many cell types and is nonpathogenic to humans. AAV type vectors can transport about 4 to 5 kb and wild type AAV is known to stably insert into chromosome 19. Vectors which contain this site specific integration property are preferred. An especially preferred embodiment of this type of vector is the P4.1 C vector produced by Avigen, San Francisco, Calif., which can contain the herpes simplex virus thymidine kinase gene, HSV-tk, or a marker gene, such as the gene encoding the green fluorescent protein, GFP.

In another type of AAV virus, the AAV contains a pair of inverted terminal repeats (ITRs) which flank at least one cassette containing a promoter which directs cell-specific expression operably linked to a heterologous gene. Heterologous in this context refers to any nucleotide sequence or gene which is not native to the AAV or B19 parvovirus. Typically the AAV and B19 coding regions have been deleted, resulting in a safe, noncytotoxic vector. The AAV ITRs, or modifications thereof, confer infectivity and site-specific integration, but not cytotoxicity, and the promoter directs cell-specific expression. U.S. Pat. No. 6,261,834 is herein incorporated by reference in its entirety for material related to the AAV vector.

The inserted genes in viral and retroviral vectors usually contain promoters, or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Other useful systems include, for example, replicating and host-restricted non-replicating vaccinia virus vectors. In addition, the disclosed nucleic acid sequences can be delivered to a target cell in a non-nucleic acid based system. For example, the disclosed polynucleotides can be delivered through electroporation, or through lipofection, or through calcium phosphate precipitation. The delivery mechanism chosen will depend in part on the type of cell targeted and whether the delivery is occurring for example in vivo or in vitro.

Thus, the compositions can comprise, in addition to the disclosed expression vectors, lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subjects lung cells. Regarding liposomes, see, e.g., Brigham et al. Am. J. Resp. Cell. Mol. Biol. 1:95-100 (1989); Felgner et al. Proc. Natl. Acad. Sci USA 84:7413-7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

### E. Delivery of Compositions

In the methods described herein, delivery of the compositions to cells can be via a variety of mechanisms. As defined above, disclosed herein are compositions comprising any one or more of the peptides, nucleic acids, vectors and/or antibodies described herein can be used to produce a composition which can also include a carrier such as a pharmaceutically acceptable carrier. For example, disclosed are pharmaceutical compositions, comprising the peptides disclosed herein, and a pharmaceutically acceptable carrier.

For example, the compositions described herein can comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. Examples of carriers include dimyristoylphosphatidyl (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A. R. Gennaro, Mack Publishing Company, Easton, Pa. 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

### F. Methods of Targeting

1. Targeting a Peptide

Disclosed are methods of targeting a peptide to mitochondria comprising introducing a peptide to a cell, wherein the peptide comprises a full length p53 peptide and a MTS, wherein the MTS comprises a Bak or Bax MTS. In some aspects, the MTS is a Bak, or Bax MTS. A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the Bak or Bax MTS consists of SEQ ID NO:15 or 14, respectively.

Also disclosed are methods of targeting a peptide to mitochondria comprising introducing a peptide to a cell, wherein the peptide comprises a partial p53 peptide and a MTS. In some aspects, the MTS comprises a Bcl-XL, Bak, or Bax MTS. In some aspects, the MTS is a Bcl-XL, Bak, or Bax MTS. For example, a Bcl-XL MTS can comprise the amino acid sequence RKGQERFNRWFLTGMTVAGVVLLGSLFSRK (SEQ ID NO:13). A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects the Bcl-XL, Bak, or Bax MTS consists of SEQ ID NO:13, 15, or 14, respectively.

Disclosed are methods of targeting a peptide to mitochondria comprising introducing a peptide to a cell, wherein the peptide comprises a partial p53 peptide and a MTS, wherein the MTS is not a TOM, OTC, or CCO MTS.

Disclosed are methods of targeting a peptide to mitochondria comprising introducing a peptide to a cell, wherein the peptide comprises a partial p53 peptide and a MTS, wherein the partial p53 peptide consists of the DNA binding domain of p53. In some aspects, the partial p53 peptide consists of amino acids 102-292 of SEQ ID NO:24.

Disclosed are methods of targeting a peptide to mitochondria comprising introducing a peptide to a cell, wherein the peptide comprises a partial p53 peptide and a MTS, wherein the partial p53 peptide comprises the DNA binding domain of p53. In some aspects, the DNA binding domain of p53 consists of amino acids 102-292 of SEQ ID NO:24.

Disclosed are methods of targeting a peptide to mitochondria comprising introducing a peptide to a cell, wherein the peptide comprises a partial p53 peptide and a MTS, wherein the partial p53 peptide comprises the DNA binding domain of p53, wherein the partial p53 peptide further comprises a MDM2 binding domain, a proline-rich domain, a tetramerization domain, or a transactivation domain of p53. The partial p53 peptide can also comprise the N-terminal region (amino acids 1-101), C-terminal region (amino acids 293-393 of SEQ ID NO:24), nuclear localization signals (for example, amino acids 305-322 of SEQ ID NO:24), or a negative regulatory region (amino acids 363-393 of SEQ ID NO:24).

Also disclosed are methods of targeting any of the disclosed peptides herein to mitochondria comprising introducing or administering the peptide to a subject, wherein the subject comprises mitochondria.

Disclosed are methods of targeting a peptide to mitochondria comprising introducing any of the disclosed herein nucleic acid sequences capable of encoding one or more of the disclosed peptides. For example, disclosed are methods of targeting a peptide to mitochondria comprising introducing a nucleic acid sequence to a cell, wherein the nucleic acid sequence is capable of encoding a peptide, wherein the peptide comprises a full length p53 peptide and a MTS, wherein the MTS comprises a Bak or Bax MTS. In some aspects, the MTS is a Bak, or Bax MTS.

In some aspects, methods of targeting a peptide to mitochondria comprising introducing a nucleic acid sequence to a cell, wherein the nucleic acid sequence is capable of encoding a peptide, wherein the peptide comprises a partial p53 peptide and a MTS are disclosed.

2. Targeting a Composition

Disclosed are methods of targeting a composition to mitochondria comprising introducing a composition to a subject, wherein the composition comprises a peptide, wherein the peptide comprises a full length p53 peptide and a MTS, wherein the MTS comprises a Bak or Bax MTS. In some aspects, the MTS is a Bak, or Bax MTS. A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the Bak or Bax MTS consists of SEQ ID NO:15 or 14, respectively.

Disclosed are methods of targeting a composition to mitochondria comprising introducing a composition to a subject, wherein the composition comprises a peptide, wherein the peptide comprises a partial p53 peptide and a MTS. In some aspects, the MTS comprises a Bcl-XL, Bak, or Bax MTS. For example, a Bcl-XL MTS can comprise the amino acid sequence RKGQERFNRWFLTGMTVAGVVLLGSLFSRK (SEQ ID NO:13). A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the MTS is a Bcl-XL, Bak, or Bax MTS. Thus, in some aspects, the Bcl-XL, Bak or Bax MTS consists of SEQ ID NO:13, 15, or 14, respectively.

Disclosed are methods of targeting a composition to mitochondria comprising introducing a composition to a subject, wherein the composition comprises a peptide, wherein the peptide comprises a partial p53 peptide and a MTS, wherein the MTS is not a TOM, OTC, or CCO MTS.

Disclosed are methods of targeting a composition to mitochondria comprising introducing a composition to a subject, wherein the composition comprises a peptide, wherein the peptide comprises a partial p53 peptide and a MTS, wherein the partial p53 peptide consists of the DNA binding domain of p53. In some aspects, the partial p53 peptide consists of amino acids 102-292 of SEQ ID NO:24.

Disclosed are methods of targeting a composition to mitochondria comprising introducing a composition to a subject, wherein the composition comprises a peptide, wherein the peptide comprises a partial p53 peptide and a MTS, wherein the partial p53 peptide comprises the DNA binding domain of p53. In some aspects, the DNA binding domain of p53 consists of amino acids 102-292 of SEQ ID NO:24.

Disclosed are methods of targeting a composition to mitochondria comprising introducing a composition to a subject, wherein the composition comprises a peptide, wherein the peptide comprises a partial p53 peptide and a MTS, wherein the partial p53 peptide comprises the DNA binding domain of p53, wherein the partial p53 peptide further comprises a MDM2 binding domain, a proline-rich domain, a tetramerization domain, or a transactivation domain of p53.

Methods of targeting a composition to mitochondria comprising introducing a composition to a subject disclosed herein, include methods wherein the subject can be human, non-human mammals, or cells. Therefore, introducing a composition to a subject refers to administering the composition to a human subject, a non-human mammal, or to a cell. Introducing a composition to a cell can be in vitro or in vivo administration. In vivo administration or introduction includes indirect introduction of a composition to a cell. For example, a composition administered to a human can be indirectly introduced to the cells within the human body.

Thus, also disclosed are any of the disclosed methods of targeting a composition to mitochondria comprising introducing a composition to a cell. For example, disclosed are methods of targeting a composition to mitochondria comprising introducing a composition to a cell, wherein the composition comprises a peptide, wherein the peptide comprises a full length p53 peptide and a MTS, wherein the MTS comprises a Bak or Bax MTS. In some aspects, the MTS is a Bak, or Bax MTS. Methods of targeting a composition to mitochondria comprising introducing a composition to a cell can occur through indirect introduction to the cell.

Disclosed are methods of targeting a composition to mitochondria comprising introducing any of the disclosed herein compositions, wherein the compositions comprise a nucleic acid sequence capable of encoding one or more of the disclosed peptides. For example, disclosed are methods of targeting a composition to mitochondria comprising introducing a composition to a cell, wherein the composition comprises a nucleic acid sequence capable of encoding a peptide, wherein the peptide comprises a full length p53 peptide and a MTS, wherein the MTS comprises a Bak or Bax MTS. In some aspects, the MTS is a Bak, or Bax MTS.

In some aspects, methods of targeting a composition to mitochondria comprising introducing a composition to a cell, wherein the composition comprises a nucleic acid sequence capable of encoding a peptide, wherein the peptide comprises a partial p53 peptide and a MTS are disclosed

### G. Methods of Inducing Apoptosis

1. Administering a Peptide

Disclosed are methods of inducing apoptosis comprising administering a peptide comprising a full length p53 peptide and a MTS, wherein the MTS is a Bak or Bax MTS. A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). Induction of apoptosis can occur through the Bak or Bax pathway.

Also disclosed are methods of inducing apoptosis comprising administering a peptide comprising a partial p53 peptide and a MTS. In some aspects, the MTS comprises a Bcl-XL, Bak, or Bax MTS. For example, a Bcl-XL MTS can comprise the amino acid sequence RKGQERFNRWFLTGMTVAGVVLLGSLFSRK (SEQ ID NO:13). A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the MTS is a Bcl-XL, Bak, or Bax MTS. For example, the Bcl-XL, Bak, or Bax MTS consists of SEQ ID NO:13, 15, or 14, respectively.

Also disclosed are methods of inducing apoptosis comprising administering a peptide comprising a partial p53 peptide and a MTS, wherein the MTS is not TOM, OTC, or CCO.

Disclosed are methods of inducing apoptosis comprising administering a peptide comprising a partial p53 peptide and a MTS, wherein the partial p53 peptide comprises the DNA binding domain of p53. In some aspects, the DNA binding domain of p53 consists of amino acids 102-292 of SEQ ID NO:24.

Administering a peptide refers to direct or indirect administration of the peptide to a cell. For example, indirect administration can comprise administering the peptide to a subject, wherein the subject comprises a cell. Subjects can comprise humans and non-human mammals.

Disclosed are methods of inducing apoptosis comprising introducing any of the disclosed herein nucleic acid sequences capable of encoding one or more of the disclosed peptides. For example, disclosed are methods of inducing apoptosis comprising introducing a nucleic acid sequence to a cell, wherein the nucleic acid sequence is capable of encoding a peptide, wherein the peptide comprises a full length p53 peptide and a MTS, wherein the MTS comprises a Bak or Bax MTS. In some aspects, the MTS is a Bak, or Bax MTS.

In some aspects, methods of inducing apoptosis comprising introducing a nucleic acid sequence to a cell, wherein the nucleic acid sequence is capable of encoding a peptide, wherein the peptide comprises a partial p53 peptide and a MTS are disclosed.

2. Administering a Composition

Disclosed are methods of inducing apoptosis comprising administering a composition, wherein the composition comprises a peptide, wherein the peptide comprises a full length p53 peptide and a MTS, wherein the MTS is a Bak or Bax MTS. A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). Induction of apoptosis can occur through the Bak or Bax pathway.

Also disclosed are methods of inducing apoptosis comprising administering a composition, wherein the composition comprises a peptide, wherein the peptide comprises a partial p53 peptide and a MTS. In some aspects, the MTS comprises a Bcl-XL, Bak, or Bax MTS. For example, a Bcl-XL MTS can comprise the amino acid sequence RKGQERFNRWFLTGMTVAGVVLLGSLFSRK (SEQ ID NO:13). A Bak MTS can comprise the amino acid sequence GNGPILNVLVVLGVVLLGQFVVRRFFKS (SEQ ID NO:15). A Bax MTS can comprise the amino acid sequence GTPTWQTVTIFVAGVLTASLTIWKKMG (SEQ ID NO:14). In some aspects, the MTS is a Bcl-XL, Bak, or Bax MTS. For example, the Bcl-XL, Bak, or Bax MTS consists of SEQ ID NO:13, 15, or 14, respectively.

Also disclosed are methods of inducing apoptosis comprising administering a composition, wherein the composition comprises a peptide, wherein the peptide comprises a partial p53 peptide and a MTS, wherein the MTS is not a TOM, OTC, or CCO MTS.

Disclosed are methods of inducing apoptosis comprising administering a composition, wherein the composition comprises a peptide, wherein the peptide comprises a partial p53 peptide and a MTS, wherein the partial p53 peptide comprises the DNA binding domain of p53. In some aspects, the DNA binding domain of p53 consists of amino acids 102-292 of SEQ ID NO:24.

Methods of inducing apoptosis comprising administering a composition comprising a peptide refers to direct or indirect administration of the composition to a cell. For example, indirect administration can comprise administering the composition to a subject, wherein the subject comprises a cell. Subjects can comprise humans and non-human mammals.

Disclosed are methods of inducing apoptosis comprising introducing any of the disclosed herein compositions comprising a nucleic acid sequence capable of encoding one or more of the disclosed peptides. For example, disclosed are methods of inducing apoptosis comprising introducing a composition to a cell, wherein the composition comprises a nucleic acid sequence capable of encoding a peptide, wherein the peptide comprises a full length p53 peptide and a MTS, wherein the MTS comprises a Bak or Bax MTS. In some aspects, the MTS is a Bak, or Bax MTS.

In some aspects, methods of inducing apoptosis comprising introducing a composition to a cell, wherein the compositions comprises a nucleic acid sequence capable of encoding a peptide, wherein the peptide comprises a partial p53 peptide and a MTS are disclosed.

### H. Methods of Inducing Homo-Oligomerization

Disclosed are methods of inducing homo-oligomerization of Bak or Bax comprising administering a peptide comprising the DNA binding domain of p53 and a MTS, wherein the MTS is a Bak or Bax MTS. Peptides can comprise a partial p53 peptide or a full length p53 peptide.

Homo-oligomerization of Bak or Bax is a step involved in apoptosis by forming pores in the mitochondrial membrane. Thus, methods of inducing homo-oligomerization of Bak or Bax are useful for aiding in apoptosis.

### I. Methods of Treating Hyperproliferative Disorders

1. Treating with Peptide

Disclosed are methods of treating a hyperproliferative disorder in a patient comprising administering to the patient a peptide, wherein the peptide comprises a full length p53 peptide and a MTS, wherein the MTS comprises a Bak or Bax MTS. In some aspects, the MTS is a Bak, or Bax MTS.

Disclosed are methods of treating a hyperproliferative disorder in a patient comprising administering to the patient a peptide, wherein the peptide comprises a partial p53 peptide and a MTS. For example, the MTS can comprise a Bcl-XL, Bak, or Bax MTS. In some aspects, the MTS is a Bcl-XL, Bak, or Bax MTS.

Disclosed are methods of treating a hyperproliferative disorder in a patient comprising administering to the patient a peptide, wherein the peptide comprises a partial p53 peptide and a MTS, wherein the partial p53 peptide comprises the DNA binding domain of p53.

Disclosed are methods of treating a hyperproliferative disorder in a patient comprising administering to the patient a peptide, wherein the peptide comprises a partial p53 peptide and a MTS, wherein the hyperproliferative disorder is cancer.

Disclosed are methods of treating a hyperproliferative disorder in a patient comprising administering to the patient a peptide, wherein the peptide comprises a full length p53 peptide and a MTS, wherein the MTS comprises a Bak or Bax MTS, wherein the hyperproliferative disorder is cancer. For example, the cancer can be breast or ovarian cancer.

Hyperproliferative disorders include cancer and non-cancer hyperproliferative disorders. Cancers include, but are not limited to brain, lung, squamous cell, bladder, gastric, pancreatic, breast, head, neck, renal, kidney, ovarian, prostate, colorectal, endometrial, esophageal, testicular, gynecological and thyroid cancer. Non-cancer hyperproliferative disorders include, but are not limited to, benign hyperplasia of the skin (e.g., psoriasis), restenosis, or prostate (e.g., benign prostatic hypertrophy (BPH)), age-related macular degeneration, Crohn's disease, cirrhosis, chronic inflammatory-related disorders, proliferative diabetic retinopathy, proliferative vitreoretinopathy, retinopathy of prematurity, granulomatosis, immune hyperproliferation associated with organ or tissue transplantation, an immunoproliferative disease or disorder, e.g., inflammatory bowel disease, rheumatoid arthritis, systemic lupus erythematosus (SLE), vascular hyperproliferation secondary to retinal hypoxia, or vasculitis.

Disclosed are methods of treating a hyperproliferative disorder in a patient comprising administering to the patient a peptide, wherein the peptide comprises a full length p53 peptide and a MTS, wherein the MTS comprises a Bak or Bax MTS, further comprising co-administering an anti-cancer agent. In some aspects, the MTS is a Bak, or Bax MTS.

Disclosed are methods of treating a hyperproliferative disorder in a patient comprising administering to the patient a peptide, wherein the peptide comprises a partial p53 peptide and a MTS. For example, the MTS can comprise a Bcl-XL, Bak, or Bax MTS, further comprising co-administering an anti-cancer agent.

The disclosed methods of treating a hyperproliferative disorder in a patient further comprising co-administering an anti-cancer agent, wherein the anti-cancer agent comprises paclitaxel or carboplatin. Anti-cancer agents are compounds useful in the treatment of cancer. Examples of anti-cancer agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-I1 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosf amide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gammall and calicheamicin omegall (see, e.g., Agnew, Chem Intl. Ed. Engl., 33: 183-186 (1994)); dynemicin, including dynemicin A; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including ADRIAMYCIN®, morpholino-doxorubicin, cyanomorpholino-doxorubicin, 2-pyrrolino-doxorubicin, doxorubicin HCl liposome injection (DOXIL®) and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate, gemcitabine (GEMZAR®), tegafur (UFTORAL®), capecitabine (XELODA®), an epothilone, and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethane; vindesine (ELDISEME®, FILDESIN®); dacarbazine; mannomustine; mitobronitol; mitolactol; pipobroman; gacytosine; arabinoside (“Ara-C”); thiotepa; taxoids, e.g., paclitaxel (TAXOL®), albumin-engineered nanoparticle formulation of paclitaxel (ABRAXANE™), and doxetaxel (TAXOTERE®); chloranbucil; 6-thioguanine; mercaptopurine; methotrexate; platinum analogs such as cisplatin and carboplatin; vinblastine (VELB AN®); platinum; etoposide (VP-16); ifosf amide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Any of the disclosed peptides can be used in the methods of treating a hyperproliferative disorder.

2. Treating with Nucleic Acid Sequence

Disclosed are methods of treating a hyperproliferative disorder in a patient comprising administering to the patient a nucleic acid sequence, wherein the nucleic acid sequence is capable of encoding a full length p53 peptide and a MTS, wherein the MTS comprises a Bak or Bax MTS. In some aspects, the MTS is a Bak, or Bax MTS.

Also disclosed are methods of treating a hyperproliferative disorder in a patient comprising administering to the patient a nucleic acid sequence, wherein the nucleic acid sequence is capable of encoding a partial p53 peptide and a MTS. The MTS can comprise a Bcl-XL, Bak or Bax MTS.

Methods of treating a hyperproliferative disorder in a patient comprising administering to the patient a nucleic acid sequence can comprise administering the nucleic acid sequence to the patient using a viral vector. Viral vectors include, but are not limited to, an adenoviral vector, lentiviral vector, and adeno-associated vectors.

Methods of treating hyperproliferative disorders comprise administering any of the disclosed nucleic acid sequences.

3. Treating with Compositions

Disclosed are methods of treating a hyperproliferative disorder in a patient comprising administering to the patient a composition, wherein the composition comprises a peptide, wherein the peptide comprises a full length p53 peptide and a MTS, wherein the MTS comprises a Bak or Bax MTS. In some aspects, the MTS is a Bak, or Bax MTS.

Disclosed are methods of treating a hyperproliferative disorder in a patient comprising administering to the patient a composition, wherein the composition comprises a peptide, wherein the peptide comprises a partial p53 peptide and a MTS. For example, the MTS can comprise a Bcl-XL, Bak, or Bax MTS. In some aspects, the MTS is a Bcl-XL, Bak, or Bax MTS.

Disclosed are methods of treating a hyperproliferative disorder in a patient comprising administering to the patient a composition, wherein the composition comprises a peptide, wherein the peptide comprises a partial p53 peptide and a MTS, wherein the partial p53 peptide comprises the DNA binding domain of p53.

Disclosed are methods of treating a hyperproliferative disorder in a patient comprising administering to the patient a composition, wherein the composition comprises a peptide, wherein the peptide comprises a partial p53 peptide and a MTS, wherein the hyperproliferative disorder is cancer.

Disclosed are methods of treating a hyperproliferative disorder in a patient comprising administering to the patient a composition, wherein the composition comprises a peptide, wherein the peptide comprises a full length p53 peptide and a MTS, wherein the MTS comprises a Bak or Bax MTS, wherein the hyperproliferative disorder is cancer. For example, the cancer can be breast or ovarian cancer. In some aspects, the MTS is a Bak, or Bax MTS.

Also disclosed are methods of treating a hyperproliferative disorder in a patient comprising administering to the patient a composition, wherein the composition comprises a nucleic acid sequence, wherein the nucleic acid sequence is capable of encoding a full length p53 peptide and a MTS, wherein the MTS comprises a Bak or Bax MTS.

Also disclosed are methods of treating a hyperproliferative disorder in a patient comprising administering to the patient a composition, wherein the composition comprises a nucleic acid sequence, wherein the nucleic acid sequence is capable of encoding a partial p53 peptide and a MTS. The MTS can comprise a Bcl-XL, Bak or Bax MTS.

Methods of treating a hyperproliferative disorder in a patient comprising administering to the patient a composition, wherein the composition comprises a nucleic acid sequence can comprise administering the nucleic acid sequence to the patient using a viral vector. Viral vectors include, but are not limited to, an adenoviral vector, lentiviral vector, and adeno-associated vectors.

Methods of treating hyperproliferative disorders comprise administering any of the disclosed compositions comprising any of the disclosed nucleic acid sequences.

### J. Cells

Also disclosed herein are host cells transformed or transfected with an expression vector comprising the nucleic acid sequences described elsewhere herein. Also disclosed are host cells comprising the expression vectors described herein. For example, disclosed is a host cell comprising an expression vector comprising the nucleic acid sequences described elsewhere herein, operably linked to a control element. Host cells can be eukaryotic or prokaryotic cells. For example, a host cell can be a mammalian cell. Also disclosed are recombinant cells comprising the disclosed nucleic acid sequences or peptides. Further disclosed are recombinant cells producing the disclosed peptides.

Disclosed are recombinant cells comprising one or more of the nucleic acid sequences disclosed herein.

Disclosed are recombinant cells comprising one or more of the nucleic acid sequences capable of producing any of the peptides disclosed herein.

For example, disclosed are T47D, H1373, SKOV-3 and HeLa cells comprising one or more of the nucleic acid sequences disclosed herein. Further disclosed are T47D, H1373, SKOV-3 and HeLa cells comprising one or more of the nucleic acid sequences capable of producing any of the peptides disclosed herein.

### K. Transgenics

Disclosed are transgenic, non-human subjects comprising the nucleic acid sequences disclosed herein which are capable of encoding the peptides disclosed herein. For example, disclosed are transgenic, non-human subjects comprising a nucleic acid sequence, wherein the nucleic acid sequence is capable of encoding a full length p53 peptide and a MTS, wherein the MTS comprises a Bak or Bax MTS. A nucleic acid sequence capable of encoding a Bak MTS can comprise 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:26). A nucleic acid sequence capable of encoding a Bax MTS can comprise 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:23). In some aspects, the MTS is a Bak or Bax MTS. For example, in some aspects the Bak or Bax MTS is encoded by SEQ ID NO:26 or 23, respectively.

Also disclosed are transgenic, non-human subjects comprising nucleic acid sequences, wherein the nucleic acid sequences are capable of encoding a partial p53 peptide and a MTS. Disclosed are nucleic acid sequences, wherein the nucleic acid sequences are capable of encoding a partial p53 peptide and a MTS, wherein the MTS is not a TOM, OTC, or CCO MTS.

Disclosed are transgenic, non-human subjects comprising nucleic acid sequences, wherein the nucleic acid sequences are capable of encoding a partial p53 peptide and a MTS, wherein the partial p53 peptide comprises the DNA binding domain of p53. In some aspects, the DNA binding domain of p53 consists of amino acids 102-292 of SEQ ID NO:24. For example, the DNA binding domain of p53 can be SSSVPSQ KTYQGSYGFR LGFLHSGTAK SVTCTYSPAL NKMFCQLAKT CPVQLWVDST PPPGTRVRAM AIYKQSQHMT EVVRRCPHHE RCSDSDGLAP PQHLIRVEGN LRVEYLDDRN TFRHSVVVPY EPPEVGSDCT TIHYNYMCNS SCMGGMNRRP ILTIITLEDS SGNLLGRNSF EVRVCACPGR DRRTEEENLR KKGE (SEQ ID NO:25).

Disclosed are transgenic, non-human subjects comprising nucleic acid sequences, wherein the nucleic acid sequences are capable of encoding a partial p53 peptide and a MTS, wherein the MTS comprises a Bcl-XL, Bak, or Bax MTS. Nucleic acid sequences capable of encoding a MTS can comprise the sequences of 5′AGAAAGGGCCAGGAGAGATTCAACAGATGGTTCCTGACCGGCATGACCGTGGC CGGCGTGGTGCTGCTGGGCAGCCTGTTCAGCAGAAAGTGA-3′ (SEQ ID NO:27), 5′GATCCTCCTACTTTGGGACGCCCACGTGGCAGACCGTGACCATCTTTGTGGCGG GAGTGCTCACCGCCTCACTCACCATCTGGAAGAAGATGGGCTGAG-3′ (SEQ ID NO:26), or 5′GATCCGGCAATGGTCCCATCCTGAACGTGCTGGTGGTTCTGGGTGTGGTTCTGT TGGGCCAGTTTGTGGTACGAAGATTCTTCAAATCATGAG-3′ (SEQ ID NO:23). In some aspects, the MTS is a Bcl-XL, Bak or Bax MTS. For example, in some aspects the Bcl-XL, Bak, or Bax MTS is encoded by SEQ ID NO:27, 26, or 23, respectively.

### L. Antibodies

Disclosed are monoclonal antibodies that specifically bind to any of the disclosed peptides herein. For example, disclosed are monoclonal antibodies that specifically bind to a peptide comprising a full length p53 peptide and a mitochondrial targeting signal (MTS), wherein the MTS is a Bak or Bax MTS.

Disclosed are monoclonal antibodies that specifically bind to a peptide comprising a partial p53 peptide and a MTS. For example, disclosed are monoclonal antibodies that specifically bind to a peptide comprising a partial p53 peptide and a MTS, wherein the MTS comprises a Bcl-XL, Bak, or Bax MTS. In some aspects, the MTS is a Bak, or Bax MTS.

### M. Kits

The materials described above as well as other materials can be packaged together in any suitable combination as a kit useful for performing, or aiding in the performance of, the disclosed method. It is useful if the kit components in a given kit are designed and adapted for use together in the disclosed method. For example disclosed are kits for producing vectors, the kit comprising any of the disclosed nucleic acid sequences. The kits also can contain a viral vector.

### EXAMPLES

### N. Example 1

### The DNA Binding Domain of p53 is Sufficient to Trigger a Potent Apoptotic Response at the Mitochondria

1. Introduction

The tumor suppressor p53 is one of the most commonly mutated genes in all cancers. Although nuclear-mediated transcriptional activity has been extensively characterized, mitochondrial targeting of p53 has yet to be fully exploited as a therapeutic approach. The main advantage of targeting p53 to the mitochondria is its ability to trigger a rapid apoptotic response, while in the nucleus p53 first has to form a tetramer, bind to DNA, and initiate transcription of various apoptotic genes. As a consequence of stress, p53 translocates to the mitochondria and initiates apoptosis through mitochondrial outer membrane permeabilization (MOMP). Mitochondrial p53 directly interacts with anti- and pro-apoptotic members of the Bcl-2 family of proteins located in the mitochondrial outer membrane. In apoptosis resistant cells, the anti-apoptotic members, Bcl-XL, Bcl-2 and Mcl-1 form heterodimers with pro-apoptotic proteins Bak and Bax, preventing apoptosis. To trigger MOMP, p53 binds to Bcl-XL, Bcl-2 and Mcl-1 and frees pro-apoptotic Bak and Bax allowing them to oligomerize. Homo-tetramer formation of Bak and Bax in the mitochondrial outer membrane triggers the release of various pro-apoptotic proteins such as cytochrome c. APAF-1 and cytochrome c form the apoptosome and activate caspase-9 that can initiate the caspase cascade resulting in programmed cell death.

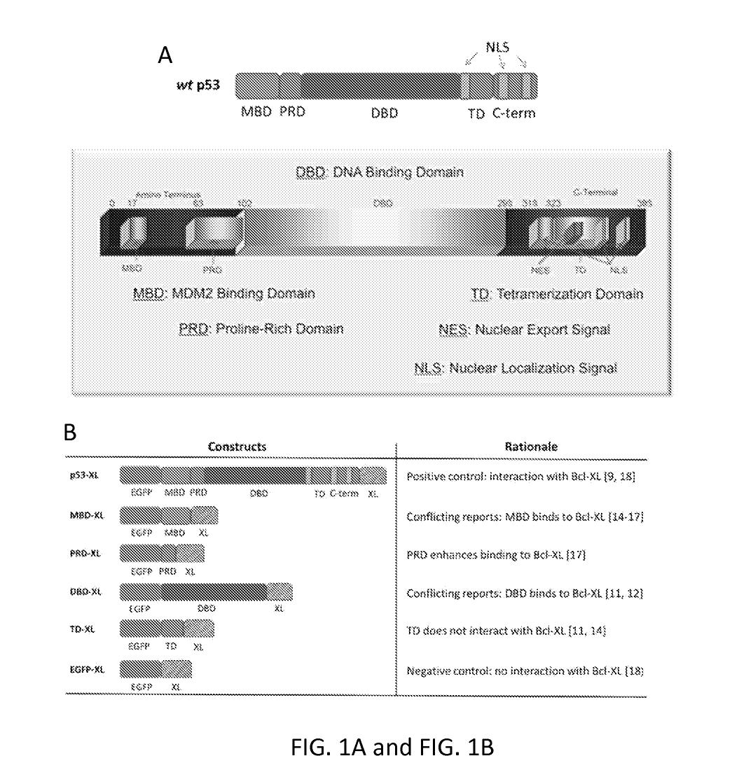

It is unclear which domains of p53 are directly responsible for triggering apoptosis at the mitochondria, presumably by interacting with anti-apoptotic Bcl-XL. The structure of p53 can be divided into amino terminus, DNA binding domain (DBD) and C-terminal region (FIG. 1A). The amino terminus consists of the MDM2 binding domain (MBD) and the proline-rich domain (PRD). The C-terminal region encloses the tetramerization domain (TD) and three nuclear localization signals (NLS) (FIG. 1A). It has been reported that the DBD binds to anti-apoptotic Bcl-XL in the mitochondrial outer membrane and the PRD functions as an enhancer that improves this binding. However, the MBD has been also proposed as a binding partner for Bcl-XL which is enhanced by the PRD.

This study shows that a smaller domain of p53 is capable of inducing apoptosis similar to full length p53 when targeted to the mitochondria. This involved the fusing of different domains of p53 (MBD, PRD, DBD, TD) to the mitochondrial targeting signal (MTS) from Bcl-XL (abbreviated XL) to ensure mitochondrial targeting (FIG. 1B).

2. Materials and Methods

i. Cell Lines and Transient Transfections

1471.1 murine adenocarcinoma cells (gift of G. Hager, NCI, NIH), T47D human ductal breast epithelial tumor cells (ATCC, Manassas, Va.), MCF-7 human breast adenocarcinoma cells (ATCC), MDA-MB-231 human breast adenocarcinoma cells, HeLa human epithelial cervical adenocarcinoma cells (ATCC), and H1373 human non-small lung carcinoma were grown as monolayers in DMEM (1471.1) and RPMI (T47D, MCF-7, MDA-MB-231, HeLa, H1373) (Invitrogen, Carlsbad, Calif.) supplemented with 10% FBS (Invitrogen), 1% penicillin-streptomycin (Invitrogen), 1% glutamine (Invitrogen) and 0.1% gentamycin (Invitrogen). T47D and MCF-7 cells were additionally supplemented with 4 mg/L insulin (Sigma, St. Louis, Mo.). Cells were maintained in a 5% CO2 incubator at 37° C. 3.0×105 cells for T47D and MCF-7 cells, 1.0×105 cells for MDA-MB-231 and HeLa, 2.0×105 for H1373 were seeded in 6-well plates (Greiner Bio-One, Monroe, N.C.). Different amounts of cells were plated to account for varying cell growth rates in order to maximize transfection efficiency. Approximately 24 h after seeding, transfection was performed using 1 pmol of DNA per well and Lipofectamine 2000 (Invitrogen) following the manufacturer's recommendations.

ii. Plasmid Construction

The main plasmids used in this work are depicted in FIG. 1B.

pEGFP-p53ΔC-XL (p53ΔC-XL): The DNA encoding p53ΔC (amino acids 1-322), a truncated version of wt-p53 that lacks the C-terminus, was amplified via PCR with the primers 5′-GCGCGCGCGCTCCGGAATGGAGGAGCCGCAGTCA-3′ (SEQ ID NO:1) and 5′-GCGCGCGCGCGGTACCTCATGGTTTCTTCTTTGGCTGGGG-3′ (SEQ ID NO:2) using previously subcloned pEGFP-p53 as the template DNA. p53ΔC was cloned into pEGFP-XL (E-XL) using BspEI and KpnI sites.

pEGFP-DBD-XL (DBD-XL): The DNA encoding the DBD was amplified via PCR from pEGFP-p53-XL (p53-XL) using 5′-CCGGGCCCGCGGTCCGGAACCTACCAGGGCAGCTACG-3′ (SEQ ID NO:3) and 5′-CCGGGCCCGCGGGGTACCTTTCTTGCGGAGATTCTCTTCCT-3′ (SEQ ID NO:4) and cloned into E-XL using BspEI and KpnI sites.

pEGFP-PRD-DBD-XL (PRD-DBD-XL): The DNA encoding the PRD-DBD was amplified using PCR from p53-XL with the primers 5′-GCGCGCGCGCGGTACCGCTCCCAGAATGCCAGAGGC-3′ (SEQ ID NO:5) and 5′-GCGCGCGCGCGGATCCTTTCTTGCGGAGATTCTCTT-3′ (SEQ ID NO:6) and cloned into E-XL (18) at the KpnI and BamHI site.

pEGFP-TD-XL (TD-XL): The DNA encoding the TD was amplified via PCR from previously subcloned p53-XL using 5′-GCGCGCGCGCGGGATCCGGCTGGATGGAGAATATTTCACCCTTCA-3′ (SEQ ID NO:7) and 5′-GCGCGCGCGCGGGAtCCTCACCCAGCCTGGGCATCCTT-3′ (SEQ ID NO:8) and cloned into E-XL (18) at the BamHI site.

pEGFP-MBD-PRD-XL (MBD-PRD-XL): Previously subcloned p53-XL was mutated via site-directed mutagenesis using the QuikChange II XL Site directed Mutagenesis Kit (Agilent, Santa Clara, Calif.) using 5′ TCCCTTCCCAGAAAAGGTACCAGGGCAGCTACGGT-3′ (SEQ ID NO:9) and its reverse complement to introduce an additional KpnI site (mutations underlined). Then the DBD and C-terminus were digested out using KpnI. Additionally, a frame shift mutation was corrected (one base pair deletion) by mutating the cloned plasmid using 5′-TCGAGCTATGGAAACATTTTCAGACCTATGGAAACTACTTCCTGAACGGAATTCT G-3′ (SEQ ID NO:10) and its complementary strand via site-directed mutagenesis.

pEGFP-PRD-XL (PRD-XL): MBD-PRD-XL was mutated via site-directed mutagenesis using 5′ TTCACTGAAGACCCAGGTCCATCCGGAGCTCCCAGAATGCCAGA-3′ (SEQ ID NO:11) and its complementary strand to introduce an additional BspEI site. The MBD was cut out with BspEI to create PRD-XL

pEGFP-CC (E-CC): pEGFP-CC was subcloned as before.

pBFP-Bcl-XL (BFP-Bcl-XL): Bcl-XL was digested out from pSFFV-neo-Bcl-XL with EcoRI and cloned into the EcoRI site of the pTagBFP-C vector (Evrogen, Moscow, Russia). A frame shift mutation was conducted (one base pair addition) by mutating the cloned plasmid using 5′-TCTCGAGCTCAAGCTTCGAATTCATTGGACAATGG-3′ (SEQ ID NO:12) and its complementary strand via site-directed mutagenesis.

iii. Mitochondrial Staining, Microscopy, and Image Analysis

Before live-cell imaging and mitochondrial staining of transfected cells was performed, media in live cell chambers was replaced with phenol red-free DMEM (Invitrogen) for 1471.1 cells or phenol red-free RPMI (Invitrogen) for T47D and MCF-7 cells containing 10% charcoal stripped fetal bovine serum (CS-FBS, Invitrogen). Cells were incubated with 150 nM MitoTracker Red FM (Invitrogen) for 15 min at 37° C. and protected from light. As previously described, images were acquired using an Olympus IX71F fluorescence microscope (Scientific Instrument Company, Aurora, Colo.) with high quality (HQ) narrow band GFP filter (ex, HQ480/20 nm; em, HQ510/20 nm) and HQ:TRITC filter (ex, HQ545/30; em, HQ620/60) from Chroma Technology (Brattleboro, Vt.) with a 40× PlanApo oil immersion objective (NA 1.00) on an F-View Monochrome CCD camera.

ImageJ software and JACoP plugin was used to analyze images for mitochondrial stain overlap with EGFP fusion constructs. As previously, JACoP was used to generate the colocalization statistic [i.e., Pearson's correlation coefficient (PCC) post Costes' automatic threshold algorithm]. PCC evaluates correlation between pairs of individual pixels from EGFP and MitoTracker stained cells. The higher the PCC value, the higher the correlation. According to Costes a PCC value of 0.6 or greater determines colocalization between a cellular compartment and the designed protein. Spatial representations of pixel intensity correlation have been generated using Colocalization Colormap (ImageJ) for increased visual clarity of mitochondrial localization of the EGFP-fused constructs. Microscopy was repeated in triplicate (n=3), and 10 cells were analyzed for each construct.

iv. 7-AAD Assay

Transfected T47D, MCF-7, MDA-MB-231, HeLa and H1373 cells were pelleted and resuspended in 500 μL PBS (Invitrogen) containing 1 μM 7-aminoactinomycin D (7-AAD) (Invitrogen) for 30 min prior to analysis following the recommended protocol from the manufacturer. The assay was performed 48 h after transfection for T47D, MCF-7 and H1373 and 24 h after transfection for MDA-MB-231 and HeLa. Only EGFP positive cells were analyzed by using the FACS Canto-II (BD-BioSciences) with FACS Diva software. EGFP and 7-AAD were excited with the 488 nm laser, and were detected at 507 nm and 660 nm, respectively. Independent transfections of each construct were tested three times (n=3).

v. Annexin V Assay

48 h after transfection, T47D cells were pelleted and resuspended in 400 μL of annexin-V binding buffer (Invitrogen) and incubated with 5 μL of annexin-APC (annexin-V conjugated to allophycocyanin, Invitrogen) for 15 min as before. Only transfected cells were analyzed as mentioned in 7-AAD assay. EGFP and APC were excited at 488 nm and 635 nm wavelengths, respectively and detected at their corresponding 507 nm and 660 nm wavelengths. Independent transfections of each construct were tested three times (n=3).

vi. TUNEL Assay

T47D cells were harvested 48 h after transfection. In situ Death Detection Kit, TMR red (Roche, Mannheim, Germany) was used following manufacturer's recommendations as before. Cells were resuspended in PBS (Invitrogen) and analyzed via the FACSAria-II (BD-Biosciences, University of Utah Core Facility). EGFP and TMR red were excited at 488 nm and 563 nm, respectively, and FACSDiva software was used to analyze the data. Independent transfections of each construct were tested three times (n=3).

vii. Colony Forming Assay (CFA)

Transfected T47D cells were harvested 24 h post transfection and resuspended in RPMI (Invitrogen) at a concentration of 3.0×105 cells/mL. The Cytoselect® 96-well cell transformation assay (Cell Biolabs, San Diego, Calif.) was used following manufacturer's recommendations. Equal amount of 1.2% Agar Solution, 2×DMEM/20% FBS media, and cell suspension (1:1:1) were mixed and 75 μL of the mixture was added to a 96-well plate containing a solidified base agar layer (50 μL of previously solidified 1.2% Agar Solution), and allowed to solidify at 4° C. for 15 min. The following steps were performed according to the manufacture's recommendations. A Spectra Max M2 plate reader (Molecular Devices, Sunnyvale, Calif.) was used to detect fluorescence using a 485/520 nm filter set. Independent transfections of each construct were tested three times (n=3).

viii. TMRE Assay

36 h after transfection T47D cells were incubated with 100 nM tetramethylrhodamineethylester (TMRE) (Invitrogen) for 30 min at 37° C. T47D cells were pelleted and resuspended in 300 μL annexin-V binding buffer (1×) (Invitrogen). Only EGFP positive cells were analyzed by using the FACS Canto-II (BD-BioSciences) with FACS Diva software. EGFP was excited with the 488 nm laser with emission filter 530/35 and TMRE was excited with the 561 nm laser with the emission filter 585/15. Mitochondrial depolarization (loss in TMRE intensity) correlates with an increase in MOMP. Independent transfections of each construct were tested three times (n=3).

ix. Caspase-9 Assay

T47D cells were probed 48 h after transfection using SR FLICA Caspase-9 Assay Kit (Immunochemistry Technologies, Bloomington, Minn.). Cells were incubated with SR FLICA Caspase-9 reagent for 60 min per manufacturer's recommendations, pelleted and resuspended in 300 μL 1× wash buffer (Immunochemistry Technologies). Only EGFP positive cells were analyzed by using the FACS Canto-II (BD-BioSciences) with FACS Diva software. EGFP and FLICA were excited with the 488 nm (emission filter 530/35) and the 561 laser (emission filter 585/15), respectively. Independent transfections of each construct were tested three times (n=3).

x. Co-Immunoprecipitation (Co-IP)

Anti-GFP antibody (ab290, Abcam) was coupled to dynabeads using Dynabeads Antibody Coupling Kit (Invitrogen). 24 h post transfection, T47D cells were prepared using the Dynabeads Co-Immunoprecipitation Kit (Invitrogen). Cell pellets were lysed using extraction buffer B (1×IP, 100 nM NaCl, 2 mM MgCl2, 1 mM DTT, 1% protease inhibitor). The lysate was incubated for 30 min at 4° C. with 1.5 mg of dynabeads coupled with anti-GFP antibody, and co-IP was performed per the company's protocol. The final protein complex was denatured and western blot was performed by using Bcl-XL antibody (ab 2568, Abcam).

xi. Rescue Experiment Using BFP-Bcl-XL

T47D cells were co-transfected with 1 pmol of EGFP constructs and 1 pmol of BFP-Bcl-XL (BFP tag is necessary for gating Bcl-XL transfected cells). 48 h after transfection the 7-AAD assay was performed as described above. FACSCanto-II (BD-BioSciences) and FACSDiva software were used for EGFP and BFP gating. Excitation was set at 488 nm, and detected at 507 nm and 660 nm for EGFP and 7-AAD, respectively. BFP was excited at 405 nm and detected at 457 nm. Independent transfections of each construct were tested three times (n=3).

xii. Statistical Analysis:

All experiments were conducted in a triplicate (n=3). Statistical significance was determined by one-way analysis of variance (ANOVA) with Tukey's or Bonferroni's post test as indicated in figure legends; Student t-test was used to analyze the rescue experiment data. The degree of colocalization was analyzed using odds ratio with Pearson's Chi-square. A p value <0.05 was considered significant.

3. Results

i. Mitochondrial Localization of Single Domain Constructs

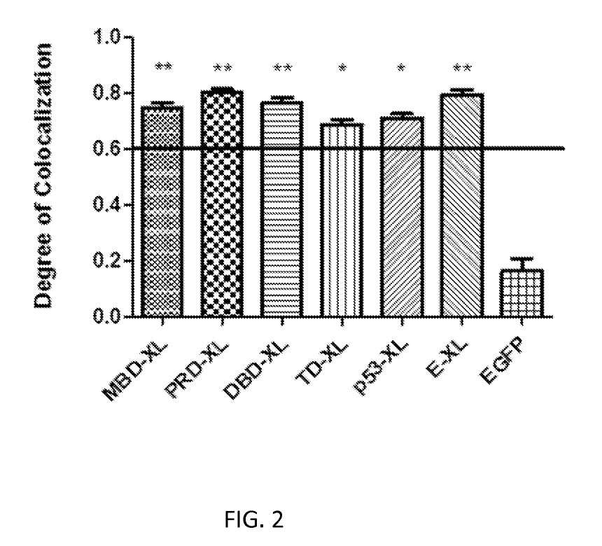

Different domains of p53 (FIG. 1B) were fused to the MTS from Bcl-XL (abbreviated XL) and tested for their mitochondrial localization. Mitochondrial targeting of these constructs (FIG. 1) was determined by fluorescence microscopy as previously. It was shown that 1471.1 cells, which are large in size, spread well, and are optimal for microscopy. However, similar microscopy results were observed in T47D cells. FIG. 2 shows colocalization of the EGFP fused constructs with mitochondria which were generated using Pearson's correlation coefficient (PCC) following the example of Bolte and Cordelières and graphed for each construct. PCC values range from +1 (perfect correlation) to −1 (anticorrelation), and a PCC value of zero represents random distribution. Costes et al. have shown that a PCC of 0.6 or greater defines colocalization, or co-compartmentalization (FIG. 2B). FIG. 2 shows that all designed single domain constructs translocate into the mitochondria, as expected. EGFP served as negative control for colocalization analysis, and there was no colocalization between EGFP alone and the mitochondria. Even though p53 is a nuclear protein containing three nuclear localization signals (NLSs), the XL MTS is strong enough to overcome nuclear targeting and directs p53-XL to the mitochondria (FIG. 1).

ii. Screening the Mitochondrial Activity of Different p53 Domains Via 7-AAD

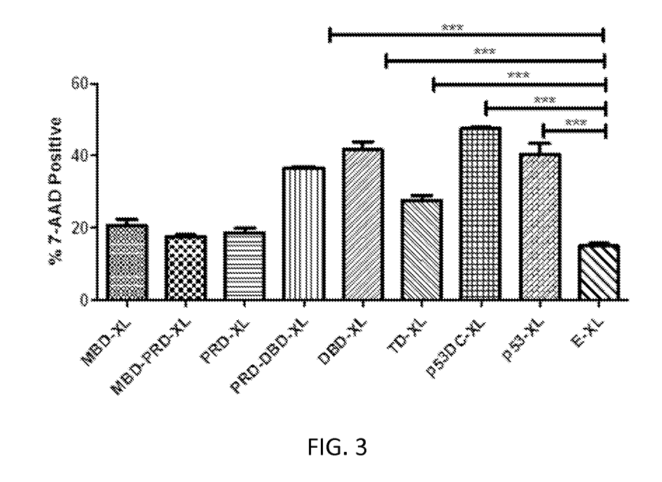

To determine if a subdomain of p53 was capable of evoking a similar apoptotic activity as wild type p53, different domains of p53 (FIG. 1B) fused to XL and combinations of them were tested for apoptosis using the 7-AAD viability assay in T47D human breast cancer cells. 7-AAD is a late apoptosis/necrosis assay which allows for distinguishing between apoptotic/necrotic (ruptured plasma membrane) and healthy (intact plasma membrane) cells. If the plasma membrane is disrupted, the 7-AAD dye intercalates with nuclear DNA of apoptotic/necrotic cells. FIG. 3 demonstrates that all constructs containing DBD (PRD-DBD-XL, DBD-XL, and p53ΔC-XL) are statistically higher than the negative control E-XL. Additionally, these three constructs are not statistically different from p53-XL (positive control) indicating that all constructs containing the DBD show similar apoptotic potential to p53-XL (FIG. 3). Further, MBD-XL, MBD-PRD-XL, and PRD-XL are not statistically significant from the negative control E-XL indicating no apoptotic activity (FIG. 3). Interestingly, TD-XL is statistically different from the negative controls but is also significantly lower than p53-XL (FIG. 3).

Data from FIG. 3 illustrate that there is no difference in activity between the single domains (MBD-, PRD-, DBD-, TD; 1^{st}, 3^{rd}, 5^{th}, 6^{th} bars, respectively) versus combinations of the domains (MBD-PRD, PRD-DBD, p53ΔC; 2^{nd}, 4^{th}, 7^{th} bars, respectively) when fused to XL. Therefore, the single domain constructs (i.e. MBD-XL, PRD-XL, DBD-XL, and TD-XL) were used for the remaining experiments.

iii. Exploring the Apoptotic Potential of Designed Constructs

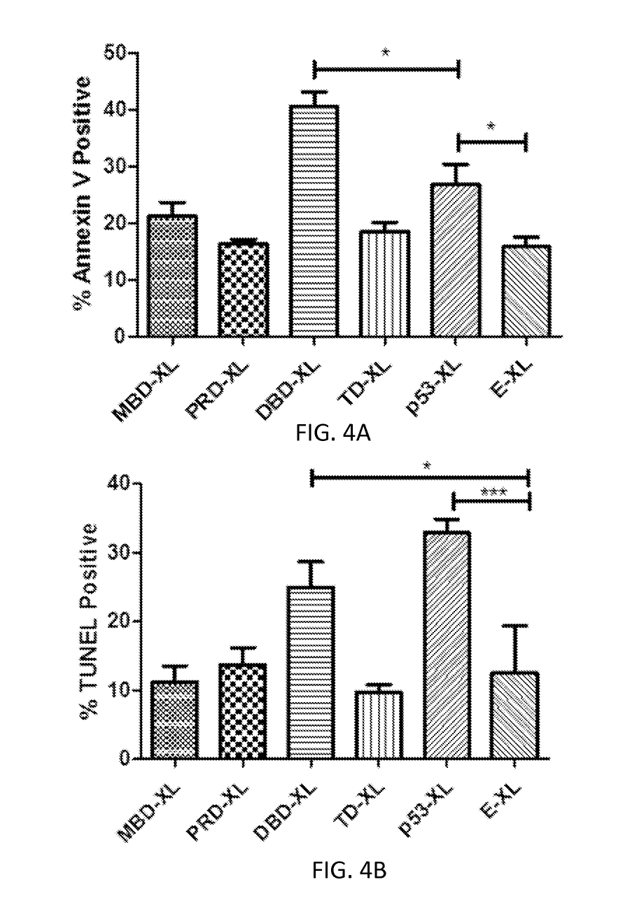

To test the apoptotic potential of the designed single domain constructs (FIG. 1B), the externalization of phoshatidylserine on the cell surface of apoptotic cells was measured via annexin V staining 48 h after transfection. DBD-XL showed a significantly higher apoptotic response than p53-XL (FIG. 4A). Additionally, both constructs were significantly higher than the negative control E-XL whereas MBD-XL, PRD-XL and TD-XL were not statistically significant from the negative control (FIG. 4A).

Further, the fragmentation of nuclear DNA was measured utilizing terminal deoxynucleotidyl transferase dUTP labeling (TUNEL) which tags the terminal end of nucleic acids. DNA fragmentation occurs when a cell is undergoing apoptosis, and the cellular DNA is cleaved by caspases. The TUNEL assay was conducted 48 h after transfection. FIG. 4B shows that both p53-XL and DBD-XL have similar activities and are significantly higher than E-XL while MBD-XL, PRD-XL and TD-XL are not statistically different from the negative control.

iv. Testing the Oncogenic Potential

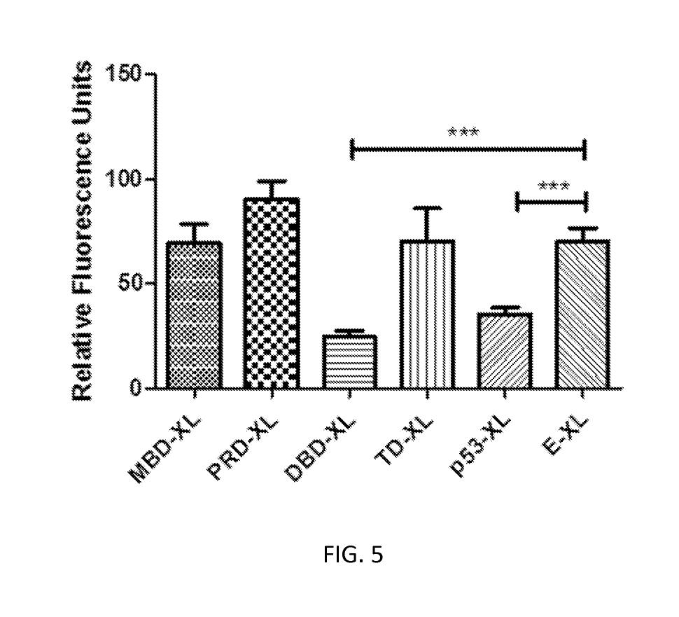

To test the potential of designed constructs to inhibit the transforming ability of cancer cells, a colony forming assay was carried out in T47D cells eight days after treatment. As expected, p53-XL and DBD-XL showed significant decrease in transformative ability of T47D cells represented by fewer colonies (reduction in relative fluorescence units) compared to E-XL (FIG. 5). All the other small domain constructs failed to reduce cell proliferation similar to the negative control E-XL.

v. The Ability of DBD-XL to Induce Late Stage Apoptosis is not Cell Line Specific

To ensure that the ability of DBD-XL to induce apoptosis is not cell line- or cancer cell type specific, a 7-AAD assay was conducted in breast cancer cells (MCF-7, MDA-MB-231), cervical adenocarcinoma cells (HeLa) and human non-small cell lung adenocarcinoma (H1373). T47D and MDA-MB-231 both express mutant p53, with the mutations restricted to the DBD (L194F in T47D and R280L in MDA-MB-231). These mutations reduce the activity of tumor suppressor activity substantially and cause these cells to be more resistant to apoptosis than MCF-7 and HeLa. Additionally, MCF-7 harbor mislocalized p53 in the cytoplasm, HeLa have endogenous wt-p53 and H1373 are p53 null.

Since MDA-MB-231 and HeLa are highly proliferating cells, both cell lines were assayed 24 h after transfection while T47D, MCF-7 and H1373 cells were assayed 48 h post transfection (optimal time points determined empirically).

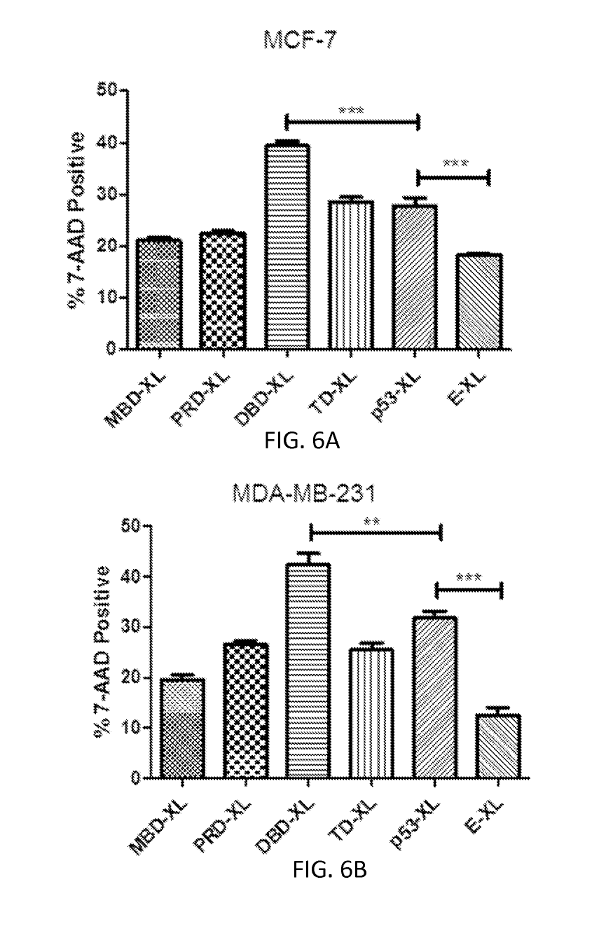

Interestingly, DBD-XL showed significantly higher apoptotic activity compared to p53-XL in MCF-7 (FIG. 6A), MDA-MB-231(FIG. 6B) and H1373 (FIG. 6D). In HeLa cells, DBD-XL (and PRD-XL) were both statistically significant from p53-XL (FIG. 5C). These results are consistent with the apoptosis data from T47D cells (FIGS. 3, 4A, and 4B) and show that DBD-XL is capable of inducing late stage apoptosis in four cell lines which differ in their endogenous p53 status, similarly to p53-XL.

vi. The Apoptotic Activity of DBD-XL is Triggered Via the Mitochondrial/Intrinsic Pathway

To determine that constructs-induced apoptotic effects are through the mitochondria TMRE and caspase-9 assays were performed. The DBD-XL was the only single domain construct that showed the same or higher apoptotic potential as p53-XL consistently in all tested cell lines. Therefore, only the mechanism for DBD-XL compared to p53-XL was investigated for further studies.

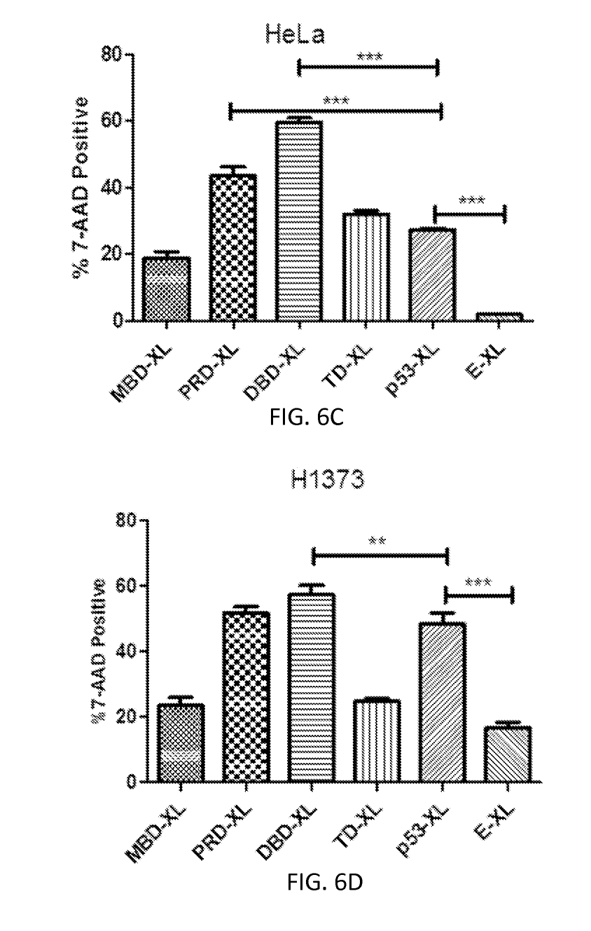

To ensure that DBD-XL causes MOMP in the same manner as p53-XL, the TMRE assay (analogous to the JC-1 assay) was used. TMRE is a cell-permeant, cationic, red-orange fluorescent dye that rapidly accumulates in mitochondria of living cells due to the negative mitochondrial membrane potential (ΔΨm) of intact mitochondria compared to cytosol. Mitochondrial depolarization results in a loss of TMRE from mitochondria and a decrease in mitochondrial fluorescence intensity (FI). The mitochondrial membrane permeabilization (loss of FI) was illustrated as % MOMP induction on the y-axis. DBD-XL and p53-XL have similar activity and are significantly higher than E-XL (FIG. 7A).

Further the activation of caspase-9 was measured. Caspase-9 is only triggered through the intrinsic apoptotic pathway. Once cytochrome c is released from the mitochondria, caspase-9 is the first effector caspase downstream of cytochrome c. Caspase-9 itself cleaves the peptide sequence leucine-glutamic acid-histidine-aspartic acid (LEHD) which is used in the caspase-9 assay to measure the intrinsic apoptotic pathway. DBD-XL and p53-XL show higher caspase-9 activation than E-XL (FIG. 7B). However, p53-XL triggers caspase-9 activation significantly more than DBD-XL (FIG. 7B).

vii. Investigating the Apoptotic Mechanism Via Co-IP and Overexpression of Bcl-XL

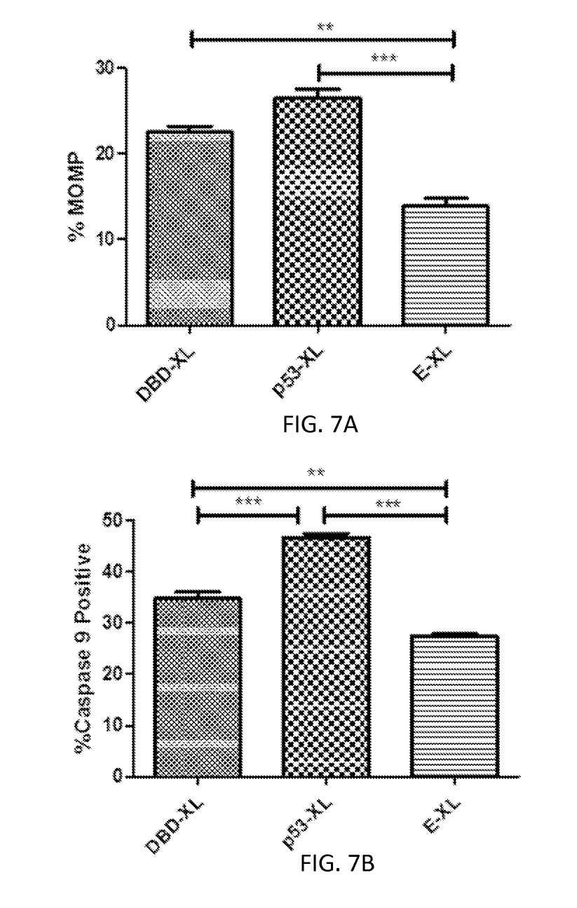

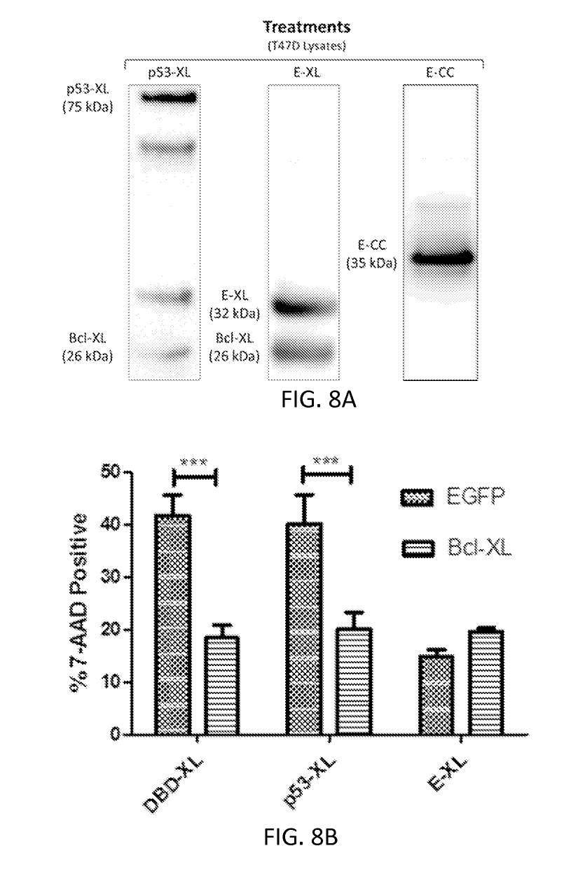

To explore the apoptotic mechanism of the constructs, a co-IP was conducted (FIG. 8A). p53-XL, E-XL and E-CC (a negative control that does not contain the XL signal) were transfected into T47D cells. T47D cells express the highest amount of endogenous Bcl-XL protein compared to MCF-7, MDA-MB-231 and HeLa (FIG. 9). Approximately 24 h after transfection cells were lysed and incubated with anti-GFP antibody. A western blot was performed against EGFP (which is fused to all the constructs) and against Bcl-XL. Endogenous Bcl-XL (26 kDa) was expected to co-immunoprecipitate with exogenous p53-XL (75 kDa) due to its ability to induce apoptosis, while Bcl-XL should not co-immunoprecipitate with the negative control E-XL. Surprisingly, Bcl-XL co-immunoprecipitated with E-XL (32 kDa) just as p53-XL did (FIG. 8A, lane 1 and 2). To address if the binding is due to the mitochondrial targeting signal which was originally taken from the Bcl-XL protein, another negative control E-CC was used, which does not contain a MTS. EGFP (27 kDa) could not be used as a negative control because it is too close in size to Bcl-XL (26 kDa) and would not be distinguishable on the gel. Bcl-XL did not co-immunoprecipitate with E-CC (FIG. 8A, lane 3) implying that the binding of E-XL to Bcl-XL was due to the XL mitochondrial targeting signal.

To further explore the apoptotic mechanism of DBD-XL and p53-XL at the mitochondria, Bcl-XL was overexpressed in T47D cells. The apoptotic activity was measured by 7-AAD. Cells transfected with just p53-, DBD-, or E-XL were compared to cells cotransfected with either of these constructs and with BFP-Bcl-XL. It was expected that the apoptotic potential of the constructs that are undergoing apoptosis through the p53/Bcl-XL pathway would be rescued by Bcl-XL overexpression. Indeed, DBD-XL and p53-XL apoptotic activities were significantly reduced when BFP-Bcl-XL was cotransfected (FIG. 8B). However, E-XL was not rescued by cotransfection of BFP-Bcl-XL (FIG. 8B).

4. Discussion

It has been shown that targeting p53 to anti-apoptotic Bcl-XL is best achieved by using the MTS from Bcl-XL. Additionally, it was validated that the XL signal is the only MTS that has no inherent toxicity by itself since it is targeting the outer surface of the mitochondrial outer membrane. Mitochondrial targeting of proteins to this region does not disrupt the sensitive balance of the mitochondria as reported with other MTSs. As an approach to determine which domain of p53 is capable of inducing apoptosis similar to p53-XL, different domains of p53 were fused to XL.

The designed constructs translocate to the mitochondria (FIG. 2) and that any construct that contains the DBD of p53 is capable of inducing apoptosis similar to wt p53 (FIG. 3). It has been suggested that a combination of different domains of p53 is necessary for its apoptotic function and interaction with Bcl-XL at the mitochondria. For instance, the PRD is thought to enhance the binding of p53 to Bcl-XL. However, the current data (FIG. 3) clearly validates that the DBD region without the PRD region of p53 is sufficient to induce the full mitochondrial apoptotic function of p53. Even though the PRD was reported to enhance the binding of MBD and DBD to Bcl-XL, it did not have any effect on increasing the apoptotic potential. Hence, individual domains of p53 (FIG. 1B) instead of combinations of domains were used in the remaining apoptotic assays.

Since the 7-AAD assay (FIG. 3) does not distinguish between apoptotic and necrotic cells, early apoptosis assays (annexin V and TUNEL) were conducted to verify that the designed constructs are causing cell death via apoptosis and not necrosis. Indeed, DBD-XL showed the same (FIG. 4B) or higher (FIG. 4A) apoptotic activity compared to p53-XL in human breast cancer cells (T47D). To further validate the tumor suppressor function of the constructs and their ability to inhibit proliferation, a colony forming assay was conducted. As expected, DBD-XL showed similar reduction in transformative ability as p53-XL in T47D breast carcinoma cells (FIG. 5).

To ensure that the increase in apoptotic activity is not cell line, cancer type or p53 status dependent, four different cancer cell lines (MCF-7, MDA-MB-231, HeLa, H1373) were tested. Surprisingly, DBD-XL induces late stage apoptosis significantly higher than p53-XL in all tested cell lines except T47D (FIG. 6). The p53/MDM2 pathway might offer an explanation to why DBD-XL shows higher apoptotic activity compared to p53-XL. MDM2, an ubiquitin ligase (E3) binds to the MBD domain of p53 and helps to transfer ubiquitin from E2 to lysine residues on the carboxy terminus of p53. Ubiquinated p53 is dragged to the proteasome for degradation. DBD-XL canevade degradation by MDM2 since it lacks the MBD and C-terminal domain, allowing for higher stability and consequently increased apoptotic activity. Hagn et al. showed that the amino acids of p53 responsible for interacting with Bcl-XL are located in the DBD of p53 (Gly117, Ser121, Cys176, His178, Asn239, Met243, Arg248, Gly279, and Arg280) and the contact sites on Bcl-XL are residues Ser18, Tyr22, Ser23, Gln26, and Ser28 in helix 1 and 2, Ile114 between helix 3 and 4, and Val155, Asp156, and Glu158 in helix 5. Consequently, DBD-XL contains the residues important for interaction with Bcl-XL while lacking the domains responsible for degradation.

Alternatively, the anti-oxidative role of p53 might offer an explanation to why p53-XL shows lower apoptotic activity compared to DBD-XL. In healthy cells, basal p53 expression limits oxidative stress and promotes cell survival. p53 upregulates the expression of genes involved in the oxidative stress survival pathways such as GPX1, SOD2, ALDH4A1, INP1, TIGAR, Hi95 and PA26. Even though all designed constructs translocate into the mitochondria (FIG. 2), a small fraction could still enter the nucleus. It has been previously shown that p53-XL retains some residual transcriptional activity. Unlike p53-XL which contains full length p53, DBD-XL is not capable of transcribing genes because it lacks the TD to form the transcriptionally active tetrameric p53 and the PRD which enhances transcription of various genes. This could provide another explanation why DBD-XL (which does not activate gene expression) shows higher apoptosis than p53-XL (which could upregulate the expression of genes involved in preventing oxidative stress).

Furthermore, the “mitochondrial priming theory” indicates that some cancer cells such as MCF-7 cells are inherently more sensitive to cytotoxic drugs than other cells. This response correlates with the sensitive balance of anti- and pro-apoptotic Bcl-2 family members at the mitochondrial outer membrane. It is known that T47D, MCF-7, MDA-MB-231 and HeLa express anti-apoptotic Bcl-XL. Therefore, the expression levels of Bcl-XL in T47D, MCF-7, MDA-MB-231 and HeLa were compared (FIG. 9). Indeed, T47D cells had the highest expression level of Bcl-XL confirming that they are “less primed” and more resistant to apoptosis.

Since the DBD-XL shows similar or higher apoptotic activity (measured by TUNEL, annexin V and 7-AAD) compared to p53-XL consistently in every tested cell line (FIGS. 3, 4, and 6), the effect on cell death due to a mitochondrial dependent mechanism was examined. DBD-XL triggers more caspase-9 activation than the negative control E-XL (FIG. 7B) but surprisingly less caspase-9 induction than p53-XL (FIG. 7B). Even though p53-XL caspase activity is higher, this is a transient effect that is not reflected in the more “final” apoptotic assays (FIG. 3,4,6). Additionally, a certain threshold of caspase 9 activation achieved by DBD-XL can be sufficient to induce cell death. Furthermore, DBD-XL induces MOMP to the same extent as p53-XL, indicating that DBD-XL dependent apoptosis occurs through the intrinsic apoptotic pathway and can be through a direct interaction with Bcl-XL (FIG. 7A). As described above, p53-XL can be degraded via the proteasome. Once MDM-2, an ubiquitin ligase, binds to the MBD of p53, the C-terminal region of p53 becomes ubiquitinated and p53 is dragged into the proteasome for degradation. This can explain why initially p53-XL causes more caspase-9 activation (FIG. 7A) but this difference in activity is not reflected in the more “final” apoptosis assays where DBD-XL shows even higher apoptosis activity compared to p53-XL (FIG. 6).

In an effort to determine if the apoptotic potential of the designed constructs is due to their interaction with Bcl-XL or if it is independent of the p53/Bcl-XL pathway, a co-IP and a rescue experiment achieved by overexpressing Bcl-XL were conducted. Interestingly, Bcl-XL co-immunoprecipitated with the “negative control” E-XL in the same manner as p53-XL (FIG. 8A). The interaction with Bcl-XL can be independent of p53 due to the XL MTS which will directly target every protein that contains XL to Bcl-XL. To investigate this hypothesis, E-CC (a negative control lacking the XL signal) was created. As expected, E-CC did not bind to Bcl-XL (FIG. 8A), confirming that the XL signal is responsible for the interaction with Bcl-XL.

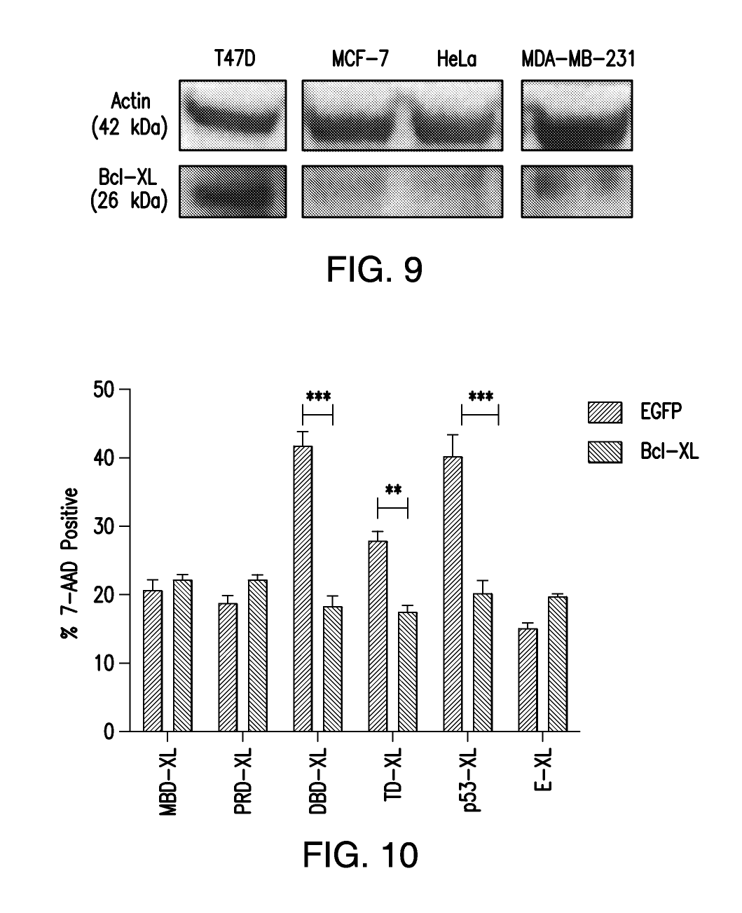

To prove indirectly that the apoptotic mechanism of p53- and DBD-XL are through direct interaction of p53 and DBD with Bcl-XL, a rescue experiment using overexpressed Bcl-XL was conducted. As expected the apoptotic activity of MBD-, PRD- and E-XL was not altered by Bcl-XL overexpression (FIG. 10). However, DBD-XL, p53-XL (and even TD-XL) demonstrated reduction in apoptotic potential, further demonstrating the necessity of Bcl-XL for apoptosis initiation (FIG. 8B, FIG. 10). Even though TD-XL showed significantly lower cell death compared to p53-XL, it was still significantly higher than the negative control E-XL, and was still rescued by Bcl-XL. It could be speculated that TD-XL binds to endogenous, mutant p53 through its TD and drags it to the mitochondria where it potentially interacts with Bcl-XL and triggers marginal apoptosis. Even though endogenous, mutant p53 is transcriptionally inactive in T47D cells due to the presence of the L194F mutation, this mutant p53 can still be active at the mitochondria, since the L194 residue is not involved in the interaction between p53 and Bcl-XL.

In summary, DBD-XL shows the same (T47D) or higher (MCF-7, MDA-MB-231, HeLa, H1373) apoptotic activity compared to p53-XL. Mechanistic studies indicate that DBD-XL can bind and trigger apoptosis similar to p53 through the Bcl-XL dependent pathway. The data highlights that DBD (about half the size of full length p53) can be used instead of p53 for achieving apoptosis at the mitochondria when fused to the MTS from Bcl-XL. The benefit of decreasing the overall size of p53 by half while still maintaining full apoptotic activity allows for better drug delivery options. DBD-XL can be used as a therapeutic in vivo using adenoviral drug delivery. In conclusion, DBD-XL can be used to trigger a potent, rapid apoptotic response in various cancer cell lines (including breast, cervical and lung carcinomas) with different p53 status, and is an alternative to wt-p53 gene therapy. Importantly, the mechanism of DBD-XL-mediated apoptosis is distinctly different from conventional wild type p53 cancer therapy.

### O. Example 2

### Delivery of a Monomeric p53 Subdomain with Mitochondrial Targeting Signals from Pro-Apoptotic Bak or Bax

1. Introduction

The tumor suppressor p53 exhibits distinct functions at the cytoplasm, the nucleus and the mitochondria. Under normal conditions, the E3 ligase murine double minute 2 (MDM2) binds to the MDM2 binding domain (MBD) of p53 prompting polyubiquitination of terminal lysines on the C-terminus of p53, which marks p53 for proteasomal degradation. Upon stress induction, such as DNA damage or ER stress, cytoplasmic p53 translocates either to the nucleusor to the mitochondria. Three nuclear localization signals (NLS) in the C-terminus of p53 are responsible for p53 nuclear localization. In the nucleus, p53 forms a tetramer via its tetramerization domain (TD) allowing its DNA binding domain (DBD) to bind to DNA activating various genes that are involved in apoptosis, DNA repair and cell cycle arrest.

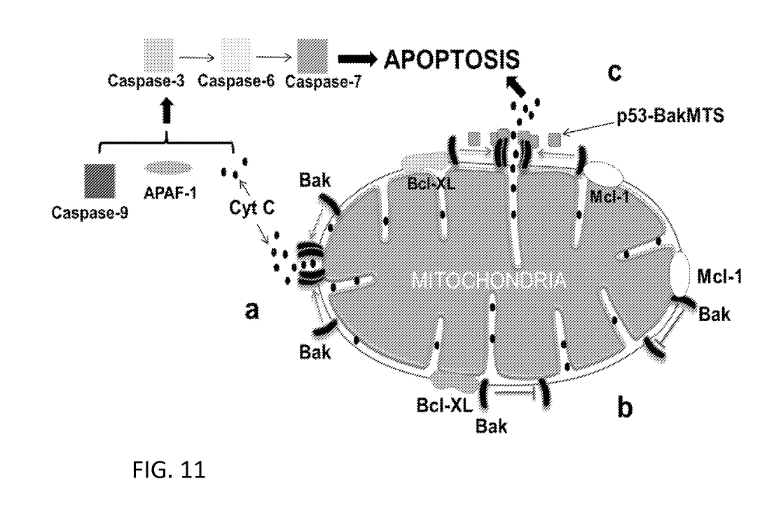

Although p53 does not contain a mitochondrial targeting signal (MTS), it can still translocate to the mitochondria. Machenko et al. postulated that MDM2 triggers dimer formation and mono-ubiquitination of cytoplasmic p53 resulting in mitochondrial import via herpes virus-associated ubiquitin-specific protease. At the mitochondrial outer membrane, p53 directly interacts with pro-apoptotic (Bak or Bax) and anti-apoptotic Bcl-2 family members (Bcl-XL, Bcl-2, Mcl-1, Bcl-w, and A1)through a sequential mechanism first binding anti-apoptotic Bcl-2 proteins followed by binding to pro-apoptotic Bak(FIG. 11) or Bax. Activation of Bak (FIG. 11) or Bax leads to homotetramer formation, which causes cytochrome c release from the intermembrane space. Binding of cytochrome c to APAF-1 stimulates the assembly of a hepameric, wheel-like structure known as the apoptosome. The apoptosome activates the initiator caspase-9 which initiates the executioner apoptotic caspase-3 and caspase-7 (FIG. 11). Their proteolytic activity leads to nuclear fragmentation, chromatin condensation and cell shrinking, also known as programmed cell death or apoptosis.

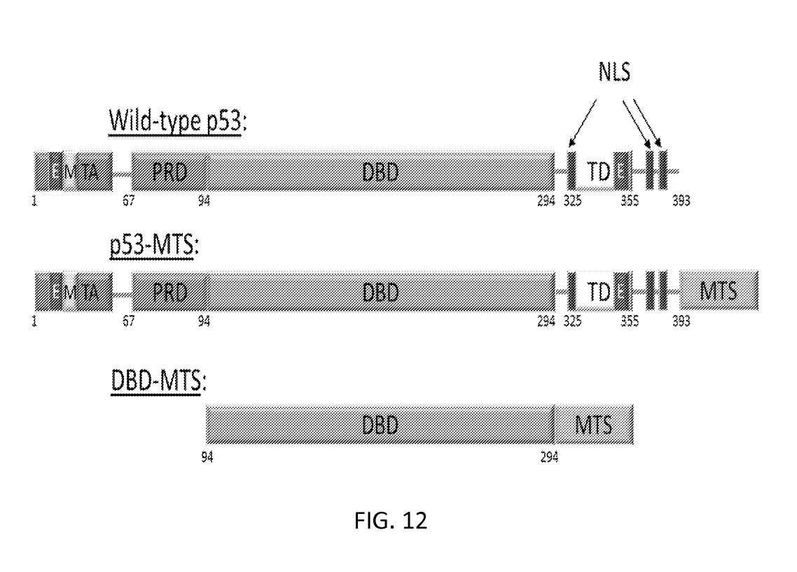

In cancer cells, overexpression of Bcl-2, Bcl-XL and Mcl-1 correlates with more aggressive phenotypes and leads to chemotherapy resistance. Many agents have been identified to target the anti-apoptotic Bcl-2 family members such as navitoclax (inhibits Bcl-2, Bcl-XL, and Bcl-w) and ABT-199 (inhibits Bcl-2). These therapeutics initiate apoptosis by neutralizing anti-apoptotic proteins at the mitochondria thus allowing the pro-apoptotic Bcl-2 family members Bak or Bax to homo-oligomerize leading to apoptosis. However, these inhibitors do not inactivate anti-apoptotic Mcl-1. Overexpression of Mcl-1 is linked to reduced response to chemotherapy and poor prognosis which limits the therapeutic options for navitoclax and ABT-199. Mcl-1 (and to a certain extent Bcl-XL) is the main inhibitor of Bak while Bax is mainly inhibited by Bcl-2 and Bcl-w. This study is to directly activate pro-apoptotic Bak and Bax by targeting p53 to the mitochondria using Bak's or Bax's own MTSs (FIG. 12).

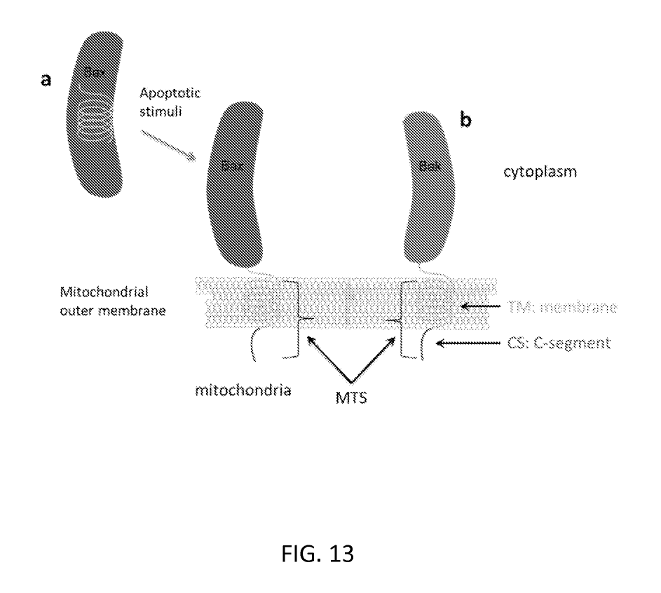

The MTSs of Bak or Bax are located on the C-terminal hydrophobic regions of these proteins. The C-terminus contains the transmembrane domain (TM) and the C-segment (CS) as in FIG. 13 and Table 1. The TM inserts both proteins into the mitochondrial outer membrane (tail anchored proteins) with at least two of the basic amino acids in the CS being necessary for the insertion (FIG. 12). Bax, which is in the cytoplasm, sequesters its TM in its hydrophobic surface groove. Once an apoptotic stimuli occurs, the TM gets externalized, targets Bax to the mitochondria, and inserts itself into the mitochondrial outer membrane (FIG. 13). However, Bak is always present at the mitochondrial outer membrane sequestered by Mcl-1 (and Bcl-XL) (FIG. 11; 13).

As mentioned before wt p53 does not contain a MTS. Therefore, this study examines mitochondrial targeting of p53 by fusing the MTSs from Bak or Bax to p53. Murphy and colleagues have reported that wt p53 is required to be in a dimeric or tetrameric form in order to activate pro-apoptotic Bak. In addition, the DBD has been reported to interact with pro-apoptotic Bak and inhibit anti-apoptotic Bcl-XL and Bcl-2. Here, the finding that the DBD in isolation with a MTS from Bak or Bax is sufficient to induce apoptosis in different cancer cells is shown.

TABLE 1

| | | Mitochondrial |
|---|---|---|
| Protein | Compartment | targeting sequences |
| Bax | Outer | GTPTWQTVTIFVAGVLTASLTIWKKMG |
| | mitochondrial | |
| | membrane | |
| Bak | Outer | GNGPILNVLVVLGVVLLGQFVVRRFFKS |
| | mitochondrial | |
| | membrane | |

Amino acid sequence of the MTSs from Bax and Bak protein.
Bold letters depict the TM domain and underlined letters
illustrate the CS base pairs.

2. Material and Methods

i. Cell Lines and Transient Transfections

1471.1 murine adenocarcinoma cells (a kind gift of G. Hager, NCI, NIH), T47D human ductal breast epithelial tumor cells (ATCC, Manassas, Va.), H1373 human non-small cell lung carcinoma cells (a kind gift from Dr. Andrea Bild, University of Utah), SKOV-3 human ovarian adenocarcinoma cells (a kind gift from Dr. Margit Janat-Amsbury, University of Utah) and HeLa human epithelial cervical adenocarcinoma cells (ATCC) were grown as monolayers in DMEM (1471.1, SKOV-3) or RPMI (T47D, H1373, HeLa) (Invitrogen, Carlsbad, Calif.) supplemented with 10% FBS (Invitrogen), 1% penicillin-streptomycin (Invitrogen), 1% glutamine (Invitrogen) and 0.1% gentamycin (Invitrogen) and maintained in a 5% CO₂ incubator at 37° C. T47D media was additionally supplemented with 4 mg/L insulin (Sigma, St. Louis, Mo.). For microscopy 7.5×10⁴ cells for 1471.1 cells were seeded in a 2 well live cell chamber. For apoptosis assays 3.0×10⁵ cells for T47D, 1.0×10⁵ cells for HeLa, 2.0×10⁵ for H1373 and SKOV-3 were seeded in 6-well plates (Greiner Bio-One, Monroe, N.C.). To account for varying cell growth rates, different amounts of cells were plated in live cell chambers and 6-well plates. Following the manufacturer's recommendations 24 h after seeding, transfections were performed using 1 pmol of DNA per well (unless otherwise indicated) and Lipofectamine 2000 (Invitrogen).

ii. Plasmid Construction

pEGFP-p53-BakMTS (p53-BakMTS):

An oligonucleotide encoding the MTS from Bak (5′-GATCCGGCAATGGTCCCATCCTGAACGTGCTGGTGGTTCTGGGTGTGGTTCTGTT GGGCCAGTTTGTGGTACGAAGATTCTTCAAATCATGAG-3′ (SEQ ID NO:26)) was annealed to its reverse complementary strand and fused to the C-terminus of EGFP-p53 using the BamHI restriction sites (NEB, Ipswich, Mass.).

pEGFP-BakMTS (E-BakMTS):

The annealed oligonucleotide encoding the MTS from Bak was fused to the C-terminus of EGFP-C1 vector (Clontech, Mountain View, Calif.) using the BamHI (NEB) restriction sites.

pEGFP-DBD-BakMTS (DBD-BakMTS):

The DNA encoding the DBD was amplified via PCR from previously subcloned pEGFP-p53 using 5′-CCGGGCCCGCGGTCCGGAACCTACCAGGGCAGCTACG-3′ (SEQ ID NO:16) and 5′-CCGGGCCCGCGGGGTACCTTTCTTGCGGAGATTCTCTTCCT-3′ (SEQ ID NO:17) and cloned between EGFP and Bak MTS into the multiple cloning site of E-BakMTS using BspEI (NEB) and KpnI (NEB) sites.

pEGFP-p53 K120A, R248A, R273A, R280A, E285A, E287A-Bak (p53m6-BakMTS) and pEGFP-DBD K120A, R248A, R273A, R280A, E285A, E287A-Bak (DBDm6-BakMTS): K120A, R248A, R273A, R280A, E285A, and E287A mutations were introduced in p53-BakMTS and DBD-BakMTSusing the QuickChange II XL Site-Directed Mutagenesis Kit (Agilent, Santa Clara, Calif.). The primers listed below and their reverse complements were used to introduce the K120A mutation 5′-CATTCTGGGACAGCCGCGTCTGTGACTTGCAC-3′ (SEQ ID NO:18); the R248A mutation 5′-CAGTTCCTGCATGGGCGGCATGAACGCGAGGCCCATCCT-3′ (SEQ ID NO:19); the R273A mutation 5′-GGGACGGAACAGCTTTGAGGTGGCTGTTTGTGCCTGTCCT-3′ (SEQ ID NO:28); the R280A mutation 5′-TTTGTGCCTGTCCTGGGGCAGACCGGCGCACA-3′ (SEQ ID NO:20); and the E285A, E287A mutations 5′-ACCGGCGCACAGCGGAAGCGAATCTCCGC-3′ (SEQ ID NO:21) (underlined sequence indicate mutation sites).

pEGFP-p53K120E-BakMTS (p53K120E-BakMTS) and pEGFP-DBDK120E-BakMTS (DBDK120E-BakMTS):

The K120E mutation was introduced into p53-BakMTS and DBD-BakMTS via QuickChange II XL Site-Directed Mutagenesis Kit (Agilent) using 5′-GCATTCTGGGACAGCCGAGTCTGTGACTTGCACGTA-3′ (SEQ ID NO:22) and its reverse complement.

pEGFP-p53-BaxMTS (p53-BaxMTS):

An oligonucleotide encoding the MTS from Bax 5′-GATCCTCCTACTTTGGGACGCCCACGTGGCAGACCGTGACCATCTTTGTGGCGGG AGTGCTCACCGCCTCACTCACCATCTGGAAGAAGATGGGCTGAG-3′ (SEQ ID NO:23) was annealed to its reverse complementary strand and fused to the C-terminus of EGFP-p53 using the BamHI (NEB) restriction sites.

pEGFP-BaxMTS (E-BaxMTS):

The annealed oligonucleotide encoding the MTS from Bax was fused to the C-terminus of EGFP-C1 vector (Clontech) using the BamHI (NEB) restriction sites.

pEGFP-DBD-BaxMTS (DBD-BaxMTS):

The DNA encoding the DBD was amplified as mentioned above and cloned between EGFP and Bax MTS into the multiple cloning site of E-Bax using BspEI (NEB) and KpnI (NEB) sites.

iii. Mitochondrial Staining and Microscopy

Prior to microscopy, media in live cell chambers was replaced with media containing 10% charcoal stripped fetal bovine serum (CS-FBS, Invitrogen). To stain the mitochondria, cells were incubated with 150 nM of MitoTracker Red FM (Invitrogen) for 15 min at 37° C. and protected from light prior to imaging. All images of 1471.1 and T47D live cells were acquired as previously with an Olympus IX71F fluorescence microscope (Scientific Instrument Company, Aurora, Colo.) with high quality (HQ) narrow band GFP filter (ex, HQ480/20 nm; em, HQ510/20 nm) and HQ:TRITC filter (ex, HQ545/30; em, HQ620/60) from Chroma Technology (Brattleboro, Vt.) with a 40× PlanApo oil immersion objective (NA 1.00) on an F-View Monochrome CCD camera.

iv. Image Analysis

Images were analyzed by using JACoP plugin in ImageJ software. PCC values were generated using Pearson's correlation coefficient (PCC) with post Costes' automatic threshold algorithm. PCC depends on both the pixel intensity and overlap of signals. A PCC of +1 represents complete colocalization of EGFP constructs with mitochondria; a PCC of −1 represents anti-correlation, and PCC of 0 correlates to random distribution. Bolte and Cordelières defined PCC values equal to 0.6 or above to be colocalized. For images analysis, experiments were performed three times (n=3) with 10 cells analyzed per n and per construct.

v. 7-AAD Assay

Transfected T47D, H1373, SKOV-3 and HeLa cells were pelleted and re-suspended in 500 μL PBS (Invitrogen) containing 1 μM 7-aminoactinomycin D (7-AAD) (Invitrogen) for 30 min prior to analysis. T47D and H1373 cells were analyzed 48 h after transfection, while SKOV-3 and HeLa cells were analyzed 24 h after transfection (time points optimized empirically). Only EGFP positive cells were assayed using the FACS Canto-II (BD-BioSciences) with FACS Diva software as previously. EGFP and 7-AAD were excited at 488 nm, and detected at 507 nm and 660 nm, respectively. Independent transfections of each construct were assayed three times (n=3). The highest value (EGFP positive cells stained with 7-AAD) was set at 100%, and the lowest at 0% (relative 7-AAD) as previously.

vi. Reporter Gene Assay

3.5 μg of p53-BakMTS, E-BakMTS, p53-BaxMTS, E-BaxMTS, wt p53 or EGFP were co-transfected with 3.5 μg of p53-Luc Cis-Reporter (Agilent Technologies) encoding the firefly luciferase gene and 0.35 μg of pRL-SV40 plasmid encoding Renilla luciferase (Promega, Madison, Wis.) to normalize for transfection efficiency in T47D cells using the Dual-Glo Luciferase assay system as previously. Luminescence was detected 24 h post transfection using PlateLumino (Stratec Biomedical Systems, Birkenfeld, Germany). Independent transfections of each constructs were assayed three times (n=3). The highest value was set at 100% and lowest value (untreated cells) was set as 0% (relative luminescence).

vii. TMRE Assay

36 h after transfection T47D cells were incubated with 100 nM tetramethylrhodamine, ethylester (TMRE) (Invitrogen) for 30 min at 37° C., pelleted and resuspended in 300 μL annexin-V binding buffer (1×) (Invitrogen). The FACS Canto-II with FACS Diva software was used to analyze only EGFP positive cells (excited at 488 nm with emission 530/35) stained with TMRE (excited at 561 nm laser with the emission 585/15). Loss in TMRE intensity represents mitochondrial depolarization, which correlates with an increase in mitochondrial outer membrane permeabilization (MOMP). Independent transfections of each construct were assayed three times (n=3). The highest MOMP value was set as 100% and the lowest as 0% (relative MOMP) as before.

viii. Caspase-9 Assay

As previously described, 48 h after transfection, T47D cells were tested with SR FLICA Caspase-9 Assay Kit (Immunochemistry Technologies, Bloomington, Minn.). Cells were incubated with SR FLICA Caspase-9 reagent for 60 min, pelleted and re-suspended in 300 μL, 1× wash buffer (Immunochemistry Technologies). The FACS Canto-II with FACS Diva software was used to analyzed only EGFP positive cells stained with caspase-9, both were excited with the 488 nm (emission filter 530/35) and the 561 nm laser (emission filter 585/15), respectively. Independent transfections of each construct were assayed three times (n=3). The highest value (EGFP positive cells stained with caspase-9) was set as 100% and the lowest as 0% (relative caspase-9).

ix. Statistical Analysis

All experiments were done in triplicate (n=3). One-way analysis of variance (ANOVA) with Bonferroni's post test was used to determine statistical significance as indicated in figure legend. To determine the degree of colocalization odds ratio with Pearson's Chi-square was applied comparing each PCC value with PCC of 0.6. A p value of <0.6 was considered significant.

3. Results

i. Colocalization of Designed Constructs with the Mitochondria

Since all designed constructs are tagged to EGFP, their mitochondrial localization was determined using fluorescence microscopy in murine adenocarcinoma cells (1471.1). 1471.1 cells were chosen for the microscopy study because their large size allows for clear distinction between nucleus, cytoplasm and mitochondria. Similar results have been observed for T47D cells (data not shown).

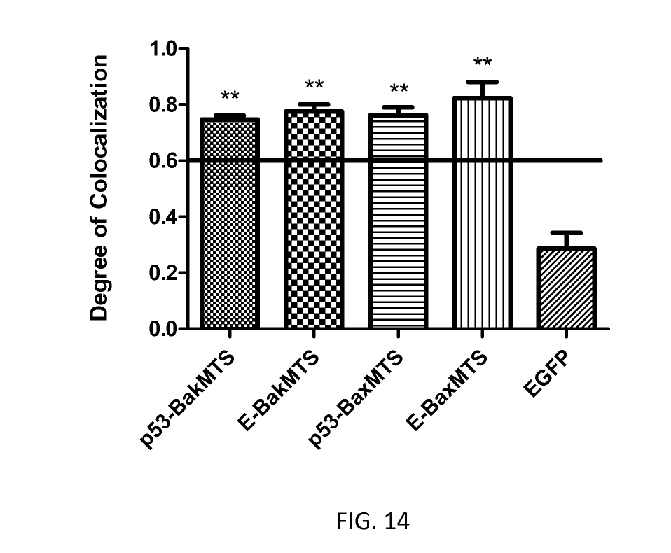

Colocalization of EGFP tagged constructs with the mitochondrial compartment was illustrated by graphing the generated PCC values for each construct as shown in FIG. 14. PCC values of 0.6 or greater represent colocalization of EGFP tagged constructs with mitochondria.p53-BakMTS, E-BakMTS, p53-BaxMTS and E-BaxMTS have a significantly higher PCC value than 0.6. The negative control EGFP shows random distribution (PCC=0.29) (FIG. 14). Since wt p53 is a transcription factor containing three nuclear localization signals (NLSs), the main fraction of wt p53 localizes to the nucleus as previously shown. However, MTSs derived from the pro-apoptotic Bak or Bax protein are capable of overcoming the three NLSs. These Bak and Bax MTSs are capable of targeting EGFP fused to p53 to the mitochondria (FIG. 14).

ii. p53-BakMTS and p53-BaxMTS Induce Late Stage Apoptosis

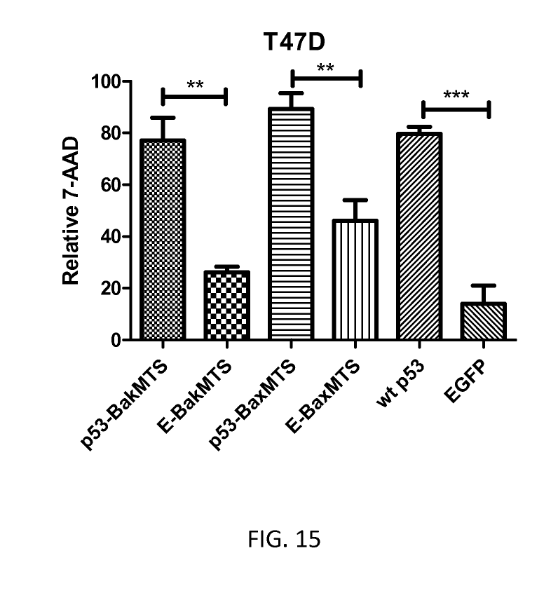

The ability of p53-BakMTS and p53-BaxMTS to induce apoptosis was tested via 7-AAD assay in T47D breast cancer cells. The 7-AAD dye intercalates into double-stranded DNA of apoptotic/necrotic cells which have a disrupted cell membrane. However, it is not capable of penetrating the intact cell membrane of living cells. p53-BakMTS, p53-BaxMTS and wt p53 (FIG. 15; compare 1^{st}, 3^{rd}, and 5^{th} bars)show a significant apoptotic effect compared to their corresponding negative controls E-BakMTS, E-BaxMTS and EGFP respectively (FIG. 15; compare 2^{nd}, 4^{th}, and 6^{th} bars).

The apoptotic response induced by p53-BakMTS and p53-BaxMTS is similar to wt p53 (FIG. 15; compare 1^{st}, 3^{rd}, and 5^{th} bars). Additionally, MTS negative controls, E-BakMTS and E-BaxMTS, show minimal activity similar to the nontoxic EGFP negative control (FIG. 15; compare 2^{nd}, 4^{th} 4 and 6^{th} bars), which indicates no inherent mitochondrial toxicity for these MTSs by themselves.

iii. p53-BakMTS and p53-BaxMTS do not Trigger Apoptosis Through the Nuclear but Through the Mitochondrial Apoptotic Pathway

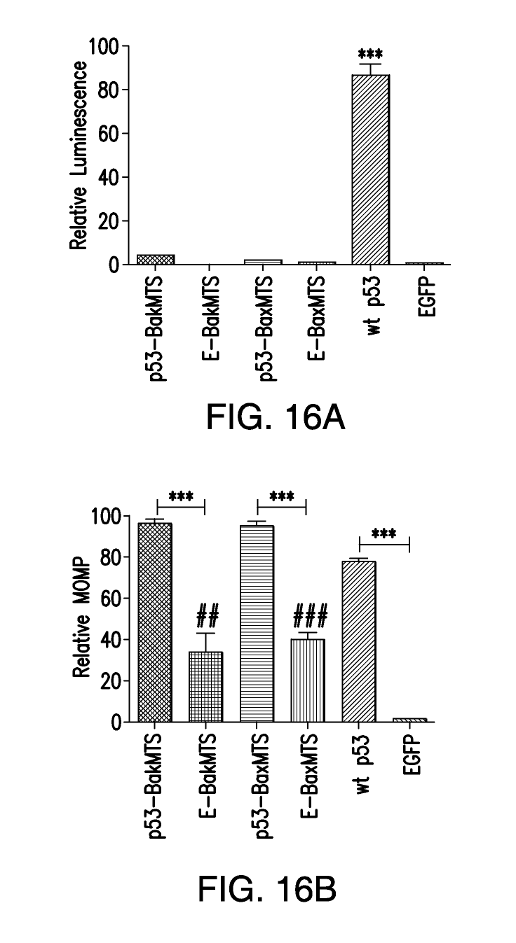

To validate that the apoptotic activity of p53-BakMTS and p53-BaxMTS is not due to transcriptional activity at the nucleus, a p53 reporter dual luciferase assay was conducted in T47D cells. The cis reporter system relies on a synthetic promoter which consists of repeats of the transcription recognition consensus for p53 (TGCCTGGACTTGCCTGG)₁₄. Nuclear activity was represented as relative luminescence. Endogenous p53 is a nuclear protein and exhibits most of its tumor suppressor functions as a transcription factor. wt p53 shows high transcriptional activity (FIG. 16a;5^{th} bar). p53-BakMTS, E-BakMTS, p53-BaxMTS, E-BaxMTS (FIG. 16a; compare 1^{st}, 2^{nd}, 3^{rd} and 4^{th} bars) show no nuclear activity similar to that of the negative control EGFP (FIG. 16a; 6^{th} bar). This indicates that the induction of apoptosis of the mitochondrial constructs p53-BakMTS and p53-BaxMTS seen in FIG. 15 (5^{th} bar) is not due to transcriptionally active p53.

To explore if p53-BakMTS and p53-BaxMTS initiate apoptosis through the intrinsic apoptotic pathway, two major hallmarks for the mitochondrial apoptotic pathway, mitochondrial outer membrane permeabilization (MOMP) and caspase-9 induction, were measured.

The TMRE assay is a direct measurement of MOMP. Homo-oligomerization of Bak or Bax triggers MOMP which results in a decrease in mitochondrial membrane potential. Cationic dyes such as TMRE accumulate in the mitochondria of healthy cells due to the higher negative charge seen in the mitochondria compared to cytoplasm. MOMP results in a loss of TMRE from mitochondria and can be measured via flow cytometry. Apoptotic cells are identified by a loss of TMRE fluorescence intensity and are represented as % MOMP induction on and y-axis (FIG. 16b).

The ability of caspase-9 to cleave the peptide sequence leucine-glutamic acid-histidine-aspartic acid determines caspase-9 activity in apoptotic cells. When the mitochondrial outer membrane ruptures, cytochrome c is released from the intermembrane space. Cytochrome c and Apaf-1 form the apoptosome and activate caspase-9 as shown in FIG. 11. Caspase-9 activation was measured via the caspase-9 assay.

p53-BakMTS, p53-BaxMTS and wt p53 (FIG. 16b, and c; compare 1st, 3^{rd}, and 5^{th} bars) show a significant effect on MOMP and caspase-9 activation compared to their corresponding controls E-BakMTS, E-BaxMTS and EGFP (FIG. 16 b, and c; compare 2^{nd}, 4^{rd}, and 6^{th} bars). The negative controls E-BakMTS and E-BaxMTS show higher MOMP and caspase-9 activation compared to non-toxic EGFP (FIG. 16b, and c; compare 2^{nd}, 4^{rd}, and 6^{th} bars).

iv. DBD-BakMTS and DBD-BaxMTS Induce Late Stage Apoptosis in a Similar Manner as p53-BakMTS and p53-BaxMTS

Pietsch et al. showed that p53 must form a dimer or a tetramer to activate Bak oligomerization. Additionally, the DBD has been reported to interact with pro-apoptotic Bakand inhibit anti-apoptotic Bcl-XL and Bcl-2.

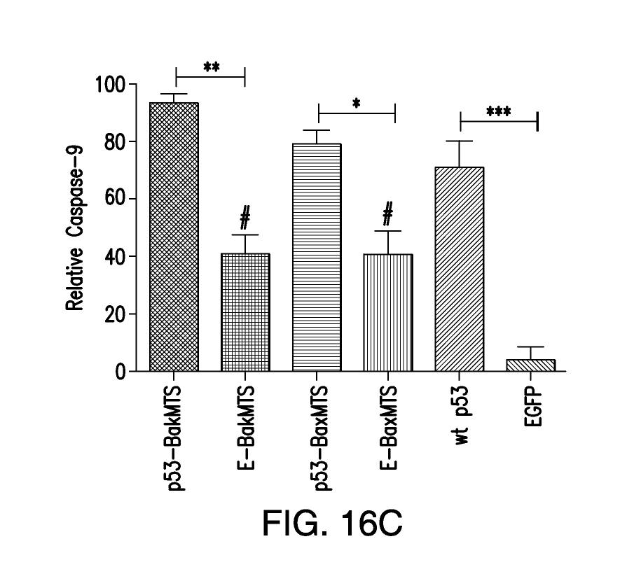

Similar levels of 7-AAD positive staining (apoptosis) were detected between cells transfected with DBD-BakMTS compared to p53-BakMTS (FIG. 17a), and cells transfected with DBD-BaxMTS and p53-BaxMTS (FIG. 17b). Additionally, all of these constructs had significantly higher apoptosis compared to cells transfected with MTS negative controls (E-BakMTS, E-BaxMTS) (FIGS. 17a and b). DBD fused to Bak or Bax MTS induces a similar apoptotic response as full length p53 fused to these MTSs (FIGS. 7a and b).

v. The Activity of Our Re-Engineered Mitochondrially Targeted p53 Constructs in Different Cancer Cell Types

To confirm that the apoptotic potential of the designed constructs causes apoptosis in other cell lines besides T47D breast cancer cells (which express mutant p53 with a L194F point mutation in the DBD of p53), a 7-AAD assay was conducted in non-small cell lung cancer cells (H1373), ovarian cancer cells (SKOV-3) and cervical carcinoma cells (HeLa). H1373 and SKOV-3 cells are p53 null while HeLa cells have endogenous wt p53 (Tbl. II).

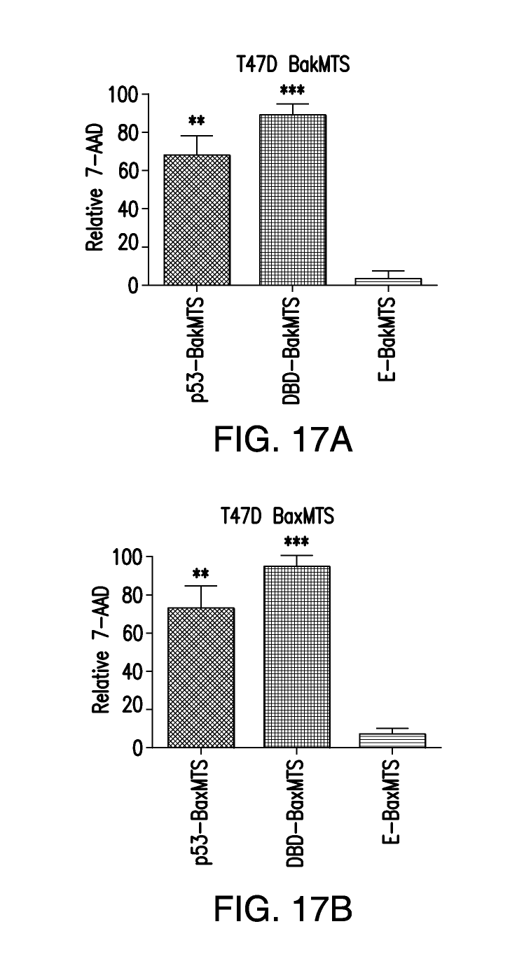

In H1373 cells, both p53-BakMTS and DBD-BakMTS apoptotic activities were statistically higher from their negative control E-BakMTS (FIG. 18a; compare 1^{st}, 2^{nd}, and 3^{rd} bars). As expected, wt p53 showed significantly higher apoptosis compared to EGFP (FIGS. 18a and b; compare 4^{st}, and 5^{st} bars). However, only DBD-BaxMTS activity was significantly higher than E-BaxMTS (FIG. 18b; compare 2^{nd} and 3^{rd} bars) and there was no significant difference between p53-BaxMTS and E-BaxMTS (FIG. 18b; compare 1^{st} and 3^{rd} bars). Additionally, DBD-BakMTS and DBD-BaxMTS had significantly higher activities compared to their positive controls p53-BakMTS and p53-BaxMTS (FIGS. 18a and b; compare 1^{st} and 2^{nd} bars).

In SKOV-3 cells, p53-BakMTS and DBD-BakMTS activities were significantly higher than their negative control E-BakMTS (FIG. 18c; compare 1^{st}, 2^{nd}, and 3^{rd} bars). p53-BaxMTS and DBD-BaxMTS activities were only significant when compared to EGFP but no to E-BaxMTS (FIG. 18d; compare 1^{st}, 2^{nd}, 3^{rd}, and 5^{th} bars). The activity of wt p53 was similar to nontoxic EGFP in SKOV-3 cells (FIGS. 18c and d; compare 4^{th} and 5^{th} bars).

In HeLa cells, p53-BakMTS, DBD-BakMTS (FIG. 18e; compare 1^{st} and 2^{nd} bars), p53-BaxMTS, DBD-BaxMTS (FIG. 18f; compare 1^{st}, and 2^{nd} bars) and wt p53 (FIGS. 18e and f; 4^{th} bars) apoptotic activities were significant from their corresponding negative controls E-BakMTS (FIG. 18e; 3^{rd} bar), E-BaxMTS (FIG. 18f; 3^{rd} bar) and EGFP(FIG. 18e, and f; 5^{th} bar)respectively. DBD-BakMTS showed a trend of higher apoptotic activity in HeLa cells compared to p53-BakMTS (FIG. 18e; compare 1^{st} and 2^{nd} bars). In addition, DBD-BaxMTS was significantly higher than p53-BaxMTS (FIG. 18f; compare 1^{st} and 2^{nd} bars).

p53 is known to induce a conformational change in Bax that triggers its oligomerization and mitochondrial permeabilization through a hit-and-run type mechanism. However, this p53 interaction with Bax is transient, and the specific interacting residues are not known. On the other hand, it is known that p53 interacts with the Bak protein via amino acids K120, R248, R273, R280, E285 and E287 in p53. Therefore these residues will be mutated to determine if this is a Bak specific interaction. Since p53-BakMTS and DBD-BakMTS showed consistently higher apoptotic activities than their MTS control in all tested cell lines (FIGS. 7a, 8a,c,e; compare 1st, 2nd, and 3rd bars), the apoptotic mechanism of the Bak MTS constructs were examined.

TABLE 2
Comparison of T47D, H1373, SKOV-3 and HeLa in terms of p53status
and cancer type.

| Cell line | p53 status | Cancer type |
|---|---|---|
| T47D | mutated p53 (L194F) | mammary ductal carcinoma |
| H1373 | null | non-small cell lung carcinoma |
| SKOV-3 | null | ovarian adenocarcinoma |
| HeLa | wild-type | cervical adenocarcinoma |

vi. Exploring the Interaction Between p53-BakMTS, DBD-BakMTS and Pro-Apoptotic Bak Protein

It has been reported that p53 interacts with pro-apoptotic Bak protein via amino acids K120, R248, R273, R280, E285 and E287, all of which are found in the DBD of p53. To verify that the apoptotic potential of p53-BakMTS and DBD-BakMTS is through the p53/Bak specific pathway, all sites of p53 (K120A, R248A, R273A, R280A, E285A, E287A) that contact the pro-apoptotic Bak protein were mutated to alanine. The constructs with these six mutations were named p53m6-BakMTS and DBDm6-BakMTS.

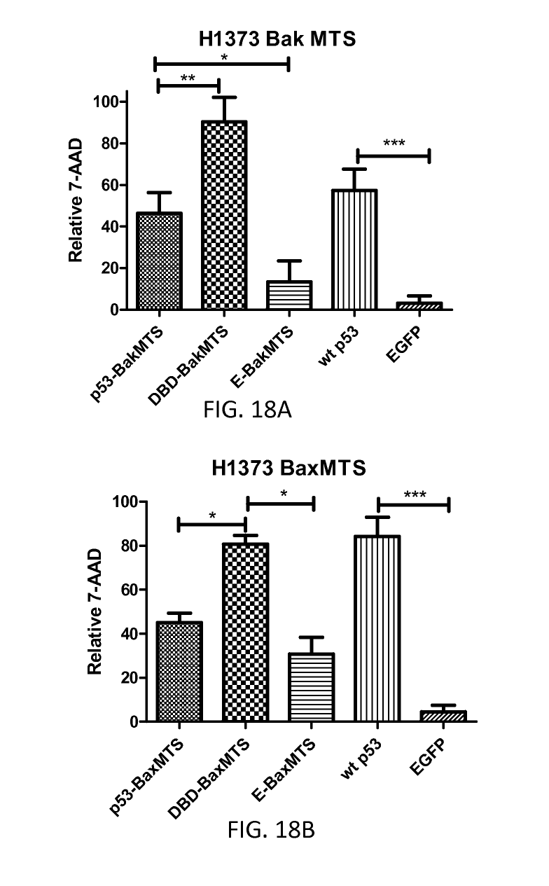

Mutating all six of these residues in p53-BakMTS to eliminate binding to pro-apoptotic Bak protein resulted in a complete loss of p53-BakMTS activity (FIG. 19a;4^{th} bar). p53m6-BakMTS and DBDm6-BakMTS activities are not significantly different from their negative control E-BakMTSindicatingBak dependent apoptosis (FIG. 19a; compare 3^{rd}, and 5^{th} bars).

As mentioned above p53 interacts with Bak via its DBD (residues K120, R248, R273, R280, E285, E287). However, p53 also interacts with anti-apoptotic Bcl-XL through the following residues G117, 5121, C176, H178, N239, M243, R248, G279, and R280. Therefore, R248 and R280 localized in the DBD of p53 can interact with Bak and Bcl-XL. To exclude any Bcl-XL specific interaction of the designed constructs, only K120 was mutated in the p53-BakMTS and DBD-BakMTS plasmid to glutamic acid. The K120 residue only interacts with Bak not with Bcl-XL.

Mutating the positively charged lysine 120 to negatively charged glutamic acid (K120E) in p53-BakMTS and DBD-BakMTS resulted in complete loss of apoptotic activity (FIG. 19b; 4^{th} and 5^{th} bars). p53K120E-BakMTS and DBDK120E-BakMTS activities are not statistically significant from the negative control E-BakMTS (FIG. 19b; compare 3^{rd}, 4^{th}, and 5^{th} bars), indicating a p53/Bak dependent apoptotic mechanism.

4. Discussion

Targeting p53 to the mitochondria is sufficient to trigger a rapid apoptotic response. Previously, the focus was to target p53 to different mitochondrial compartments concluding that targeting it to the outer surface of the mitochondrial membrane is the only compartment that leads to p53-dependent apoptosis, rather than non-specific mitochondrial toxicity. So far, only anti-apoptotic binding partners (such as Bcl-XL and Bcl-2) on the outer membrane have been addressed for p53 specific targeting. These constructs sequester anti-apoptotic Bcl-2 family members and therefore indirectly activate Bak and Bax. This study targets p53 directly to Bak and Bax proteins.

Fusing p53 to the MTSs derived from the pro-apoptotic Bak (p53-BakMTS) or Bax (p53-BaxMTS) proteins resulted in localization of these constructs to the mitochondria (FIG. 14) and induction of apoptosis (FIGS. 15; 16b and c; 7). The tumor suppressor p53 is a nuclear protein containing three NLSs. When targeting p53 to the mitochondria, the chosen MTS must counteract these NLSs. Previously, targeting was done with p53 with the MTSs from ornithine transcarbamylase (OTC), cytochrome c oxidase (CCO), translocase of the outer membrane (TOM) and Bcl-XL (XL). Strong MTSs from TOM and XL are capable of overcoming the NLSs in the p53 protein while the weak MTS from CCO and the medium strength MTS from OTC are not strong enough to ensure entire mitochondrial targeting. Here, it is shown that MTSs from Bak and Bax are capable of counteracting the three NLSs in wt p53 and can be considered to be strong MTSs (FIG. 14). Additionally, EGFP fused to MTSs from Bak (E-BakMTS) or Bax (E-BaxMTS) showed minimal inherent toxicity which indicates that apoptotic activity of p53-BakMTS and p53-BaxMTS are p53 dependent and not due to MTS toxicity (FIG. 15).

Since mitochondrial localization and apoptotic potential of p53-BakMTS and p53-BaxMTS were confirmed, whether the apoptotic response occurs mainly through the mitochondrial pathway or through residual nuclear activity was examined. As expected, wt p53 showed high nuclear activity, while p53-BakMTS and p53-BaxMTS showed no transcriptional activity, indicating that the apoptotic function of p53-BakMTS and p53-BaxMTS is transcriptionally independent (FIG. 16a).

Further, it was demonstrated that the apoptotic activity of p53-BakMTS and p53-BaxMTS is through the intrinsic apoptotic pathway. Mitochondrial outer membrane permeabilization (MOMP) and caspase-9 activation can only be initiated via the intrinsic apoptotic pathway. In fact, p53-BakMTS and p53-BaxMTS triggered permeabilization of the mitochondrial outer membrane and induced caspase-9 activation confirming the involvement of the intrinsic apoptotic pathway (FIG. 16b,c).

Whether a single domain of p53 (DBD) is sufficient to trigger apoptosis or if full length p53 is essential for cell death induction was examined. Although the TD was reported to be essential for wt p53 function to exert its apoptotic effect via Bak oligomerization, the DBD in isolation with a MTS from Bak or Bax is sufficient to induce apoptosis (FIG. 17). Murphy and colleagues reported that p53 must form a dimer or a tetramer for initiating Bak oligomerization. However, this data is consistent with previous findings that monomeric p53 or just the DBD is sufficient to trigger mitochondrial dependent apoptosis. Moreover, the DBD of p53 (specifically through residues L120, R248, R273, R280, G285 and G287) has been shown to be the domain responsible for binding to Bak. DBD fused to MTS is sufficient to trigger apoptosis can be that since wt p53 does not have a MTS, mitochondrial import of wt p53 is only possible through dimerization and monoubiquitination via MDM2. Forcing the DBD of p53 to be in close proximity to Bak via the Bak targeting signal can trigger an interaction with Bak via the previously reported residues in the DBD region leading to activation of the apoptotic pathway. In wt p53 the lack of a Bak MTS does not allow the interaction to take place. These results indicate that the Bak MTS fused to DBD can replace MBD and TD for mitochondrial import and is sufficient to cause Bak homo-oligomerization and apoptosis.

To ensure that this finding is not a T47D cell specific effect, the DBD-BakMTS and DBD-BaxMTS were tested in three different cancer cell lines (FIG. 18; Tbl. 2). In fact, DBD-BakMTS showed even significantly higher (FIG. 18a) or trending higher (FIGS. 17a,18c and e) activity compared to p53-BakMTS. A reason for the higher activity of DBD-BakMTS over full length p53-BakMTS is that DBD-BakMTS is lacking the MBD and C-terminus which are essential for the p53 degradation pathway. MDM2 binds to MBD of p53 and initiates polyubiquitination of the C-terminus causing proteasomal degradation. As DBD-BakMTS lacks the MBD and C-terminus, it can avoid polyubiquitination and subsequent proteasomal degradation, thus making it more stable than p53-BakMTS.

Unlike constructs fused to Bak MTS, Bax tagged constructs showed inconsistent apoptotic activity profiles (FIGS. 17 and 18). In SKOV-3, p53-BaxMTS and DBD-BaxMTS activities were not significantly different from their negative control E-BaxMTS (FIG. 18d), and in H1373 p53-BaxMTS did not show higher activity than E-BaxMTS (FIG. 18b). A possible explanation for this is that Bax is constantly shuttled between cytoplasm and mitochondria. Mitochondrial p53 might lack the ability to activate cytoplasmic Bax while the fraction of Bax which is present at the mitochondria might not be sufficient to induce apoptosis in certain cell lines. This offers an explanation why p53-BaxMTS and DBD-BaxMTS show inconsistent results (FIG. 17a; 18b,d, and f). Unlike Bax, Bak is mainly present at the mitochondria and sequestered by anti-apoptotic Mcl-1 and to a certain extent by Bcl-XL.

The apoptotic mechanism of p53-BakMTS and DBD-BakMTS was then investigated. p53-BakMTS causes apoptosis through a p53/Bak specific pathway, which was confirmed by mutating amino acids of p53 DBD (K120, R248, R273, R280, E285, E287) that are known to interact with the pro-apoptotic Bak protein to abolish any p53/Bak specific interactions. In fact, mutating these amino acids showed a complete loss of p53-BakMTS and DBD-BakMTS function (FIG. 19a).

Besides pro-apoptotic Bak, p53 also interacts with anti-apoptotic Bcl-XL through its DBD (residues G117, S121, C176, H178, N239, M243, R248, G279, and R280). p53 interacts through amino acids R248 and R280 with Bak and Bax. To further validate that the apoptotic mechanism is mainly dependent on pro-apoptotic Bak protein and not Bcl-XL, the p53K120E-BakMTS and DBDK120E-BakMTS were created. This K120 residue is known to be significant and specific for the p53/Bak interaction. K120E mutation in p53-BakMTS and DBD-BakMTS resulted in dramatic loss of activity suggesting involvement of Bak/specific p53 pathway (FIG. 19b).

5. Conclusion

In summary, the data shows that fusing p53 to MTSs from Bak or Bax results in mitochondrial localization and activation of an intrinsic apoptotic response. DBD-BakMTS and p53-BakMTS show apoptosis in breast, non-small cell lung, ovarian and cervical carcinomas in a p53/Bak dependent manner. p53-BakMTS and DBD-BakMTSin anadenoviral drug delivery can be used in orthotropic breast cancer and ovarian cancer. Mitochondrially targeted p53, which does not dimerize nor activate genes in the nucleus, simply has a direct apoptotic effect. Therefore, functional, mitochondrially targeted monomeric p53 re-introduced into cancer cells would act as a “sledgehammer,” effective under any circumstances regardless of genetics or the pathway upon which the cancer develops.

### P. Example 3

p53 is a transcription factor that stimulates a network of signals through two apoptotic signaling pathways: the extrinsic pathway (nuclear transcriptional activation) through death receptors and the intrinsic pathway through the mitochondria. While much work using p53 has exploited the extrinsic pathway, the intrinsic pathway is more appealing, due to its rapid, direct apoptotic effects at the mitochondria and absence of inactivation by the dominant negative effect (dimerization and inactivation by mutant wt p53 in cancer cells). p53 directed to the mitochondria functions as a monomer (does not require dimerization). Its rapid effects represent the shortest pathway for executing p53 death signaling, which triggers a wave of caspase activation and apoptosis.

Mitochondrially targeted domains of p53 have never been attempted for ovarian cancer therapy, unlike wt p53. Despite some promising pre-clinical studies, a high profile report in Lancet reported the reasons for the failure of wt p53 in ovarian cancer: 1) multiple genetic changes in cancer and epigenetic dysregulations; 2) dominant negative cross talk between ectopic wild-type p53 and dominant p53 mutants, p63, or p73; and 3) problems in targeting tumor cells with adenoviral vectors due to heterogeneity or lack of expression of CAR and integrin co-receptors in ovarian tumors and adenovirus-neutralizing antibodies. Due to these substantial reasons for failure, wt p53 for ovarian cancer has since largely been abandoned. To overcome these problems this study exploits the non-transcriptional apoptotic pathway of p53 and uses an effective delivery strategy. A small, monomeric domain of p53 with a mitochondrial targeting signal (MTS) that is highly potent, that kills any cancer cell regardless of p53 status or genetics, and bypasses the dominant negative effect (is not deactivated by endogenous p53, p63, or p73) can be engineered.

p53 attached to a MTS that targets the outer membrane of the mitochondria is efficient in inducing a direct apoptotic effect, independent of transcriptional activity or dimerization of p53; moreover, it has been identified that targeting the DNA binding domain (DBD) of p53 is sufficient (and sometimes more efficient than wt p53) in inducing a direct apoptotic effect at the mitochondria. Mitochondrial p53 inhibits the function of pro- and anti-apoptotic Bcl-2 family members, leading to mitochondrial outer membrane permeabilization, and subsequent apoptosis. p53-MTS constructs, using MTSs from Bcl-2 family member proteins (Bcl-XL, Bak, or Bax) to directly trigger apoptosis can be used. The advantage of using a mitochondrial targeted protein encoded by a gene, rather than a cytotoxic agent is the ability to incorporate a promoter for cancer specific expression of that protein. Ultimately, correction of the p53 pathway and activation of apoptosis can be a universal approach: functional, mitochondrially targeted monomeric p53 re-introduced into cancer cells can act as a “sledgehammer,” effective under any circumstances (regardless of genetics or the pathway upon which the cancer develops).

Despite susceptibility of some cancer cells to mitochondrially targeted agents (“mitochondrial priming”), the advantage of targeting a gene to the mitochondria over a cytotoxic agent is the ability to incorporate cancer specificity with a cancer-specific promoter (only expressed in, and therefore only toxic in, cancer cells).

Current gene therapy with p53 has focused solely on its role as a transcription factor. Targeting p53 to the mitochondria for cancer therapy has been attempted by Moll, but did not achieve clinical translation. Various MTSs have been compared for their ability to induce p53-mediated apoptosis, and found that Bcl-XL MTS was superior to others (including Moll's constructs). Higher rates of apoptosis were seen in several cancer lines using p53-Bcl-XL MTS compared to Moll.

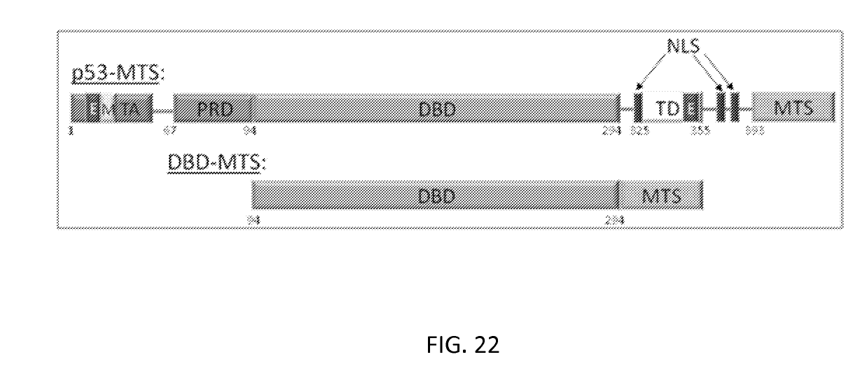

DBD alone induces apoptosis. DBD is the smallest domain of p53 required to induce apoptosis at the mitochondria. Reports have implicated various domain(s) of p53 as directly involved in triggering apoptosis. Although the tetramerization domain (TD) was reported to be essential for full length p53 to exert its apoptotic effect (via Bak oligomerization), it was determined here that the DBD in isolation with a MTS derived from any of 3 Bcl-2 family members (Bcl-XL MTS, Bak MTS or Bax MTS) is sufficient to induce apoptosis in many cancer cell types (FIG. 25). Omitting TD is also appealing since it precludes binding to endogenous p53 in cancer cells, and therefore cannot be transdominantly inactivated; it is the TD that allows endogenous p53 to dimerize with exogenous wt p53.

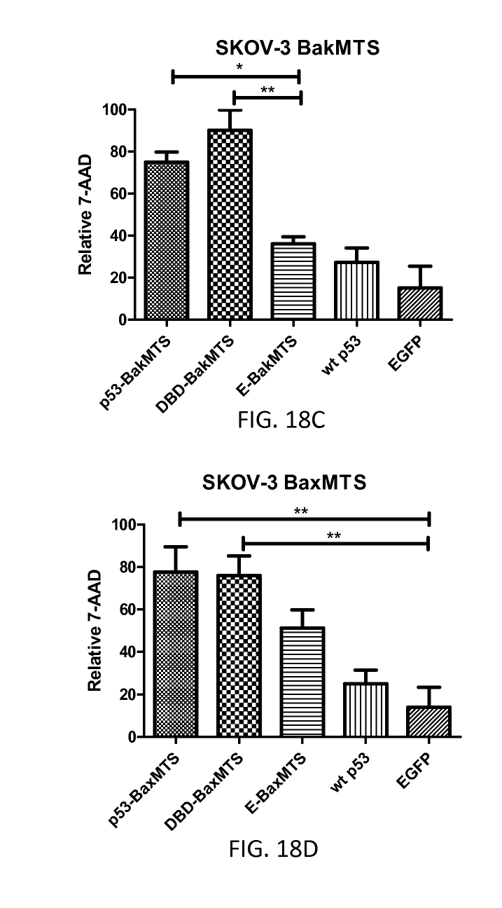

While anti-cancer research using p53 as a therapeutic has exploited the extrinsic, transcriptionally-dependent apoptotic pathway, the intrinsic apoptotic pathway may be more ideal due to its rapid, direct apoptotic effects at the mitochondria, and absence of inactivation by the dominant negative effect (dimerization and inactivation by mutant p53 in cancer cells). p53 directed to the mitochondria is shortest pathway for executing p53 death signaling which triggers a wave of caspase-3 activation and apoptosis. Efforts have been focused on p53 domains and their functions, and the minimal domain of p53 required to cause apoptosis when fused to an optimal MTS that targets the outer mitochondrial membrane has been defined. This proposal builds on the basic mechanistic studies of the interaction of p53 domains at the mitochondria with an entirely novel p53-MTS (using the Bak MTS). DBD-MTS can be tested alone and in combo with chemotherapeutics first in ovarian cancer cell lines before proceeding with a new metastatic ovarian cancer mouse model. FIG. 20 is a summary of the mechanism of action of these drugs which rationalize their use in synergistic cell killing.

Metastasis and recurrence: p53 is the central mediator of apoptosis, which is thought to be a safeguard system for preventing metastasis. Loss of p53 is associated with metastasis, while p53 mutation or loss have been found in recurrent ovarian cancer. Attempts at re-introducing wt p53 to address metastasis/recurrence may be fraught with difficulty, due to p53 acting at the transcriptional level. Wt p53 homo-tetramerizes and directly activates over 125 target genes. Mitochondrially targeted p53 does not dimerize nor activate genes in the nucleus. Thus, metastatic and recurrent ovarian cancer may be addressed with the use of a direct apoptogen: DBD-MTS. The monomeric domain of p53 with a MTS can be highly potent, can kill any cancer cell regardless of p53 status or genetics, and can bypass the transdominant deactivating effect.

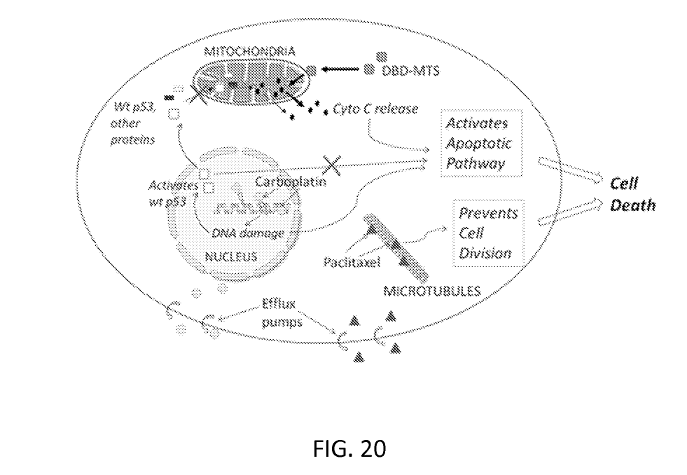

Novel rationale for selecting the optimal MTS: In addition to the Bcl-XL MTS, MTSs derived from proteins that target the OMM were explored. The rationale for using MTSs from pro-apoptotic Bcl-2 family members Bak and Bax stems from the ability of these proteins to trigger mitochondrial outer membrane permeabilization (MOMP), resulting in robust release of cytochrome C which leads to apoptosis. Results show that Bak MTS can be optimal for apoptosis (FIG. 23). In cancer cells, overexpression of Bcl-2, Bcl-XL and Mcl-1 correlate with aggressive phenotypes that are chemotherapy-resistant. Inhibitors like navitoclax and ABT-199 target anti-apoptotic Bcl-2 family members, which neutralize anti-apoptotic proteins at the mitochondria allowing pro-apoptotic Bcl-2 members Bak or Bax to homo-oligomerize, leading to apoptosis. However, these inhibitors do not inactivate anti-apoptotic Mcl-1, whose overexpression is linked to reduced response to chemotherapy and poor prognosis (including ovarian cancer), limiting the therapeutic use of these inhibitors. This approach directly activates pro-apoptotic Bak by targeting p53 to the mitochondria using Bak's own MTS. p53 activates Bak by disrupting Bak/Mcl-1 and Bak/Bcl-XL complexes. DBD-BakMTS can trigger apoptosis in cancer cells by preferential interaction with Bak (over Mcl-1 or Bcl-XL). The data indicate a robust apoptosis from DBD-BakMTS that is entirely dependent on residues in the DBD which directly interact with cellular Bak (FIG. 25).

1. A Novel Apoptotic Gene Therapy Construct (Called DBD-MTS) Based on the DBD of p53, Coupled to an Optimal Mitochondrial Targeting Signal (MTS), Capable of Activating Intrinsic Apoptosis.

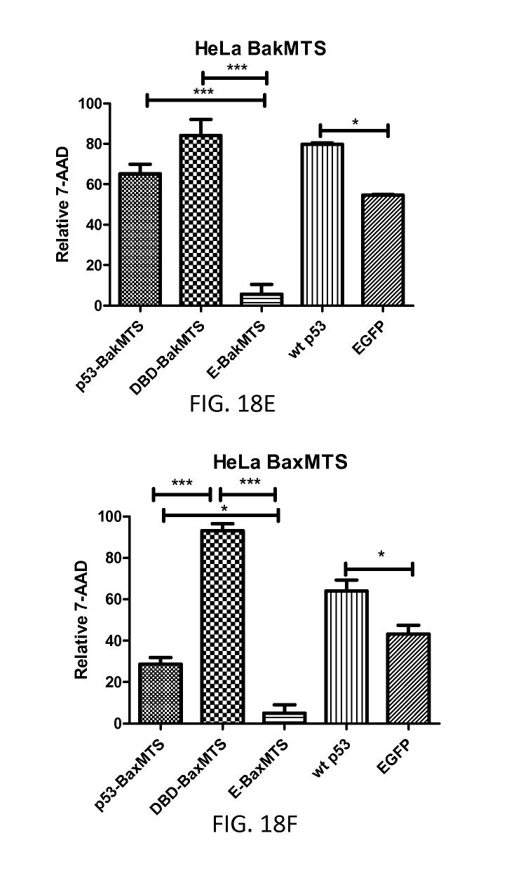

The smallest domain of p53 (excluding TD to avoid dominant negative interaction with endogenous p53) can be identified in combination with the best MTS to use to achieve apoptosis. As discussed above, the DBD of p53 as the minimal domain that can trigger apoptosis at the mitochondria (see FIG. 25). Initially this was surprising since multiple domains have been reported to be needed for p53-specific apoptosis, but our data indicate that this is not the case when isolated domains are used. Targeting the OMM with a MTS from Bcl-XL or TOM triggers p53-specific apoptosis. Exploration of other OMM MTSs led to investigation of MTSs from pro-apoptotic proteins including Bak and Bax. Preliminary data indicate that the Bak MTS may be the optimal MTS to target the DBD to cause apoptosis of cancer cells. DBD-BakMTS has a mechanism of action described in FIG. 21.

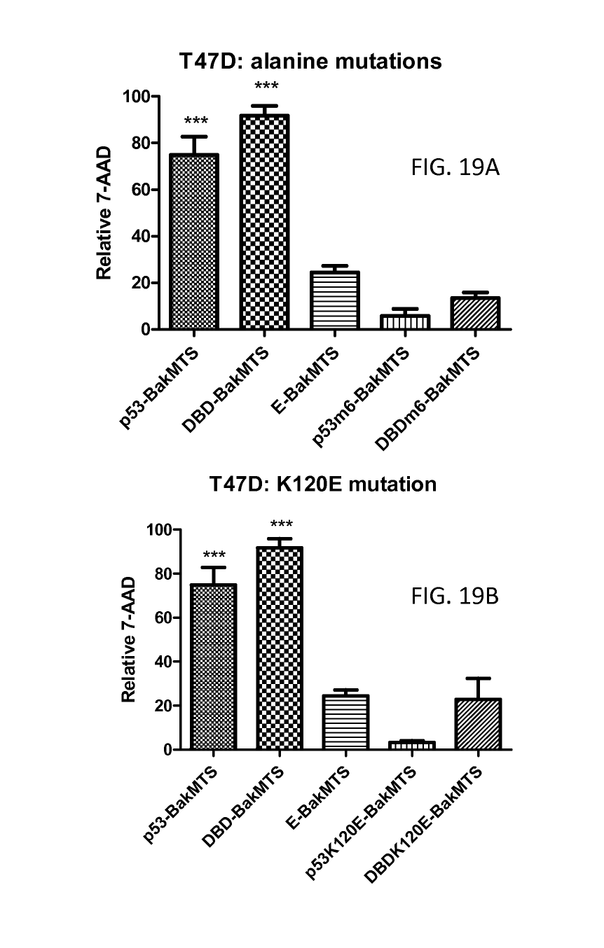

Plasmid construction: plasmids with EGFP (enhanced green fluorescent protein; CMV promoter) were subcloned (EGFP can be removed for animal studies) with Bak or Bax MTS (BakMTS and BaxMTS; negative controls), Bak or Bax MTS C-terminally attached to p53 (p53-BakMTS and p53-BakMTS), and Bak or Bax MTS C-terminally attached to the DNA binding domain of p53 (DBD-BaxMTS and DBD-BakMTS). See FIG. 22 for main constructs and MTSs. To ensure that the apoptotic effect of p53-BakMTS and DBD-BakMTS constructs occur through the p53 Bak specific pathway, all sites of p53 (K120A, R248A, R273A, R280A, E285A, E287A) that contact pro-apoptotic Bak protein were mutated to alanine (named p53m6-BakMTS and DBDm6-BakMTS). Mutation of these residues should abolish any apoptotic activity. A construct containing only a K120E mutation was also made (p53K120E-BakMTS and DBDK120E-BakMTS), since this K120E mutation is known also to abolish the p53 interaction with Bak, and is expected to abolish apoptotic activity of the constructs.

Cell lines and transient transfections: 1471.1 murine breast adenocarcinoma cells (G. Hager, NCI, NIH), T47D human breast tumor cells (contain L194F mutant p53; ATCC), H1373 human non-small cell lung carcinoma cells (p53 null cells), SKOV-3 human ovarian adenocarcinoma cells (p53 null cells) and HeLa human epithelial cervical adenocarcinoma cells (contain wt p53; from ATCC) can be grown as described above, and transfected with Lipofectamine 2000 as previously described.

For statistical analyses in all figures, n=3, and one-way ANOVA was used/will be used for all aims, with Bonferroni's post-test unless otherwise indicated; *p<0.05, **p<0.01; ***p<0.001.

Mitochondrial staining, microscopy, and image analysis: To verify mitochondrial localization of constructs, cells transfected with constructs will be stained with MitoTracker Red FM (Invitrogen) as before. Images of 1471.1 and T47D live cells (photogenic adherent cells) can be acquired with an Olympus IX71F fluorescence microscope with a F-View Monochrome CCD camera. Images can be analyzed with ImageJ using Pearson's correlation coefficient (PCC) and post Costes' algorithm. PCC≧0.6 are considered to be colocalized. Preliminary data indicate mitochondrial localization: PCC=0.75 for p53-BakMTS; PCC=0.76 for p53-BaxMTS (n=3; 10 cells each). DBD-MTSs can also be tested.

Apoptosis Assays: assays representing early, mid, and late apoptosis can be done as described above, including Caspase-9 assay (early apoptosis, cyt C release), Annexin V-APC (mid-stage apoptosis); 7-AAD assay (late apoptosis), TUNEL assay (DNA nicks/fragmentation, late apoptosis), TMRE assay (mitochondrial depolarization/mitochondrial outer membrane permeabilization). FIG. 23 indicates apoptotic activity of p53-BakMTS (1st bar) and p53-BaxMTS (3rd bar) in T47D cells.

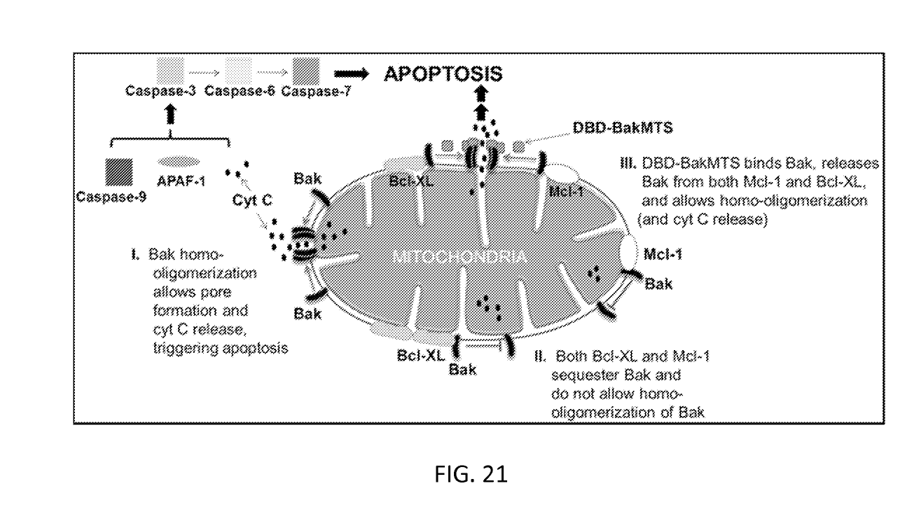

Reporter Gene Assay can verify that our mitochondrially targeted constructs do not induce transcriptional (nuclear) activity. p53-BakMTS, EGFP-BakMTS, p53-BaxMTS, and EGFP-BaxMTS plasmids will be co-transfected with p53-Luc Cis-Reporter encoding Firefly luc (Agilent), and Renilla luc internal control (Promega) in cells using the Dual-Glo Luciferase assay system as described previously. FIG. 24 shows that only wt p53 is capable of transcriptional activation in the nucleus, as expected. All other MTS-constructs demonstrate no transcriptional (luciferase) activity.

Activity of DBD-BakMTS and DBD-BaxMTS: FIG. 23 indicates apoptosis when BakMTS and BaxMTS are fused to full length wt p53; the DBD of p53 is sufficient to induce apoptosis at the mitochondria [15]. Therefore, the apoptotic activity of DBD-BakMTS and DBD-BaxMTS were tested in 3 cell lines with varying p53 status, including HeLa cervical (wt p53), H1373 lung (p53 null), and T47D breast (mutant p53) cancer cells. FIG. 25 shows that DBD-BakMTS induces robust apoptosis, similar to, or greater than wt p53 with MTS in all 3 cell lines tested. DBD-BakMTS (2nd bars in each figure; starred) outperformed DBD-BaxMTS.

Mutational Analysis of p53 DBD Residues that Interact with Bak:

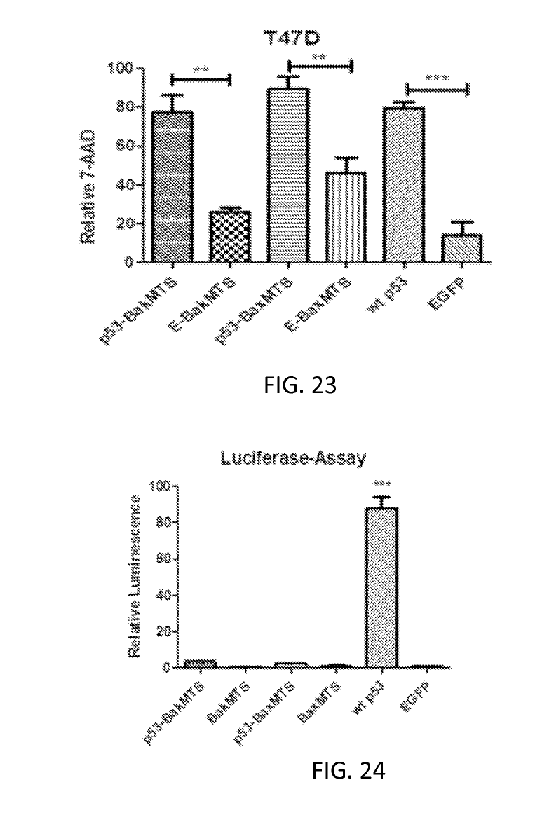

6 mutations in DBD of p53 are known to obliterate its interaction with Bak; a single point mutation (K120E) also achieves the same effect. Mutations are described in “plasmid construction.” In a study in T47D cells (FIG. 26), transfected p53m6-BakMTS and DBDm6-BakMTS (m6=mutations) show a complete loss in apoptotic activity compared to their non-mutated controls. This implies that a direct interaction between DBD and Bak is responsible for apoptosis. Co-immunoprecipitation (co-IP): p53-BakMTS or DBD-BakMTS can be transfected in cancer cells to prove that they are capable of disrupting Bak/Mcl-1 and Bak/Bcl-XL interactions (which prevent apoptosis from occurring; see FIG. 21 for schematic), and can be compared to untreated T47D cells. In untreated cells, Bak can be pulled down with a Bak-specific antibody, and probed with a Mcl-1 or Bcl-XL antibody (anti-Bak, -Bcl-XL, and -Mcl-1 Ab′ from Santa Cruz Biotech). The Bak/Mcl-1 or Bak/Bcl-XL immune complexes can be present on a SDS-PAGE gel.

In addition to late apoptosis (7-AAD, FIG. 25) DBD-BakMTS can trigger apoptosis (caspase-9, Annexin V-APC, TUNEL, TMRE) in other cell lines (HeLa, H1373, SKOV3) regardless of p53 status. No reporter gene activity is expected for DBD-BakMTS (as shown for p53-BakMTS, FIG. 24). Mutant versions (m6, FIG. 26, and K120E) of p53 or DBD which lack binding to Bak, will not be apoptotically active. p53-BakMTS and DBD-BakMTS can disrupt Bak/Mcl-1 and Bak/Bcl-XL interactions (co-IPs).

2. The Apoptotic Potential of DBD-MTS in Ovarian Cancer Cell Lines with Varying p53 Status, Individually, and in Combo with Standard of Care, Carboplatin and Paclitaxel (CPTX).

The killing potential of the optimized DBD-Bak MTS can be tested in ovarian cancer cell lines. Ovarian cancer cell lines resembling high grade serous carcinoma (HGSC) are not necessarily the most commonly used ovarian cancer cell lines in the literature; therefore, some true HGSCs can be selected, in addition to commonly used cell lines. Normal cell lines can also be tested to determine possible toxicity to non-target cells (See Table 3 for cell lines).

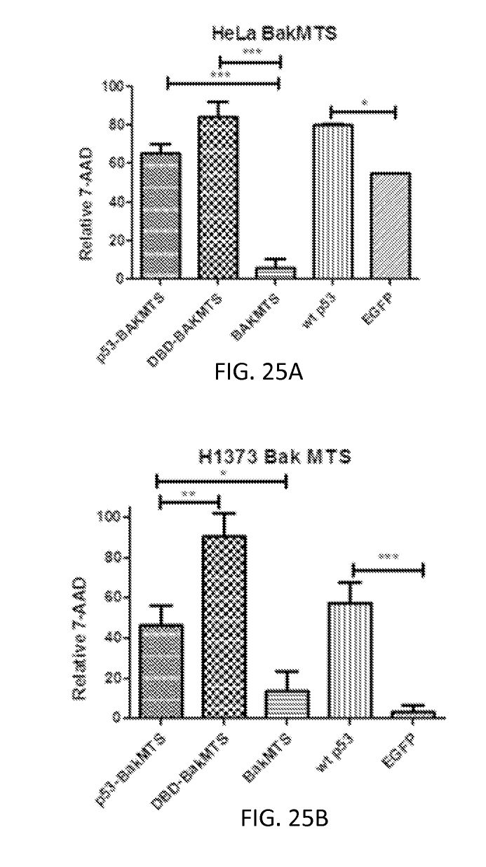

For testing of DBD-BakMTS in ovarian cancer and normal cells, assays described above can be used for cell lines in Table 3. FIG. 27 is important data indicating apoptosis using DBD-BakMTS in an ovarian adenocarcinoma cell line. For combination therapy with carboplatin and paclitaxel (CPTX), CPTX is typically given at 20,000:1 molar ratio (IC50 values from 20-50 uM), and cell viability is measured 72 h later. For these studies, sub IC50 values can be used to determine synergy between DBD-BakMTS and CPTX. While synergism can be easily detected, combination index (CI) for multiple drugs can be calculated for n drug combos at x % inhibition using a CI equation by Chou et al. where CI<1 synergism; CI=1 additive effect. While any of the activity assays can be used for determining % inhibition, a simple cytotoxicity assay (cell death by trypan blue exclusion) can initially be performed. DBD-BakMTS alone or in combo with CPTX can cause apoptosis in all ovarian cancer cell lines.

To prevent the possibility of normal cells being killed by DBD-BakMTS, a modified hTERT promoter (VP16-Ga14-WPRE) can be used that expresses only in ovarian cancer cells at a level similar to the strong CMV promoter (in original constructs). This promoter can cause expression (and apoptosis) only in cancer cells.

TABLE 3

| Ovarian | p53 | BRCA1/2 | Characteristics | |
|---|---|---|---|---|
| cancer cells | status | status | (all cell lines are human) | Source |
| Kuramochi | p53 | BRCA1 | From ovarian cancer ascites; epithelial- | JCRB (cat |
| | mutation | mutant | like morphology, likely to be HGSC [68] | #JCRB0098) |
| Caov-4 | p53 | Wt | Ovarian adenocarcinoma; derived from | ATCC cat #HTB-76 |
| | mutation | | metastatic site (fallopian tube); likely to | |
| | | | be HGSC [68] | |
| NIH:OVCAR-3 | p53 | Wt | Ovarian adenocarcinoma; epithelial; | ATCC cat #HTB- |
| | mutation | | likely to be HGSC [68] | 161 |
| OVCAR4 | p53 | Wt | Ovarian adenocarcinoma; likely HGSC | GenScript cat#210 |
| | mutation | | [68]; known to be resistant to platinum | or NCI CCR |
| SKOV3 | p53 null | Wt | Ovarian adenocarcinoma; from ascites; | ATCC cat#HTB-77 |
| | | | not likely to be HGSC [68] | |
| Normal cells: | Wt p53 | n/a | BJ: Normal fibroblasts [73]; IHOEC: | ATCC cat#CRL- |
| BJ, IHOEC | | | SV40 immortalized ovarian epithelial | 2522; abm |
| | | | cells | cat#T1074 |

Ovarian cancer cells can robustly be killed by DBD-BakMTS, and show synergy with CPTX (except OVCAR4). If normal cells apoptose with DBD-BakMTS (with constitutive CMV promoter), the modified hTERT promoter can prevent expression (and therefore killing) in these cells. Alternative cancer specific promoters include: survivin, unmodified hTERT, or ovarian cell-specific OSP-1 promoter. MTS from Bax is an alternative. DBD-BakMTS constructs can have a very rapid and potent effect (expected to occur regardless of p53 or BRCA1/2 status), eliminating entirely the need for CPTX treatment.

### Q. Example 4

The tumor suppressor p53 is one of the most frequently mutated proteins in human cancer and has been extensively targeted for cancer therapy. This resulted in wild type p53 gene therapeutic approval for the treatment of head and neck cancer in China. p53 mainly functions as a transcription factor and stimulates a variety of genes involved in the intrinsic and extrinsic apoptotic pathway by binding to p53 responsive elements as a tetramer. In cancer cells, mutations in p53 typically occur in its DNA binding domain (DBD), while its tetramerization domain remains intact. Therefore, mutant p53 can heterotetramerize with wt p53 and abolish its transcriptional activity (dominant negative effect).

While transcriptionally active wt p53 is used for gene therapy, mitochondrial p53 has not been fully exploited yet. Targeting p53 to the mitochondria causes a direct rapid apoptotic response by directly interacting with pro- and anti-apoptotic proteins at the mitochondrial outer membrane. Because the monomeric from is sufficient to interact with pro- and anti-apoptotic proteins, mitochondrial p53 is not affected by the dominant negative inactivation. To ensure mitochondrial targeting of p53, p53 was targeted to different mitochondrial compartments; mitochondrial outer membrane, inner membrane and matrix. It was demonstrated that MTSs from the mitochondrial outer membrane are optimal for p53-specific activation. In addition, it was discovered that the minimal domain of p53, its DNA binding domain (DBD), which is needed for apoptosis induction. Further studies have shown that fusing p53 or DBD to MTS from Bcl-XL causes p53/Bcl-XL specific apoptosis while fusing them to Bak results in p53/Bak specific apoptosis, emphasizing that mitochondrial targeting of p53 is highly dependent on the MTS used. Further, it has been shown that DBD fused to the MTS from Bcl-XL can overcome dominant negative inhibition in vitro, but it was unable to shrink dominant negative MDA-MB-468 tumors in an orthotropic mouse model at one dosing regimen attempted.

This study is to design apoptotic proteins based on p53 domains to create modified versions of p53. Optimizing mitochondrial targeting of p53 for cancer therapy was accomplished.

1. The p53 Protein

The tumor suppressor p53 is one of the most widely studied proteins. Over the last 30 years it has been shown that p53 is involved in a wide network of signaling pathways that involves tumorigenesis, cellular senescence, metabolism and DNA damage preventing tumorgenesis. Since its discovery, p53 has been of great interest because it is mutated in almost 50% of all human cancers. Mutations in p53 are crucial for cancer development and therefore make it an interesting target for cancer therapy.

2. Structure of Wt p53

The 393 amino acid p53 protein is encoded by the TP53 gene. It contains a N-terminus, a DNA binding domain (DBD) and a C-terminal region as shown in FIG. 12. The N-terminus consists of the transactivation domain (TA) and the proline rich domain (PRD). The TA can be further divided into MDM2 binding domain (MBD) and a nuclear export signal (NES). The C-terminus contains three nuclear localization signals (NLS)s, one nuclear export signal (NES) and the tetramerization domain (TD) as depicted in FIG. 12.

The TA is essential for either the transcriptional activity of p53 or for its degradation depending on post-transcriptional modifications occurring in the TA. When no transcriptional modifications occur, p53 is ubiquitinated via MDM2 and MDMX and degraded via the ubiquitin-dependent proteasomal pathway. On the other hand, when Thr 18 is phosphorylated, the affinity of TAD for transcriptional cofactors such as p300/CBP and its various subdomains is highly increased and p53 can exhibit its function as a transcription factor.

The PRD has a predominantly structural role. It allows for the TA to interact with transcription cofactors and components of the basal transcription machinery. The DBD, as the name implies, binds directly to DNA sequences and triggers gene transcription.

The C-terminus undergoes various posttranslational modifications and can adopt different secondary structures. Modifications on this region play complex roles so that it can interact with numerous partner proteins. The three NLSs within the C-terminus are important for localization to the nucleus where p53 exhibits its function as a transcription factor. Tetramer formation is essential for the majority of its transcriptional activity. The p53 tetramer is formed via a dimeric intermediate. Primary dimers are stabilized by an intermolecular β-sheet and helix-packing interactions. The hydrophobic helix interfaces of two such dimers form a tightly packed tetramer, which is highly thermodynamically stable.

3. Degradation of p53

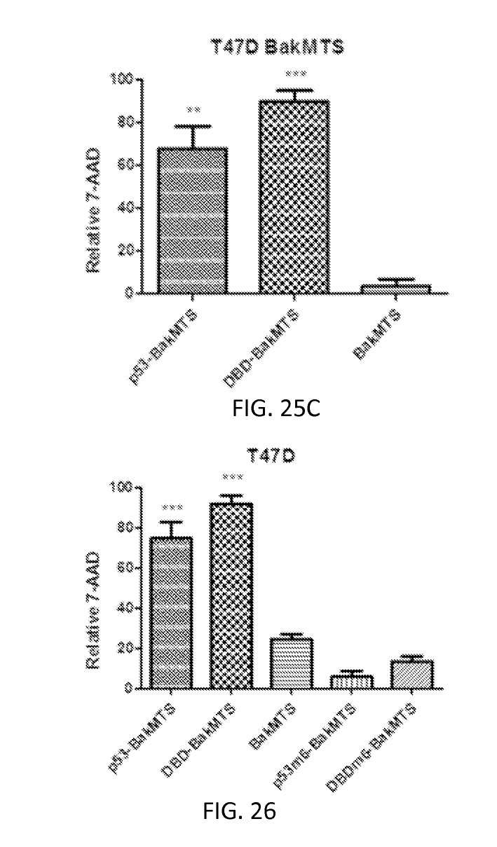

p53 is known as a transcription factor which inhibits tumor growth. It is capable of transactivating a variety of genes responsible for apoptosis, cell cycle arrest and DNA repair. Since p53 induces cell-cycle arrest and apoptosis, it has an inhibitory effect on cellular growth. Therefore, p53 needs to be regulated so normal development can take place. The major regulator of p53 is the E3 ubiquitin ligase MDM2. Even though other p53 E3 ligases have been discovered over the last couple of years, MDM2 still appears to be the physiological and primary E3 ligase regulating p53. MDM2 and p53 form an autoregulatory feedback loop in which p53 transactivates MDM2 and influences its own degradation. p53 is degraded via different degradation pathways which all eventually result in polyubiquitination and eliminations by the 26S proteasome (FIG. 28). In the nucleus, p53 binds directly to the MDM2 binding domain, monoubiquinates it and initiates nuclear export. Cytoplasmic monoubiquitinated p53 then gets polyubiquitinated by E4 factors (USE4B) or E4-like molecules (Cul4-DDB complex), and MDM2 is then sent to the proteasome for degradation (FIG. 28). Additionally, other E-ligases (Pirh2) can facilitate polyubiquitination and proteasomal degradation of p53 with no involvement of MDM2. Furthermore, it has been reported that MDM2 can form a heterodimer with another protein MDMX facilitating polyubiquitination and proteasomal degradation (FIG. 28). MDMX and MDM2 show low amino acid sequence overlap but a nearly identical p53 binding domain located at their N-terminus and a C-terminal RING domain. Heterodimer formation occurs through this RING domain. MDMX alone does not have significant E3 ligase activity, but has been shown to modulate p53 via modulation of its transcriptional activity.

The regulatory effect of MDM2 on p53 can also result in negative outcomes. The MDM2 gene is amplified or overexpressed in many human cancers, consequently inactivating p53. These cancers have been associated with poor prognosis. Therefore, the interaction of p53 and MDM2 provides an interesting target for cancer therapy.

4. Regulation of Gene Transcription: Cell Cycle Arrest or Apoptosis?

p53 positively and negatively regulates the expression of responsive genes. Depending on the severity of damage to the cell, p53 decides the fate of the cell. p53 response elements (REs) are located within a few thousand nucleotides upstream or downstream from the transcription start site. It has been shown that binding affinity of p53 for its specific REs differs dramatically. Growth arrest-related genes have high affinity sites for p53 whereas proapoptotic genes are mostly associated with low affinity sites. Additionally, some REs exist in open occupied states while others do not. Since recognition of elements in Mdm2 and p21 promoters depend on non-B-DNA conformation, conformation of the DNA can also be important. Taken together these findings indicate that not all targeted genes are equally responsive to p53 and that levels of p53 protein may determine which genes to turn on or off

Mild damage to a cell is often reparable. Basal or low levels of p53 usually turn on cell cycle genes. Additionally, p53 undergoes pro-arrest modifications by ubiquitination of the Lys 320. Pro-apoptotic cofactors such as Brn3a reduce the ability of p53 to transactivate pro-apoptotic Bax whereas stimulating transcription of p21, results in cell cycle arrest. Transient cell cycle arrest allows for sufficient time to repair DNA damage and re-entry into the normal cell cycle.

When severe damage occurs, p53 levels rise dramatically and promote transactivation of pro-apoptotic genes due to pro-apoptotic posttranslational modification of the protein, such as acetylation of K120, K320 and phosphorylation of S46 in the p53 protein. DNA damage activates ASSP1 and 2 which interact with DBD of p53 and activate pro-apoptotic Bax and PIG3 genes but do not promote transcription of pro-arrest genes such as p21 or regulatory genes such as Mdm2. Another example is the crosstalk between p53 and the NF-KB subunit p52 leading to repression of the cell cycle activator p21 and activation of proapoptotic DRS and PUMA resulting again in apoptosis.

5. p53 Activates the Intrinsic and Extrinsic Apoptotic Pathway

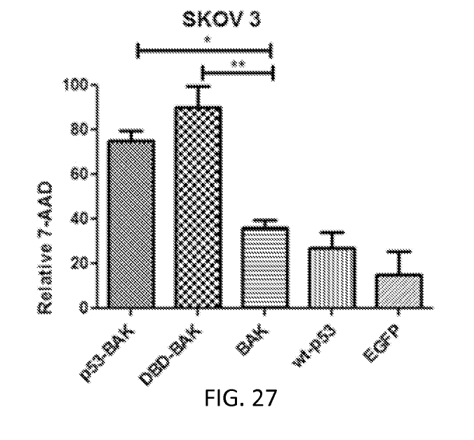

Apoptosis proceeds through intrinsic and extrinsic pathways. p53 is capable of activating both apoptotic pathways. p53 induces genes encoding the transmembrane proteins FAS, DRS and PERP (also called death receptors) which are essential for activating the extrinsic apoptotic pathway (FIG. 29). Death receptors recruit adapter molecules such as FADD, which in turn, recruit procaspase-8 monomers. Dimerization and interchain cleavage of procaspase-8 facilitates the activation of caspase-8. Caspase 8 then leads to cleavage of the inactive procaspase-3 dimer and the inactive procaspase-7 dimer via intramolecular rearrangements resulting in active caspase-3 and caspase-7 dimers leading to apoptosis (FIG. 29). However, cross talk between intrinsic and extrinsic apoptotic pathway occurs via BID which is truncated to tBid via caspase-8.

The intrinsic apoptotic pathway occurs as a result of mitochondrial outer membrane permeabilization (MOMP) which releases various proteins from the mitochondrial intermembrane space such as cytochrome c. p53 targets a key subset of Bcl-2 family genes BAX, NOXA and PUMA which once transcribed and translated into proteins promote cytochrome c release and facilitate caspase-9 activation (FIG. 29). Binding of cytochrome c and apoptotic protease-activating factor 1 (APAF1) assembles into a heptametric, wheel-like structure known as the apoptosome. The apoptosome activates the initiator caspase-9, which then initiates the executioner apoptotic caspases, caspase-3 and caspase-7 (FIG. 29). Additionally, mitochondrial release of second mitochondrial derived activator of caspase (SMAC) and OMI neutralize the caspase inhibitory function of X-linked inhibitor of apoptosis protein (XIAP). XIAP is known to bind and inactivate caspases. All of these processes contribute to DNA fragmentation and eventually apoptosis.

6. Regulation of the Intrinsic Apoptotic Pathway Via the Bcl-2 Protein Family

In healthy cells, anti-apoptotic B cell lymphoma 2 (Bcl-2) family members form heterodimers with pro-apoptotic proteins resulting in their inactivation. However, when an apoptotic stimuli occurs such as DNA damage or ER stress, anti-apoptotic Bcl-2 proteins are released from the inhibitory complexes and homooligomerize resulting in MOMP.

The Bcl-2 family members are localized on the outer surface of the mitochondrial outer membrane. As listed in table 4, the Bcl-2 family of proteins are divided into three groups based on the Bcl-2 homology (BH) domain; anti-apoptotic Bcl-2 proteins such as Bcl-2, Bcl-w, Bcl-XL, A1 and Mcl-1 consists of four BH domains (BH1-4) and a transmembrane (TM) domain. The BH domain is responsible for their anti-apoptotic function while the TM domain is for the insertion into the mitochondrial outer membrane. Pro-apoptotic Bcl-2 proteins are divided into effectors and enhancers. The effectors are Bcl-2-associated X protein (Bax), Bcl-2 antagonist or killer (Bak) and Bcl-2-related ovarian killer protein (Bok). They contain three BH domains (BH1-3) and the TM domain for membrane insertion. Unlike other Bcl-2 proteins the pro-apoptotic enhancers BCL-2 antagonist of cell death (BAD), BH3-interacting domain death agonist (BID), BCL-2-interacting killer (BIK), BCL-2-interacting mediator of cell death (BIM), BCL-2-modifying factor (BMF), BCL-2 and adenovirus E1B 19 kDa protein-interacting protein 3(BNIP3), hara-kiri (HRK), p53 unregulated modulator of apoptosis (PUMA) consist of only the BH3 domain and therefore do not insert themselves into the mitochondrial outer membrane.

TABLE 4
Classification of the different Bcl-2 protein family members with
representative members and structural domains.

| Class | Members | Structural domains |
|---|---|---|
| Anti-apoptotic | Bcl-2, Bcl-w, Bcl-XL, | BH1, BH2, BH3, |
| | A1, Mcl-1 | BH4, TM |
| Pro-apoptotic: effectors | Bak, Bax, Bok | BH1, BH2, BH3, TM |
| Pro-apoptotic: enhancers | BAD, BID, BIK, BIM, | BH3 |
| | BMF, BNIP3, HRK, | |
| | Puma | |

7. Transcriptional-Independent Activation of the Intrinsic Apoptotic Pathway by p53

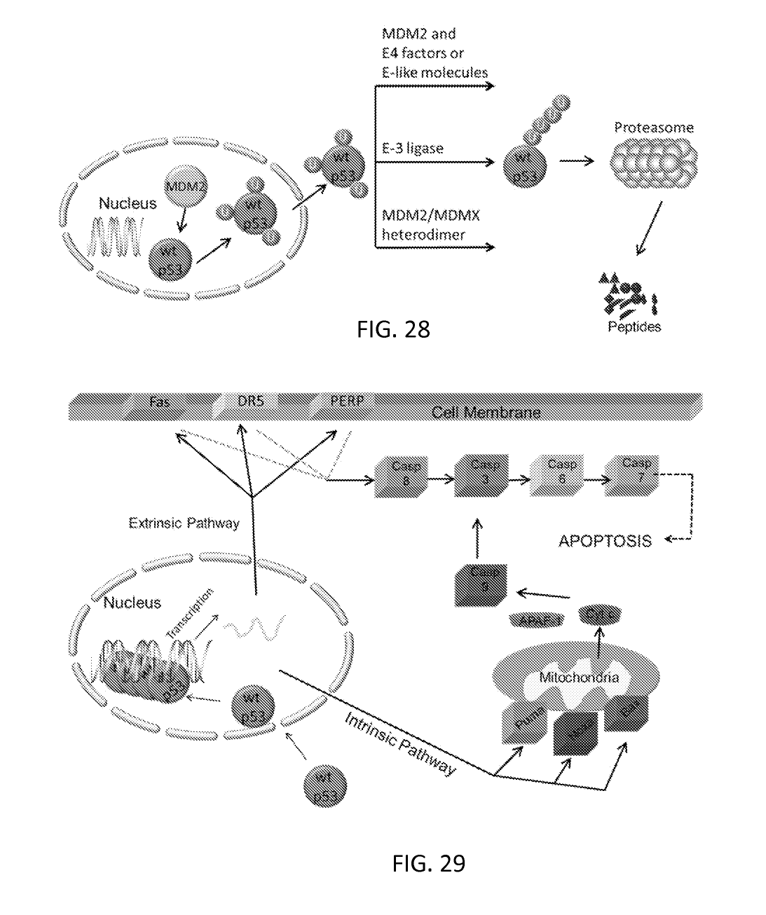

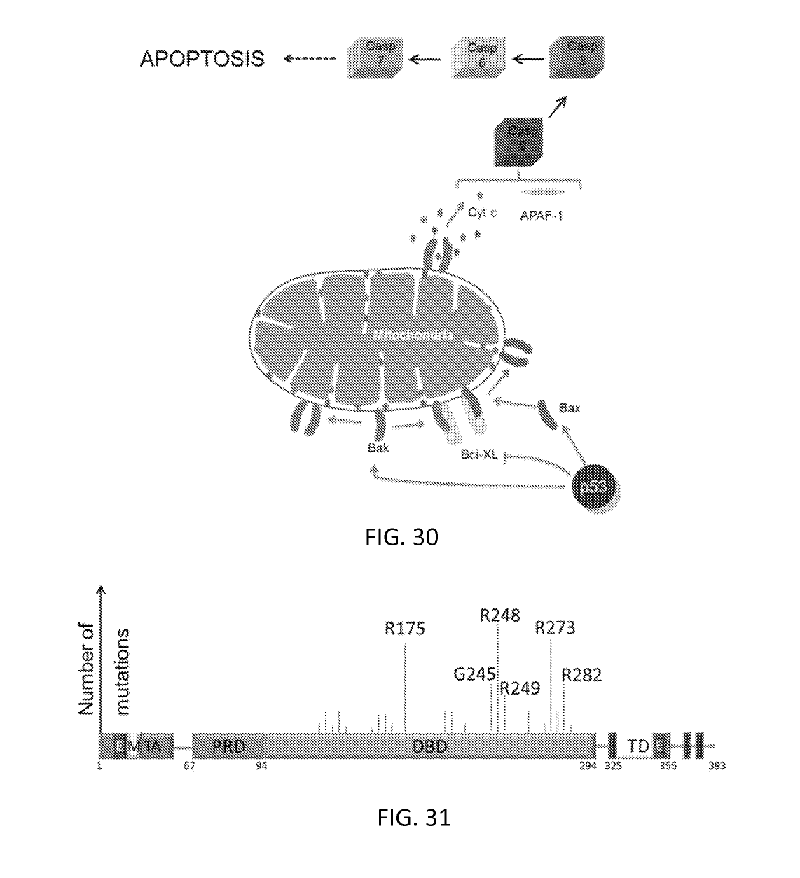

Aside from transactivating Bax, NOXA and PUMA, p53 can also directly activate the intrinsic apoptotic pathway by translocating to the mitochondria upon severe stress signal induction such as radiation. Unlike other mitochondrial proteins, p53 does not contain a mitochondrial targeting signal. It has been hypothesized that nuclear p53 gets monoubiquitinated and exported into the cytoplasm. Cytoplasmic monoubiquitinated p53 is imported into the mitochondria via the herpes virus-associated ubiquitin-specific protease (HAUSP). At the mitochondrial outer membrane p53 interacts directly with pro- and anti-apoptotic Bcl-2 family members (FIG. 30). The DBD of p53 are essential for the electrostatic interaction with anti-apoptotic Bcl-XL and Bcl-2 and pro-apoptotic Bak. The positively charged basic surface of the DBD interacts with the negatively charged BH4 domain and the loops between alpha ⅘ and ⅚ of Bcl-XL and Bcl-2. The affinity of the positively charged DBD to bind pro-apoptotic Bak is 10 times less than to Bcl-XL and Bcl-2. The lower interaction is due to the differences in structure between Bcl-2, Bcl-XL and Bak. While Bcl-XL and Bcl-2 contain a very acidic protein surface and a BH4 domain, Bak does not have a very acidic protein surface nor a BH4 domain and therefore its binding affinity to the positively charged DBD of p53 is decreased. Bax on the other hand has been shown to be activated by p53, but no actual interaction has been detected yet. Since p53 has to directly bind to and sequester Bcl-XL and Bcl-2 to liberate Bak and Bax, the affinity towards these proteins has to be higher than to Bak and Bax, while the pro-apoptotic Bcl-2 proteins Bak and Bax only need to be activated and can then form homo-oligomers. Additionally, the higher affinity towards Bcl-2 and Bcl-XL suggests a sequential mechanism. First p53 binds to Bcl-2 and Bcl-XL and then it binds to Bak and Bax. Therefore, p53 is considered a super BH3-only protein because it acts as an enabler and as an activator of pro- and anti-apoptotic mitochondrial proteins. All of these functions eventually result in MOMP and activation of the intrinsic apoptotic pathway.

8. p53 and its Function in Metabolism and Cell Growth

Besides its well characterized functions of cell cycle arrest and apoptosis, p53 has a clear role in glycolysis, autophagy, cell survival and regulation of oxidative stress, invasion and motility, cellular senescence, angiogenesis, differentiation and bone remodeling. Unlike for transactivation of apoptotic genes where high concentrations of p53 are required, low levels of p53 have been shown to be essential for normal growth, development and metabolism.

p53 has multiple functions in cellular metabolism. It is a negative regulator of glucolysis and lowers gene expression of glucose transporters, inhibits NF-KB and represses the insulin receptor promoter. Additionally, TP53-induced glycolysis and apoptosis regulator (TIGAR) lowers the glycolysis rate and promotes the pentose phosphate pathway. On the other hand, p53 promotes the more efficient tricarboxylic acid (TCA) cycle by enhancing transcription of cytochrome c oxidase 2, subunit1 of complex IV and AIF (essential for complex I function). Taken together the negative regulation of glycolysis and the promotion of the TCA cycle oppose the Warburg effect (aerobic glycolysis) which promotes cancer cells proliferation and is an additional proof for p53 tumor suppressor activity. Concerning oxidative stress, p53 has an ambivalent role. Under mild stress p53 plays an anti-oxidative role. It promotes transcription of GPX1, MnSOD, ALDH4 and TPP53INP1 all of which are antioxidant targets. Under severe stress, p53 promotes ROS which then triggers apoptosis through cytochrome c oxidation.

9. Negative Activity of p53

Besides the functions of p53 that prevent tumorgenesis (DNA-repair, cell cycle arrest, apoptosis and metabolism), the apoptotic function of p53 can also result in unfavorable outcome. p53-dependent apoptosis is the major contributor to radiation and chemotherapy induced sickness. Furthermore, the shortage of glucose and oxygen caused by ischemia results in p53 activation. This can cause stroke and myocardial infarct. Finally, in neurodegenerative diseases, p53 causes cell death in neurons and therefore worsens Parkinson's, Alzheimer's and Huntington's disease. Small molecules such as pifithrin a have therefore been developed which block p53-dependent transcriptional activity while protecting healthy cells from genotoxic stress caused by most chemotherapeutics. Pifithrin a can also protect neuronal cell death.

10. Inactivation of p53 in Cancer Via Mutations

When p53 was discovered in 1979, it was first thought to be an oncogene. The observation that many tumors produce high levels of p53 while normal cells harbor low or undetectable levels suggested that this hypothesis was true. Ten years after its discovery, it was finally determined that p53 is a tumor suppressor. The first assumption of p53 being an oncogene is not surprising since p53 is mutated in around 50% of all tumors, and mutated p53 has oncogenic potential that differs completely from wild type activity. The mutations occurring in p53 are unique among tumor suppressors. While most tumor suppressors are inactivated by deletion or truncating mutations, TP53 is inactivated in 74% of cases by a single monoallelic missense mutation resulting in formation of a stable full length protein.

Mutations in TP53 differ in their frequency depending on the type of cancer. In haematopoietic malignancies about 10% and in breast cancer about 30% of p53 shows mutations. However, in ovarian, colorectal and head and neck cancers, p53 is mutated 50% to 70% of the time. The majority of TP53 mutations take place in the DNA-binding domain of p53. The tetramerization domain of p53 is usually not mutated; therefore mutated p53 can form heteroteramers with wt p53 and inactivate wt p53 function: this is referred to as the dominant negative effect. Additionally, p53 mutants can also inactivate p53 family members p63 and p73, which are usually not mutated in human cancer.

In general, TP53 mutations can be classified as conformational and DNA contact mutations. Conformational mutations either cause local (R249S; G245S) or global (R175H; R282W) disruptions of the protein structure. DNA contact mutants obliterate p53 binding to specific DNA-sequences and therefore abolish its transcriptional activity. Additionally, these contact mutants cause dominant negative inhibition and are responsible for new oncogenic functions such as drug-resistance, survival and metastasis. The mechanism of mutant p53 function is multifaceted: binding to DNA, altering gene expression, binding to transcription factors to enhance or prevent their function, or interacting with proteins to alter their function directly.

11. p53 Therapeutics

Targeting p53 for cancer therapy is either achieved by directly reintroducing wt p53 into cancer cells via gene therapy, activating p53 and its family members via small molecules and peptides, or using immunotherapy.

i. p53 Gene Therapy

“The first p53 based gene therapy in humans was conducted in 1996. This trial used a retroviral vector containing wild type p53 with an actin promoter for the treatment of non-small cell lung carcinoma. In this study three patients showed tumor regression and three other patients showed tumor growth stabilization (Roth et al. 1996). China was the first country which approved a p53 adenovirus for gene therapy, Gendicine™ SiBiono, in combination with radiotherapy for head and neck squamous cell cancer in 2004 (Shi & Zheng 2009). Gendicine™ is a recombinant serotype 5 adenovirus with the E1 region replaced by the p53 expressing cassette (with a Rous sarcoma virus promoter). The adenovirus particles infect tumor target cells carrying therapeutic p53 (Peng 2005). Clinical trials for Gendicine™ showed that in combination with radiation therapy it caused partial or complete tumor regression (Peng 2005; Xin 2006). There were also some clinical trials for Gendicine™ in advanced liver cancer, lung cancer and other advanced solid tumors (Peng 2005). It should be kept in mind that China's State Food and Drug Administration (SFDA) has different standards for the approval of a cancer drug compared to the U.S. FDA and the European Medicine Agency (EMA). Gendicine™ was approved in China on the basis of tumor shrinkage. The U.S. FDA and the EMA require novel cancer drugs to extend the lifetime of the treated patients (Guo & Xin 2006). Another p53 product is Gendicine™ from Shanghai SunwayBiotech, an oncolytic virus. Gendicine™ was approved for the treatment of head and neck cancer in China in 2006 (Yu & Fang 2007). It is a replicative adenovirus 2/adenovirus 5 hybrid with deletion in E1B55K and E3B (Raty et al. 2008). This oncolytic virus was expected to infect and lyse cancer cells only and not affect normal cells (Guo et al. 2008). Even though clinical studies showed that it was not specific for cancer cells, it did, however, kill tumor cells preferentially (Garber 2006). Phase I/II trials showed little dose-limiting toxicity (Lockley et al. 2006) and the combination of Gendicine™ with chemotherapy showed greater tumor shrinkage in patients with head and neck cancer, compared to chemotherapy alone. It should be kept in mind that like Gendicine™, Oncorine™ was also approved by the SFDA based on objective response rate, not on survival (Garber 2006). Nevertheless, all the available data concerning p53 and its proven function as tumor suppressor qualifies it as an adjuvant treatment with radiotherapy or chemotherapy.” (Matissek K J B R, Davis J R, Lim C S. Choosing Targets for Gene Therapy. Targets for Gene Therapy 2011 July.)

ii. Activating Wt p53

About 50% of all tumors retain wild-type p53 function that is inhibited by increased degradation or proteins that interact with p53. The most famous example is the cis-imidazoline compound Nutlin-3a. It interacts with the p53 binding pocket of MDM2 and consequently disrupts the p53-MDM2 interaction resulting in p53 activation and tumor shrinkage. On the other hand, the small molecule RITA (reactivation of p53 and induction of tumor cell apoptosis) binds directly to p53, preventing MDM2 binding and promoting a strong apoptotic effect on tumors.

Furthermore, several siRNA approaches have been investigated for wt p53 activation. The viral E6 protein from the human papilloma virus binds and targets p53 for inactivation and degradation. SiRNA targeting of E6 inactivates E6 and triggers p53 mediated response. SiRNA targeting of MDM2 can also stabilize and activate p53.

Another way to stabilize wt p53 is through post transcriptional modifications. Acetylated p53 is more stable and cannot be degraded via the MDM2 degradation pathway. Therefore, inhibiting protein-deacetylating activities of proteins such as SirT1 and SirT2 (members of the sirtuin family) could stabilize p53. The small molecule inhibitor Tenovin-1 and its more water-soluble analog Tenovin-6 both prevent protein-deacetylating activities of SirT1 and SirT2.

iii. Reactivating p53 Mediated Response in Cancers with Mutated p53 Status

The challenge in targeting mutant p53 is that it is a heterogeneous target because of the broad range of mutations occurring in human tumors. Drugs developed in the last couple of years mainly focused on reactivating specific variants of mutant p53 to achieve wt p53 like function. One such drug is the carbazole derivative PhiKa083, which binds only to the unstable Y220C mutant, raises its melting temperature and reactivates its function. The Y220C mutation accounts for 75000 patients per year. On the other hand contact and conformational mutants can both be rescued via an ellipticine derivate, 9-hydroxy-ellipticine, which induces G1 arrest and triggers G1 phase-restricted apoptosis in a mutant p53-dependent manner.

The small molecules PRIMA (p53 reactivation and induction of massive apoptosis) and MIRA (mutant p53-dependent induction of rapid apoptosis) both can reactivate mutant p53. PRIMA rescues DNA contact mutants and structural mutants by forming adducts with thiols in mutant p53 core domain. This covalent modification reactivates mutant p53 and induces apoptosis in tumor cells. MIRA restores wt conformation and function of mutant p53 and is more potent than PRIMA. The maleimide group in MIRA reacts with thiol and amino groups in proteins and stabilizes the native fold of p53.

Further, p53-mediated response in tumors containing mutated p53 can be activated not by restoring p53, but instead by its family member p73. In human cancers, p73 is usually not mutated. The small molecule RETRA (reactivation of transcriptional reporter activity) releases p73 from the inhibitory p73/p53mut complex which produces a p53-like tumor suppressor response. Therefore, RETRA increases p21 and PUMA transcription and eventually triggers a delay of tumor formation in xenograft tumor model.

iv. p53 Based Immunotherapy

Some cancer patients develop antibodies against p53. Targeting mutant p53 triggers an antigen-specific cytotoxic T-lymphocyte mediated immune response. A phase I and II clinical trial for colorectal cancer showed a p53-specific immune response when patients were treated with a p53 specific synthetic long peptide (p53-SLP).

In summary, many different approaches have been used to target the p53 pathway for cancer therapy. However, all have critical disadvantages. When reintroducing wt p53 via gene therapy into cancer cells that harbor mutant p53, endogenous wt p53 can face dominant negative inhibition and oncogenic gain of function of mutant p53. This is also the reason why small molecules such as Nutlin-3 that attempt to reactivate functional wt p53 can be indirectly inactivated by mutant p53. Therefore, this approach is only beneficial for patients with wt p53. Additionally, p53 is a very heterogeneous target, and some drugs such as PhiKa083 work for a very small subset of patients. The function of all the drugs listed in this section is highly dependent on the p53 status of the cancer, which require personalized medicine in treating each cancer patient individually.

Targeting p53 directly to the mitochondria can be achieved using an optimal mitochondrial targeting signal (MTS). Since p53 exhibits its rapid, direct apoptotic function at the mitochondria in its monomeric form, regardless of p53 status, it can be effective under any circumstances.

TABLE 5
Summary of p53 therapeutics with their
mechanism of action and p53 status

| | | | Application |
|---|---|---|---|
| | | | dependent on |
| Therapeutic | | Mechanism of action | p53 status |
| gene | Gendicine ™ | Similar to endogenous | wt p53, p53 null |
| | | wt p53 | |
| | Oncorine ™ | Similar to endogenous | wt p53, p53 null |
| | | wt p53 | |
| siRNA | siRNA to E6 | Inactivates E6; p53 | wt p53 |
| | | mediated apoptosis | |
| | siRNA to | Prevents p53 | wt p53 |
| | MDM2 | degradation | |
| Small | Nutlin-3a | Binds to MDM2; | wt p53 |
| molecule | | stabilizes | |
| | RITA | Binds to p53; stabilizes | wt p53 |
| | | p53 | |
| | Tenovin-1 or 6 | Inhibits p53 | wt p53 |
| | | deacetylation: stabilizes | |
| | | p53 | |
| | PhiKa 083 | Binds to p53Y220Cmut; | p53Y220Cmut |
| | | raises melting | |
| | | temperature | |
| | Ellipticine | Induces G1 arrest; G1 | p53mut |
| | derivate | restricted apoptosis | |
| | PRIMA | Forms thiol adducts with | p53mut |
| | | p53mut core domain; | |
| | | stabilizes folding | |
| | MIRA | Maleimide group | p53mut |
| | | reactivates thiols and | |
| | | amines in p53 mut; | |
| | | stabilizes its folding | |
| | RETRA | Releases p73 from | p53mut |
| | | inhibitory p73/p53mut | |
| | | complex | |
| | p53-SLP | Activate p53/T- | Developed |
| | | lymphocyte mediated | antibodies |
| | | immune response | against p53 |

12. Mitochondrial Targeting of p53 for Cancer Therapy

Wild type p53 has been used almost for a decade in cancer gene therapy. It was approved for the treatment of head and neck cancer in China under the trade name Gendacine® and Oncorine®. In the U.S., there are several clinical trials ongoing with wild-type p53 mostly in combination with other chemotherapeutics. All these gene therapy approaches have focused mainly on p53's role as a transcription factor. Moll and colleagues have attempted targeting p53 to the mitochondria for cancer therapy but did not achieve clinical translation.

p53 does not contain a mitochondrial targeting signal. Moll and colleagues suggested that MDM2 triggers monoubiquitination of p53 which results in nuclear export. Cytoplasmic monoubiquitinated p53 is imported into the mitochondrial via the herpes virus-associated ubiquitin-specific protease. At the mitochondria p53 triggers the intrinsic apoptotic pathway by interacting with anti (Bcl-XL, Mcl-1)- and pro-apoptotic (Bak, Bax) Bcl-2 protein family members. At the mitochondrial outer membrane, p53 interacts first with anti-apoptotic Bcl-2 proteins by sequestering them. Then it activates pro-apoptotic Bak and Bax, triggers their homo-oligomerization, resulting in cytochorome c release, caspase activation and eventually apoptosis.

Different mitochondrial targeting signals (MTS)s were evaluated for their ability to induce p53-mediated apoptosis. MTS from XL has no internal toxicity and therefore is the best MTS to target p53 to the mitochondria by causing p53-dependent apoptosis while Moll claims that the MTS from the ornithine transcarbomylase (OTC) is the best to target p53 to the mitochondria. However, it has been demonstrated that the OTC signal has internal toxicity. Further, whether a p53 subdomain is sufficient to trigger apoptosis at the mitochondria through p53-specific interaction with anti-apoptotic Bcl-XL and pro-apoptotic Bak was investigated.

Mitochondrial p53 is superior to wild type p53 in three ways. First, mitochondrial p53 directly interacts with pro- and anti-apoptotic proteins at the mitochondrial outer membrane and triggers the intrinsic apoptotic pathway. wt p53 usually acts as a transcription factor and needs to transactivate its targeted genes first. Therefore, mitochondrially targeted p53 causes a more rapid apoptotic response compared to wild type p53. Second, mitochondrial p53 solely induces apoptosis while wt p53 has the ability to transactivate genes involved in cell cycle arrest, DNA repair and metabolism which might not have a beneficial effect in cancer therapy. Third, transcriptional activity of p53 is highly dependent on tetramer formation. In cancer cells, p53 mutations occur in the DNA binding domain of p53 while the tetramerization domain (TD) remains active forming wt/mut heterotetramers (described previously as dominant negative effect). In contrast to tetrameric transcriptionally active p53, mitochondrial p53 is mostly monomeric and can be unaffected by dominant negative inhibition.

13. The Mitochondrial Compartment and its Import Machinery

The mitochondria is known to be involved in the synthesis of ATP and in numerous other metabolic processes including biosynthesis of vitamin cofactors, amino acids, fatty acids, and iron-sulphur clusters. Additionally, mitochondria are also known as the central regulator of the intrinsic apoptotic pathway. The mitochondrion consists of an outer membrane surrounding an inner membrane and two aquaosis compartments intermembrane space (IMS) and matrix. IMS harbors cytotoxic proteins such as cytochodrome c and SMAC/diabolo while the matrix is essential for citric acid cycle and fatty acid-oxidation. Even though the mitochondrion encloses their own genome, most of the mitochondrial polypeptides are encoded in the nuclear genome. Mitochondrial proteins are synthesized in the cytosol and imported into the mitochondria. For proper translocation and membrane insertion of these proteins, the mitochondrial membranes contain specific machinery for mitochondrial import. The two mitochondrial membranes contain two major import receptors. The translocase of the outer mitochondrial membrane complex is localized as the name implies in the mitochondrial membrane. It contains seven different subunits, the receptors Tom20, Tom22, Tom70; the channel-forming protein Tom40 and the small Tom proteins Tom5, Tom6, Tom7. The Tom 20 receptor recognizes the mitochondrial targeting signal (MTS) of the mitochondrial protein, guides it to Tom22 which than targets it to the translocase of the inner membrane (TIM). The TIM complex consists of two functional modules the membrane-integrated translocase unit (Tim23, Tim17, Tim50) and the presequence-translocase-associated import-motor complex (PAM complex). The ATP-powered PAM complex is a multiprotein complex consisting of mitochondrial heat-shock protein-70 (mtHsp70) and its essential cofactors.

There are two main classes of mitochondrial targeting signals, N-terminal presequences and tail-anchored sequences. Most of the matrix and some of the inner and intermembrane space proteins have the N-terminal presequences consisting of 10-30 amino acids which form an α-helix. One side of the helix has a hydrophobic surface and the other side is positively charged. The MTSs are recognized and imported by the TOM complex and the TIM complex. Once they reach the matrix, matrix-localized processing peptidase cleave the MTS from the remaining protein. Tail-anchored proteins are usually found on the mitochondrial outer membrane. They consist of a signal membrane insertion sequence at their C-terminus and display a large N-terminal portion to the cytosol. Examples of tail-anchored proteins are the pro- and anti-apoptotic Bcl-2 proteins such as Bcl-XL, MCl-1, Bcl-2, Bak and Bax to mention a few.

14. Adenoviral Drug Delivery

There are two major types of gene delivery vehicles: viral and non-viral vectors. Non-viral gene delivery is potentially a safer approach but limited due to inefficiency. Conversely, viral vectors allow efficient gene transfer with some safety risks. Two viral vectors are used in clinical trials; retrovirus and adenovirus. Retrovirus has the advantage of having a permanent effect on the infected cells since the gene-load is inserted in the genome of the host cells. This advantage represents a double-edged sword: on one hand it is highly efficient but on the other hand it integrates randomly into the patient's genome and can therefore cause additional malignancies. Since we do not need a permanent genomic change and only want to cause cancer cell apoptosis, we decided to proceed with adenoviral drug delivery which only has an immediate effect and therefore does not integrate into the host's genome. The disadvantage of adenoviral drug delivery is the development of antibodies against the virus. For targeting a local tumor in breast cancer, intratumoral injections can be used for adenoviral gene therapy in vivo.

### R. Example 5

### Delivery of a Monomeric p53 with Mitochondrial Targeting Signals from Anti-Apoptotic Bcl-XL to Overcome Dominant Negative Inhibition of Mutant p53

1. Introduction

The tumor suppressor p53 has been the focus of intensive cancer-based research for more than three decades. This resulted in adenovirally delivered wt p53 being approved for gene therapy in China under the trade names Gendicine and Oncorine. Additionally, there are various clinical trials for p53 based cancer therapy around the world. However, limitations of its use are due to dominant negative inactivation of wt p53 by endogenous mutant p53. Mutations in the p53 gene typically occur in the DNA binding domain (DBD) of p53, while the tetramerization (TD) domain usually remains active. Therefore, wt p53 can form heterotetramers with mutant p53 which abolishes the transcriptional activity of the wt p53/mutp53 heterotetramer complex. This is, for example, a problem in triple negative breast cancer (TNBC) where 60-88% of TNBC have mutated p53 and would exhibit dominant negative inhibition if wt p53 was reintroduced. Mitochondrial p53 could circumvent this problem, and can represent a major advancement in treatment of TNBC.

p53 targeted to the mitochondria interacts with pro- and anti-apoptotic Bcl-2 proteins at the mitochondrial outer membrane and causes a rapid apoptotic response. The DBD of p53 delivered to the mitochondria by a mitochondrial targeting signal is sufficient to cause apoptosis. Since DBD lacks the TD which is essential for tetramer formation, p53 initiates apoptosis at the mitochondria likely without tetamerization (e.g., as a monomer). Monomeric p53 can evade dominant negative inhibition of endogenous mutant p53. p53-XL and DBD-XL can be delivered adenovirally and apoptosis can be shown in vitro and in vivo.

Nielsen et al. have already attempted to use adenovirally delivered wt p53 to treat xenografted triple negative breast cancer MDA-MB-468 tumors in mice. However, due to the dominant negative inhibition of mutant p53, wt p53 was unable to cause tumor shrinkage and only resulted in tumor growth inhibition. Intratumorally injected adenoviral p53-XL and DBD-XL can be used in an orthotropic breast cancer mouse model. This can result in cell apoptosis and tumor shrinkage since the constructs are not affected by dominant negative inhibition by mutant p53.

2. Methods

i. Cell Lines

H1373 human non-small cell lung carcinoma cells were grown as monolayers in RPMI (Invitrogen, Carlsbad, Calif.) supplemented with 10% fetal bovine serum (Invitrogen), 1% penicillin-streptomycin-glutamine (Invitrogen), and 0.1% gentamicin (Invitrogen). HEK293 human embryonic kidney (ATCC) and MDA-MB-468 human breast adenocarcinoma cells (ATCC) were grown as monolayers in DMEM (Invitrogen) supplemented with 10% fetal bovine serum, 1% penicillin-streptomycin-glutamine, and 0.1% gentamicin. MDA-MB-468 cells were also supplemented with 1% MEM non-essential amino acids (Invitrogen). All cells were incubated in 5% CO2 at 37° C. The cells were seeded for transduction at a density of 3.0×105 cells in 6-well plates (Greiner Bio-One, Monroe, N.C.). Viral transductions were carried out immediately after seeding the cells.

ii. Recombinant Adenovirus Production

Replication-deficient recombinant adenovirus serotype 5 (Ad) constructs were created by inserting PCR amplified p53-XL (Ad-p53-XL) or DBD-XL (Ad-DBD-XL) into a cassette under the control of the CMV promoter. Prior to insertion, these constructs were PCR amplified with primers containing 15 base pair homology with a linearized pAdenoX vector (Clontech) based on an In-Fusion® HD Cloning Kit (Clontech). Empty vector served as negative control (Ad-ZsGreen). For visualization the Adeno-X® Adenoviral Expression System 3 contains a separate CMV promoter for ZsGreen1 expression. The adenoviral vector plasmids containing our constructs were transformed into Stellar® competent cells (Clontech). For packaging and amplification, viral DNA was purified linearized and transfected into HEK293 cells. Viral particles were isolated from HEK293 cells by freeze-thawing, purified using Adeno-X® Mega Purification Kit (Clontech), and dialyzed against storage and proper tonicity buffer (2.5% glycerol (w/v), 25 mM NaCl, and 20 mM Tris-HCl, pH 7.4). Following the manufacturer's recommendation, flow cytometry was used to determine the viral titer.

iii. Overexpression of Mutant p53 Using Lipofectamine Transfection

H1373 cells were cotransfected with 1 pmol of the transdominant mutant pTagBFP-mut-p53 (R248W) and 1 pmol of previously designed plasmids wt p53, p53-XL, E-XL or EGFP. To create pTagBFP-mut-p53 (R248W) the following primers were used 5′-CTGCATGGGCGGCATGAACTGGAGGCCCATCCTCACCA-3′ and 5′-TGGTGAGGATGGGCCTCCAGTTCATGCCGCCCATGCAG-3′. p53-XL was designed by cloning wt p53 and MTS from anti-apoptotic Bcl-XL into EGFP-C1 vector. E-XL was created by inserting MTS from XL into EGFP-C1 vector. Lipofectamine was used as the transfection reagent as before. 48 h post transfection, cells were stained as described in the 7-AAD assay and gated for EGFP and BFP using the FACSCanto-II (BD-BioSciences) and FACSDiva software. Excitation for BFP was set at 405 nm and detected at 457 nm. The means from three separate experiments (n=3) were analyzed using two-way ANOVA with Bonferroni's post hoc test.

iv. 7-AAD Assay

48 h after transfection MDA-MB-468 cells were stained with 7-aminoactinomycin D (7-AAD, Invitrogen). Excitation was set at 488 nm and detected at 507 nm and 780 nm for ZsGreen1 and 7-AAD, respectively using the FACSCanto-II (BD-BioSciences) and FACSDiva software. The means from three separate experiments (n=3) were analyzed using one-way ANOVA with Bonferroni's post hoc test.

v. Western Blotting

MDA-MB-468 were harvested 24 h post infection, pelleted and resuspended in 200 μL lysis buffer (62.5 mM Tris-HCl, 2% w/v SDS, 10% glycerol, 1% protease inhibitor). Standard western blotting procedures were followed using primary antibodies to detect caspase-9, and actin as a loading control. The primary antibodies anti-caspase-9 (#7237P, Cell Signaling Technology) and anti-actin (rabbit, ab1801, Abcam) were detected with anti-rabbit (#7074S, Cell Signaling Technology) antibodies before the addition of SuperSignal West Pico chemiluminescent substrate (Thermo Scientific, Waltham, Mass.). The FluorChem FC2 imager and software (Alpha Innotech, Santa Clara, Calif.) was used to detect the signal.

vi. In Vivo Experiments

Female nu/nu athymic mice (4-6 weeks old, Jackson Laboratories) were injected subcutaneously into the mammary fat pad with human MDA-MB-468 cells (1×10⁷ cells/mouse in 100 μl of serum-free RPMI-1640 medium). After tumors reached the mean size of 50 mm3, Ad-p53-XL, Ad-DBD-XL and Ad-ZsGreen1 were intratumorally injected on days 0-4 and 7-11. A dose of 5.0×10⁸ pfu in 50 μL volume was administrated. Tumor volumes were measured daily using Vernier calipers along the longest width (W) and the corresponding perpendicular length (L) using V=(L×(0.5W)2) to calculate tumor volume. Mice were sacrificed and tumors and organs were harvested 24 h after the last injection.

3. Results

i. Mitochondrial p53 can Bypass Dominant Negative Inhibition In Vitro

To determine if mitochondrial targeted p53-XL and DBD-XL can escape the dominant negative effect of wt p53, first a rescue experiment was conducted in H1373, and second, MDA-MB-468 cells that naturally harbor a dominant negative mutant p53 (R273H mutation) were tested.

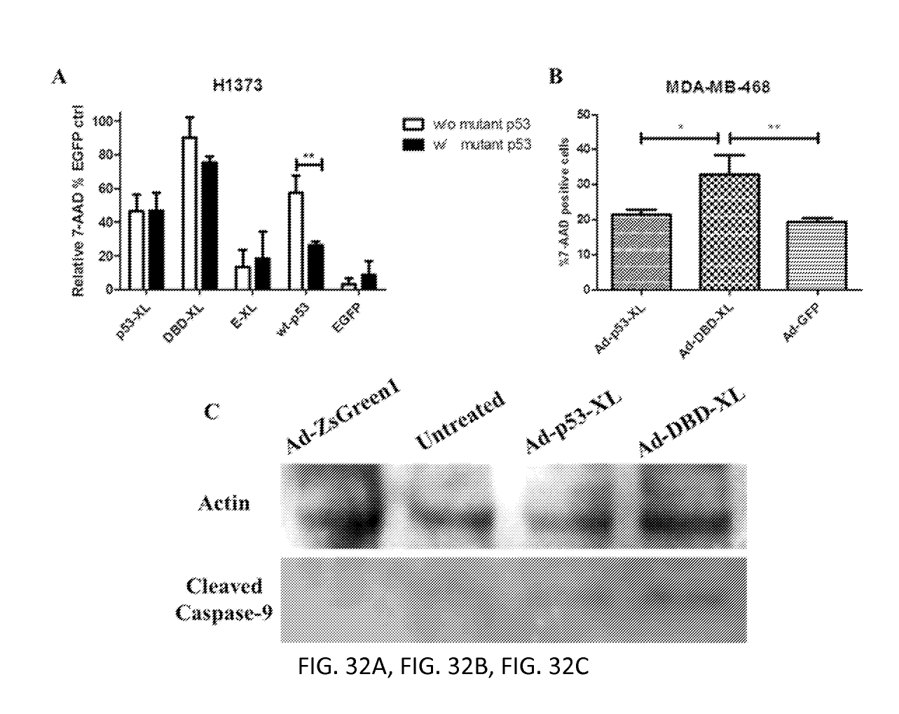

A mutant p53 construct was designed with R248W mutation (called p53R248Wmut) which is known to exhibit a strong dominant negative effect. Human non-small cell lung carcinoma cells H1373 (p53 null) were transfected with the constructs (p53-XL, DBD-XL, E-XL, wt p53, or EGFP), with or without the p53R248Wmut, and conducted a 7-AAD assay on these groups. In the absence of p53R248Wmut, p53-XL, DBD-XL and wt p53 showed significantly higher apoptosis compared to their negative controls E-XL and EGFP (FIG. 32A, white set of bars). When co-transfected with p53R248Wmut, p53-XL and DBD-XL are capable of rescuing apoptotic activity while the apoptotic activity of wt p53 is dramatically impaired (FIG. 32A, black set of bars) indicating that mitochondrial targeting of p53 can overcome dominant negative inhibition of mutant p53.

To determine if mitochondrial p53 is capable of inducing apoptosis in a cell line that naturally harbors a dominant negative mutation, MDA-MB-468 cells were infected with the designed viral constructs and a 7-AAD assay and a caspase-3/7 western blot were conducted. Triple negative breast cancer cells MDA-MB-468 harbor the R273H mutation which is considered to have a strong dominant negative effect on wt p53. For the H1373 rescue experiment, lipofectamine transfection of plasmid DNA was used. However, MDA-MB-468 are resistant to lipofectamine transfection. Therefore, Ad-p53-XL, Ad-DBD-XL and Ad-ZsGreen1 adenoviral constructs were constructed. For the previous plasmids (non-viral plasmids), EGFP was directly fused to the construct; for adenoviral constructs, ZsGreen1 is co-expressed with the protein of interest.

When MDA-MB-468 cells were infected with Ad-p53-XL, Ad-DBD-XL and Ad-ZsGreen1, only Ad-DBD-XL showed higher activity than Ad-ZsGreen1 in the 7-AAD assay (FIG. 32B). Surprisingly Ad-p53-XL was not significantly different from Ad-ZsGreen1 control.

To determine if the induced apoptotic effect of Ad-DBD-XL is through the mitochondrial/intrinsic apoptotic pathway, caspase-9 was detected in MDA-MB-468 cells via western blotting. Caspase-9 can only be activated via the intrinsic apoptotic pathway. A representative cropped image of the western blot is shown in FIG. 32C. Ad-DBD-XL showed caspase-9 induction while Ad-p53-XL only shows a faint band and negative controls Ad-ZsGreen1 and untreated MDA-MB-468 cells did not show caspase-9 activity.

ii. Testing the Effect of Mitochondrial p53 in an Orthotropic Breast Cancer Model, In Vivo

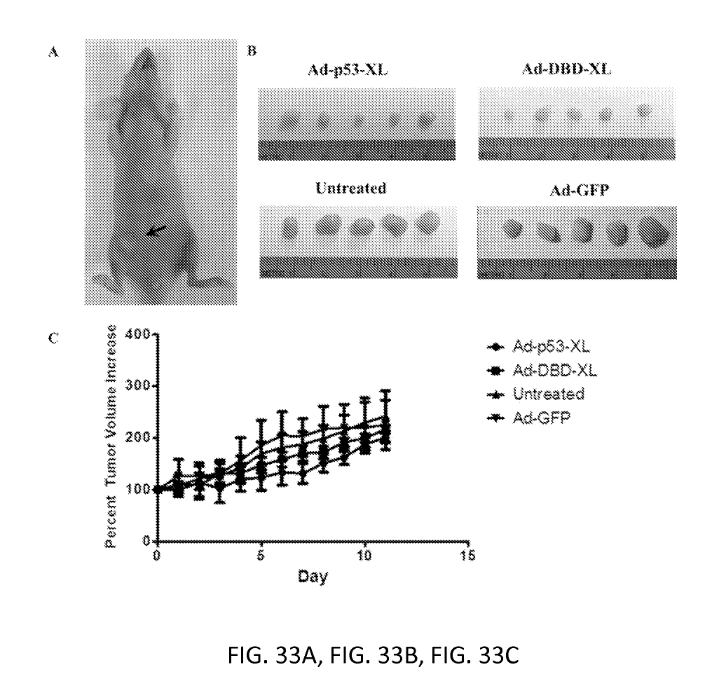

MDA-MB-468 cells were injected into the mouse mammary fat pad, and allowed tumors to grow to 50 mm³. 5.0×10⁸ pfu of Ad-p53-XL, Ad-DBD-XL and Ad-ZsGreen were then injected intratumorally on day 0-4 and 7-11. FIG. 33A shows a representative picture of a tumor-bearing mouse (black arrow indicates tumor site). The tumors were harvested 24 hours after the last treatment. Excised tumors of Ad-p53-XL, Ad-DBD-XL, untreated and Ad-ZsGreen1 are shown in FIG. 33B.

Although Ad-p53-XL and Ad-DBD-XL tumors appear smaller than the controls, their tumor sizes were smaller to begin with. In fact, tumor size measurements in FIG. 33C revealed that there were no differences between treatment groups and untreated showing that mitochondrial p53 is unable to evade dominant negative inhibition in this model.

iii. Discussion

It was previously shown that targeting p53 to the mitochondria results in apoptosis in a variety of cancer cells. The DBD of p53 is sufficient to cause apoptosis at the mitochondria. In this study, mitochondrially targeted p53 was shown to be capable of overcoming dominant negative inhibition of endogenous mutant p53 in vitro and in vivo.

Therefore, dominant mutant p53R248W was overexpressed in p53 null H1373 cells (FIG. 32A) and then apoptosis assays were conducted in triple negative MDA-MB-468 cells (FIG. 32B, C) that naturally harbor dominant negative R273H mutant. In H1373 cells, p53-XL and DBD-XL retain the same apoptotic activity with or without overexpressing the dominant negative mutant, while apoptotic activity of wt p53 is dramatically decreased (FIG. 32A). However, only Ad-DBD-XL was active in the dominant negative MDA-MB-468 cell line while Ad-p53-XL showed the same inactivity as the negative control Ad-ZsGreen1 (FIG. 32B). p53-XL can be sequestered via its TD by endogenous p53 in MDA-MB-468 cells. Unlike p53-XL, DBD-XL does not contain a TD and is therefore not inhibited by endogenous p53.

Further, whether apoptotic activity of DBD-XL is through the intrinsic apoptotic pathway was examined. Thus, caspase-9 induction which can only be activated through the intrinsic apoptotic pathway was determined. Again, Ad-DBD-XL showed caspase-9 activation while Ad-p53-XL only showed minimal capsase-9 activity (FIG. 32C). In summary, these results indicate that DBD-XL can bypass dominant negative inhibition of mutant p53.

Even though in vitro results looked promising, mitochondrial p53 was not capable of reducing MDA-MB-468 tumor size (FIG. 33C). It seems that the p53-specific intrinsic apoptotic response by p53-XL and DBD-XL in MDA-MB-468 cells is not sufficient to result in inhibition of tumor growth or tumor shrinkage in this particular mouse breast cancer model. The dosing regimen can be the reason for the treatment failure. At day 5-6 when no drug was injected tumors started to grow again indicating a transient effect of apoptosis by mitochondrial p53 (FIG. 33C). The dose used in this study is the same as for wt p53 therapy in MDA-MB-468. However, wt p53 and mitochondrial p53 have different apoptotic profiles. While mitochondrial p53 induces apoptosis solely through intrinsic apoptotic pathway, wt p53 activates the intrinsic and extrinsic pathway. Because mitochondrial p53 only induces the intrinsic apoptotic pathway, the effective dose can be higher than wt p53 therapy. In addition, the R273H mutant expressed in MDA-MB-468 has a very aggressive cancer profile. In fact, knock-in mice harboring this mutation (p53R273H/-mice) develop more carcinomas and have more invasive and metastatic properties than p53 knock-out mice. Due to differences in apoptotic mechanism and aggressiveness of MDA-MB-468 the dose of mitochondrial p53 can be increased and given more frequently, or combined with another cancer therapeutic.

It has been shown that in response to chemotherapy or radiation treatment p53-mediated apoptosis causes tumor regression and transfecting these cell lines with wt p53 increases the sensitivity to chemotherapy. The potency of mitochondrial p53 can be enhanced by combination with a chemotherapeutic that targets the nucleus and intercalates with nuclear DNA (antracycline), interferes with DNA replication (alkylating agent) or abolishes DNA/RNA synthesis (antimetabolite). The current treatment plan of breast cancer already involves a variety of cytotoxic drugs such as antracycline (doxorubicin, epirubicin), alkylating agents (cyclophosphamide, methotrexate) and antimetabolites (fluorouracil). A dual approach combining mitochondrial p53 with antracycline, alkylating agent or antimetabolite has the advantage of targeting two cellular organelles which are highly involved in apoptosis resulting in higher apoptosis induction.

Mitochondrial targeting of p53 can be further optimized by fusing it to MTS from pro-apoptotic Bak. As previously shown, the MTS is responsible for directing p53 specifically to the protein where the MTS is taken from. Every protein that is tagged to XL can translocate to anti-apoptotic Bcl-XL while p53 fused to BakMTS can translocate to pro-apoptotic Bak. When p53 is targeted to Bcl-XL, it will primarily inhibit Bcl-XL and is less effective on other anti-apoptotic proteins such as Bcl-2 and Mcl-1. In cells mainly expressing Bcl-XL with low Bcl-2 and Mcl-1 levels, neutralizing Bcl-XL results in Bak homooligomerization and apoptosis. However, in aggressive cancers, Bcl-2, Bcl-XL and Mcl-1 are often overexpressed. Therefore, the amount of p53-XL and DBD-XL might not be efficient enough to sequester anti-apoptotic Bcl-2 proteins and to ensure Bak and Bax homo-oligomerization. This again indicates the need to increase the dose of p53-XL and DBD-XL.

Directly targeting the pro-apoptotic Bak or Bax can be used instead of targeting Bcl-XL. Bak and Bax are directly responsible for pore formation at the mitochondria. While anti-apoptotic Bcl-2 proteins such as Bcl-XL need to be sequestered, pro-apoptotic Bak or Bax just needs to be activated by a transient interaction and then can form pores at mitochondrial outer membrane. This can be due to differences in subcellular localization of these proteins. Bax is constantly shuttled between mitochondria and cytoplasm. Therefore the amount present at the mitochondria might be not sufficient to initiate apoptosis. Bak on the other hand is always present at the mitochondria and is activated when p53 is targeted to the mitochondria with MTS from Bak, triggering caspase activation and apoptosis.

In fact, we have shown that p53 or DBD fused to the MTS from pro-apoptotic Bak was superior over wt p53 and the chimeric p53 construct named p53-CC in p53 null ovarian cancer cells SKOV-3. This can be due to a defect in the transcriptional apoptotic pathway of p53 in SKOV-3 cells. Targeting p53 to the mitochondria can be used to treat ovarian cancer therapy.

### S. Example 6

### DBD Fused to Mitochondrial Targeting Signal from Bak for Ovarian Cancer Targeting

Previously, it was discovered the minimal domain of p53, its DNA binding domain (DBD), is needed for apoptosis and a rationally selected MTS with optimal activity. MTSs from the mitochondrial outer membrane (MOM) are optimal for p53-specific activation. Using MTSs from pro-apoptotic Bcl-2 family members Bak can trigger mitochondrial outer membrane permeabilization (MOMP), resulting in activation of the intrinsic apoptotic pathway. Bak MTS can be used for DBD targeting and apoptosis.

Mitochondrial p53 inhibits anti-apoptotic (Bcl-2, Bcl-XL, Mcl-1) and activates pro-apoptotic (Bak, Bax) Bcl-2 family members leading to mitochondrial outer membrane permeabilization (MOMP) and resulting in apoptosis. In cancer cells, overexpression of Bcl-2, Bcl-XL and Mcl-1 correlates with more aggressive phenotypes and leads to chemotherapy resistance. Many agents have been identified to target the anti-apoptotic Bcl-2 family members. These therapeutics cause apoptosis through neutralizing anti-apoptotic proteins at the mitochondria allowing the pro-apoptotic Bcl-2 family members Bak or Bax to homo-oligmomerize initiating apoptosis. However, these inhibitors do not target all anti-apoptotic Bcl-2 proteins; in fact most of them (Navitoclax, ABT-199) are selective to Bcl-2. This limits its use since many cancers overexpress Bcl-XL and Mcl-1. Therefore, directly activating pro-apoptotic Bak by targeting p53 to the mitochondria using Bak's own MTS can be done. p53 activates Bak by disrupting Bak/Mcl-1 and Bak/Bcl-XL complexes. DBD-BakMTS can trigger apoptosis in cancer cells by preferential interaction with Bak (over Mcl-1 or Bcl-XL).

The data indicate a robust apoptosis from DBD-BakMTS that is entirely dependent on residues in the DBD that directly interact with cellular Bak. The minimal domain of p53 (DBD) fused to MTS from pro-apoptotic Bak which is localized at the mitochondrial outer membrane results in apoptosis in a variety of cancer cell lines. Further, DBD-BakMTS triggers apoptosis in ovarian cancer cells SKOV-3 while wt p53 and p53-CC are incapable of inducing apoptosis in this cell line. Since wt p53 and p53-CC mainly cause cell death through transactivating apoptotic genes, SKOV-3 can have a defect in p53 transcription machinery.

DBD-BakMTS can be used for ovarian cancer therapy. Ovarian cancer has shown lack of progress in treatment, since mortality rates of ovarian cancer have not improved over 40 years. The Cancer Genome Atlas Research network identified a variety of genomic changes in ovarian cancer. While p53 gene mutations occur in more than 96% of ovarian serous tumors, recurrence in mutations in other genes were not noted, or only had a low prevalence. Therefore, p53 is an excellent target for ovarian cancer therapy.

Ovarian cancer cells with different p53 status can highlight that apoptotic activity of DBD-BakMTS is not dependent on p53 status. Since SKOV-3 ovarian adenocarcinoma cells from metastatic ascites which are p53 null were already tested, ovarian cancer cells harboring mutant p53 (OVCAR-3 and Caov-4) and wt p53 (A2780) can be tested. Apoptotic activity can be determined by using previously described TMRE-, caspase-9, annexin V and 7-AAD assays.

To explore the effect of DBD-BakMTS on normal cells, BJ normal fibroblasts and immortalized ovarian epithelial cells (IHOEC) can be tested. Since DBD-BakMTS is lacking the MBD and C-terminus the DBD-BakMTS is not subject to the p53/MDM-2 degradation pathway. In wt p53, E-3 ligase MDM2 binds to the MBD of wt p53 which triggers monoubiquitations. Then, MDM2 and cofactors (E4 factors, E like molecules) or other E-3 ligases promote polyubiquitination of C-terminal lysines resulting in proteasomal degradation. While wt p53 is known to be non-toxic to normal cells, DBD-BakMTS can show some toxicity to normal cells due to the lack of MBD and C-terminus and hence, lack of degradation. If normal cells undergo apoptosis with DBD-BakMTS, cancer specific promoters can be used to prevent this. Cancer specific promoters include survivin, unmodified HTERT or ovarian cell-specific OSP-1 promoter, which can prevent expression in normal cells and hence apoptosis in these cells. Therefore, the use of these promoters can ensure expression of DBD-BakMTS mainly in cancer cells and minimize death in normal cells.

Further, the activity of DBD-BakMTS can be tested in a syngeneic orthotropic metastatic mouse model. The metastatic ovarian cancer model can be generated by injecting ID8 cells into left ovarian bursa. Prior to initiating treatment, tumors can be grown to approximately a size of 2-2.5 cm³. This animal model closely replicates characteristics and hallmarks seen in human ovarian cancer by primary epithelial ovarian tumors, secondary peritoneal metastases and ascites production. DBD-BakMTS can be delivered by water soluble lipopolymer (WSLP). Heterogeneity or lack of expression of CAR and integrin co-receptors in ovarian tumors and adenovirus-neutralizing antibodies are major limitations to ovarian cancer adenoviral drug delivery. WSLP completed a Phase I clinical trial for ovarian cancer. Plasmid DNA encoding DBD-BakMTS can be intraperitoneal (I.P.) injected with WSLP into the new syngeneic orthotropic metastatic mouse ovarian cancer model, alone or in combination with carboplatin and paclitaxel. Various studies indicate that I.P. is superior over intravenous (I.V.) delivery. Higher concentrations of cytotoxic agents can be infused into the peritoneal cavity than would be tolerated systemically. We chose I.P. to be able to increase the dosing in the ovarian cancer model. Additionally, I.P. allowed for sustained exposure of tumor implants to antitumor agents while normal tissues, such as bone marrow, are significantly less exposed. Fewell et al. used 10-250 μg of WSLP-IL-12 plasmids per injection when using an IP ovarian tumor mouse model. Due to the difference in mechanism of action between IL-12 (takes time to induce IFN-α) and p53-MTS (rapid apoptosis expected), frequent dosing but a greater effect can be used. Plasmid amount of DBD-BakMTS can be determined first in vitro by conducting apoptosis assays in ID8 mouse ovarian cancer cells after that concentration and dosing regimen can be optimized in an animal study.

DBD-BakMTS can be combined with standard ovarian chemotherapeutics. The combination of carboplatin and paclitaxel are both first line therapy for ovarian cancer but both not completely effective. Platinum resistant cancer recurs in 25% of patients within 6 months; platinum requires functional p53 protein for efficient induction of apoptosis, and loss of p53 function enhances resistance to cytotoxic agents. DBD delivered to the mitochondria via MTS from Bak can enhance/synergize with standard chemotherapy.

DBD-BakMTS with or without carboplatin and paclitaxel can be used for treating women diagnosed with late stage (>III) or recurrent high grade serous ovarian cancer and can result in a new clinical trial potent enough to prevent metastatic disease recurrence.

### T. Example 8

### Targeting DBD-Bak for Lung Cancer Therapy

The p53-based therapeutic approaches can be applicable to other types of cancers as well, including lung cancer which is the leading cause of cancer in the United States. In fact, the overall 5-year survival rate is only about 15%. Studies have shown that p53 is mutated in up to 70% of lung cancers. Therefore, p53 is an excellent target for lung cancer therapy.

Targeting the DNA binding domain (DBD) of p53 is sufficient (and sometimes more efficient than wt p53) in inducing a direct apoptotic effect at the mitochondria in H1373 human non-small cell lung carcinoma cells. Re-engineered, mitochondrially targeted p53 can be effective against lung cancer. Mitochondrially targeted p53 can be delivered as a protein formulated as a dry powder for inhalation.

The lung is a good target for drug delivery because drugs can be delivered via inhalation. It is wildly known that proteins can be absorbed through the lungs. One example is insulin which was the first peptide approved for inhalation therapy by the U.S. Food and Drug Administration in January 2006. However, Exubera was discontinued in October 2007. The reason for the discontinuation was mainly the high price. Other insulin delivery alternatives (injectable, pen, etc.) are less costly. One approach is to deliver DBD-Bak as a dry power protein in a similar manner as Exubera using the AERx Pulmonary Drug Delivery System. This system converts large particles (protein agglomerates) into a fine particle aerosol. By delivering DBD-Bak specifically to lung cancer cells, side effects can be minimized.

Lung cancer is only one other possible type of cancer to target with the mitochondrially targeted p53. Other types of cancers can be targeted as well. This approach highlights mitochondrial targeted DBD again as a “sledgehammer,” effective under any circumstances, regardless of genetics or the pathway upon which the cancer develops.

Those skilled in the art will recognize, or be able to ascertain using no more than routine experimentation, many equivalents to the specific embodiments of the method and compositions described herein. Such equivalents are intended to be encompassed by the following claims.

### REFERENCES

1. Goh A M, Coffill C R, Lane D P. The role of mutant p53 in human cancer. J Pathol. 2011 January; 223(2):116-26.
2. Sherr C J. Principles of tumor suppression. Cell. 2004 Jan. 23; 116(2):235-46.
3. Martins C P, Brown-Swigart L, Evan G I. Modeling the therapeutic efficacy of p53 restoration in tumors. Cell. 2006 Dec. 29; 127(7):1323-34.
4. Xin H. Chinese gene therapy. Gendicine's efficacy: hard to translate. Science. 2006 Nov. 24; 314(5803):1233.
5. Peng Z. Current status of gendicine in China: recombinant human Ad-p53 agent for treatment of cancers. Hum Gene Ther. 2005 September; 16(9):1016-27.
6. Palacios G, Crawford H C, Vaseva A, Moll U M. Mitochondrially targeted wild-type p53 induces apoptosis in a solid human tumor xenograft model. Cell Cycle. 2008 Aug. 15; 7(16):2584-90.
7. Fuster J J, Sanz-Gonzalez S M, Moll U M, Andres V. Classic and novel roles of p53: prospects for anticancer therapy. Trends Mol Med. 2007 May; 13(5):192-9.
8. Perfettini J L, Kroemer R T, Kroemer G. Fatal liaisons of p53 with Bax and Bak. Nat Cell Biol. 2004 May; 6(5):386-8.
9. Vaseva A V, Moll U M. The mitochondrial p53 pathway. Biochim Biophys Acta. 2009 May; 1787(5):414-20.
10. Joerger A C, Fersht A R. The tumor suppressor p53: from structures to drug discovery. Cold Spring Harb Perspect Biol. 2010 June; 2(6):a000919.
11. Hagn F, Klein C, Demmer O, Marchenko N, Vaseva A, Moll U M, et al. BclxL changes conformation upon binding to wild-type but not mutant p53 DNA binding domain. J Biol Chem. 2010 Jan. 29; 285(5):3439-50.
12. Petros A M, Gunasekera A, Xu N, Olejniczak E T, Fesik S W. Defining the p53 DNA-binding domain/Bcl-x(L)-binding interface using NMR. FEBS Left. 2004 Feb. 13; 559(1-3):171-4.
13. Tomita Y, Marchenko N, Erster S, Nemajerova A, Dehner A, Klein C, et al. W T p53, but not tumor-derived mutants, bind to Bcl2 via the DNA binding domain and induce mitochondrial permeabilization. J Biol Chem. 2006 Mar. 31; 281(13):8600-6.
14. Bharatham N, Chi S W, Yoon H S. Molecular basis of Bcl-X(L)-p53 interaction: insights from molecular dynamics simulations. PLoS One. 2011; 6(10):e26014.
15. Chipuk J E, Maurer U, Green D R, Schuler M. Pharmacologic activation of p53 elicits Bax-dependent apoptosis in the absence of transcription. Cancer Cell. 2003 November; 4(5):371-81.
16. Ha R I, Won E Y, Shin J S, Jang M, Ryu K S, Bae K H, et al. Molecular mimicry-based repositioning of nutlin-3 to anti-apoptotic Bcl-2 family proteins. J Am Chem Soc. 2011 Feb. 9; 133(5):1244-7.
17. Xu H, Tai J, Ye H, Kang C B, Yoon H S. The N-terminal domain of tumor suppressor p53 is involved in the molecular interaction with the anti-apoptotic protein Bcl-Xl. Biochem Biophys Res Commun. 2006 Mar. 24; 341(4):938-44.
18. Mossalam M, Matissek K J, Okal A, Constance J E, Lim C S. Direct induction of apoptosis using an optimal mitochondrially targeted p53. Mol Pharm. 2012 May 7; 9(5):1449-58.
19. Dixon A S, Pendley S S, Bruno B J, Woessner D W, Shimpi A A, Cheatham T E, 3rd, et al. Disruption of Bcr-Abl coiled coil oligomerization by design. J Biol Chem. 2011 Aug. 5; 286(31):27751-60.
20. Dixon A S, Miller G D, Bruno B J, Constance J E, Woessner D W, Fidler T P, et al. Improved coiled-coil design enhances interaction with Bcr-Abl and induces apoptosis. Mol Pharm. 2012 Jan. 1; 9(1):187-95.
21. Dixon A S, Kakar M, Schneider K M, Constance J E, Paullin B C, Lim C S. Controlling subcellular localization to alter function: Sending oncogenic Bcr-Abl to the nucleus causes apoptosis. J Control Release. 2009 Dec. 16; 140(3):245-9.
22. Bolte S, Cordelieres F P. A guided tour into subcellular colocalization analysis in light microscopy. J Microsc. 2006 December; 224(Pt 3):213-32.
23. Constance J E, Despres S D, Nishida A, Lim C S. Selective targeting of c-Abl via a cryptic mitochondrial targeting signal activated by cellular redox status in leukemic and breast cancer cells. Pharm Res. 2012 August; 29(8):2317-28.
24. Constance J E, Woessner D W, Matissek K J, Mossalam M, Lim C S. Enhanced and selective killing of chronic myelogenous leukemia cells with an engineered BCR-ABL binding protein and imatinib. Mol Pharm. 2012 Nov. 5; 9(11):3318-29.
25. Costes S V, Daelemans D, Cho E H, Dobbin Z, Pavlakis G, Lockett S. Automatic and quantitative measurement of protein-protein colocalization in live cells. Biophys J. 2004 June; 86(6):3993-4003.
26. Adler J, Parmryd I. Quantifying colocalization by correlation: the Pearson correlation coefficient is superior to the Mander's overlap coefficient. Cytometry A. 2010 August; 77(8):733-42.
27. Davis J R, Mossalam M, Lim C S. Controlled access of p53 to the nucleus regulates its proteasomal degradation by MDM2. Mol Pharm. 2013 Apr. 1; 10(4):1340-9.
28. Jaskolski F, Mulle C, Manzoni O J. An automated method to quantify and visualize colocalized fluorescent signals. J Neurosci Methods. 2005 Jul. 15; 146(1):42-9.
29. Krohn A J, Wahlbrink T, Prehn J H. Mitochondrial depolarization is not required for neuronal apoptosis. J Neurosci. 1999 Sep. 1; 19(17):7394-404.
30. Grabarek J, Amstad P, Darzynkiewicz Z. Use of fluorescently labeled caspase inhibitors as affinity labels to detect activated caspases. Hum Cell. 2002 March; 15(1):1-12.
31. Grabarek J, Darzynkiewicz Z. In situ activation of caspases and serine proteases during apoptosis detected by affinity labeling their enzyme active centers with fluorochrome-tagged inhibitors. Exp Hematol. 2002 September; 30(9):982-9.
32. Schmid I, Krall W J, Uittenbogaart C H, Braun J, Giorgi J V. Dead cell discrimination with 7-amino-actinomycin D in combination with dual color immunofluorescence in single laser flow cytometry. Cytometry. 1992; 13(2):204-8.
33. Serrano M J, Sanchez-Rovira P, Algarra I, Jaen A, Lozano A, Gaforio J J. Evaluation of a gemcitabine-doxorubicin-paclitaxel combination schedule through flow cytometry assessment of apoptosis extent induced in human breast cancer cell lines. Jpn J Cancer Res. 2002 May; 93(5):559-66.
34. Vermes I, Haanen C, Steffens-Nakken H, Reutelingsperger C. A novel assay for apoptosis. Flow cytometric detection of phosphatidylserine expression on early apoptotic cells using fluorescein labelled Annexin V. J Immunol Methods. 1995 Jul. 17; 184(1):39-51.
35. Loo D T, Rillema J R. Measurement of cell death. Methods Cell Biol. 1998; 57:251-64.
36. Nigro J M, Baker S J, Preisinger A C, Jessup J M, Hostetter R, Cleary K, et al. Mutations in the p53 gene occur in diverse human tumour types. Nature. 1989 Dec. 7; 342(6250):705-8.
37. Bartek J, Iggo R, Gannon J, Lane D P. Genetic and immunochemical analysis of mutant p53 in human breast cancer cell lines. Oncogene. 1990 June; 5(6):893-9.
38. Mooney L M, Al-Sakkaf K A, Brown B L, Dobson P R. Apoptotic mechanisms in T47D and MCF-7 human breast cancer cells. Br J Cancer. 2002 Oct. 7; 87(8):909-17.
39. Goodrum F D, Ornelles D A. p53 status does not determine outcome of E1B 55-kilodalton mutant adenovirus lytic infection. J Virol. 1998 December; 72(12):9479-90.
40. Bodner S M, Minna J D, Jensen S M, D'Amico D, Carbone D, Mitsudomi T, et al. Expression of mutant p53 proteins in lung cancer correlates with the class of p53 gene mutation. Oncogene. 1992 April; 7(4):743-9.
41. Jayaraman S. Flow cytometric determination of mitochondrial membrane potential changes during apoptosis of T lymphocytic and pancreatic beta cell lines: comparison of tetramethylrhodamineethylester (TMRE), chloromethyl-X-rosamine (H2-CMX-Ros) and MitoTracker Red 580 (MTR580). J Immunol Methods. 2005 Nov. 30; 306(1-2):68-79.
42. O'Reilly C M, Fogarty K E, Drummond R M, Tuft R A, Walsh J V, Jr. Quantitative analysis of spontaneous mitochondrial depolarizations. Biophys J. 2003 November; 85(5):3350-7.
43. Ricci J E, Gottlieb R A, Green D R. Caspase-mediated loss of mitochondrial function and generation of reactive oxygen species during apoptosis. J Cell Biol. 2003 Jan. 6; 160(1):65-75.
44. Chowdhury I, Tharakan B, Bhat G K. Caspases—an update. Comp Biochem Physiol B Biochem Mol Biol. 2008 September; 151(1):10-27.
45. Yin Q, Park H H, Chung J Y, Lin S C, Lo Y C, da Graca L S, et al. Caspase-9 holoenzyme is a specific and optimal procaspase-3 processing machine. Mol Cell. 2006 Apr. 21; 22(2):259-68.
46. Mihara M, Erster S, Zaika A, Petrenko O, Chittenden T, Pancoska P, et al. p53 has a direct apoptogenic role at the mitochondria. Mol Cell. 2003 March; 11(3):577-90.
47. Power S D, Lochrie M A, Patterson T E, Poyton R O. The nuclear-coded subunits of yeast cytochrome c oxidase. II. The amino acid sequence of subunit VIII and a model for its disposition in the inner mitochondrial membrane. J Biol Chem. 1984 May 25; 259(10):6571-4.
48. Mossalam M, Dixon A S, Lim C S. Controlling subcellular delivery to optimize therapeutic effect. Ther Deliv. 2010 July; 1(1):169-93.
49. Schmidt O, Pfanner N, Meisinger C. Mitochondrial protein import: from proteomics to functional mechanisms. Nat Rev Mol Cell Biol. 2010 September; 11(9):655-67.
50. Nalepa G, Rolfe M, Harper J W. Drug discovery in the ubiquitin-proteasome system. Nat Rev Drug Discov. 2006 July; 5(7):596-613.
51. Haupt Y, Maya R, Kazaz A, Oren M. Mdm2 promotes the rapid degradation of p53. Nature. 1997 May 15; 387(6630):296-9.
52. Maddocks O D, Vousden K H. Metabolic regulation by p53. J Mol Med (Berl). 2011 March; 89(3):237-45.
53. Hussain S P, Amstad P, He P, Robles A, Lupold S, Kaneko I, et al. p53-induced up-regulation of MnSOD and GPx but not catalase increases oxidative stress and apoptosis. Cancer Res. 2004 Apr. 1; 64(7):2350-6.
54. Yoon K A, Nakamura Y, Arakawa H. Identification of ALDH4 as a p53-inducible gene and its protective role in cellular stresses. J Hum Genet. 2004; 49(3):134-40.
55. Cano C E, Gommeaux J, Pietri S, Culcasi M, Garcia S, Seux M, et al. Tumor protein 53-induced nuclear protein 1 is a major mediator of p53 antioxidant function. Cancer Res. 2009 Jan. 1; 69(1):219-26.
56. Bensaad K, Tsuruta A, Selak M A, Vidal M N, Nakano K, Bartrons R, et al. TIGAR, a p53-inducible regulator of glycolysis and apoptosis. Cell. 2006 Jul. 14; 126(1):107-20.
57. Budanov A V, Karin M. p53 target genes sestrin1 and sestrin2 connect genotoxic stress and mTOR signaling. Cell. 2008 Aug. 8; 134(3):451-60.
58. Ni Chonghaile T, Sarosiek K A, Vo T T, Ryan J A, Tammareddi A, Moore Vdel G, et al. Pretreatment mitochondrial priming correlates with clinical response to cytotoxic chemotherapy. Science. 2011 Nov. 25; 334(6059):1129-33.
59. Constance J E, Lim C S. Targeting malignant mitochondria with therapeutic peptides. Ther Deliv. 2012 August; 3(8):961-79.
60. Yamashita H, Nishio M, Fujii Y, Iwase H. Dominant-negative Stat5 inhibits growth and induces apoptosis in T47D-derived tumors in nude mice. Cancer Sci. 2004 August; 95(8):662-5.
61. Shi J, Zhou Y, Huang H C, Mitchison T J. Navitoclax (ABT-263) accelerates apoptosis during drug-induced mitotic arrest by antagonizing Bcl-xL. Cancer Res. 2011 Jul. 1; 71(13):4518-26.
62. Li H, Piao L, Xu P, Ye W, Zhong S, Lin S H, et al. Liposomes containing (−)-gossypol-enriched cottonseed oil suppress Bcl-2 and Bcl-xL expression in breast cancer cells. Pharm Res. 2011 December; 28(12):3256-64.
63. Jeffrey P D, Gonna S, Pavletich N P. Crystal structure of the tetramerization domain of the p53 tumor suppressor at 1.7 angstroms. Science. 1995 Mar. 10; 267(5203):1498-502.
64. Haupt S, Berger M, Goldberg Z, Haupt Y. Apoptosis—the p53 network. J Cell Sci. 2003 Oct. 15; 116(Pt 20):4077-85.
65. Davis J R, Mossalam M, Lim C S. Controlled access of p53 to the nucleus regulates its proteasomal degradation by MDM2. Mol Pharm. 2013 Apr. 1; 10(4):1340-9.
66. Weinberg R L, Freund S M, Veprintsev D B, Bycroft M, Fersht A R. Regulation of DNA binding of p53 by its C-terminal domain. J Mol Biol. 2004 Sep. 17; 342(3):801-11.
67. Mihara M, Erster S, Zaika A, Petrenko O, Chittenden T, Pancoska P, et al. p53 has a direct apoptogenic role at the mitochondria. Mol Cell. 2003 March; 11(3):577-90.
68. Shaulsky G, Goldfinger N, Ben-Ze'ev A, Rotter V. Nuclear accumulation of p53 protein is mediated by several nuclear localization signals and plays a role in tumorigenesis. Mol Cell Biol. 1990 December; 10(12):6565-77.
69. Marchenko N D, Wolff S, Erster S, Becker K, Moll U M. Monoubiquitylation promotes mitochondrial p53 translocation. EMBO J. 2007 Feb. 21; 26(4):923-34.
70. Perfettini J L, Kroemer R T, Kroemer G. Fatal liaisons of p53 with Bax and Bak. Nat Cell Biol. 2004 May; 6(5):386-8.
71. Chipuk J E, Kuwana T, Bouchier-Hayes L, Droin N M, Newmeyer D D, Schuler M, et al. Direct activation of Bax by p53 mediates mitochondrial membrane permeabilization and apoptosis. Science. 2004 Feb. 13; 303(5660):1010-4.
72. Leu J I, Dumont P, Hafey M, Murphy M E, George D L. Mitochondrial p53 activates Bak and causes disruption of a Bak-Mcl1 complex. Nat Cell Biol. 2004 May; 6(5):443-50.
73. Hagn F, Klein C, Demmer O, Marchenko N, Vaseva A, Moll U M, et al. BclxL changes conformation upon binding to wild-type but not mutant p53 DNA binding domain. J Biol Chem. 2010 Jan. 29; 285(5):3439-50.
74. Tomita Y, Marchenko N, Erster S, Nemajerova A, Dehner A, Klein C, et al. W T p53, but not tumor-derived mutants, bind to Bcl2 via the DNA binding domain and induce mitochondrial permeabilization. J Biol Chem. 2006 Mar. 31; 281(13):8600-6.
75. Chipuk J E, Fisher J C, Dillon C P, Kriwacki R W, Kuwana T, Green D R. Mechanism of apoptosis induction by inhibition of the anti-apoptotic BCL-2 proteins. Proc Natl Acad Sci USA. 2008 Dec. 23; 105(51):20327-32.
76. Tait S W, Green D R. Mitochondria and cell death: outer membrane permeabilization and beyond. Nat Rev Mol Cell Biol. 2010 September; 11(9):621-32.
77. Chowdhury I, Tharakan B, Bhat G K. Caspases—an update. Comp Biochem Physiol B Biochem Mol Biol. 2008 September; 151(1):10-27.
78. Minn A J, Rudin C M, Boise L H, Thompson C B. Expression of bcl-xL can confer a multidrug resistance phenotype. Blood. 1995 Sep. 1; 86(5):1903-10.
79. Yoshino T, Shiina H, Urakami S, Kikuno N, Yoneda T, Shigeno K, et al. Bcl-2 expression as a predictive marker of hormone-refractory prostate cancer treated with taxane-based chemotherapy. Clin Cancer Res. 2006 Oct. 15; 12(20 Pt 1):6116-24.
80. Green D R, Walczak H. Apoptosis therapy: driving cancers down the road to ruin. Nat Med. 2013 February; 19(2):131-3.
81. Kang M H, Reynolds C P. Bcl-2 inhibitors: targeting mitochondrial apoptotic pathways in cancer therapy. Clin Cancer Res. 2009 Feb. 15; 15(4):1126-32.
82. Willis S N, Chen L, Dewson G, Wei A, Naik E, Fletcher J I, et al. Proapoptotic Bak is sequestered by Mcl-1 and Bcl-xL, but not Bcl-2, until displaced by BH3-only proteins. Genes Dev. 2005 Jun. 1; 19(11):1294-305.
83. Ku B, Liang C, Jung J U, Oh B H. Evidence that inhibition of BAX activation by BCL-2 involves its tight and preferential interaction with the BH3 domain of BAX. Cell Res. 2011 April; 21(4):627-41.
84. Schinzel A, Kaufmann T, Schuler M, Martinalbo J, Grubb D, Bomer C. Conformational control of Bax localization and apoptotic activity by Pro168. J Cell Biol. 2004 Mar. 29; 164(7):1021-32.
85. Ferrer P E, Frederick P, Gulbis J M, Dewson G, Kluck R M. Translocation of a Bak C-terminus mutant from cytosol to mitochondria to mediate cytochrome C release: implications for Bak and Bax apoptotic function. PLoS One. 2012; 7(3):e31510.
86. Nechushtan A, Smith C L, Hsu Y T, Youle R J. Conformation of the Bax C-terminus regulates subcellular location and cell death. EMBO J. 1999 May 4; 18(9):2330-41.
87. Pietsch E C, Leu J I, Frank A, Dumont P, George D L, Murphy M E. The tetramerization domain of p53 is required for efficient BAK oligomerization. Cancer Biol Ther. 2007 October; 6(10):1576-83.
88. Pietsch E C, Perchiniak E, Canutescu A A, Wang G, Dunbrack R L, Murphy M E. Oligomerization of BAK by p53 utilizes conserved residues of the p53 DNA binding domain. J Biol Chem. 2008 Jul. 25; 283(30):21294-304.
89. Mossalam M, Matissek K J, Okal A, Constance J E, Lim C S. Direct induction of apoptosis using an optimal mitochondrially targeted p53. Mol Pharm. 2012 May 7; 9(5):1449-58.
90. Matissek K J, Mossalam M, Okal A, Lim C S. The DNA Binding Domain of p53 Is Sufficient To Trigger a Potent Apoptotic Response at the Mitochondria. Mol Pharm. 2013 Sep. 6.
91. Constance J E, Woessner D W, Matissek K J, Mossalam M, Lim C S. Enhanced and selective killing of chronic myelogenous leukemia cells with an engineered BCR-ABL binding protein and imatinib. Mol Pharm. 2012 Nov. 5; 9(11):3318-29.
92. Costes S V, Daelemans D, Cho E H, Dobbin Z, Pavlakis G, Lockett S. Automatic and quantitative measurement of protein-protein colocalization in live cells. Biophys J. 2004 June; 86(6):3993-4003.
93. Bolte S, Cordelieres F P. A guided tour into subcellular colocalization analysis in light microscopy. J Microsc. 2006 December; 224(Pt 3):213-32.
94. Okal A, Mossalam M, Matissek K J, Dixon A S, Moos P J, Lim C S. A Chimeric p53 Evades Mutant p53 Transdominant Inhibition in Cancer Cells. Mol Pharm. 2013 Sep. 9.
95. Schmid I, Krall W J, Uittenbogaart C H, Braun J, Giorgi J V. Dead cell discrimination with 7-amino-actinomycin D in combination with dual color immunofluorescence in single laser flow cytometry. Cytometry. 1992; 13(2):204-8.
96. Yahagi N, Shimano H, Matsuzaka T, Najima Y, Sekiya M, Nakagawa Y, et al. p53 Activation in adipocytes of obese mice. J Biol Chem. 2003 Jul. 11; 278(28):25395-400.
97. Wurstle M L, Laussmann M A, Rehm M. The central role of initiator caspase-9 in apoptosis signal transduction and the regulation of its activation and activity on the apoptosome. Exp Cell Res. 2012 Jul. 1; 318(11):1213-20.
98. Darzynkiewicz Z, Bruno S, Del Bino G, Gorczyca W, Hotz M A, Lassota P, et al. Features of apoptotic cells measured by flow cytometry. Cytometry. 1992; 13(8):795-808.
99. Yin Q, Park H H, Chung J Y, Lin S C, Lo Y C, da Graca L S, et al. Caspase-9 holoenzyme is a specific and optimal procaspase-3 processing machine. Mol Cell. 2006 Apr. 21; 22(2):259-68.
100. Nigro J M, Baker S J, Preisinger A C, Jessup J M, Hostetter R, Cleary K, et al. Mutations in the p53 gene occur in diverse human tumour types. Nature. 1989 Dec. 7; 342(6250):705-8.
101. Bodner S M, Minna J D, Jensen S M, D'Amico D, Carbone D, Mitsudomi T, et al. Expression of mutant p53 proteins in lung cancer correlates with the class of p53 gene mutation. Oncogene. 1992 April; 7(4):743-9.
102. Yaginuma Y, Westphal H. Abnormal structure and expression of the p53 gene in human ovarian carcinoma cell lines. Cancer Res. 1992 Aug. 1; 52(15):4196-9.
103. Goodrum F D, Ornelles D A. p53 status does not determine outcome of E1B 55-kilodalton mutant adenovirus lytic infection. J Virol. 1998 December; 72(12):9479-90.
104. Nalepa G, Rolfe M, Harper J W. Drug discovery in the ubiquitin-proteasome system. Nat Rev Drug Discov. 2006 July; 5(7):596-613.
105. Haupt Y, Maya R, Kazaz A, Oren M. Mdm2 promotes the rapid degradation of p53. Nature. 1997 May 15; 387(6630):296-9.
106. Schellenberg B, Wang P, Keeble J A, Rodriguez-Enriquez R, Walker S, Owens T W, et al. Bax exists in a dynamic equilibrium between the cytosol and mitochondria to control apoptotic priming. Mol Cell. 2013 Mar. 7; 49(5):959-71.
107. Edlich F, Banerjee S, Suzuki M, Cleland M M, Arnoult D, Wang C, et al. Bcl-x(L) retrotranslocates Bax from the mitochondria into the cytosol. Cell. 2011 Apr. 1; 145(1):104-16.
108. Levine A J, Oren M. The first 30 years of p53: growing ever more complex. Nat Rev Cancer. 2009 October; 9(10):749-58.
109. Olivier M, Hollstein M, Hainaut P. TP53 mutations in human cancers: origins, consequences, and clinical use. Cold Spring Harb Perspect Biol. 2010 January; 2(1):a001008.
110. Jackson J G, Lozano G. The mutant p53 mouse as a pre-clinical model. Oncogene. 2013 Sep. 12; 32(37):4325-30.
111. Joerger A C, Fersht A R. The tumor suppressor p53: from structures to drug discovery. Cold Spring Harb Perspect Biol. 2010 June; 2(6):a000919.
112. Kussie P H, Gonna S, Marechal V, Elenbaas B, Moreau J, Levine A J, et al. Structure of the MDM2 oncoprotein bound to the p53 tumor suppressor transactivation domain. Science. 1996 Nov. 8; 274(5289):948-53.
113. Zhang Y, Xiong Y. A p53 amino-terminal nuclear export signal inhibited by DNA damage-induced phosphorylation. Science. 2001 Jun. 8; 292(5523):1910-5.
114. Shaulsky G, Goldfinger N, Ben-Ze'ev A, Rotter V. Nuclear accumulation of p53 protein is mediated by several nuclear localization signals and plays a role in tumorigenesis. Mol Cell Biol. 1990 December; 10(12):6565-77.
115. Stommel J M, Marchenko N D, Jimenez G S, Moll U M, Hope T J, Wahl G M. A leucine-rich nuclear export signal in the p53 tetramerization domain: regulation of subcellular localization and p53 activity by NES masking. EMBO J. 1999 Mar. 15; 18(6):1660-72.
116. Lee W, Harvey T S, Yin Y, Yau P, Litchfield D, Arrowsmith C H. Solution structure of the tetrameric minimum transforming domain of p53. Nat Struct Biol. 1994 December; 1(12):877-90.
117. Thut C J, Chen J L, Klemm R, Tjian R. p53 transcriptional activation mediated by coactivators TAFII40 and TAFII60. Science. 1995 Jan. 6; 267(5194):100-4.
118. Lill N L, Grossman S R, Ginsberg D, DeCaprio J, Livingston D M. Binding and modulation of p53 by p300/CBP coactivators. Nature. 1997 Jun. 19; 387(6635):823-7.
119. Wang X, Jiang X. Mdm2 and MdmX partner to regulate p53. FEBS Lett. 2012 May 21; 586(10):1390-6.
120. Ferreon J C, Lee C W, Arai M, Martinez-Yamout M A, Dyson H J, Wright P E. Cooperative regulation of p53 by modulation of ternary complex formation with CBP/p300 and HDM2. Proc Natl Acad Sci USA. 2009 Apr. 21; 106(16):6591-6.
121. Toledo F, Lee C J, Krummel K A, Rodewald L W, Liu C W, Wahl G M. Mouse mutants reveal that putative protein interaction sites in the p53 proline-rich domain are dispensable for tumor suppression. Mol Cell Biol. 2007 February; 27(4):1425-32.
122. Pavletich N P, Chambers K A, Pabo C O. The DNA-binding domain of p53 contains the four conserved regions and the major mutation hot spots. Genes Dev. 1993 December; 7(12B):2556-64.
123. Toledo F, Wahl G M. Regulating the p53 pathway: in vitro hypotheses, in vivo veritas. Nat Rev Cancer. 2006 December; 6(12):909-23.
124. Kitayner M, Rozenberg H, Kessler N, Rabinovich D, Shaulov L, Haran T E, et al. Structural basis of DNA recognition by p53 tetramers. Mol Cell. 2006 Jun. 23; 22(6):741-53.
125. Mateu M G, Sanchez Del Pino M M, Fersht A R. Mechanism of folding and assembly of a small tetrameric protein domain from tumor suppressor p53. Nat Struct Biol. 1999 February; 6(2):191-8.
126. Mateu M G, Fersht A R. Nine hydrophobic side chains are key determinants of the thermodynamic stability and oligomerization status of tumour suppressor p53 tetramerization domain. EMBO J. 1998 May 15; 17(10):2748-58.
127. Matissek K J B R, Davis J R, Lim C S. Choosing Targets for Gene Therapy. Targets for Gene Therapy 2011 July.
128. Marine J C, Lozano G. Mdm2-mediated ubiquitylation: p53 and beyond. Cell Death Differ. 2010 January; 17(1):93-102.
129. Barak Y, Juven T, Haffner R, Oren M. mdm2 expression is induced by wild type p53 activity. EMBO J. 1993 February; 12(2):461-8.
130. Brooks C L, Gu W. p53 ubiquitination: Mdm2 and beyond. Mol Cell. 2006 Feb. 3; 21(3):307-15.
131. Li M, Brooks C L, Wu-Baer F, Chen D, Baer R, Gu W. Mono- versus polyubiquitination: differential control of p53 fate by Mdm2. Science. 2003 Dec. 12; 302(5652): 1972-5.
132. Hakem A, Bohgaki M, Lemmers B, Tai E, Salmena L, Matysiak-Zablocki E, et al. Role of Pirh2 in mediating the regulation of p53 and c-Myc. PLoS Genet. 2011 November; 7(11):e1002360.
133. Tanimura S, Ohtsuka S, Mitsui K, Shirouzu K, Yoshimura A, Ohtsubo M. MDM2 interacts with MDMX through their RING finger domains. FEBS Lett. 1999 Mar. 19; 447(1):5-9.
134. Shvarts A, Steegenga W T, Riteco N, van Laar T, Dekker P, Bazuine M, et al. MDMX: a novel p53-binding protein with some functional properties of MDM2. EMBO J. 1996 Oct. 1; 15(19):5349-57.
135. Onel K, Cordon-Cardo C. MDM2 and prognosis. Mol Cancer Res. 2004 January; 2(1):1-8.
136. Aylon Y, Oren M. Living with p53, dying of p53. Cell. 2007 Aug. 24; 130(4):597-600.
137. Inga A, Storici F, Darden T A, Resnick M A. Differential transactivation by the p53 transcription factor is highly dependent on p53 level and promoter target sequence. Mol Cell Biol. 2002 December; 22(24):8612-25.
138. Braastad C D, Han Z, Hendrickson E A. Constitutive DNase I hypersensitivity of p53-regulated promoters. J Biol Chem. 2003 Mar. 7; 278(10):8261-8.
139. Kim E, Deppert W. The complex interactions of p53 with target DNA: we learn as we go. Biochem Cell Biol. 2003 June; 81(3):141-50.
140. Budram-Mahadeo V, Morris P J, Latchman D S. The Brn-3a transcription factor inhibits the pro-apoptotic effect of p53 and enhances cell cycle arrest by differentially regulating the activity of the p53 target genes encoding Bax and p21(CIP1/Waf1). Oncogene. 2002 Sep. 5; 21(39):6123-31.
141. Sykes S M, Mellert H S, Holbert M A, Li K, Marmorstein R, Lane W S, et al. Acetylation of the p53 DNA-binding domain regulates apoptosis induction. Mol Cell. 2006 Dec. 28; 24(6):841-51.
142. Le Cam L, Linares L K, Paul C, Julien E, Lacroix M, Hatchi E, et al. E4F1 is an atypical ubiquitin ligase that modulates p53 effector functions independently of degradation. Cell. 2006 Nov. 17; 127(4):775-88.
143. Shmueli A, Oren M. Mdm2: p53's lifesaver? Mol Cell. 2007 Mar. 23; 25(6):794-6.
144. Sullivan A, Lu X. ASPP: a new family of oncogenes and tumour suppressor genes. Br J Cancer. 2007 Jan. 29; 96(2):196-200.
145. Schumm K, Rocha S, Caamano J, Perkins N D. Regulation of p53 tumour suppressor target gene expression by the p52 NF-kappaB subunit. EMBO J. 2006 Oct. 18; 25(20):4820-32.
146. Attardi L D, Reczek E E, Cosmas C, Demicco E G, McCurrach M E, Lowe S W, et al. PERP, an apoptosis-associated target of p53, is a novel member of the PMP-22/gas3 family. Genes Dev. 2000 Mar. 15; 14(6):704-18.
147. Boatright K M, Renatus M, Scott F L, Sperandio S, Shin H, Pedersen I M, et al. A unified model for apical caspase activation. Mol Cell. 2003 February; 11(2):529-41.
148. Boatright K M, Salvesen G S. Mechanisms of caspase activation. Curr Opin Cell Biol. 2003 December; 15(6):725-31.
149. Roy S, Nicholson D W. Cross-talk in cell death signaling. J Exp Med. 2000 Oct. 16; 192(8):F21-5.
150. Desagher S, Martinou J C. Mitochondria as the central control point of apoptosis. Trends Cell Biol. 2000 September; 10(9):369-77.
151. Haupt S, Berger M, Goldberg Z, Haupt Y. Apoptosis—the p53 network. J Cell Sci. 2003 Oct. 15; 116(Pt 20):4077-85.
152. Riedl S J, Salvesen G S. The apoptosome: signalling platform of cell death. Nat Rev Mol Cell Biol. 2007 May; 8(5):405-13.
153. Martins L M, Iaccarino I, Tenev T, Gschmeissner S, Totty N F, Lemoine N R, et al. The serine protease Omi/HtrA2 regulates apoptosis by binding XIAP through a reaper-like motif. J Biol Chem. 2002 Jan. 4; 277(1):439-44.
154. Janicke R U, Sprengart M L, Wati M R, Porter A G. Caspase-3 is required for DNA fragmentation and morphological changes associated with apoptosis. J Biol Chem. 1998 Apr. 17; 273(16):9357-60.
155. Zha H, Aime-Sempe C, Sato T, Reed J C. Proapoptotic protein Bax heterodimerizes with Bcl-2 and homodimerizes with Bax via a novel domain (BH3) distinct from BH1 and BH2. J Biol Chem. 1996 Mar. 29; 271(13):7440-4.
156. Antonsson B, Montessuit S, Sanchez B, Martinou J C. Bax is present as a high molecular weight oligomer/complex in the mitochondrial membrane of apoptotic cells. J Biol Chem. 2001 Apr. 13; 276(15):11615-23.
157. Chipuk J E, Moldoveanu T, Llambi F, Parsons M J, Green D R. The BCL-2 family reunion. Mol Cell. 2010 Feb. 12; 37(3):299-310.
158. Willis S N, Adams J M. Life in the balance: how BH3-only proteins induce apoptosis. Curr Opin Cell Biol. 2005 December; 17(6):617-25.
159. Zheng J Y, Tsai Y C, Kadimcherla P, Zhang R, Shi J, Oyler G A, et al. The C-terminal transmembrane domain of Bcl-xL mediates changes in mitochondrial morphology. Biophys J. 2008 Jan. 1; 94(1):286-97.
160. Reed J C. Double identity for proteins of the Bcl-2 family. Nature. 1997 Jun. 19; 387(6635):773-6.
161. Chipuk J E, Green D R. How do BCL-2 proteins induce mitochondrial outer membrane permeabilization? Trends Cell Biol. 2008 April; 18(4):157-64.
162. Ferrer P E, Frederick P, Gulbis J M, Dewson G, Kluck R M. Translocation of a Bak C-terminus mutant from cytosol to mitochondria to mediate cytochrome C release: implications for Bak and Bax apoptotic function. PLoS One. 2012; 7(3):e31510.
163. Tait S W, Green D R. Mitochondria and cell death: outer membrane permeabilization and beyond. Nat Rev Mol Cell Biol. 2010 September; 11(9):621-32.
164. Mihara M, Erster S, Zaika A, Petrenko O, Chittenden T, Pancoska P, et al. p53 has a direct apoptogenic role at the mitochondria. Mol Cell. 2003 March; 11(3):577-90.
165. Marchenko N D, Wolff S, Erster S, Becker K, Moll U M. Monoubiquitylation promotes mitochondrial p53 translocation. EMBO J. 2007 Feb. 21; 26(4):923-34.
166. Marchenko N D, Moll U M. The role of ubiquitination in the direct mitochondrial death program of p53. Cell Cycle. 2007 Jul. 15; 6(14):1718-23.
167. Hagn F, Klein C, Demmer O, Marchenko N, Vaseva A, Moll U M, et al. BclxL changes conformation upon binding to wild-type but not mutant p53 DNA binding domain. J Biol Chem. 2010 Jan. 29; 285(5):3439-50.
168. Tomita Y, Marchenko N, Erster S, Nemajerova A, Dehner A, Klein C, et al. W T p53, but not tumor-derived mutants, bind to Bcl2 via the DNA binding domain and induce mitochondrial permeabilization. J Biol Chem. 2006 Mar. 31; 281(13):8600-6.
169. Perfettini J L, Kroemer R T, Kroemer G. Fatal liaisons of p53 with Bax and Bak. Nat Cell Biol. 2004 May; 6(5):386-8.
170. Petros A M, Gunasekera A, Xu N, Olejniczak E T, Fesik S W. Defining the p53 DNA-binding domain/Bcl-x(L)-binding interface using NMR. FEBS Left. 2004 Feb. 13; 559(1-3):171-4.
171. Sot B, Freund S M, Fersht A R. Comparative biophysical characterization of p53 with the pro-apoptotic BAK and the anti-apoptotic BCL-xL. J Biol Chem. 2007 Oct. 5; 282(40):29193-200.
172. Vaseva A V, Moll U M. The mitochondrial p53 pathway. Biochim Biophys Acta. 2009 May; 1787(5):414-20.
173. Chipuk J E, Kuwana T, Bouchier-Hayes L, Droin N M, Newmeyer D D, Schuler M, et al. Direct activation of Bax by p53 mediates mitochondrial membrane permeabilization and apoptosis. Science. 2004 Feb. 13; 303(5660):1010-4.
174. Wolff S, Erster S, Palacios G, Moll U M. p53's mitochondrial translocation and MOMP action is independent of Puma and Bax and severely disrupts mitochondrial membrane integrity. Cell Res. 2008 July; 18(7):733-44.
175. Vousden K H, Prives C. Blinded by the Light: The Growing Complexity of p53. Cell. 2009 May 1; 137(3):413-31.
176. Maddocks O D, Vousden K H. Metabolic regulation by p53. J Mol Med (Berl). 2011 March; 89(3):237-45.
177. Bensaad K, Tsuruta A, Selak M A, Vidal M N, Nakano K, Bartrons R, et al. TIGAR, a p53-inducible regulator of glycolysis and apoptosis. Cell. 2006 Jul. 14; 126(1):107-20.
178. Shen L, Sun X, Fu Z, Yang G, Li J, Yao L. The fundamental role of the p53 pathway in tumor metabolism and its implication in tumor therapy. Clin Cancer Res. 2012 Mar. 15; 18(6):1561-7.
179. Vousden K H, Ryan K M. p53 and metabolism. Nat Rev Cancer. 2009 October; 9(10):691-700.
180. Watanabe H, Ohta S, Kumon Y, Sakaki S, Sakanaka M. Increase in p53 protein expression following cortical infarction in the spontaneously hypertensive rat. Brain Res. 1999 Aug. 7; 837(1-2):38-45.
181. Liu P, Xu B, Cavalieri T A, Hock C E. Pifithrin-alpha attenuates p53-mediated apoptosis and improves cardiac function in response to myocardial ischemia/reperfusion in aged rats. Shock. 2006 December; 26(6):608-14.
182. Bretaud S, Allen C, Ingham P W, Bandmann O. p53-dependent neuronal cell death in a DJ-1-deficient zebrafish model of Parkinson's disease. J Neurochem. 2007 March; 100(6):1626-35.
183. Culmsee C, Landshamer S. Molecular insights into mechanisms of the cell death program: role in the progression of neurodegenerative disorders. Curr Alzheimer Res. 2006 September; 3(4):269-83.
184. Bae B I, Xu H, Igarashi S, Fujimuro M, Agrawal N, Taya Y, et al. p53 mediates cellular dysfunction and behavioral abnormalities in Huntington's disease. Neuron. 2005 Jul. 7; 47(1):29-41.
185. Culmsee C, Zhu X, Yu Q S, Chan S L, Camandola S, Guo Z, et al. A synthetic inhibitor of p53 protects neurons against death induced by ischemic and excitotoxic insults, and amyloid beta-peptide. J Neurochem. 2001 April; 77(1):220-8.
186. Brosh R, Rotter V. When mutants gain new powers: news from the mutant p53 field. Nat Rev Cancer. 2009 October; 9(10):701-13.
187. Peller S, Rotter V. TP53 in hematological cancer: low incidence of mutations with significant clinical relevance. Hum Mutat. 2003 March; 21(3):277-84.
188. Coles C, Condie A, Chetty U, Steel C M, Evans H J, Prosser J. p53 mutations in breast cancer. Cancer Res. 1992 Oct. 1; 52(19):5291-8.
189. Schuijer M, Berns E M. TP53 and ovarian cancer. Hum Mutat. 2003 March; 21(3):285-91.
190. Iacopetta B. TP53 mutation in colorectal cancer. Hum Mutat. 2003 March; 21(3):271-6.
191. Blons H, Laurent-Puig P. TP53 and head and neck neoplasms. Hum Mutat. 2003 March; 21(3):252-7.
192. Hollstein M, Sidransky D, Vogelstein B, Harris C C. p53 mutations in human cancers. Science. 1991 Jul. 5; 253(5015):49-53.
193. Kim E, Deppert W. The versatile interactions of p53 with DNA: when flexibility serves specificity. Cell Death Differ. 2006 June; 13(6):885-9.
194. Milner J, Medcalf E A, Cook A C. Tumor suppressor p53: analysis of wild-type and mutant p53 complexes. Mol Cell Biol. 1991 January; 11(1):12-9.
195. Milner J, Medcalf E A. Cotranslation of activated mutant p53 with wild type drives the wild-type p53 protein into the mutant conformation. Cell. 1991 May 31; 65(5):765-74.
196. Sigal A, Rotter V. Oncogenic mutations of the p53 tumor suppressor: the demons of the guardian of the genome. Cancer Res. 2000 Dec. 15; 60(24):6788-93.
197. Bergamaschi D, Gasco M, Hiller L, Sullivan A, Syed N, Trigiante G, et al. p53 polymorphism influences response in cancer chemotherapy via modulation of p73-dependent apoptosis. Cancer Cell. 2003 April; 3(4):387-402.
198. Lang G A, Iwakuma T, Suh Y A, Liu G, Rao V A, Parant J M, et al. Gain of function of a p53 hot spot mutation in a mouse model of Li-Fraumeni syndrome. Cell. 2004 Dec. 17; 119(6):861-72.
199. Cho Y, Gonna S, Jeffrey P D, Pavletich N P. Crystal structure of a p53 tumor suppressor-DNA complex: understanding tumorigenic mutations. Science. 1994 Jul. 15; 265(5170):346-55.
200. Muller P A, Vousden K H. p53 mutations in cancer. Nat Cell Biol. 2013 January; 15(1):2-8.
201. Vassilev L T, Vu B T, Graves B, Carvajal D, Podlaski F, Filipovic Z, et al. In vivo activation of the p53 pathway by small-molecule antagonists of MDM2. Science. 2004 Feb. 6; 303(5659):844-8.
202. Issaeva N, Bozko P, Enge M, Protopopova M, Verhoef L G, Masucci M, et al. Small molecule RITA binds to p53, blocks p53-HDM-2 interaction and activates p53 function in tumors. Nat Med. 2004 December; 10(12):1321-8.
203. Jiang M, Milner J. Selective silencing of viral gene expression in HPV-positive human cervical carcinoma cells treated with siRNA, a primer of RNA interference. Oncogene. 2002 Sep. 5; 21(39):6041-8.
204. Yu Y, Sun P, Sun L C, Liu G Y, Chen G H, Shang L H, et al. Downregulation of MDM2 expression by RNAi inhibits LoVo human colorectal adenocarcinoma cells growth and the treatment of LoVo cells with mdm2siRNA3 enhances the sensitivity to cisplatin. Biochem Biophys Res Commun. 2006 Jan. 6; 339(1):71-8.
205. Lain S, Hollick J J, Campbell J, Staples O D, Higgins M, Aoubala M, et al. Discovery, in vivo activity, and mechanism of action of a small-molecule p53 activator. Cancer Cell. 2008 May; 13(5):454-63.
206. Wiman K G. Pharmacological reactivation of mutant p53: from protein structure to the cancer patient. Oncogene. 2010 Jul. 29; 29(30):4245-52.
207. Sugikawa E, Hosoi T, Yazaki N, Gamanuma M, Nakanishi N, Ohashi M. Mutant p53 mediated induction of cell cycle arrest and apoptosis at G1 phase by 9-hydroxyellipticine. Anticancer Res. 1999 July-August; 19(4B):3099-108.
208. Piantino C B, Reis S T, Viana N I, Silva I A, Morais D R, Antunes A A, et al. Prima-1 induces apoptosis in bladder cancer cell lines by activating p53. Clinics (Sao Paulo). 2013; 68(3):297-303.
209. Saha M N, Qiu L, Chang H. Targeting p53 by small molecules in hematological malignancies. J Hematol Oncol. 2013; 6:23.
210. Bykov V J, Issaeva N, Zache N, Shilov A, Hultcrantz M, Bergman J, et al. Reactivation of mutant p53 and induction of apoptosis in human tumor cells by maleimide analogs. J Biol Chem. 2005 Aug. 26; 280(34):30384-91.
211. Kravchenko J E, Ilyinskaya G V, Komarov P G, Agapova L S, Kochetkov D V, Strom E, et al. Small-molecule RETRA suppresses mutant p53-bearing cancer cells through a p73-dependent salvage pathway. Proc Natl Acad Sci USA. 2008 Apr. 29; 105(17):6302-7.
212. Speetjens F M, Kuppen P J, Welters M J, Essahsah F, Voet van den Brink A M, Lantrua M G, et al. Induction of p53-specific immunity by a p53 synthetic long peptide vaccine in patients treated for metastatic colorectal cancer. Clin Cancer Res. 2009 Feb. 1; 15(3):1086-95.
213. Lane D P, Cheok C F, Lain S. p53-based cancer therapy. Cold Spring Harb Perspect Biol. 2010 September; 2(9):a001222.
214. Suzuki K, Matsubara H. Recent advances in p53 research and cancer treatment. J Biomed Biotechnol. 2011; 2011:978312.
215. Palacios G, Crawford H C, Vaseva A, Moll U M. Mitochondrially targeted wild-type p53 induces apoptosis in a solid human tumor xenograft model. Cell Cycle. 2008 Aug. 15; 7(16):2584-90.
216. Palacios G, Moll U M. Mitochondrially targeted wild-type p53 suppresses growth of mutant p53 lymphomas in vivo. Oncogene. 2006 Oct. 5; 25(45):6133-9.
217. Talos F, Petrenko O, Mena P, Moll U M. Mitochondrially targeted p53 has tumor suppressor activities in vivo. Cancer Res. 2005 Nov. 1; 65(21):9971-81.
218. Erster S, Moll U M. Stress-induced p53 runs a direct mitochondrial death program: its role in physiologic and pathophysiologic stress responses in vivo. Cell Cycle. 2004 December; 3(12):1492-5.
219. Jeffrey P D, Gonna S, Pavletich N P. Crystal structure of the tetramerization domain of the p53 tumor suppressor at 1.7 angstroms. Science. 1995 Mar. 10; 267(5203):1498-502.
220. Okal A, Mossalam M, Matissek K J, Dixon A S, Moos P J, Lim C S. A chimeric p53 evades mutant p53 transdominant inhibition in cancer cells. Mol Pharm. 2013 Oct. 7; 10(10):3922-33.
221. Mackenzie S, McIntosh L. Higher plant mitochondria. Plant Cell. 1999 April; 11(4):571-86.
222. Bowsher C G, Tobin A K. Compartmentation of metabolism within mitochondria and plastids. J Exp Bot. 2001 April; 52(356):513-27.
223. Jones A. Does the plant mitochondrion integrate cellular stress and regulate programmed cell death? Trends Plant Sci. 2000 May; 5(5):225-30.
224. Youle R J, Karbowski M. Mitochondrial fission in apoptosis. Nat Rev Mol Cell Biol. 2005 August; 6(8):657-63.
225. Logan D C. The mitochondrial compartment. J Exp Bot. 2007; 58(1):1225-43.
226. Fulda S, Galluzzi L, Kroemer G. Targeting mitochondria for cancer therapy. Nat Rev Drug Discov. 2010 June; 9(6):447-64.
227. Leaver C J, Hack E, Forde B G. Protein synthesis by isolated plant mitochondria. Methods Enzymol. 1983; 97:476-84.
228. Gray M W, Burger G, Lang B F. Mitochondrial evolution. Science. 1999 Mar. 5; 283(5407):1476-81.
229. Unseld M, Marienfeld J R, Brandt P, Brennicke A. The mitochondrial genome of Arabidopsis thaliana contains 57 genes in 366,924 nucleotides. Nat Genet. 1997 January; 15(1):57-61.
230. Whelan J, Glaser E. Protein import into plant mitochondria. Plant Mol Biol. 1997 March; 33(5):771-89.
231. Wiedemann N, Frazier A E, Pfanner N. The protein import machinery of mitochondria. J Biol Chem. 2004 Apr. 9; 279(15):14473-6.
232. Hill K, Model K, Ryan M T, Dietmeier K, Martin F, Wagner R, et al. Tom40 forms the hydrophilic channel of the mitochondrial import pore for preproteins [see comment]. Nature. 1998 Oct. 1; 395(6701):516-21.
233. Kunkele K P, Heins S, Dembowski M, Nargang F E, Benz R, Thieffry M, et al. The preprotein translocation channel of the outer membrane of mitochondria. Cell. 1998 Jun. 12; 93(6):1009-19.
234. van Wilpe S, Ryan M T, Hill K, Maarse A C, Meisinger C, Brix J, et al. Tom22 is a multifunctional organizer of the mitochondrial preprotein translocase. Nature. 1999 Sep. 30; 401(6752):485-9.
235. Abe Y, Shodai T, Muto T, Mihara K, Torii H, Nishikawa S, et al. Structural basis of presequence recognition by the mitochondrial protein import receptor Tom20. Cell. 2000 Mar. 3; 100(5):551-60.
236. Brix J, Dietmeier K, Pfanner N. Differential recognition of preproteins by the purified cytosolic domains of the mitochondrial import receptors Tom20, Tom22, and Tom70. J Biol Chem. 1997 Aug. 15; 272(33):20730-5.
237. Geissler A, Chacinska A, Truscott K N, Wiedemann N, Brandner K, Sickmann A, et al. The mitochondrial presequence translocase: an essential role of Tim50 in directing preproteins to the import channel. Cell. 2002 Nov. 15; 111(4):507-18.
238. Rehling P, Brandner K, Pfanner N. Mitochondrial import and the twin-pore translocase. Nat Rev Mol Cell Biol. 2004 July; 5(7):519-30.
239. Endo T, Kohda D. Functions of outer membrane receptors in mitochondrial protein import. Biochim Biophys Acta. 2002 Sep. 2; 1592(1):3-14.
240. Kemper C, Habib S J, Engl G, Heckmeyer P, Dimmer K S, Rapaport D. Integration of tail-anchored proteins into the mitochondrial outer membrane does not require any known import components. J Cell Sci. 2008 Jun. 15; 121(Pt 12):1990-8.
241. Wattenberg B, Lithgow T. Targeting of C-terminal (tail)-anchored proteins: understanding how cytoplasmic activities are anchored to intracellular membranes. Traffic. 2001 January; 2(1):66-71.
242. Bohni P C, Daum G, Schatz G. Import of proteins into mitochondria. Partial purification of a matrix-located protease involved in cleavage of mitochondrial precursor polypeptides. J Biol Chem. 1983 Apr. 25; 258(8):4937-43.
243. von Heijne G. Mitochondrial targeting sequences may form amphiphilic helices. EMBO J. 1986 June; 5(6):1335-42.
244. Roise D, Schatz G. Mitochondrial presequences. J Biol Chem. 1988 Apr. 5; 263(10):4509-11.
245. Schinzel A, Kaufmann T, Schuler M, Martinalbo J, Grubb D, Bomer C. Conformational control of Bax localization and apoptotic activity by Pro168. J Cell Biol. 2004 Mar. 29; 164(7):1021-32.
246. Wilfling F, Weber A, Potthoff S, Vogtle F N, Meisinger C, Paschen S A, et al. BH3-only proteins are tail-anchored in the outer mitochondrial membrane and can initiate the activation of Bax. Cell Death Differ. 2012 August; 19(8):1328-36.
247. Zhao X, Ghaffari S, Lodish H, Malashkevich V N, Kim P S. Structure of the Bcr-Abl oncoprotein oligomerization domain. Nat Struct Biol. 2002 February; 9(2):117-20.
248. Taylor C M, Keating A E. Orientation and oligomerization specificity of the Bcr coiled-coil oligomerization domain. Biochemistry. 2005 Dec. 13; 44(49):16246-56.
249. Yu W, Fang H. Clinical trials with oncolytic adenovirus in China. Curr Cancer Drug Targets. 2007 March; 7(2):141-8.
250. Shi J, Zheng D. An update on gene therapy in China. Curr Opin Mol Ther. 2009 October; 11(5):547-53.
251. Li Y, Li L J, Zhang S T, Wang L J, Zhang Z, Gao N, et al. In vitro and clinical studies of gene therapy with recombinant human adenovirus-p53 injection for oral leukoplakia. Clin Cancer Res. 2009 Nov. 1; 15(21):6724-31.
252. Ginn S L, Alexander I E, Edelstein M L, Abedi M R, Wixon J. Gene therapy clinical trials worldwide to 2012—an update. J Gene Med. 2013 February; 15(2):65-77.
253. Al-Dosari M S, Gao X. Nonviral gene delivery: principle, limitations, and recent progress. AAPS J. 2009 December; 11(4):671-81.
254. Nayerossadat N, Maedeh T, Ali P A. Viral and nonviral delivery systems for gene delivery. Adv Biomed Res. 2012; 1:27.
255. Thomas C E, Ehrhardt A, Kay M A. Progress and problems with the use of viral vectors for gene therapy. Nat Rev Genet. 2003 May; 4(5):346-58.
256. Vorburger S A, Hunt K K. Adenoviral gene therapy. Oncologist. 2002; 7(1):46-59.
257. Cotrim A P, Baum B J. Gene therapy: some history, applications, problems, and prospects. Toxicol Pathol. 2008 January; 36(1):97-103.
258. Rein D T, Breidenbach M, Curiel D T. Current developments in adenovirus-based cancer gene therapy. Future Oncol. 2006 February; 2(1):137-43.
259. Erster S, Mihara M, Kim R H, Petrenko O, Moll U M. In vivo mitochondrial p53 translocation triggers a rapid first wave of cell death in response to DNA damage that can precede p53 target gene activation. Mol Cell Biol. 2004 August; 24(15):6728-41.
260. Erster S, Moll U M. Stress-induced p53 runs a transcription-independent death program. Biochem Biophys Res Commun. [Review]. 2005 Jun. 10; 331(3):843-50.
261. Mihara M, Erster S, Zaika A, Petrenko O, Chittenden T, Pancoska P, et al. p53 has a direct apoptogenic role at the mitochondria. Mol Cell. 2003 March; 11(3):577-90.
262. Chipuk J E, Kuwana T, Bouchier-Hayes L, Droin N M, Newmeyer D D, Schuler M, et al. Direct activation of Bax by p53 mediates mitochondrial membrane permeabilization and apoptosis. Science. 2004 Feb. 13; 303(5660):1010-4.
263. Zhao Y, Chaiswing L, Velez J M, Batinic-Haberle I, Colburn N H, Oberley T D, et al. p53 translocation to mitochondria precedes its nuclear translocation and targets mitochondrial oxidative defense protein-manganese superoxide dismutase. Cancer Res. 2005 May 1; 65(9):3745-50.
264. Canugovi C, Maynard S, Bayne A C, Sykora P, Tian J, de Souza-Pinto N C, et al. The mitochondrial transcription factor A functions in mitochondrial base excision repair. DNA Repair. 2010 Oct. 5; 9(10):1080-9.
265. Vogelstein B, Lane D, Levine A J. Surfing the p53 network. Nature. 2000 Nov. 16; 408(6810):307-10.
266. El-Deiry W S, Harper J W, O'Connor P M, Velculescu V E, Canman C E, Jackman J, et al. WAF1/CIP1 is induced in p53-mediated G1 arrest and apoptosis. Cancer Res. 1994 Mar. 1; 54(5):1169-74.
267. Harris S L, Levine A J. The p53 pathway: positive and negative feedback loops. Oncogene. 2005 Apr. 18; 24(17):2899-908.
268. Marchenko N D, Zaika A, Moll U M. Death signal-induced localization of p53 protein to mitochondria. A potential role in apoptotic signaling. J Biol Chem. 2000 May 26; 275(21):16202-12.
269. Tomita Y, Marchenko N, Erster S, Nemajerova A, Dehner A, Klein C, et al. W T p53, but not tumor-derived mutants, bind to Bcl2 via the DNA binding domain and induce mitochondrial permeabilization. J Biol Chem. 2006 Mar. 31; 281(13):8600-6.
270. Chipuk J E, Maurer U, Green D R, Schuler M. Pharmacologic activation of p53 elicits Bax-dependent apoptosis in the absence of transcription. Cancer Cell. 2003 November; 4(5):371-81.
271. Leu J I, Dumont P, Hafey M, Murphy M E, George D L. Mitochondrial p53 activates Bak and causes disruption of a Bak-Mcl1 complex. Nat Cell Biol. 2004 May; 6(5):443-50.
272. Pietsch E C, Leu J I, Frank A, Dumont P, George D L, Murphy M E. The tetramerization domain of p53 is required for efficient BAK oligomerization. Cancer Biol Ther. [Research Support, N.I.H., Extramural]. 2007 October; 6(10):1576-83.
273. Green D R, Reed J C. Mitochondria and apoptosis. Science. 1998 Aug. 28; 281(5381): 1309-12.
274. Reed J C. Cytochrome c: can't live with it—can't live without it. Cell. 1997 Nov. 28; 91(5):559-62.
275. Green D R, Evan G I. A matter of life and death. Cancer Cell. 2002 February; 1(1):19-30.
276. Mossalam M, Dixon A S, Lim C S. Controlling subcellular delivery to optimize therapeutic effect. Ther Deliv. 2010 Jul. 1; 1(1):169-93.
277. Mihara M, Moll U M. Detection of mitochondrial localization of p53. Methods Mol Biol. 2003; 234:203-9.
278. Shaulsky G, Goldfinger N, Ben-Ze'ev A, Rotter V. Nuclear accumulation of p53 protein is mediated by several nuclear localization signals and plays a role in tumorigenesis. Mol Cell Biol. 1990 December; 10(12):6565-77.
279. Jiang P, Du W, Heese K, Wu M. The Bad guy cooperates with good cop p53: Bad is transcriptionally up-regulated by p53 and forms a Bad/p53 complex at the mitochondria to induce apoptosis. Mol and Cel Biol. 2006 December; 26(23):9071-82.
280. Sansome C, Zaika A, Marchenko N D, Moll U M. Hypoxia death stimulus induces translocation of p53 protein to mitochondria. Detection by immunofluorescence on whole cells. FEBS Lett. 2001 Jan. 19; 488(3):110-5.
281. Dumont P, Leu J I, Della Pietra A C, 3rd, George D L, Murphy M. The codon 72 polymorphic variants of p53 have markedly different apoptotic potential. Nat Genet. 2003 March; 33(3):357-65.
282. Marchenko N D, Wolff S, Erster S, Becker K, Moll U M. Monoubiquitylation promotes mitochondrial p53 translocation. EMBO J. 2007 Feb. 21; 26(4):923-34.
283. Von Heijne G. Mitochondrial targeting sequences may form amphiphilic helices. EMBO J. 1986 June; 5(6):1335-42.
284. Koehler C M. New developments in mitochondrial assembly. Annu Rev Cell Dev Biol. 2004; 20:309-35.
285. Roise D, Schatz G. Mitochondrial presequences. J Biol Chem. 1988 Apr. 5; 263(10):4509-11.
286. Wiedemann N, Frazier A E, Pfanner N. The protein import machinery of mitochondria. J Biol Chem. [Review]. 2004 Apr. 9; 279(15):14473-6.
287. Kaufmann T, Schlipf S, Sanz J, Neubert K, Stein R, Borner C. Characterization of the signal that directs Bcl-x(L), but not Bcl-2, to the mitochondrial outer membrane. J Cell Biol. 2003 Jan. 6; 160(1):53-64.
288. Pollack M, Leeuwenburgh C. Apoptosis and aging: role of the mitochondria. J Gerontology. 2001 November; 56(11):B475-82.
289. Lindsay J, Esposti M D, Gilmore A P. Bcl-2 proteins and mitochondria—specificity in membrane targeting for death. Biochim Biophys Acta. [Review]. 2011 April; 1813(4):532-9.
290. Rehling P, Brandner K, Pfanner N. Mitochondrial import and the twin-pore translocase. Nat Rev Mol Cell Biol. 2004 July; 5(7):519-30.
291. Power S D, Lochrie M A, Patterson T E, Poyton R O. The nuclear-coded subunits of yeast cytochrome c oxidase. II. The amino acid sequence of subunit VIII and a model for its disposition in the inner mitochondrial membrane. J Biol Chem. [Comparative StudyResearch Support, Non-U.S. Gov'tResearch Support, U.S. Gov't, P.H.S.]. 1984 May 25; 259(10):6571-4.
292. Fabrizi G M, Sadlock J, Hirano M, Mita S, Koga Y, Rizzuto R, et al. Differential expression of genes specifying two isoforms of subunit VIa of human cytochrome c oxidase. Gene. 1992 Oct. 1; 119(2):307-12.
293. Isaya G, Fenton W A, Hendrick J P, Furtak K, Kalousek F, Rosenberg L E. Mitochondrial import and processing of mutant human ornithine transcarbamylase precursors in cultured cells. Mol Cell Biol. 1988 December; 8(12):5150-8.
294. Dixon A S, Kakar M, Schneider K M, Constance J E, Paullin B C, Lim C S. Controlling subcellular localization to alter function: Sending oncogenic Bcr-Abl to the nucleus causes apoptosis. J Control Release. 2009 Dec. 16; 140(3):245-9.
295. Bolte S, Cordelieres F P. A guided tour into subcellular colocalization analysis in light microscopy. J Microscopy. 2006 December; 224(Pt 3):213-32.
296. Costes S V, Daelemans D, Cho E H, Dobbin Z, Pavlakis G, Lockett S. Automatic and quantitative measurement of protein-protein colocalization in live cells. Biophysical J. 2004 June; 86(6):3993-4003.
297. Adler J, Parmryd I. Quantifying colocalization by correlation: the Pearson correlation coefficient is superior to the Mander's overlap coefficient. Cytometry A. August; 77(8):733-42.
298. Dixon A S, Miller G D, Bruno B J, Constance J E, Woessner D W, Fidler T P, et al. Improved coiled-coil design enhances interaction with bcr-abl and induces apoptosis. Mol Pharm. 2012 Jan. 1; 9(1):187-95.
299. Jaskolski F, Mulle C, Manzoni O J. An automated method to quantify and visualize colocalized fluorescent signals. J Neuroscience Methods. 2005 Jul. 15; 146(1):42-9.
300. Schafer J M, Lee E S, O'Regan R M, Yao K, Jordan V C. Rapid development of tamoxifen-stimulated mutant p53 breast tumors (T47D) in athymic mice. Clin Cancer Res. 2000 November; 6(11):4373-80.
301. Alkhalaf M, El-Mowafy A M. Overexpression of wild-type p53 gene renders MCF-7 breast cancer cells more sensitive to the antiproliferative effect of progesterone. J Endocrinol. 2003 October; 179(1):55-62.
302. Takahashi K, Sumimoto H, Suzuki K, Ono T. Protein synthesis-dependent cytoplasmic translocation of p53 protein after serum stimulation of growth-arrested MCF-7 cells. Mol Carcinog. 1993; 8(1):58-66.
303. Mooney L M, Al-Sakkaf K A, Brown B L, Dobson P R. Apoptotic mechanisms in T47D and MCF-7 human breast cancer cells. Br J Cancer. 2002 Oct. 7; 87(8):909-17.
304. Chowdhury I, Tharakan B, Bhat G K. Caspases—an update. Comp Biochem Physiol B Biochem Mol Biol [Review]. 2008 September; 151(1):10-27.
305. Yin Q, Park H H, Chung J Y, Lin S C, Lo Y C, da Graca L S, et al. Caspase-9 holoenzyme is a specific and optimal procaspase-3 processing machine. Mol Cell. 2006 Apr. 21; 22(2):259-68.
306. Loo D T, Rillema J R. Measurement of cell death. Meth Cell Biol. [Review]. 1998; 57:251-64.
307. Koopman G, Reutelingsperger C P, Kuijten G A, Keehnen R M, Pals S T, van Oers M H. Annexin V for flow cytometric detection of phosphatidylserine expression on B cells undergoing apoptosis. Blood. 1994 Sep. 1; 84(5):1415-20.
308. Vermes I, Haanen C, Steffens-Nakken H, Reutelingsperger C. A novel assay for apoptosis. Flow cytometric detection of phosphatidylserine expression on early apoptotic cells using fluorescein labelled Annexin V. J Immun Meth. 1995 Jul. 17; 184(1):39-51.
309. Metkar S S, Wang B, Catalan E, Anderluh G, Gilbert R J, Pardo J, et al. Perforin rapidly induces plasma membrane phospholipid flip-flop. PLoS One. 2011; 6(9):e24286.
310. Schmid I, Krall W J, Uittenbogaart C H, Braun J, Giorgi J V. Dead cell discrimination with 7-amino-actinomycin D in combination with dual color immunofluorescence in single laser flow cytometry. Cytometry. 1992; 13(2):204-8.
311. Serrano M J, Sanchez-Rovira P, Algarra I, Jaen A, Lozano A, Gaforio J J. Evaluation of a gemcitabine-doxorubicin-paclitaxel combination schedule through flow cytometry assessment of apoptosis extent induced in human breast cancer cell lines. Jap J Cancer Res. [Research Support, Non-U.S. Gov't]. 2002 May; 93(5):559-66.
312. Komarov P G, Komarova E A, Kondratov R V, Christov-Tselkov K, Coon J S, Chemov M V, et al. A chemical inhibitor of p53 that protects mice from the side effects of cancer therapy. Science. 1999 Sep. 10; 285(5434):1733-7.
313. Liu X, Chua C C, Gao J, Chen Z, Landy C L, Hamdy R, et al. Pifithrin-alpha protects against doxorubicin-induced apoptosis and acute cardiotoxicity in mice. Am J Physiol. 2004 March; 286(3):H933-9.
314. Hagn F, Klein C, Demmer O, Marchenko N, Vaseva A, Moll U M, et al. BclxL changes conformation upon binding to wild-type but not mutant p53 DNA binding domain. J Biol Chem. 2010 Jan. 29; 285(5):3439-50.
315. Strom E, Sathe S, Komarov P G, Chernova O B, Pavlovska I, Shyshynova I, et al. Small-molecule inhibitor of p53 binding to mitochondria protects mice from gamma radiation. Nat Chem Biol. 2006 September; 2(9):474-9.
316. Vaseva A V, Moll U M. The mitochondrial p53 pathway. Biochim Biophys Acta. 2009 May; 1787(5):414-20.
317. Schmidt O, Pfanner N, Meisinger C. Mitochondrial protein import: from proteomics to functional mechanisms. Nat Rev Mol Cell Biol. 2010 September; 11(9):655-67.
318. Endo T, Kohda D. Functions of outer membrane receptors in mitochondrial protein import. Biochim Biophys Acta. 2002 Sep. 2; 1592(1):3-14.
319. Dolezal P, Likic V, Tachezy J, Lithgow T. Evolution of the molecular machines for protein import into mitochondria. Science. 2006 Jul. 21; 313(5785):314-8.
320. Mattaj I W, Englmeier L. Nucleocytoplasmic transport: the soluble phase. Annu Rev Biochem. 1998; 67:265-306.
321. Gorlich D, Kutay U. Transport between the cell nucleus and the cytoplasm. Annu Rev Cell Dev Biol. 1999; 15:607-60.
322. Davis J R, Kakar M, Lim C S. Controlling protein compartmentalization to overcome disease. Pharm Res. 2007 January; 24(1):17-27.
323. Sattler M, Liang H, Nettesheim D, Meadows R P, Harlan J E, Eberstadt M, et al. Structure of Bcl-xL-Bak peptide complex: recognition between regulators of apoptosis. Science. [Research Support, U.S. Gov't, P.H.S.]. 1997 Feb. 14; 275(5302):983-6.
324. Galluzzi L, Morselli E, Kepp O, Tajeddine N, Kroemer G. Targeting p53 to mitochondria for cancer therapy. Cell Cycle. 2008 Jul. 1; 7(13):1949-55.
325. Goh A M, Coffill C R, Lane D P. The role of mutant p53 in human cancer. J Pathol. 2011 January; 223(2):116-26.
326. Sherr C J. Principles of tumor suppression. Cell. 2004 Jan. 23; 116(2):235-46.
327. Martins C P, Brown-Swigart L, Evan G I. Modeling the therapeutic efficacy of p53 restoration in tumors. Cell. 2006 Dec. 29; 127(7):1323-34.
328. Xin H. Chinese gene therapy. Gendicine's efficacy: hard to translate. Science. 2006 Nov. 24; 314(5803):1233.
329. Peng Z. Current status of gendicine in China: recombinant human Ad-p53 agent for treatment of cancers. Hum Gene Ther. 2005 September; 16(9):1016-27.
330. Palacios G, Crawford H C, Vaseva A, Moll U M. Mitochondrially targeted wild-type p53 induces apoptosis in a solid human tumor xenograft model. Cell Cycle. 2008 Aug. 15; 7(16):2584-90.
331. Fuster J J, Sanz-Gonzalez S M, Moll U M, Andres V. Classic and novel roles of p53: prospects for anticancer therapy. Trends Mol Med. 2007 May; 13(5):192-9.
332. Perfettini J L, Kroemer R T, Kroemer G. Fatal liaisons of p53 with Bax and Bak. Nat Cell Biol. 2004 May; 6(5):386-8.
333. Vaseva A V, Moll U M. The mitochondrial p53 pathway. Biochim Biophys Acta. 2009 May; 1787(5):414-20.
334. Joerger A C, Fersht A R. The tumor suppressor p53: from structures to drug discovery. Cold Spring Harb Perspect Biol. 2010 June; 2(6):a000919.
335. Hagn F, Klein C, Demmer O, Marchenko N, Vaseva A, Moll U M, et al. BclxL changes conformation upon binding to wild-type but not mutant p53 DNA binding domain. J Biol Chem. 2010 Jan. 29; 285(5):3439-50.
336. Petros A M, Gunasekera A, Xu N, Olejniczak E T, Fesik S W. Defining the p53 DNA-binding domain/Bcl-x(L)-binding interface using NMR. FEBS Left. 2004 Feb. 13; 559(1-3):171-4.
337. Tomita Y, Marchenko N, Erster S, Nemajerova A, Dehner A, Klein C, et al. W T p53, but not tumor-derived mutants, bind to Bcl2 via the DNA binding domain and induce mitochondrial permeabilization. J Biol Chem. 2006 Mar. 31; 281(13):8600-6.
338. Bharatham N, Chi S W, Yoon H S. Molecular basis of Bcl-X(L)-p53 interaction: insights from molecular dynamics simulations. PLoS One. 2011; 6(10):e26014.
339. Chipuk J E, Maurer U, Green D R, Schuler M. Pharmacologic activation of p53 elicits Bax-dependent apoptosis in the absence of transcription. Cancer Cell. 2003 November; 4(5):371-81.
340. Ha J H, Won E Y, Shin J S, Jang M, Ryu K S, Bae K H, et al. Molecular mimicry-based repositioning of nutlin-3 to anti-apoptotic Bcl-2 family proteins. J Am Chem Soc. 2011 Feb. 9; 133(5):1244-7.
341. Xu H, Tai J, Ye H, Kang C B, Yoon H S. The N-terminal domain of tumor suppressor p53 is involved in the molecular interaction with the anti-apoptotic protein Bcl-Xl. Biochem Biophys Res Commun. 2006 Mar. 24; 341(4):938-44.
342. Mossalam M, Matissek K J, Okal A, Constance J E, Lim C S. Direct induction of apoptosis using an optimal mitochondrially targeted p53. Mol Pharm. 2012 May 7; 9(5):1449-58.
343. Dixon A S, Pendley S S, Bruno B J, Woessner D W, Shimpi A A, Cheatham T E, 3rd, et al. Disruption of Bcr-Abl coiled coil oligomerization by design. J Biol Chem. 2011 Aug. 5; 286(31):27751-60.
344. Dixon A S, Miller G D, Bruno B J, Constance J E, Woessner D W, Fidler T P, et al. Improved coiled-coil design enhances interaction with Bcr-Abl and induces apoptosis. Mol Pharm. 2012 Jan. 1; 9(1):187-95.
345. Dixon A S, Kakar M, Schneider K M, Constance J E, Paullin B C, Lim C S. Controlling subcellular localization to alter function: Sending oncogenic Bcr-Abl to the nucleus causes apoptosis. J Control Release. 2009 Dec. 16; 140(3):245-9.
346. Bolte S, Cordelieres F P. A guided tour into subcellular colocalization analysis in light microscopy. J Microsc. 2006 December; 224(Pt 3):213-32.
347. Constance J E, Despres S D, Nishida A, Lim C S. Selective targeting of c-Abl via a cryptic mitochondrial targeting signal activated by cellular redox status in leukemic and breast cancer cells. Pharm Res. 2012 August; 29(8):2317-28.
348. Constance J E, Woessner D W, Matissek K J, Mossalam M, Lim C S. Enhanced and selective killing of chronic myelogenous leukemia cells with an engineered BCR-ABL binding protein and imatinib. Mol Pharm. 2012 Nov. 5; 9(11):3318-29.
349. Costes S V, Daelemans D, Cho E H, Dobbin Z, Pavlakis G, Lockett S. Automatic and quantitative measurement of protein-protein colocalization in live cells. Biophys J. 2004 June; 86(6):3993-4003.
350. Adler J, Parmryd I. Quantifying colocalization by correlation: the Pearson correlation coefficient is superior to the Mander's overlap coefficient. Cytometry A. 2010 August; 77(8):733-42.
351. Davis J R, Mossalam M, Lim C S. Controlled access of p53 to the nucleus regulates its proteasomal degradation by MDM2. Mol Pharm. 2013 Apr. 1; 10(4):1340-9.
352. Jaskolski F, Mulle C, Manzoni O J. An automated method to quantify and visualize colocalized fluorescent signals. J Neurosci Methods. 2005 Jul. 15; 146(1):42-9.
353. Krohn A J, Wahlbrink T, Prehn J H. Mitochondrial depolarization is not required for neuronal apoptosis. J Neurosci. 1999 Sep. 1; 19(17):7394-404.
354. Grabarek J, Amstad P, Darzynkiewicz Z. Use of fluorescently labeled caspase inhibitors as affinity labels to detect activated caspases. Hum Cell. 2002 March; 15(1):1-12.
355. Grabarek J, Darzynkiewicz Z. In situ activation of caspases and serine proteases during apoptosis detected by affinity labeling their enzyme active centers with fluorochrome-tagged inhibitors. Exp Hematol. 2002 September; 30(9):982-9.
356. Schmid I, Krall W J, Uittenbogaart C H, Braun J, Giorgi J V. Dead cell discrimination with 7-amino-actinomycin D in combination with dual color immunofluorescence in single laser flow cytometry. Cytometry. 1992; 13(2):204-8.
357. Serrano M J, Sanchez-Rovira P, Algarra I, Jaen A, Lozano A, Gaforio J J. Evaluation of a gemcitabine-doxorubicin-paclitaxel combination schedule through flow cytometry assessment of apoptosis extent induced in human breast cancer cell lines. Jpn J Cancer Res. 2002 May; 93(5):559-66.
358. Vermes I, Haanen C, Steffens-Nakken H, Reutelingsperger C. A novel assay for apoptosis. Flow cytometric detection of phosphatidylserine expression on early apoptotic cells using fluorescein labelled Annexin V. J Immunol Methods. 1995 Jul. 17; 184(1):39-51.
359. Loo D T, Rillema J R. Measurement of cell death. Methods Cell Biol. 1998; 57:251-64.
360. Nigro J M, Baker S J, Preisinger A C, Jessup J M, Hostetter R, Cleary K, et al. Mutations in the p53 gene occur in diverse human tumour types. Nature. 1989 Dec. 7; 342(6250):705-8.
361. Bartek J, Iggo R, Gannon J, Lane D P. Genetic and immunochemical analysis of mutant p53 in human breast cancer cell lines. Oncogene. 1990 June; 5(6):893-9.
362. Mooney L M, Al-Sakkaf K A, Brown B L, Dobson P R. Apoptotic mechanisms in T47D and MCF-7 human breast cancer cells. Br J Cancer. 2002 Oct. 7; 87(8):909-17.
363. Goodrum F D, Ornelles D A. p53 status does not determine outcome of E1B 55-kilodalton mutant adenovirus lytic infection. J Virol. 1998 December; 72(12):9479-90.
364. Bodner S M, Minna J D, Jensen S M, D'Amico D, Carbone D, Mitsudomi T, et al. Expression of mutant p53 proteins in lung cancer correlates with the class of p53 gene mutation. Oncogene. 1992 April; 7(4):743-9.
365. Jayaraman S. Flow cytometric determination of mitochondrial membrane potential changes during apoptosis of T lymphocytic and pancreatic beta cell lines: comparison of tetramethylrhodamineethylester (TMRE), chloromethyl-X-rosamine (H2-CMX-Ros) and MitoTracker Red 580 (MTR580). J Immunol Methods. 2005 Nov. 30; 306(1-2):68-79.
366. O'Reilly C M, Fogarty K E, Drummond R M, Tuft R A, Walsh J V, Jr. Quantitative analysis of spontaneous mitochondrial depolarizations. Biophys J. 2003 November; 85(5):3350-7.
367. Ricci J E, Gottlieb R A, Green D R. Caspase-mediated loss of mitochondrial function and generation of reactive oxygen species during apoptosis. J Cell Biol. 2003 Jan. 6; 160(1):65-75.
368. Chowdhury I, Tharakan B, Bhat G K. Caspases—an update. Comp Biochem Physiol B Biochem Mol Biol. 2008 September; 151(1):10-27.
369. Yin Q, Park H H, Chung J Y, Lin S C, Lo Y C, da Graca L S, et al. Caspase-9 holoenzyme is a specific and optimal procaspase-3 processing machine. Mol Cell. 2006 Apr. 21; 22(2):259-68.
370. Mihara M, Erster S, Zaika A, Petrenko O, Chittenden T, Pancoska P, et al. p53 has a direct apoptogenic role at the mitochondria. Mol Cell. 2003 March; 11(3):577-90.
371. Power S D, Lochrie M A, Patterson T E, Poyton R O. The nuclear-coded subunits of yeast cytochrome c oxidase. II. The amino acid sequence of subunit VIII and a model for its disposition in the inner mitochondrial membrane. J Biol Chem. 1984 May 25; 259(10):6571-4.
372. Mossalam M, Dixon A S, Lim C S. Controlling subcellular delivery to optimize therapeutic effect. Ther Deliv. 2010 July; 1(1):169-93.
373. Schmidt O, Pfanner N, Meisinger C. Mitochondrial protein import: from proteomics to functional mechanisms. Nat Rev Mol Cell Biol. 2010 September; 11(9):655-67.
374. Nalepa G, Rolfe M, Harper J W. Drug discovery in the ubiquitin-proteasome system. Nat Rev Drug Discov. 2006 July; 5(7):596-613.
375. Haupt Y, Maya R, Kazaz A, Oren M. Mdm2 promotes the rapid degradation of p53. Nature. 1997 May 15; 387(6630):296-9.
376. Maddocks O D, Vousden K H. Metabolic regulation by p53. J Mol Med (Berl). 2011 March; 89(3):237-45.
377. Hussain S P, Amstad P, He P, Robles A, Lupold S, Kaneko I, et al. p53-induced up-regulation of MnSOD and GPx but not catalase increases oxidative stress and apoptosis. Cancer Res. 2004 Apr. 1; 64(7):2350-6.
378. Yoon K A, Nakamura Y, Arakawa H. Identification of ALDH4 as a p53-inducible gene and its protective role in cellular stresses. J Hum Genet. 2004; 49(3):134-40.
379. Cano C E, Gommeaux J, Pietri S, Culcasi M, Garcia S, Seux M, et al. Tumor protein 53-induced nuclear protein 1 is a major mediator of p53 antioxidant function. Cancer Res. 2009 Jan. 1; 69(1):219-26.
380. Bensaad K, Tsuruta A, Selak M A, Vidal M N, Nakano K, Bartrons R, et al. TIGAR, a p53-inducible regulator of glycolysis and apoptosis. Cell. 2006 Jul. 14; 126(1):107-20.
381. Budanov A V, Karin M. p53 target genes sestrin1 and sestrin2 connect genotoxic stress and mTOR signaling. Cell. 2008 Aug. 8; 134(3):451-60.
382. Ni Chonghaile T, Sarosiek K A, Vo T T, Ryan J A, Tammareddi A, Moore Vdel G, et al. Pretreatment mitochondrial priming correlates with clinical response to cytotoxic chemotherapy. Science. 2011 Nov. 25; 334(6059):1129-33.
383. Constance J E, Lim C S. Targeting malignant mitochondria with therapeutic peptides. Ther Deliv. 2012 August; 3(8):961-79.
384. Yamashita H, Nishio M, Fujii Y, Iwase H. Dominant-negative Stat5 inhibits growth and induces apoptosis in T47D-derived tumors in nude mice. Cancer Sci. 2004 August; 95(8):662-5.
385. Shi J, Zhou Y, Huang H C, Mitchison T J. Navitoclax (ABT-263) accelerates apoptosis during drug-induced mitotic arrest by antagonizing Bcl-xL. Cancer Res. 2011 Jul. 1; 71(13):4518-26.
386. Li H, Piao L, Xu P, Ye W, Zhong S, Lin S H, et al. Liposomes containing (−)-gossypol-enriched cottonseed oil suppress Bcl-2 and Bcl-xL expression in breast cancer cells. Pharm Res. 2011 December; 28(12):3256-64.
387. Jeffrey P D, Gonna S, Pavletich N P. Crystal structure of the tetramerization domain of the p53 tumor suppressor at 1.7 angstroms. Science. 1995 Mar. 10; 267(5203):1498-502.
388. Haupt S, Berger M, Goldberg Z, Haupt Y. Apoptosis—the p53 network. J Cell Sci. 2003 Oct. 15; 116(Pt 20):4077-85.
389. Davis J R, Mossalam M, Lim C S. Controlled access of p53 to the nucleus regulates its proteasomal degradation by MDM2. Mol Pharm. 2013 Apr. 1; 10(4):1340-9.
390. Weinberg R L, Freund S M, Veprintsev D B, Bycroft M, Fersht A R. Regulation of DNA binding of p53 by its C-terminal domain. J Mol Biol. 2004 Sep. 17; 342(3):801-11.
391. Mihara M, Erster S, Zaika A, Petrenko O, Chittenden T, Pancoska P, et al. p53 has a direct apoptogenic role at the mitochondria. Mol Cell. 2003 March; 11(3):577-90.
392. Shaulsky G, Goldfinger N, Ben-Ze'ev A, Rotter V. Nuclear accumulation of p53 protein is mediated by several nuclear localization signals and plays a role in tumorigenesis. Mol Cell Biol. 1990 December; 10(12):6565-77.
393. Marchenko N D, Wolff S, Erster S, Becker K, Moll U M. Monoubiquitylation promotes mitochondrial p53 translocation. EMBO J. 2007 Feb. 21; 26(4):923-34.
394. Perfettini J L, Kroemer R T, Kroemer G. Fatal liaisons of p53 with Bax and Bak. Nat Cell Biol. 2004 May; 6(5):386-8.
395. Chipuk J E, Kuwana T, Bouchier-Hayes L, Droin N M, Newmeyer D D, Schuler M, et al. Direct activation of Bax by p53 mediates mitochondrial membrane permeabilization and apoptosis. Science. 2004 Feb. 13; 303(5660):1010-4.
396. Leu J I, Dumont P, Hafey M, Murphy M E, George D L. Mitochondrial p53 activates Bak and causes disruption of a Bak-Mcl1 complex. Nat Cell Biol. 2004 May; 6(5):443-50.
397. Hagn F, Klein C, Demmer O, Marchenko N, Vaseva A, Moll U M, et al. BclxL changes conformation upon binding to wild-type but not mutant p53 DNA binding domain. J Biol Chem. 2010 Jan. 29; 285(5):3439-50.
398. Tomita Y, Marchenko N, Erster S, Nemajerova A, Dehner A, Klein C, et al. W T p53, but not tumor-derived mutants, bind to Bcl2 via the DNA binding domain and induce mitochondrial permeabilization. J Biol Chem. 2006 Mar. 31; 281(13):8600-6.
399. Chipuk J E, Fisher J C, Dillon C P, Kriwacki R W, Kuwana T, Green D R. Mechanism of apoptosis induction by inhibition of the anti-apoptotic BCL-2 proteins. Proc Natl Acad Sci USA. 2008 Dec. 23; 105(51):20327-32.
400. Tait S W, Green D R. Mitochondria and cell death: outer membrane permeabilization and beyond. Nat Rev Mol Cell Biol. 2010 September; 11(9):621-32.
401. Chowdhury I, Tharakan B, Bhat G K. Caspases—an update. Comp Biochem Physiol B Biochem Mol Biol. 2008 September; 151(1):10-27.
402. Minn A J, Rudin C M, Boise L H, Thompson C B. Expression of bcl-xL can confer a multidrug resistance phenotype. Blood. 1995 Sep. 1; 86(5):1903-10.
403. Yoshino T, Shiina H, Urakami S, Kikuno N, Yoneda T, Shigeno K, et al. Bcl-2 expression as a predictive marker of hormone-refractory prostate cancer treated with taxane-based chemotherapy. Clin Cancer Res. 2006 Oct. 15; 12(20 Pt 1):6116-24.
404. Green D R, Walczak H. Apoptosis therapy: driving cancers down the road to ruin. Nat Med. 2013 February; 19(2):131-3.
405. Kang M H, Reynolds C P. Bcl-2 inhibitors: targeting mitochondrial apoptotic pathways in cancer therapy. Clin Cancer Res. 2009 Feb. 15; 15(4):1126-32.
406. Willis S N, Chen L, Dewson G, Wei A, Naik E, Fletcher J I, et al. Proapoptotic Bak is sequestered by Mcl-1 and Bcl-xL, but not Bcl-2, until displaced by BH3-only proteins. Genes Dev. 2005 Jun. 1; 19(11):1294-305.
407. Ku B, Liang C, Jung J U, Oh B H. Evidence that inhibition of BAX activation by BCL-2 involves its tight and preferential interaction with the BH3 domain of BAX. Cell Res. 2011 April; 21(4):627-41.
408. Schinzel A, Kaufmann T, Schuler M, Martinalbo J, Grubb D, Borner C. Conformational control of Bax localization and apoptotic activity by Pro168. J Cell Biol. 2004 Mar. 29; 164(7):1021-32.
409. Ferrer P E, Frederick P, Gulbis J M, Dewson G, Kluck R M. Translocation of a Bak C-terminus mutant from cytosol to mitochondria to mediate cytochrome C release: implications for Bak and Bax apoptotic function. PLoS One. 2012; 7(3):e31510.
410. Nechushtan A, Smith C L, Hsu Y T, Youle R J. Conformation of the Bax C-terminus regulates subcellular location and cell death. EMBO J. 1999 May 4; 18(9):2330-41.
411. Pietsch E C, Leu J I, Frank A, Dumont P, George D L, Murphy M E. The tetramerization domain of p53 is required for efficient BAK oligomerization. Cancer Biol Ther. 2007 October; 6(10):1576-83.
412. Pietsch E C, Perchiniak E, Canutescu A A, Wang G, Dunbrack R L, Murphy M E. Oligomerization of BAK by p53 utilizes conserved residues of the p53 DNA binding domain. J Biol Chem. 2008 Jul. 25; 283(30):21294-304.
413. Mossalam M, Matissek K J, Okal A, Constance J E, Lim C S. Direct induction of apoptosis using an optimal mitochondrially targeted p53. Mol Pharm. 2012 May 7; 9(5):1449-58.
414. Matissek K J, Mossalam M, Okal A, Lim C S. The DNA Binding Domain of p53 Is Sufficient To Trigger a Potent Apoptotic Response at the Mitochondria. Mol Pharm. 2013 Sep. 6.
415. Constance J E, Woessner D W, Matissek K J, Mossalam M, Lim C S. Enhanced and selective killing of chronic myelogenous leukemia cells with an engineered BCR-ABL binding protein and imatinib. Mol Pharm. 2012 Nov. 5; 9(11):3318-29.
416. Costes S V, Daelemans D, Cho E H, Dobbin Z, Pavlakis G, Lockett S. Automatic and quantitative measurement of protein-protein colocalization in live cells. Biophys J. 2004 June; 86(6):3993-4003.
417. Bolte S, Cordelieres F P. A guided tour into subcellular colocalization analysis in light microscopy. J Microsc. 2006 December; 224(Pt 3):213-32.
418. Okal A, Mossalam M, Matissek K J, Dixon A S, Moos P J, Lim C S. A Chimeric p53 Evades Mutant p53 Transdominant Inhibition in Cancer Cells. Mol Pharm. 2013 Sep. 9.
419. Schmid I, Krall W J, Uittenbogaart C H, Braun J, Giorgi J V. Dead cell discrimination with 7-amino-actinomycin D in combination with dual color immunofluorescence in single laser flow cytometry. Cytometry. 1992; 13(2):204-8.
420. Yahagi N, Shimano H, Matsuzaka T, Najima Y, Sekiya M, Nakagawa Y, et al. p53 Activation in adipocytes of obese mice. J Biol Chem. 2003 Jul. 11; 278(28):25395-400.
421. Wurstle M L, Laussmann M A, Rehm M. The central role of initiator caspase-9 in apoptosis signal transduction and the regulation of its activation and activity on the apoptosome. Exp Cell Res. 2012 Jul. 1; 318(11):1213-20.
422. Darzynkiewicz Z, Bruno S, Del Bino G, Gorczyca W, Hotz M A, Lassota P, et al. Features of apoptotic cells measured by flow cytometry. Cytometry. 1992; 13(8):795-808.
423. Yin Q, Park H H, Chung J Y, Lin S C, Lo Y C, da Graca L S, et al. Caspase-9 holoenzyme is a specific and optimal procaspase-3 processing machine. Mol Cell. 2006 Apr. 21; 22(2):259-68.
424. Nigro J M, Baker S J, Preisinger A C, Jessup J M, Hostetter R, Cleary K, et al. Mutations in the p53 gene occur in diverse human tumour types. Nature. 1989 Dec. 7; 342(6250):705-8.
425. Bodner S M, Minna J D, Jensen S M, D'Amico D, Carbone D, Mitsudomi T, et al. Expression of mutant p53 proteins in lung cancer correlates with the class of p53 gene mutation. Oncogene. 1992 April; 7(4):743-9.
426. Yaginuma Y, Westphal H. Abnormal structure and expression of the p53 gene in human ovarian carcinoma cell lines. Cancer Res. 1992 Aug. 1; 52(15):4196-9.
427. Goodrum F D, Ornelles D A. p53 status does not determine outcome of E1B 55-kilodalton mutant adenovirus lytic infection. J Virol. 1998 December; 72(12):9479-90.
428. Erster S, Moll U M. Stress-induced p53 runs a direct mitochondrial death program: its role in physiologic and pathophysiologic stress responses in vivo. Cell Cycle. 2004 December; 3(12):1492-5.
429. Nalepa G, Rolfe M, Harper J W. Drug discovery in the ubiquitin-proteasome system. Nat Rev Drug Discov. 2006 July; 5(7):596-613.
430. Haupt Y, Maya R, Kazaz A, Oren M. Mdm2 promotes the rapid degradation of p53. Nature. 1997 May 15; 387(6630):296-9.
431. Schellenberg B, Wang P, Keeble J A, Rodriguez-Enriquez R, Walker S, Owens T W, et al. Bax exists in a dynamic equilibrium between the cytosol and mitochondria to control apoptotic priming. Mol Cell. 2013 Mar. 7; 49(5):959-71.
432. Edlich F, Banerjee S, Suzuki M, Cleland M M, Arnoult D, Wang C, et al. Bcl-x(L) retrotranslocates Bax from the mitochondria into the cytosol. Cell. 2011 Apr. 1; 145(1):104-16.
433. Yu W, Fang H. Clinical trials with oncolytic adenovirus in China. Curr Cancer Drug Targets. 2007 March; 7(2):141-8.
434. Shi J, Zheng D. An update on gene therapy in China. Curr Opin Mol Ther. 2009 October; 11(5):547-53.
435. Lane D P, Cheok C F, Lain S. p53-based cancer therapy. Cold Spring Harb Perspect Biol. 2010 September; 2(9):a001222.
436. Sigal A, Rotter V. Oncogenic mutations of the p53 tumor suppressor: the demons of the guardian of the genome. Cancer Res. 2000 Dec. 15; 60(24):6788-93.
437. Milner J, Medcalf E A, Cook A C. Tumor suppressor p53: analysis of wild-type and mutant p53 complexes. Mol Cell Biol. 1991 January; 11(1):12-9.
438. Hollstein M, Sidransky D, Vogelstein B, Harris C C. p53 mutations in human cancers. Science. 1991 Jul. 5; 253(5015):49-53.
439. Kim E, Deppert W. The versatile interactions of p53 with DNA: when flexibility serves specificity. Cell Death Differ. 2006 June; 13(6):885-9.
440. Srivastava S, Wang S, Tong Y A, Hao Z M, Chang E H. Dominant negative effect of a germ-line mutant p53: a step fostering tumorigenesis. Cancer Res. 1993 Oct. 1; 53(19):4452-5.
441. Kern S E, Pietenpol J A, Thiagalingam S, Seymour A, Kinzler K W, Vogelstein B. Oncogenic forms of p53 inhibit p53-regulated gene expression. Science. 1992 May 8; 256(5058):827-30.
442. Turner N, Moretti E, Siclari O, Migliaccio I, Santarpia L, D'Incalci M, et al. Targeting triple negative breast cancer: is p53 the answer? Cancer Treat Rev. 2013 August; 39(5):541-50.
443. Palacios G, Crawford H C, Vaseva A, Moll U M. Mitochondrially targeted wild-type p53 induces apoptosis in a solid human tumor xenograft model. Cell Cycle. 2008 Aug. 15; 7(16):2584-90.
444. Vaseva A V, Moll U M. The mitochondrial p53 pathway. Biochim Biophys Acta. 2009 May; 1787(5):414-20.
445. Mossalam M, Matissek K J, Okal A, Constance J E, Lim C S. Direct induction of apoptosis using an optimal mitochondrially targeted p53. Mol Pharm. 2012 May 7; 9(5):1449-58.
446. Matissek K J, Mossalam M, Okal A, Lim C S. The DNA Binding Domain of p53 Is Sufficient To Trigger a Potent Apoptotic Response at the Mitochondria. Mol Pharm. 2013 Sep. 6.
447. Li Y, Li L J, Zhang S T, Wang L J, Zhang Z, Gao N, et al. In vitro and clinical studies of gene therapy with recombinant human adenovirus-p53 injection for oral leukoplakia. Clin Cancer Res. 2009 Nov. 1; 15(21):6724-31.
448. Ginn S L, Alexander I E, Edelstein M L, Abedi M R, Wixon J. Gene therapy clinical trials worldwide to 2012—an update. J Gene Med. 2013 February; 15(2):65-77.
449. Nielsen L L, Dell J, Maxwell E, Armstrong L, Maneval D, Catino J J. Efficacy of p53 adenovirus-mediated gene therapy against human breast cancer xenografts. Cancer Gene Ther. 1997 March-April; 4(2):129-38.
450. Nielsen L L, Lipari P, Dell J, Gurnani M, Hajian G. Adenovirus-mediated p53 gene therapy and paclitaxel have synergistic efficacy in models of human head and neck, ovarian, prostate, and breast cancer. Clin Cancer Res. 1998 April; 4(4):835-46.
451. Okal A, Mossalam M, Matissek K J, Dixon A S, Moos P J, Lim C S. A chimeric p53 evades mutant p53 transdominant inhibition in cancer cells. Mol Pharm. 2013 Oct. 7; 10(10):3922-33.
452. Matissek K J, Mossalam M, Okal A, Lim C S. The DNA binding domain of p53 is sufficient to trigger a potent apoptotic response at the mitochondria. Mol Pharm. 2013 Oct. 7; 10(10):3592-602.
453. Di Como C J, Gaiddon C, Prives C. p73 function is inhibited by tumor-derived p53 mutants in mammalian cells. Mol Cell Biol. 1999 February; 19(2):1438-49.
454. Brachova P, Thiel K W, Leslie K K. The consequence of oncomorphic TP53 mutations in ovarian cancer. Int J Mol Sci. 2013; 14(9):19257-75.
455. Bodner S M, Minna J D, Jensen S M, D'Amico D, Carbone D, Mitsudomi T, et al. Expression of mutant p53 proteins in lung cancer correlates with the class of p53 gene mutation. Oncogene. 1992 April; 7(4):743-9.
456. Lim L Y, Vidnovic N, Ellisen L W, Leong C O. Mutant p53 mediates survival of breast cancer cells. Br J Cancer. 2009 Nov. 3; 101(9):1606-12.
457. Wang W, Cheng B, Miao L, Mei Y, Wu M. Mutant p53-R273H gains new function in sustained activation of EGFR signaling via suppressing miR-27a expression. Cell Death Dis. 2013; 4:e574.
458. Chowdhury I, Tharakan B, Bhat G K. Caspases—an update. Comp Biochem Physiol B Biochem Mol Biol. 2008 September; 151(1):10-27.
459. Mihara M, Erster S, Zaika A, Petrenko O, Chittenden T, Pancoska P, et al. p53 has a direct apoptogenic role at the mitochondria. Mol Cell. 2003 March; 11(3):577-90.
460. Ding Y, Wen Y, Spohn B, Wang L, Xia W, Kwong K Y, et al. Proapoptotic and antitumor activities of adenovirus-mediated p202 gene transfer. Clin Cancer Res. 2002 October; 8(10):3290-7.
461. Perfettini J L, Kroemer R T, Kroemer G. Fatal liaisons of p53 with Bax and Bak. Nat Cell Biol. 2004 May; 6(5):386-8.
462. Haupt S, Berger M, Goldberg Z, Haupt Y. Apoptosis—the p53 network. J Cell Sci. 2003 Oct. 15; 116(Pt 20):4077-85.
463. Olive K P, Tuveson D A, Ruhe Z C, Yin B, Willis N A, Bronson R T, et al. Mutant p53 gain of function in two mouse models of Li-Fraumeni syndrome. Cell. 2004 Dec. 17; 119(6):847-60.
464. El-Deiry W S. The role of p53 in chemosensitivity and radiosensitivity. Oncogene. 2003 Oct. 20; 22(47):7486-95.
465. Gurnani M, Lipari P, Dell J, Shi B, Nielsen L L. Adenovirus-mediated p53 gene therapy has greater efficacy when combined with chemotherapy against human head and neck, ovarian, prostate, and breast cancer. Cancer Chemother Pharmacol. 1999; 44(2):143-51.
466. Minn A J, Rudin C M, Boise L H, Thompson C B. Expression of bcl-xL can confer a multidrug resistance phenotype. Blood. 1995 Sep. 1; 86(5):1903-10.
467. Perego P, Righetti S C, Supino R, Delia D, Caserini C, Carenini N, et al. Role of apoptosis and apoptosis-related proteins in the cisplatin-resistant phenotype of human tumor cell lines. Apoptosis. 1997; 2(6):540-8.
468. Reed J C. Bcl-2-family proteins and hematologic malignancies: history and future prospects. Blood. 2008 Apr. 1; 111(7):3322-30.
469. Yoshino T, Shiina H, Urakami S, Kikuno N, Yoneda T, Shigeno K, et al. Bcl-2 expression as a predictive marker of hormone-refractory prostate cancer treated with taxane-based chemotherapy. Clin Cancer Res. 2006 Oct. 15; 12(20 Pt 1):6116-24.
470. Schellenberg B, Wang P, Keeble J A, Rodriguez-Enriquez R, Walker S, Owens T W, et al. Bax exists in a dynamic equilibrium between the cytosol and mitochondria to control apoptotic priming. Mol Cell. 2013 Mar. 7; 49(5):959-71.
471. Edlich F, Banerjee S, Suzuki M, Cleland M M, Arnoult D, Wang C, et al. Bcl-x(L) retrotranslocates Bax from the mitochondria into the cytosol. Cell. 2011 Apr. 1; 145(1):104-16.
472. Willis S N, Chen L, Dewson G, Wei A, Naik E, Fletcher J I, et al. Proapoptotic Bak is sequestered by Mcl-1 and Bcl-xL, but not Bcl-2, until displaced by BH3-only proteins. Genes Dev. 2005 Jun. 1; 19(11):1294-305.
473. Schuijer M, Berns E M. TP53 and ovarian cancer. Hum Mutat. 2003 March; 21(3):285-91.
474. Peller S, Rotter V. TP53 in hematological cancer: low incidence of mutations with significant clinical relevance. Hum Mutat. 2003 March; 21(3):277-84.
475. Coles C, Condie A, Chetty U, Steel C M, Evans H J, Prosser J. p53 mutations in breast cancer. Cancer Res. 1992 Oct. 1; 52(19):5291-8.
476. Muller P A, Vousden K H. p53 mutations in cancer. Nat Cell Biol. 2013 January; 15(1):2-8.
477. Brosh R, Rotter V. When mutants gain new powers: news from the mutant p53 field. Nat Rev Cancer. 2009 October; 9(10):701-13.
478. Hollstein M, Sidransky D, Vogelstein B, Harris C C. p53 mutations in human cancers. Science. 1991 Jul. 5; 253(5015):49-53.
479. Kim E, Deppert W. The versatile interactions of p53 with DNA: when flexibility serves specificity. Cell Death Differ. 2006 June; 13(6):885-9.
480. Milner J, Medcalf E A, Cook A C. Tumor suppressor p53: analysis of wild-type and mutant p53 complexes. Mol Cell Biol. 1991 January; 11(1):12-9.
481. Srivastava S, Wang S, Tong Y A, Hao Z M, Chang E H. Dominant negative effect of a germ-line mutant p53: a step fostering tumorigenesis. Cancer Res. 1993 Oct. 1; 53(19):4452-5.
482. Kern S E, Pietenpol J A, Thiagalingam S, Seymour A, Kinzler K W, Vogelstein B. Oncogenic forms of p53 inhibit p53-regulated gene expression. Science. 1992 May 8; 256(5058):827-30.
483. Mateu M G, Sanchez Del Pino M M, Fersht A R. Mechanism of folding and assembly of a small tetrameric protein domain from tumor suppressor p53. Nat Struct Biol. 1999 February; 6(2):191-8.
484. Mateu M G, Fersht A R. Nine hydrophobic side chains are key determinants of the thermodynamic stability and oligomerization status of tumour suppressor p53 tetramerization domain. EMBO J. 1998 May 15; 17(10):2748-58.
485. Waterman M J, Waterman J L, Halazonetis T D. An engineered four-stranded coiled coil substitutes for the tetramerization domain of wild-type p53 and alleviates transdominant inhibition by tumor-derived p53 mutants. Cancer Res. 1996 Jan. 1; 56(1):158-63.
486. Wichmann C, Becker Y, Chen-Wichmann L, Vogel V, Vojtkova A, Herglotz J, et al. Dimer-tetramer transition controls RUNX1/ETO leukemogenic activity. Blood. 2010 Jul. 29; 116(4):603-13.
487. Jeffrey P D, Gonna S, Pavletich N P. Crystal structure of the tetramerization domain of the p53 tumor suppressor at 1.7 angstroms. Science. 1995 Mar. 10; 267(5203):1498-502.
488. Mossalam M, Matissek K J, Okal A, Constance J E, Lim C S. Direct induction of apoptosis using an optimal mitochondrially targeted p53. Mol Pharm. 2012 May 7; 9(5):1449-58.
489. Dixon A S, Kakar M, Schneider K M, Constance J E, Paullin B C, Lim C S. Controlling subcellular localization to alter function: Sending oncogenic Bcr-Abl to the nucleus causes apoptosis. J Control Release. 2009 Dec. 16; 140(3):245-9.
490. Nielsen L L, Lipari P, Dell J, Gurnani M, Hajian G. Adenovirus-mediated p53 gene therapy and paclitaxel have synergistic efficacy in models of human head and neck, ovarian, prostate, and breast cancer. Clin Cancer Res. 1998 April; 4(4):835-46.
491. Shaulsky G, Goldfinger N, Ben-Ze'ev A, Rotter V. Nuclear accumulation of p53 protein is mediated by several nuclear localization signals and plays a role in tumorigenesis. Mol Cell Biol. 1990 December; 10(12):6565-77.
492. Mooney L M, Al-Sakkaf K A, Brown B L, Dobson P R. Apoptotic mechanisms in T47D and MCF-7 human breast cancer cells. Br J Cancer. 2002 Oct. 7; 87(8):909-17.
493. Nigro J M, Baker S J, Preisinger A C, Jessup J M, Hostetter R, Cleary K, et al. Mutations in the p53 gene occur in diverse human tumour types. Nature. 1989 Dec. 7; 342(6250):705-8.
494. Loo D T, Rillema J R. Measurement of cell death. Methods Cell Biol. 1998; 57:251-64.
495. Koopman G, Reutelingsperger C P, Kuijten G A, Keehnen R M, Pals S T, van Oers M H. Annexin V for flow cytometric detection of phosphatidylserine expression on B cells undergoing apoptosis. Blood. 1994 Sep. 1; 84(5):1415-20.
496. Schmid I, Krall W J, Uittenbogaart C H, Braun J, Giorgi J V. Dead cell discrimination with 7-amino-actinomycin D in combination with dual color immunofluorescence in single laser flow cytometry. Cytometry. 1992; 13(2):204-8.
497. Lim L Y, Vidnovic N, Ellisen L W, Leong C O. Mutant p53 mediates survival of breast cancer cells. Br J Cancer. 2009 Nov. 3; 101(9):1606-12.
498. Bodner S M, Minna J D, Jensen S M, D'Amico D, Carbone D, Mitsudomi T, et al. Expression of mutant p53 proteins in lung cancer correlates with the class of p53 gene mutation. Oncogene. 1992 April; 7(4):743-9.
499. Di Como C J, Gaiddon C, Prives C. p73 function is inhibited by tumor-derived p53 mutants in mammalian cells. Mol Cell Biol. 1999 February; 19(2):1438-49.
500. Dong P, Tada M, Hamada J, Nakamura A, Moriuchi T, Sakuragi N. p53 dominant-negative mutant R273H promotes invasion and migration of human endometrial cancer HHUA cells. Clin Exp Metastasis. 2007; 24(6):471-83.
501. Lee W, Harvey T S, Yin Y, Yau P, Litchfield D, Arrowsmith C H. Solution structure of the tetrameric minimum transforming domain of p53. Nat Struct Biol. 1994 December; 1(12):877-90.
502. Chandar N, Billig B, McMaster J, Novak J. Inactivation of p53 gene in human and murine osteosarcoma cells. Br J Cancer. 1992 February; 65(2):208-14.
503. Toledo F, Wahl G M. Regulating the p53 pathway: in vitro hypotheses, in vivo veritas. Nat Rev Cancer. 2006 December; 6(12):909-23.
504. Alexis J D, Wang N, Che W, Lerner-Marmarosh N, Sahni A, Korshunov V A, et al. Bcr kinase activation by angiotensin II inhibits peroxisome-proliferator-activated receptor gamma transcriptional activity in vascular smooth muscle cells. Circ Res. 2009 Jan. 2; 104(1):69-78.
505. Maru Y, Witte O N. The BCR gene encodes a novel serine/threonine kinase activity within a single exon. Cell. 1991 Nov. 1; 67(3):459-68.
506. Nielsen L L, Dell J, Maxwell E, Armstrong L, Maneval D, Catino J J. Efficacy of p53 adenovirus-mediated gene therapy against human breast cancer xenografts. Cancer Gene Ther. 1997 March-April; 4(2):129-38.
507. Giono L E, Manfredi J J. Mdm2 is required for inhibition of Cdk2 activity by p21, thereby contributing to p53-dependent cell cycle arrest. Mol Cell Biol. 2007 June; 27(11):4166-78.
508. Dixon A S, Pendley S S, Bruno B J, Woessner D W, Shimpi A A, Cheatham T E, 3rd, et al. Disruption of Bcr-Abl coiled coil oligomerization by design. J Biol Chem. 2011 Aug. 5; 286(31):27751-60.
509. Galea C, Bowman P, Kriwacki R W. Disruption of an intermonomer salt bridge in the p53 tetramerization domain results in an increased propensity to form amyloid fibrils. Protein Sci. 2005 December; 14(12):2993-3003.
510. Budram-Mahadeo V, Morris P J, Latchman D S. The Brn-3a transcription factor inhibits the pro-apoptotic effect of p53 and enhances cell cycle arrest by differentially regulating the activity of the p53 target genes encoding Bax and p21(CIP1/Waf1). Oncogene. 2002 Sep. 5; 21(39):6123-31.
511. Aylon Y, Oren M. Living with p53, dying of p53. Cell. 2007 Aug. 24; 130(4):597-600.
512. Jackson J G, Pant V, Li Q, Chang L L, Quintas-Cardama A, Garza D, et al. p53-mediated senescence impairs the apoptotic response to chemotherapy and clinical outcome in breast cancer. Cancer Cell. 2012 Jun. 12; 21(6):793-806.
513. Palacios G, Crawford H C, Vaseva A, Moll U M. Mitochondrially targeted wild-type p53 induces apoptosis in a solid human tumor xenograft model. Cell Cycle. 2008 Aug. 15; 7(16):2584-90.
514. Vaseva A V, Moll U M. The mitochondrial p53 pathway. Biochim Biophys Acta. 2009 May; 1787(5):414-20.
515. Shaulsky G, Goldfinger N, Ben-Ze'ev A, Rotter V. Nuclear accumulation of p53 protein is mediated by several nuclear localization signals and plays a role in tumorigenesis. Mol Cell Biol. 1990 December; 10(12):6565-77.
516. Marchenko N D, Wolff S, Erster S, Becker K, Moll U M. Monoubiquitylation promotes mitochondrial p53 translocation. EMBO J. 2007 Feb. 21; 26(4):923-34.
517. Marchenko N D, Moll U M. The role of ubiquitination in the direct mitochondrial death program of p53. Cell Cycle. 2007 Jul. 15; 6(14):1718-23.
518. Costes S V, Daelemans D, Cho E H, Dobbin Z, Pavlakis G, Lockett S. Automatic and quantitative measurement of protein-protein colocalization in live cells. Biophys J. 2004 June; 86(6):3993-4003.
519. Constance J E, Woessner D W, Matissek K J, Mossalam M, Lim C S. Enhanced and selective killing of chronic myelogenous leukemia cells with an engineered BCR-ABL binding protein and imatinib. Mol Pharm. 2012 Nov. 5; 9(11):3318-29.
520. Constance J E, Despres S D, Nishida A, Lim C S. Selective targeting of c-Abl via a cryptic mitochondrial targeting signal activated by cellular redox status in leukemic and breast cancer cells. Pharm Res. 2012 August; 29(8):2317-28.
521. Rehling P, Brandner K, Pfanner N. Mitochondrial import and the twin-pore translocase. Nat Rev Mol Cell Biol. 2004 July; 5(7):519-30.
522. Schellenberg B, Wang P, Keeble J A, Rodriguez-Enriquez R, Walker S, Owens T W, et al. Bax exists in a dynamic equilibrium between the cytosol and mitochondria to control apoptotic priming. Mol Cell. 2013 Mar. 7; 49(5):959-71.
523. Edlich F, Banerjee S, Suzuki M, Cleland M M, Arnoult D, Wang C, et al. Bcl-x(L) retrotranslocates Bax from the mitochondria into the cytosol. Cell. 2011 Apr. 1; 145(1):104-16.
524. Willis S N, Chen L, Dewson G, Wei A, Naik E, Fletcher J I, et al. Proapoptotic Bak is sequestered by Mcl-1 and Bcl-xL, but not Bcl-2, until displaced by BH3-only proteins. Genes Dev. 2005 Jun. 1; 19(11):1294-305.
525. Wang X, Jiang X. Mdm2 and MdmX partner to regulate p53. FEBS Left. 2012 May 21; 586(10):1390-6.
526. Mossalam M, Matissek K J, Okal A, Constance J E, Lim C S. Direct induction of apoptosis using an optimal mitochondrially targeted p53. Mol Pharm. 2012 May 7; 9(5):1449-58.
527. Matissek K J, Mossalam M, Okal A, Lim C S. The DNA Binding Domain of p53 Is Sufficient To Trigger a Potent Apoptotic Response at the Mitochondria. Mol Pharm. 2013 Sep. 6.
528. Zhao X, Ghaffari S, Lodish H, Malashkevich V N, Kim P S. Structure of the Bcr-Abl oncoprotein oligomerization domain. Nat Struct Biol. 2002 February; 9(2):117-20.
529. Jeffrey P D, Gonna S, Pavletich N P. Crystal structure of the tetramerization domain of the p53 tumor suppressor at 1.7 angstroms. Science. 1995 Mar. 10; 267(5203):1498-502.
530. Waterman M J, Waterman J L, Halazonetis T D. An engineered four-stranded coiled coil substitutes for the tetramerization domain of wild-type p53 and alleviates transdominant inhibition by tumor-derived p53 mutants. Cancer Res. 1996 Jan. 1; 56(1):158-63.
531. Gully C P, Zhang F, Chen J, Yeung J A, Velazquez-Torres G, Wang E, et al. Antineoplastic effects of an Aurora B kinase inhibitor in breast cancer. Mol Cancer. 2010; 9:42.
532. Zhu W, Wei L, Zhang H, Chen J, Qin X. Oncolytic adenovirus armed with IL-24 inhibits the growth of breast cancer in vitro and in vivo. J Exp Clin Cancer Res. 2012; 31:51.
533. Jackson J G, Pant V, Li Q, Chang L L, Quintas-Cardama A, Garza D, et al. p53-mediated senescence impairs the apoptotic response to chemotherapy and clinical outcome in breast cancer. Cancer Cell. 2012 Jun. 12; 21(6):793-806.
534. Matissek K J, Mossalam M, Okal A, Lim C S. The DNA binding domain of p53 is sufficient to trigger a potent apoptotic response at the mitochondria. Mol Pharm. 2013 Oct. 7; 10(10):3592-602.
535. Ferrer P E, Frederick P, Gulbis J M, Dewson G, Kluck R M. Translocation of a Bak C-terminus mutant from cytosol to mitochondria to mediate cytochrome C release: implications for Bak and Bax apoptotic function. PLoS One. 2012; 7(3):e31510.
536. Chipuk J E, Kuwana T, Bouchier-Hayes L, Droin N M, Newmeyer D D, Schuler M, et al. Direct activation of Bax by p53 mediates mitochondrial membrane permeabilization and apoptosis. Science. 2004 Feb. 13; 303(5660):1010-4.
537. Perfettini J L, Kroemer R T, Kroemer G. Fatal liaisons of p53 with Bax and Bak. Nat Cell Biol. 2004 May; 6(5):386-8.
538. Leu J I, Dumont P, Hafey M, Murphy M E, George D L. Mitochondrial p53 activates Bak and causes disruption of a Bak-Mcl1 complex. Nat Cell Biol. 2004 May; 6(5):443-50.
539. Hagn F, Klein C, Demmer O, Marchenko N, Vaseva A, Moll U M, et al. BclxL changes conformation upon binding to wild-type but not mutant p53 DNA binding domain. J Biol Chem. 2010 Jan. 29; 285(5):3439-50.
540. Tomita Y, Marchenko N, Erster S, Nemajerova A, Dehner A, Klein C, et al. W T p53, but not tumor-derived mutants, bind to Bcl2 via the DNA binding domain and induce mitochondrial permeabilization. J Biol Chem. 2006 Mar. 31; 281(13):8600-6.
541. Minn A J, Rudin C M, Boise L H, Thompson C B. Expression of bcl-xL can confer a multidrug resistance phenotype. Blood. 1995 Sep. 1; 86(5):1903-10.
542. Perego P, Righetti S C, Supino R, Delia D, Caserini C, Carenini N, et al. Role of apoptosis and apoptosis-related proteins in the cisplatin-resistant phenotype of human tumor cell lines. Apoptosis. 1997; 2(6):540-8.
543. Reed J C. Bcl-2-family proteins and hematologic malignancies: history and future prospects. Blood. 2008 Apr. 1; 111(7):3322-30.
544. Yoshino T, Shiina H, Urakami S, Kikuno N, Yoneda T, Shigeno K, et al. Bcl-2 expression as a predictive marker of hormone-refractory prostate cancer treated with taxane-based chemotherapy. Clin Cancer Res. 2006 Oct. 15; 12(20 Pt 1):6116-24.
545. Kang M H, Reynolds C P. Bcl-2 inhibitors: targeting mitochondrial apoptotic pathways in cancer therapy. Clin Cancer Res. 2009 Feb. 15; 15(4):1126-32.
546. Green D R, Walczak H. Apoptosis therapy: driving cancers down the road to ruin. Nat Med. 2013 February; 19(2):131-3.
547. Bharatham N, Chi S W, Yoon H S. Molecular basis of Bcl-X(L)-p53 interaction: insights from molecular dynamics simulations. PLoS One. 2011; 6(10):e26014.
548. Okal A, Mossalam M, Matissek K J, Dixon A S, Moos P J, Lim C S. A chimeric p53 evades mutant p53 transdominant inhibition in cancer cells. Mol Pharm. 2013 Oct. 7; 10(10):3922-33.
549. Howlader N N A, Krapcho M, Garshell J, Neyman N, Altekruse S F, Kosary C L, Yu M, Ruhl J, Tatalovich Z, Cho H, Mariotto A, Lewis D R, Chen H S, Feuer E J, Cronin K A (eds). SEER Cancer Statistics Review, 1975-2010. National Cancer Institute Bethesda, Md. 2013.
550. Lozano R, Naghavi M, Foreman K, Lim S, Shibuya K, Aboyans V, et al. Global and regional mortality from 235 causes of death for 20 age groups in 1990 and 2010: a systematic analysis for the Global Burden of Disease Study 2010. Lancet. 2012 Dec. 15; 380(9859):2095-128.
551. Mould T. An overview of current diagnosis and treatment in ovarian cancer. Int J Gynecol Cancer. 2012 May; 22 Suppl 1:S2-4.
552. Integrated genomic analyses of ovarian carcinoma. Nature. 2011 Jun. 30; 474(7353):609-15.
553. Bodner S M, Minna J D, Jensen S M, D'Amico D, Carbone D, Mitsudomi T, et al. Expression of mutant p53 proteins in lung cancer correlates with the class of p53 gene mutation. Oncogene. 1992 April; 7(4):743-9.
554. Yaginuma Y, Westphal H. Abnormal structure and expression of the p53 gene in human ovarian carcinoma cell lines. Cancer Res. 1992 Aug. 1; 52(15):4196-9.
555. Astanehe A, Arenillas D, Wasserman W W, Leung P C, Dunn S E, Davies B R, et al. Mechanisms underlying p53 regulation of PIK3CA transcription in ovarian surface epithelium and in ovarian cancer. J Cell Sci. 2008 Mar. 1; 121(Pt 5):664-74.
556. Sablina A A, Chumakov P M, Kopnin B P. Tumor suppressor p53 and its homologue p73alpha affect cell migration. J Biol Chem. 2003 Jul. 25; 278(30):27362-71.
557. Li M, Brooks C L, Wu-Baer F, Chen D, Baer R, Gu W. Mono- versus polyubiquitination: differential control of p53 fate by Mdm2. Science. 2003 Dec. 12; 302(5652): 1972-5.
558. Bossi G, Mazzaro G, Porrello A, Crescenzi M, Soddu S, Sacchi A. Wild-type p53 gene transfer is not detrimental to normal cells in vivo: implications for tumor gene therapy. Oncogene. 2004 Jan. 15; 23(2):418-25.
559. Clayman G L, el-Naggar A K, Roth J A, Zhang W W, Goepfert H, Taylor D L, et al. In vivo molecular therapy with p53 adenovirus for microscopic residual head and neck squamous carcinoma. Cancer Res. 1995 Jan. 1; 55(1):1-6.
560. Wills K N, Maneval D C, Menzel P, Harris M P, Sutjipto S, Vaillancourt M T, et al. Development and characterization of recombinant adenoviruses encoding human p53 for gene therapy of cancer. Hum Gene Ther. 1994 September; 5(9):1079-88.
561. Zhang W W, Alemany R, Wang J, Koch P E, Ordonez N G, Roth J A. Safety evaluation of AdSCMV-p53 in vitro and in vivo. Hum Gene Ther. 1995 February; 6(2):155-64.
562. Bao R, Connolly D C, Murphy M, Green J, Weinstein J K, Pisarcik D A, et al. Activation of cancer-specific gene expression by the survivin promoter. J Natl Cancer Inst. 2002 Apr. 3; 94(7):522-8.
563. Tanyi J L, Lapushin R, Eder A, Auersperg N, Tabassam F H, Roth J A, et al. Identification of tissue- and cancer-selective promoters for the introduction of genes into human ovarian cancer cells. Gynecol Oncol. 2002 June; 85(3):451-8.
564. Bao R, Selvakumaran M, Hamilton T C. Targeted gene therapy of ovarian cancer using an ovarian-specific promoter. Gynecol Oncol. 2002 February; 84(2):228-34.
565. Cho S, Sun Y, Soisson A P, Dodson M K, Peterson C M, Jarboe E A, et al. Characterization and evaluation of pre-clinical suitability of a syngeneic orthotopic mouse ovarian cancer model. Anticancer Res. 2013 April; 33(4):1317-24.
566. Zeimet A G, Marth C. Why did p53 gene therapy fail in ovarian cancer? Lancet Oncol. 2003 July; 4(7):415-22.
567. Anwer K, Kelly F J, Chu C, Fewell J G, Lewis D, Alvarez R D. Phase I trial of a formulated IL-12 plasmid in combination with carboplatin and docetaxel chemotherapy in the treatment of platinum-sensitive recurrent ovarian cancer. Gynecol Oncol. 2013 October; 131(1):169-73.
568. Armstrong D K, Bundy B, Wenzel L, Huang H Q, Baergen R, Lele S, et al. Intraperitoneal cisplatin and paclitaxel in ovarian cancer. N Engl J Med. 2006 Jan. 5; 354(1):34-43.
569. Alberts D S, Liu P Y, Hannigan E V, O'Toole R, Williams S D, Young J A, et al. Intraperitoneal cisplatin plus intravenous cyclophosphamide versus intravenous cisplatin plus intravenous cyclophosphamide for stage III ovarian cancer. N Engl J Med. 1996 Dec. 26; 335(26):1950-5.
570. Markman M, Bundy B N, Alberts D S, Fowler J M, Clark-Pearson D L, Carson L F, et al. Phase III trial of standard-dose intravenous cisplatin plus paclitaxel versus moderately high-dose carboplatin followed by intravenous paclitaxel and intraperitoneal cisplatin in small-volume stage III ovarian carcinoma: an intergroup study of the Gynecologic Oncology Group, Southwestern Oncology Group, and Eastern Cooperative Oncology Group. J Clin Oncol. 2001 Feb. 15; 19(4):1001-7.
571. Fewell J G, Matar M M, Rice J S, Brunhoeber E, Slobodkin G, Pence C, et al. Treatment of disseminated ovarian cancer using nonviral interleukin-12 gene therapy delivered intraperitoneally. J Gene Med. 2009 August; 11(8):718-28.
572. Romero I, Bast R C, Jr. Minireview: human ovarian cancer: biology, current management, and paths to personalizing therapy. Endocrinology. 2012 April; 153(4): 1593-602.
573. Reles A, Wen W H, Schmider A, Gee C, Runnebaum I B, Kilian U, et al. Correlation of p53 mutations with resistance to platinum-based chemotherapy and shortened survival in ovarian cancer. Clin Cancer Res. 2001 October; 7(10):2984-97.
574. Matzke G R WBG PLM, editor., editor. Pharmacotherapy: A pathophysiological Approach. 7th edition ed: Mc Graw Hhill Medical; 2008.
575. Brambilla E, Brambilla C. p53 and lung cancer. Pathol Biol (Paris). 1997 December; 45(10):852-63.
576. Vaughan C A, Singh S, Windle B, Yeudall W A, Frum R, Grossman S R, et al. Gain-of-Function Activity of Mutant p53 in Lung Cancer through Up-Regulation of Receptor Protein Tyrosine Kinase Axl. Genes Cancer. 2012 July; 3(7-8):491-502.
577. Okamoto H, Todo, H., Iida, K., Danjo, K. Dry powders for pulmonary delivery of peptides and proteins. Kona. 2002 71-83.
578. Bosquillon C, Preat V, Vanbever R. Pulmonary delivery of growth hormone using dry powders and visualization of its local fate in rats. J Control Release. 2004 Apr. 28; 96(2):233-44.
579. Davis S S. Delivery of peptide and non-peptide drugs through the respiratory tract. Pharm Sci Technolo Today. 1999 November; 2(11):450-6.
580. FDA USFaDA. FDA Approves First Ever Inhaled Insulin Combination Product for Treatment of Diabetes. Jan. 27, 2006.
581. Heinemann L. The failure of exubera: are we beating a dead horse? J Diabetes Sci Technol. 2008 May; 2(3):518-29.

1. A peptide comprising a full length p53 peptide and a mitochondrial targeting signal (MTS), wherein the MTS is a Bak or Bax MTS.

2. The peptide of claim 1, wherein the Bak MTS comprises the amino acid sequence GNGPILNVLVVLGVVLLGQFVVRRFFKS (SEQ ID NO:15).

3. The peptide of claim 1, wherein the Bax MTS comprises the amino acid sequence GTPTWQTVTIFVAGVLTASLTIWKKMG (SEQ ID NO:14).

4. A peptide comprising a partial p53 peptide and a MTS.

5. The peptide of claim 4, wherein the MTS comprises a Bcl-XL, Bak, or Bax MTS.

6. The peptide of claim 5, wherein the MTS comprises the amino acid sequence RKGQERFNRWFLTGMTVAGVVLLGSLFSRK (SEQ ID NO:13), GNGPILNVLVVLGVVLLGQFVVRRFFKS (SEQ ID NO:15), or GTPTWQTVTIFVAGVLTASLTIWKKMG (SEQ ID NO:14).

7. The peptide of claim 4, wherein the partial p53 peptide consists of the DNA binding domain of p53.

8. (canceled)

9. The peptide of claim 4, wherein the partial p53 peptide comprises the DNA binding domain of p53.

10. The peptide of claim 9, wherein the DNA binding domain of p53 consists of amino acids 102-292 of SEQ ID NO:24.

11. The peptide of claim 9, wherein the partial p53 peptide further comprises a MDM2 binding domain, a proline-rich domain, a tetramerization domain, or a transactivation domain of p53.

12. A nucleic acid sequence, wherein the nucleic acid sequence is capable of encoding the peptide of claim 1 or claim 4.

13.-16. (canceled)

17. A vector comprising the nucleic acid sequence of claim 12.

18.-22. (canceled)

23. A method of inducing apoptosis comprising administering a peptide, wherein the peptide comprises a full length p53 peptide and a MTS, wherein the MTS is a Bak or Bax MTS or a partial p53 peptide and a MTS.

24.-32. (canceled)

33. The peptide of claim 4, nucleic acid of claim 13, or vector of claim 17, wherein the MTS is not a TOM, OTC, or CCO MTS.

34.-47. (canceled)

48. A cell comprising the nucleic acid of claim 12.

49-51. (canceled)
